(19) United States
(12) Patent Application Publication
(10) Pub. No.: US 2002/0194646 A1
(43) Pub. Date: Dec. 19, 2002

(54) Methods of creating dwarf phenotypes in plants

(76) Inventors: Gregory P. Pogue, Vacaville, CA (US); Guy R. Della-Cioppa, Vacaville, CA (US); Gershon M. Wolfe, Davis, CA (US); Wenjin Zheng, Davis, CA (US)

Correspondence Address: HOWREY SIMON ARNOLD & WHITE, LLP, BOX 34, 301 RAVENSWOOD AVE., MENLO PARK, CA, 94025, US

(21) Appl. No.: 09/910,664

(22) Filed: Jul. 20, 2001

Related U.S. Application Data
(60) Provisional application No. 60/219,943, filed on Jul. 20, 2000.

(51) Int. Cl. A01H 5/00

(52) U.S. Cl. 800/290; 800/320; 800/317.3; 800/319

(57) ABSTRACT

The invention is directed to the application of gene sequences which cause a dwarf phenotype in plants to the fields of forestry plants, ornamental horticultural plants, medicinal plants, and Nicotiana plants which are used for purposes other than for traditional tobacco products. The invention provides cDNAs identified by the polynucleotide sequences SEQ ID NO: 1-122 that may be used to create transfected or transgenic plants exhibiting a dwarf phenotype. The invention also provides methods of creating a transfected or transgenic plant exhibiting a dwarf phenotype by expressing in the plant DNA or mRNA identified by the sequences SEQ ID NO:1-122.

### CROSS-REFERENCE TO RELATED APPLICATION

This application claims the priority benefit of provisional U.S. Patent Application Serial No. 60/219,943, filed Jul. 20, 2000, which is hereby incorporated herein by reference in its entirety.

### FIELD OF THE INVENTION

This invention relates to nucleic acids and amino acid sequences identified in multiple metabolic pathways that lead to dwarfism and stunting in plants and the use of these sequences to create dwarf varieties of any plant species. Particularly, this invention relates to the use of nucleic acids and amino acid sequences which cause dwarfing in the fields of forestry plants, ornamental horticultural plants, medicinal plants, and Nicotiana plants.

### BACKGROUND OF THE INVENTION

The strategies for increasing the productivity of plants is dependent on rapid discovery of unknown gene sequences and their function through genomics research. These discoveries will provide fundamental information necessary to engineer plants for improved grain yields and resistance to drought, pests, salt, and other extreme environmental conditions. Such advances are critical for a world population expected to double by 2050. Moreover, this information may identify genes and products encoded by genes that are useful for human and animal healthcare such as pharmaceuticals.

There has been a massive accumulation of expressed sequence tags (ESTs) as a result of recent genome research. Potential use of this sequence information is enormous once gene function is determined. Knowledge of function allows engineering of commercial plants and seeds for forestry, ornamental and horticultural plants, including any plants used to produce pharmaceutical products, and particularly plants of the genus Nicotiana for purposes other than traditional tobacco products.

Use of these sequences to convey any number of desirable traits to pharmaceutical and fiber crops and thereby increase production and building materials, medicines and chemicals for other uses. For example, gene profiling in cottonwood may lead to an understanding of the types of genes and promoters that act primarily in fiber cells. The novel sequences derived from these profiling studies may be important in genetic engineering of cottonwood fiber for increased strength. In plant breeding, gene profiling coupled to physiological trait analysis can lead to the identification of predictive markers that will be increasingly important in marker assisted breeding programs. Mining the DNA sequence of a particular crop for genes important for yield, quality, health, appearance, color, taste, etc. are applications of obvious importance for crop improvement.

The Green Revolution crops, introduced in the late 1960s and early 1970s, produce several times as much grain as the traditional varieties they replaced, and they spread rapidly. They enabled India to double its wheat crop in seven years, dramatically increasing food supplies and averting widely predicted famine. The Green Revolution's leading research achievement was to hasten the perfection of dwarf spring wheat. Though it is conventionally assumed that farmers want a tall, impressive-looking harvest, in fact shrinking wheat and other crops has often proved beneficial. When bred for short stalks, plants expend less energy growing inedible column sections and more growing valuable grain. Stout, short-stalked wheat also neatly supports its kernels, whereas tall-stalked wheat may bend over at maturity, complicating reaping. Nature has favored genes for tall stalks, because in nature plants must compete for access to sunlight. However, in high-yield agriculture, equally short-stalked plants will receive equal sunlight. Researchers are actively seeking dwarf strains of rice and other crops in order to increase agronomic yields. The identification of genes and metabolic pathways that may be modified to create rapidly growing dwarf strains would greatly accelerate this effort. Furthermore, identification of these genes and metabolic pathways in food crops may lead to the development of dwarf strains in other plant types such as forest trees, ornamental species such as ornamental and turfgrass, and plants such as Nicotiana sp. grown as hosts for biopharmaceutical manufacturing.

### SUMMARY OF THE INVENTION

The invention is directed to the application of gene sequences which cause a dwarf phenotype in plants to the fields of forestry plants, ornamental horticultural plants, medicinal plants, and Nicotiana plants which are used for purposes other than for traditional tobacco products.

The invention provides cDNAs identified by the polynucleotide sequences SEQ ID NO: 1-122 that may be used to create transfected or transgenic plants exhibiting a dwarf phenotype. These cDNAs have been identified by phenotypic screening of the Large Scale Biology's libraries over 8000 cDNAs from Arabidopsis, Nicotiana, Oryza and Papaver constructed in the GENEWARE® vector.

The invention provides methods of creating a transfected or transgenic plant exhibiting a dwarf phenotype comprising: expressing in the plant a cDNA (or its encoded mRNA) identified by a polynucleotide sequence chosen from the group consisting of SEQ ID NO: 1-122.

The invention also provides a method of creating a transfected or transgenic plant exhibiting a dwarf phenotype comprising the steps of: (a) providing a viral inoculum capable of infecting a plant comprising the cDNA (or its encoded mRNA) identified by a polynucleotide sequence chosen from the group of SEQ ID NO: 1-122; and (b) applying said viral inoculum to a plant; whereby the plant is infected and the cDNA (or its encoded mRNA) is expressed in the plant.

The methods of the invention provide for creating a transfected or transgenic plant exhibiting a dwarf phenotype in any plant type. Preferred embodiments of the invention provide methods for creating dwarf plants of ornamental and horticultural plants, medicinal plants or forest trees. A preferred embodiment provides methods for creating dwarf plants of Nicotiana sp. Another preferred embodiment provides methods for creating dwarf turfgrass.

The invention also provides methods for creating transfected or transgenic plants exhibiting a dwarf phenotype for use in biopharmaceutical manufacturing comprising: applying a viral inoculum capable of infecting a plant and comprising the DNA (or its encoded mRNA) identified by a polynucleotide sequence chosen from the group of SEQ. ID NO 1-122 to a plant that expresses a biopharmaceutical, whereby the plant is infected, exhibits a dwarf phenotype, and expresses the biopharmaceutical.

The invention also provides a transfected or transgenic plant exhibiting a dwarf phenotype made by the method comprising expressing in the plant a cDNA(or its encoded mRNA) identified by a polynucleotide sequence chosen from the group consisting of SEQ ID NO: 1-122. The invention provides for transfected or transgenic plants made by the use of this method with any plant type. Preferred embodiments are transfected or transgenic plants of ornamental and horticultural plants, medicinal plants or forest trees. Preferred embodiments include transfected or transgenic plants of Nicotiana sp and dwarf turfgrass.

The invention also provides methods of producing multiple crops of the transfected or transgenic plants expressing a cDNA(or its encoded mRNA) identified by a polynucleotide sequence chosen from the group consisting of SEQ ID NO: 1-122 and exhibiting a dwarf phenotype comprising the steps of: (a) planting a reproductive unit of the transfected or transgenic plant; (b) growing the planted reproductive unit under natural light conditions; (c) harvesting the plant; and (d) repeating steps (a) through (c) at least once in the year.

The invention provides a method of constructing and characterizing a normalized cDNA library in a viral vector. The invention further provides a method of constructing and characterizing of a normalized whole plant cDNA library in viral vectors.

The invention identifies cDNAs corresponding to genes in the trans-ketolase and carbohydrate metabolic pathways as useful for creating transfected or transgenic plants exhibiting a dwarf phenotype.

The invention also provides method of manufacturing a biopharmaceutical comprising:

### DESCRIPTION OF THE INVENTION

Before the present proteins, nucleotide sequences, and methods are described, it should be noted that this invention is not limited to the particular methodology, protocols, plants, cell lines, vectors, and reagents described herein as these may vary. It should also be understood that the terminology used herein is for the purpose of describing particular aspects of the invention, and is not intended to limit its scope which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to the “antibody” is a reference to one or more antibodies and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention.

### Definitions

“Acylate” as used herein, refers to the introduction of an acyl group into into a molecule, i.e. acylation

“Adjacent” as used herein, refers to a position in a nucleotide sequence proximate to and 5′ or 3′ to a defined sequence. Generally, adjacent means within 2 or 3 nucleotides of the site of reference.

“Agonist”, as used herein, refers to a molecule which, when bound to a gene product of interest, increases the biological or immunological activity of that gene product. Agonists may include proteins, nucleic acids, carbohydrates, or any other molecules which bind to a gene product of interest.

“Alterations” in a polynucleotide sequence, as used herein, comprise any deletions, insertions, and point mutations in the polynucleotide sequence. Included within this definition are alterations to any genomic DNA sequence corresponding to the polynucleotide sequence.

“Amino acid sequence” as used herein refers to an oligopeptide, peptide, polypeptide, or protein sequence, and fragments or portions thereof, and to naturally occurring or synthetic molecules. “Amino acid sequence” and like terms, such as “polypeptide” or “protein” as recited herein are not meant to limit the amino acid sequence to the complete, native amino acid sequence associated with the recited protein molecule.

“Amplification” as used herein refers to the production of additional copies of a nucleic acid sequence and is generally carried out using polymerase chain reaction (PCR) technologies well known in the art (Dieffenbach, C. W. and G. S. Dveksler (1995) PCR Primer, a Laboratory Manual, Cold Spring Harbor Press, Plainview, N.Y.).

“Antibody” refers to intact molecules as well as fragments thereof which are capable of specific binding to the epitopic determinant. Antibodies that bind a polypeptide of interest can be prepared using intact polypeptides or fragments as the immunizing antigen. These antigens may be conjugated to a carrier protein, if desired.

“Antigenic determinant,” “determinant group,” or “epitope of an antigenic macromolecule” as used herein, refers to any region of the macromolecule with the ability or potential to elicit, and combine with, specific antibody. Determinants exposed on the surface of the macromolecule are likely to be immunodominant, i.e. more immunogenic than other (imunorecessive) determinants which are less exposed, while some (e.g. those within the molecule) are non-immunogenic (immunosilent). As used herein, antigenic determinant refers to that portion of a molecule that makes contact with a particular antibody (i.e., an epitope). When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (i.e., the immunogen used to elicit the immune response) for binding to an antibody.

“Antisense”, as used herein, refers to nucleotide sequences which are complementary to a specific DNA or RNA sequence. The term “antisense” or “(−) sense” is used in reference to the nucleic acid strand that is complementary to the “sense” or “(+) sense” strand. The designation “negative” is sometimes used in reference to the antisense strand, and “positive” is sometimes used in reference to the sense strand. Antisense molecules may be produced by any method, including synthesis by ligating the gene of interest in a reverse orientation to a viral promoter which permits the synthesis of a complementary strand. Once introduced into a cell, the transcript of this strand may hybridize to natural sequences to block either their further transcription or translation. In this manner, mutant phenotypes may be generated.

“Anti-Sense Inhibition” as used herein, refers to a type of gene regulation based on cytoplasmic, nuclear or organelle inhibition of gene expression due to the presence in a cell of an RNA molecule complementary to at least a portion of the mRNA being translated. It is specifically contemplated that DNA molecules may be from either an RNA virus or mRNA from the host cells genome or from a DNA virus.

“Antagonist” or “inhibitor”, as used herein, refer to a molecule which, when bound to a gene product of interest, decreases the biological or immunological activity of that gene product of interest. Antagonists and inhibitors may include proteins, nucleic acids, carbohydrates, or any other molecules which bind to the gene product of interest.

“Biologically active”, as used herein, refers to a molecule having the structural, regulatory, or biochemical functions of a naturally occurring molecule.

“Cell Culture” as used herein, refers to a proliferating mass of cells which may be in either an undifferentiated or differentiated state, growing contiguously or non-contiguously.

“Chimeric plasmid” as used herein, refers to any recombinant plasmid formed (by cloning techniques) from nucleic acids derived from organisms which do not normally exchange genetic information (e.g. Escherichia coli and Saccharomyces cerevisiae).

“Chimeric Sequence” or “Chimeric Gene” as used herein, refers to a nucleotide sequence derived from at least two heterologous parts. The sequence may comprise DNA or RNA.

“Coding Sequence” as used herein, refers to a nucleic acid sequence which, when transcribed and translated, results in the formation of a cellular polypeptide or a ribonucleotide sequence which, when translated, results in the formation of a cellular polypeptide.

“Common Embryological Basis” as used herein, is intended to include all tissues which are derived from the same germinal layer, specifically the ectoderm layer, which forms during the gastrulation stage of embryogenesis. Such tissues include, but are not limited to, brain, epithelium, adrenal medulla, spinal chord, retina, ganglia and the like.

“Compatible” as used herein, refers to the capability of operating with other components of a system. A vector or plant viral nucleic acid which is compatible with a host is one which is capable of replicating in that host. A coat protein which is compatible with a viral nucleotide sequence is one capable of encapsidating that viral sequence.

“Complementary” or “Complementarity”, as used herein, refer to the Watson-Crick base-pairing of two nucleic acid sequences. For example, for the sequence 5′-AGT-3′ binds to the complementary sequence 3′-TCA-5′. Complementarity between two nucleic acid sequences may be “partial”, in which only some of the bases bind to their complement, or it may be complete as when every base in the sequence binds to it complementary base. The degree of complementarity between nucleic acid strands has significant effects on the efficiency and strength of hybridization between nucleic acid strands.

“Complementation analysis” as used herein, refers to observing the changes produced in an organism when a nucleic acid sequence is introduced into that organism after a selected gene has been deleted or mutated so that it no longer functions fully in its normal role. A complementary gene to the deleted or mutated gene can restore the genetic phenotype of the selected gene.

“Constitutive expression” as used herein refers to gene expression which features substantially constant or regularly cyclical gene transcription. Generally, genes which are constitutively expressed are substantially free of induction from an external stimulus.

“Correlates with expression of a polynucleotide”, as used herein, indicates that the detection of the presence of ribonucleic acid that is similar to and indicative of the presence of an mRNA encoding a polypeptide in a sample and thereby correlates with expression of the transcript from the polynucleotide encoding the protein.

“Deletion”, as used herein, refers to a change made in either an amino acid or nucleotide sequence resulting in the absence one or more amino acids or nucleotides, respectively.

“Differentiated cell” as used herein refers to a cell which has substantially matured to perform one or more biochemical or physiological functions.

“Dwarf Plant” as used herein, refers to a plant that is much below the height or size of its kind or related species.

“Encapsidation” as used herein, refers to the process during virion assembly in which nucleic acid becomes incorporated in the viral capsid or in a head/capsid precursor (e.g. in certain bacteriophages).

“Exon” as used herein, refers to a polynucleotide sequence in a nucleic acid that codes information for protein synthesis and that is copied and spliced together with other such sequences to form messenger RNA.

“Expression” as used herein is meant to incorporate one or more of transcription, reverse transcription and translation.

“Expressed sequence tag (EST)” as used herein refers to relatively short single-pass DNA sequences obtained from one or more ends of cDNA clones and RNA derived therefrom. They may be present in either the 5′ or the 3′ orientation. ESTs have been shown useful for identifying particular genes.

“Foreign gene” as used herein, refers to any sequence that is not native to the virus.

“Fusion protein” as used herein, refers to a protein containing amino acid sequences from each of two distinct proteins; it is formed by the expression of a recombinant gene in which two coding sequences have been joined together such that their reading frames are in phase. Hybrid genes of this type may be constructed in vitro in order to label the product of a particular gene with a protein which can be more readily assayed (e.g. a gene fused with lacZ in E. coli to obtain a fusion protein with β-galactosidase activity). Alternatively, a protein may be linked to a signal peptide to allow its secretion by the cell. The products of certain viral oncogenes are fusion proteins.

“Gene” as used herein, refers to a discrete nucleic acid sequence responsible for a discrete cellular product and/or performing one or more intercellular or intracellular functions. The term “gene”, as used herein, refers not only to the nucleotide sequence encoding a specific protein, but also to any adjacent 5′ and 3′ non-coding nucleotide sequence involved in the regulation of expression of the protein encoded by the gene of interest. These non-coding sequences include terminator sequences, promoter sequences, upstream activator sequences, regulatory protein binding sequences, and the like. These non-coding sequence gene regions may be readily identified by comparison with previously identified eukaryotic non-coding sequence gene regions. Furthermore, the person of average skill in the art of molecular biology is able to identify the nucleotide sequences forming the non-coding regions of a gene using well-known techniques such as a site-directed mutagenesis, sequential deletion, promoter probe vectors, and the like.

“Growth cycle” as used herein is meant to include the replication of a nucleus, an organelle, a cell, or an organism.

“Half-life” as used herein, refers to the time required for half of something to undergo a process (e.g. the time required for half the amount of a substance, such as a drug or radioactive tracer, in or introduced into a living system or ecosystem to be eliminated or disintegrated by natural processes.

“Heterologous” as used herein, refers to the association of a molecular or genetic element associated with a distinctly different type of molecular or genetic element.

“Host” as used herein, refers to a cell, tissue or organism capable of replicating a vector or plant viral nucleic acid and which is capable of being infected by a virus containing the viral vector or plant viral nucleic acid. This term is intended to include procaryotic and eukaryotic cells, organs, tissues or organisms, where appropriate.

“Homology” as used herein, refers to the degree of similarity between two or more nucleotide or amino-acid sequences. Homology may be partial or complete.

“Hybridization”, as used herein, refers to any process by which a strand of nucleic acid binds with a complementary or partially complementary strand through base pairing.

“Hybridization complex”, as used herein, refers to a complex formed between nucleic acid strands by virtue of hydrogen bonding, stacking or other non-covalent interactions between bases. A hybridization complex may be formed in solution or between nucleic acid sequences present in solution and nucleic acid sequences immobilized on a solid support (e.g., membranes, filters, chips, pins or glass slides to which cells have been fixed for in situ hybridization).

“Immunologically active” refers to the capability of a natural, recombinant, or synthetic gene product of interest, or any oligopeptide thereof, to bind with specific antibodies and induce a specific immune response in appropriate animals or cells.

“Induction” and the terms “induce”, “induction” and “inducible” as used herein, refer generally to a gene and a promoter operably linked thereto which is in some manner dependent upon an external stimulus, such as a molecule, in order to actively transcribed and/or translate the gene.

“Infection” as used herein refers to the ability of a virus to transfer its nucleic acid to a host or introduce a viral nucleic acid into a host, wherein the viral nucleic acid is replicated, viral proteins are synthesized, and new viral particles assembled. In this context, the terms “transmissible” and “infective” are used interchangeably herein. The term is also meant to include the ability of a selected nucleic acid sequence to integrate into a genome, chromosome or gene of a target organism.

“Insertion” or “Addition”, as used herein, refers to the replacement or addition of one or more nucleotides or amino acids, to a nucleotide or amino acid sequence, respectively.

“In cis” as used herein, indicates that two sequences are positioned on the same strand of RNA or DNA.

“In trans” as used herein, indicates that two sequences are positioned on different strands of RNA or DNA.

“Intron” as used herein refers to a polynucleotide sequence in a nucleic acid that does not code information for protein synthesis and is removed before translation of messenger RNA.

“Isolated” as used herein refers to a polypeptide, polynucleotide molecules separated not only from other peptides, DNAs, or RNAs, respectively, that are present in the natural source of the macromolecule but also from other macromolecules and preferably refers to a macromolecule found in the presence of (if anything) only a solvent, buffer, ion or other component normally present in a solution of the same. “Isolated” and “purified” do not encompass either natural materials in their native state or natural materials that have been separated into components (e.g., in an acrylamide gel) but not obtained either as pure substances or as solutions.

“Kinase” as used herein, refers to an enzyme (e.g. hexokinase and pyruvate kinase) which catalyzes the transfer of a phosphate group from one substrate (commonly ATP) to another.

“Marker” or “Genetic Marker” as used herein, refers to a genetic locus which is associated with a particular, usually readily detectable, genotype or phenotypic characteristic (e.g., an antibiotic resistance gene).

“Metabolome” as used herein, indicates the complement of relatively low molecular weight molecules that is present in a plant, plant part, or plant sample, or in a suspension or extract thereof. Examples of such molecules include, but are not limited to: acids and related compounds; mono-, di-,and tri-carboxylic acids (saturated, unsaturated, aliphatic and cyclic, aryl, alkaryl); aldo-acids, keto-acids; lactone forms; gibberellins; abscisic acid; alcohols, polyols, derivatives, and related compounds; ethyl alcohol, benzyl alcohol, menthanol; propylene glycol, glycerol, phytol; inositol, furfuryl alcohol, menthol; aldehydes, ketones, quinones, derivatives, and related compounds; acetaldehyde, butyraldehyde, benzaldehyde, acrolein, furfural, glyoxal; acetone, butanone; anthraquinone; carbohydrates; mono-, di-, tri-saccharides; alkaloids, amines, and other bases; pyridines (including nicotinic acid, nicotinamide); pyrimidines (including cytidine, thymine); purines (including guanine, adenine, xanthines/hypoxanthines, kinetin); pyrroles; quinolines (including isoquinolines); morphinans, tropanes, cinchonans; nucleotides, oligonucleotides, derivatives, and related compounds; guanosine, cytosine, adenosine, thymidine, inosine; amino acids, oligopeptides, derivatives, and related compounds; esters; phenols and related compounds; heterocyclic compounds and derivatives; pyrroles, tetrapyrroles (corrinoids and porphines/porphyrins, w/w/o metal-ion); flavonoids; indoles; lipids (including fatty acids and triglycerides), derivatives, and related compounds; carotenoids, phytoene; and sterols, isoprenoids including terpenes.

“Modulate” as used herein, refers to a change or an alteration in the biological activity of a gene product of interest. Modulation may be an increase or a decrease in protein activity, a change in binding characteristics, or any other change in the biological, functional or immunological properties of the gene product of interest.

“Movement protein” as used herein refers to a noncapsid protein required for cell to cell movement of replicons or viruses in plants.

“Multigene family” as used herein refers to a set of genes descended by duplication and variation from some ancestral gene. Such genes may be clustered together on the same chromosome or dispersed on different chromosomes. Examples of multigene families include those which encode the histones, hemoglobins, immunoglobulins, histocompatibility antigens, actins, tubulins, keratins, collagens, heat shock proteins, salivary glue proteins, chorion proteins, cuticle proteins, yolk proteins, and phaseolins.

“Non-Native” as used herein refers to any RNA or DNA sequence that does not normally occur in the cell or organism in which it is placed. Examples include recombinant plant viral nucleic acids and genes or ESTs contained therein. That is, a RNA or DNA sequence may be non-native with respect to a viral nucleic acid. Such a RNA or DNA sequence would not naturally occur in the viral nucleic acid. Also, a RNA or DNA sequence may be non-native with repect to a host organism. That is, such a RNA or DNA sequence would not naturally occur in the host organism. Conversely, the term non-native does not imply that a RNA or DNA sequence must be non-native with respect to both a viral nucleic acid and a host organism concurrently. The present invention specifically contemplates placing a RNA or DNA sequence which is native to a host organism into a viral nucleic acid in which it is non-native.

“Nucleic acid sequence” as used herein refers to a polymer of nucleotides in which the 3′ position of one nucleotide sugar is linked to the 5′ position of the next by a phosphodiester bridge. In a linear nucleic acid strand, one end typically has a free 5′ phosphate group, the other a free 3′ hydroxyl group. Nucleic acid sequences may be used herein to refer to oligonucleotides, or polynucleotides, and fragments or portions thereof, and to DNA or RNA of genomic or synthetic origin which may be single- or double-stranded, and represent the sense or antisense strand. The term is intended to encompass all nucleic acids whether naturally occurring in a particular cell or organism or non-naturally occurring in a particular cell or organism.

“Operably Linked” refers to a juxtaposition of components, particularly nucleotide sequences, such that the normal function of the components can be performed. Thus, a coding sequence that is operably linked to regulatory sequences refers to a configuration of nucleotide sequences wherein the coding sequences can be expressed under the regulatory control i.e., transcriptional and/or translational control, of the regulatory sequences.

“Organism” and “host organism” as used herein is specifically intended to include animals (including humans), plants, viruses, fungi, and bacteria.

“Origin of Assembly” as used herein, refers to a sequence where self-assembly of the viral RNA and the viral capsid protein initiates to form virions.

“Outlier Peak” as used herein, indicates a peak of a chromatogram of a test sample, or the relative or absolute detected response data, or amount or concentration data thereof. An outlier peak: 1) may have a significantly different peak height or area as compared to a like chromatogram of a control sample; or 2) be an additional or missing peak as compared to a like chromatogram of a control sample.

“Phenotype” or “Phenotypic Trait(s)” as used herein, refers to an observable property or set of properties resulting from the expression or suppression of a gene or genes.

“Plant” as used herein refers to any plant and progeny thereof, and to parts of plants including parts of plants, including seed, cuttings, tubers, fruit, flowers, branches, leaves, plant cells and other parts of any tree or other plant used in forestry, ornamental horticultural plants, medicinal plants including any plants used to produce pharmaceutical products, and plants of the genus Nicotiana which are used for purposes other than for traditional tobacco products.

“Plant Cell” as used herein, refers to the structural and physiological unit of plants, consisting of a protoplast and the cell wall.

“Plant Organ” as used herein, refers to a distinct and visibly differentiated part of a plant, such as root, stem, leaf or embryo.

“Plant Tissue” as used herein, refers to any tissue of a plant in planta or in culture. This term is intended to include a whole plant, plant cell, plant organ, protoplast, cell culture, or any group of plant cells organized into a structural and functional unit.

“Portion” as used herein, with regard to a protein (i.e. “a portion of a given protein”) refers to fragments of that protein. The fragments may range in size from four amino acid residues to the entire amino acid sequence minus one amino acid.

“Positive-sense inhibition” as used herein refers to a type of gene regulation based on cytoplasmic inhibition of gene expression due to the presence in a cell of an RNA molecule substantially homologous to at least a portion of the mRNA being translated.

“Production Cell” as used herein, refers to a cell, tissue or organism capable of replicating a vector or a viral vector, but which is not necessarily a host to the virus. This term is intended to include prokaryotic and eukaryotic cells, organs, tissues or organisms, such as bacteria, yeast, fungus and plant tissue.

“Promoter” as used herein, refers to the 5′-flanking, non-coding sequence substantially adjacent a coding sequence which is involved in the initiation of transcription of the coding sequence.

“Protoplast” as used herein, refers to an isolated plant cell without cell walls, having the potency for regeneration into cell culture or a whole plant.

“Purified” as used herein when referring to a peptide or nucleotide sequence, indicates that the molecule is present in the substantial absence of other biological macromolecular, e.g., polypeptides, polynucleic acids, and the like of the same type. The term “purified” as used herein preferably means at least 95% by weight, more preferably at least 99.8% by weight, of biological macromolecules of the same type present (but water, buffers, and other small molecules, especially molecules having a molecular weight of less than 1000 can be present). The term “pure” as used herein preferably has the same numerical limits as “purified” immediately above.

“Substantially purified” as used herein, refers to nucleic or amino acid sequences that are removed from their natural environment, isolated or separated, and are at least 60% free, preferably 75% free, and most preferably 90% free from other components with which they are naturally associated.

“Recombinant Plant Viral Nucleic Acid” as used herein, refers to a plant viral nucleic acid which has been modified to contain non-native nucleic acid sequences. These non-native nucleic acid sequences may be from any organism or purely synthetic, however, they may also include nucleic acid sequences naturally occurring in the organism into which the recombinant plant viral nucleic acid is to be introduced.

“Recombinant Plant Virus” as used herein, refers to a plant virus containing a recombinant plant viral nucleic acid.

“Regulatory region” or “Regulatory sequence” as used herein in reference to a specific gene refers to the non-coding nucleotide sequences within that gene that are necessary or sufficient to provide for the regulated expression of the coding region of a gene. Thus the term regulatory region includes promoter sequences, regulatory protein binding sites, upstream activator sequences, and the like. Specific nucleotides within a regulatory region may serve multiple functions. For example, a specific nucleotide may be part of a promoter and participate in the binding of a transcriptional activator protein.

“Replication origin” as used herein, refers to the minimal terminal sequences in linear viruses that are necessary for viral replication.

“Replicon” as used herein, refers to an arrangement of RNA sequences generated by transcription of a transgene that is integrated into the host DNA that is capable of replication in the presence of a helper virus. A replicon may require sequences in addition to the replication origins for efficient replication and stability.

“Sample”, as used herein, is used in its broadest sense. A biological sample suspected of containing a nucleic acid or fragments thereof may comprise a tissue, a cell, an extract from cells, chromosomes isolated from a cell (e.g., a spread of metaphase chromosomes), genomic DNA (in solution or bound to a solid support such as for Southern analysis), RNA (in solution or bound to a solid support such as for northern analysis), cDNA (in solution or bound to a solid support), and the like.

“Silent mutation” as used herein, refers to a mutation which has no apparent effect on the phenotype of the organism.

“Site-directed mutagenesis” as used herein, refers to the in-vitro induction of mutagenesis at a specific site in a given target nucleic acid molecule.

“Specific binding” or “specifically binding”, as used herein, in reference to the interaction of an antibody and a protein or peptide, mean that the interaction is dependent upon the presence of a particular structure (i.e., the antigenic determinant or epitope) on the protein; in other words, the antibody is recognizing and binding to a specific protein structure rather than to proteins in general.

“Stringent conditions”, as used herein, is the “stringency” which occurs within a range from about (Tₘ−5)° C. (i.e. 5 degrees below the melting temperature, Tₘ, of the probe) to about 20° to 25° C. below Tₘ. As will be understood by those of skill in the art, the stringency of hybridization may be altered in order to identify or detect identical or related polynucleotide sequences. Also as known in the art, numerous equivalent conditions may be employed to comprise either low or high stringency conditions. Factors such as the length and nature (DNA, RNA, base composition) of the sequence, nature of the target (DNA, RNA, base composition, presence in solution or immobilization, etc.), and the concentration of the salts and other components (e.g., the presence or absence of formamide, dextran sulfate and/or polyethylene glycol) are considered and the hybridization solution may be varied to generate conditions of either low or high stringency different from, but equivalent to, the above listed conditions.

“Subgenomic Promoter” as used herein, refers to a promoter of a subgenomic mRNA of a viral nucleic acid.

“Substantial Sequence Homology” as used herein, denotes nucleotide sequences that are substantially functionally equivalent to one another. Nucleotide differences between such sequences having substantial sequence homology will be de minimus in affecting function of the gene products or an RNA coded for by such sequence.

“Substitution”, as used herein, refers to a change made in an amino acid of nucleotide sequence which results in the replacement of one or more amino acids or nucleotides by different amino acids or nucleotides, respectively.

“Systemic Infection” as used herein denotes infection throughout a substantial part of an organism including mechanisms of spread other than mere direct cell inoculation but rather including transport from one infected cell to additional cells either nearby or distant.

“Transcription” as used herein, refers to the production of an RNA molecule by RNA polymerase as a complementary copy of a DNA sequence.

“Transcription termination region” as used herein, refers to the sequence that controls formation of the 3′ end of the transcript. Self-cleaving ribozymes and polyadenylation sequences are examples of transcription termination sequences.

“Transformation” as used herein, describes a process by which exogenous DNA enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

“Transposon” as used herein refers to a nucleotide sequence such as a DNA or RNA sequence which is capable of transferring location or moving within a gene, a chromosome or a genome.

“Transgenic plant” as used herein refers to a plant which contains a foreign nucleotide sequence inserted into either its nuclear genome or organellar genome.

“Transcription” as used herein refers to the production of an RNA molecule by RNA polymerase as a complementary copy of a DNA sequence or subgenomic mRNA.

“Variants” of a gene product of interest, as used herein, refers to a sequence resulting when the gene product is altered by one or more amino acids. The variant may have “conservative” changes, wherein a substituted amino acid has similar structural or chemical properties, e.g., replacement of leucine with isoleucine. More rarely, a variant may have “nonconservative” changes, e.g., replacement of a glycine with a tryptophan. Variants may also include sequences with amino acid deletions or insertions, or both. Guidance in determining which amino acid residues may be substituted, inserted, or deleted without abolishing biological or immunological activity may be found using computer programs well known in the art.

“Vector” as used herein, refers to a self-replicating DNA or RNA molecule which transfers a DNA or RNA segment between cells.

“Virion” as used herein, refers to a particle composed of viral RNA and viral capsid protein.

“Virus” as used herein, refers to an infectious agent composed of a nucleic acid encapsidated in a protein. A virus may be a mono-, di-, tri- or multi-partite virus.

### THE INVENTION

Identification and Analysis of cDNAs

The invention is based on the discovery of 122 cDNAs, identified by the polynucleotide sequences SEQ ID NO: 1-122, that may be used to create transfected or transgenic plants exhibiting a dwarf phenotype. Table 1 lists the source organism for all 122 cDNAs of the invention (as identified by its SEQ ID NO).

TABLE 1

| | | Sense or |
|---|---|---|
| SEQ ID | | Antisense |
| NO. | Source | Configuration |
| 1 | Nicotiana benthamiana | A |
| 2 | Nicotiana benthamiana | A |
| 3 | Arabidopsis thaliana | S |
| 4 | Arabidopsis thaliana | S |
| 5 | Arabidopsis thaliana | S |
| 6 | Arabidopsis thaliana | S |
| 7 | Arabidopsis thaliana | S |
| 8 | Arabidopsis thaliana | A |
| 9 | Arabidopsis thaliana | A |
| 10 | Arabidopsis thaliana | A |
| 11 | Arabidopsis thaliana | A |
| 12 | Arabidopsis thaliana | A |
| 13 | Arabidopsis thaliana | A |
| 14 | Arabidopsis thaliana | A |
| 15 | Arabidopsis thaliana | A |
| 16 | Arabidopsis thaliana | A |
| 17 | Arabidopsis thaliana | A |
| 18 | Arabidopsis thaliana | A |
| 19 | Arabidopsis thaliana | A |
| 20 | Arabidopsis thaliana | A |
| 21 | Arabidopsis thaliana | A |
| 22 | Arabidopsis thaliana | A |
| 23 | Arabidopsis thaliana | A |
| 24 | Arabidopsis thaliana | A |
| 25 | Arabidopsis thaliana | A |
| 26 | Arabidopsis thaliana | A |
| 27 | Arabidopsis thaliana | A |
| 28 | Arabidopsis thaliana | A |
| 29 | Arabidopsis thaliana | A |
| 30 | Arabidopsis thaliana | A |
| 31 | Arabidopsis thaliana | A |
| 32 | Arabidopsis thaliana | A |
| 33 | Arabidopsis thaliana | A |
| 34 | Arabidopsis thaliana | A |
| 35 | Arabidopsis thaliana | A |
| 36 | Arabidopsis thaliana | A |
| 37 | Arabidopsis thaliana | A |
| 38 | Arabidopsis thaliana | A |
| 39 | Arabidopsis thaliana | A |
| 40 | Arabidopsis thaliana | A |
| 41 | Arabidopsis thaliana | A |
| 42 | Arabidopsis thaliana | A |
| 43 | Arabidopsis thaliana | A |
| 44 | Arabidopsis thaliana | A |
| 45 | Arabidopsis thaliana | A |
| 46 | Arabidopsis thaliana | A |
| 47 | Arabidopsis thaliana | A |
| 48 | Arabidopsis thaliana | A |
| 49 | Arabidopsis thaliana | A |
| 50 | Arabidopsis thaliana | A |
| 51 | Arabidopsis thaliana | A |
| 52 | Arabidopsis thaliana | A |
| 53 | Arabidopsis thaliana | A |
| 54 | Arabidopsis thaliana | A |
| 55 | Arabidopsis thaliana | A |
| 56 | Arabidopsis thaliana | A |
| 57 | Arabidopsis thaliana | A |
| 58 | Arabidopsis thaliana | A |
| 59 | Arabidopsis thaliana | A |
| 60 | Arabidopsis thaliana | A |
| 61 | Arabidopsis thaliana | A |
| 62 | Arabidopsis thaliana | A |
| 63 | Arabidopsis thaliana | A |
| 64 | Arabidopsis thaliana | A |
| 65 | Arabidopsis thaliana | A |
| 66 | Arabidopsis thaliana | A |
| 67 | Arabidopsis thaliana | A |
| 68 | Arabidopsis thaliana | A |
| 69 | Arabidopsis thaliana | A |
| 70 | Arabidopsis thaliana | A |
| 71 | Arabidopsis thaliana | A |
| 72 | Arabidopsis thaliana | A |
| 73 | Arabidopsis thaliana | A |
| 74 | Arabidopsis thaliana | A |
| 75 | Arabidopsis thaliana | A |
| 76 | Arabidopsis thaliana | A |
| 77 | Arabidopsis thaliana | A |
| 78 | Arabidopsis thaliana | A |
| 79 | Arabidopsis thaliana | A |
| 80 | Arabidopsis thaliana | A |
| 81 | Arabidopsis thaliana | A |
| 82 | Arabidopsis thaliana | A |
| 83 | Arabidopsis thaliana | A |
| 84 | Arabidopsis thaliana | A |
| 85 | Arabidopsis thaliana | A |
| 86 | Arabidopsis thaliana | A |
| 87 | Arabidopsis thaliana | A |
| 88 | Arabidopsis thaliana | A |
| 89 | Arabidopsis thaliana | A |
| 90 | Arabidopsis thaliana | A |
| 91 | Arabidopsis thaliana | A |
| 92 | Arabidopsis thaliana | A |
| 93 | Arabidopsis thaliana | A |
| 94 | Arabidopsis thaliana | A |
| 95 | Arabidopsis thaliana | A |
| 96 | Arabidopsis thaliana | A |
| 97 | Arabidopsis thaliana | S |
| 98 | Arabidopsis thaliana | A |
| 99 | Arabidopsis thaliana | n.d. |
| 100 | Arabidopsis thaliana | n.d. |
| 101 | Arabidopsis thaliana | n.d. |
| 102 | Arabidopsis thaliana | n.d. |
| 103 | Arabidopsis thaliana | n.d. |
| 104 | Arabidopsis thaliana | n.d. |
| 105 | Arabidopsis thaliana | n.d. |
| 106 | Arabidopsis thaliana | n.d. |
| 107 | Arabidopsis thaliana | n.d. |
| 108 | Arabidopsis thaliana | n.d. |
| 109 | Arabidopsis thaliana | n.d. |
| 110 | Arabidopsis thaliana | n.d. |
| 111 | Arabidopsis thaliana | n.d. |
| 112 | Arabidopsis thaliana | A |
| 113 | Nicotiana benthamiana | A |
| 114 | Nicotiana benthamiana | A |
| 115 | Nicotiana benthamiana | A |
| 116 | ‘Nicotiana benthamiana | S |
| 117 | Oryza japonica | S |
| 118 | Oryza japonica | S |
| 119 | Oryza indica | S |
| 120 | Oryza indica | S |
| 121 | Papaver rhoeas | S |
| 122 | Oryza japonica | S |

The 122 cDNAs of the invention were identified by phenotypic screening and bioinformatic analysis of libraries of over 8000 cDNAs from Arabidopsis, Nicotiana, Oryza and Papaver constructed in the GENEWARE® vector. Table 1 lists whether the cDNA insert is in the sense (S) or antisense (A) configuration in the GENEWARE® vector used for the phenotypic screening. The use of the GENEWARE® vector in the field of genomics has been described in PCT WO 99/36516 published Jul. 22, 1999, which is herein incorporated by reference for all purposes. The general phenotypic screening method (described in greater detail below) involves constructing a GENEWARE® viral nucleic acid vector from each clone of a normalized cDNA library of interest. Each GENEWARE® vector is then used to create an infectious viral unit which is applied to the individual plants of interest. Inoculation with GENEWARE® viral nucleic acid vectors results in a high rate of systemic infection of plants. The TMV based viral vector identified as PBSG1057 which has the ablility to transfect plants has been deposited under the Budapest Treaty at the AFCC and is designated ATCC #203981. Infected (and uninfected) plants are grown under identical conditions and an automated visual phenotypic analysis is conducted of each plant. The phenotypic data including descriptive of various parts of each plant is entered into a matrix-style database created using LIMS software. Once in the database, the phenotypic results are linked to the sequence data and bioinformatic analysis associated with each of the GENEWARE® vector (i.e. each cDNA in the library).

Out of over 8000 Nicotiana benthamiana plants infected by the GENEWARE®, 111 were discovered that exhibited a dwarf phenotype. Sequence analysis of these cDNAs (as described in greater detail below) yielded the identifying nucleic acid sequences SEQ. ID. NOs. 1-111. Bioinformatic analysis of these sequences using BLAST and other methods (described in greater detail below) yielded E.C. annotations for a large number of these sequences.

Further bioinformatic analysis of the 111 polynucleotide sequences identified an additional 34 cDNAs that may also function to cause dwarf phenotype in plants. Pfam analysis (described in greater detail below) of the 111 cDNAs identified SEQ ID NO:95 and 102 as members of the transketolase functional family, and the pfkb carbohydrate kinase family, respectively. Using this information, the 11 additional sequences (identified by SEQ ID NO: 112-122) were discovered in the LSBC GENEWARE® libraries that are either a member of the transketolase having the same metabolic activity as SEQ ID NO. 95, or a member pfkb carbohydrate kinase families having the same metabolic activity as SEQ ID NO. 102.

Following the identification of plants exhibiting the dwarf phenotype, biochemical analyses of tissue may be carried out in order to ascertain further details of the expressed cDNAs function. Methods including GC/MS analysis and Maldi-TOF analysis of the tissue have been carried out (described in greater detail below) and yield information on the profile of metabolites and proteins present in the infected plant's tissue. The results of these biochemical analyses are linked to the phenotype, sequence, and other bioinformatic data associated with each of the GENEWARE® vector. Using these biochemical analysis methods, and associated data processing techniques, the identification of at least one variation in the metabolome of an infected (versus an uninfected) plant may ascribe a function to the cDNA of interest.

According to the present invention, the dwarf phenotype may be created in a wide variety of plants or plant cell systems using the cDNAs identified by SEQ ID NO:1-122 and the various transformation methods described. In preferred embodiments, target plants and plant cells for engineering include, but are not limited to, monocotyledonous and dicotyledonous plants, including horticultural and ornamental plants (e.g., the grass and turfgrass species, and flowering plants such as petunia, rose, chrysanthemum), conifers and pine trees (e.g., pine, fir, spruce species, and including Abies sp., Acer glabrum, Pinus sp., Alnus sp., Arbutus arizonica, Betula occidentalis, Cedrus sp., Cryptomeriajaponica, Cupressus sp., Eucalyptus sp., Ginkgo biloba, Juniperus sp., Libocedrus decurrens, Liriodendron tulipifera, Lithocarpus densiflora, Metasequoia glyptostroboides, P. ponderosa var. scopulorum, Picea sp., Platanus sp., Populus sp., Pseudotsuga sp., Purshia tridentata, Quercus sp., Sequoia sp., Taxus brevifolia, Thuja sp., Torreya californica, Tsuga heterophylla, Umbellularia californica); plants used in phytoremediation (e.g., heavy metal accumulating plants), medicinal plants (e.g. Solanaceae, Atropa belladonna, Duboisia myoporides, Hyoscymus niger, Scopolina atropoides, Solanum tuberosum, Eschscholtzia californica, Berberis stolonifera, Papaver somniferum) and plants used for experimental purposes (e.g., Arabidopsis thaliana, Nicotiana sp.).

For a more complete listing of medicinal plants see Table 2. Another treatment of medicinal herbs can be found in, “1999 PDR for Herbal Medicines” 2nd edition, editors, Joerg Gruenwald et al.,, Medical Economics Company, Montvale, N.J., which is herein incorporated by reference for all purposes.

TABLE 2

| Medicinal Plant | Medicinal Plant |
|---|---|
| Abies lasiocarpa | Juglans major |
| Abies excelsa | Juniperus communis |
| Abronia wootonii | Juniperus monosperma |
| Acacia arabica | Juniperus sibirica |
| Acacia catechu | Kallstroemia grandiflora |
| Acacia constricta | Kallstroemia spp. |
| Acacia greggii | Kalmia angustifolia |
| Acacia senegal | Kalmia latifolia |
| Acalypha californica | Kalmia microphylla |
| Acalypha lindheimeri | Kalmia polifolia |
| Achillea lanulosa | Karwinskia humboldtiana |
| Achillea millefolium | Krameria grayi |
| Achlys triphylla | Krameria lanceolata |
| Aconitum columbianum | Krameria parvifolia |
| Acorus calamus | Lactuca serriola |
| Actaea alba | Lamium amplexicaule |
| Actea rubra | Larrea tridentata |
| Adiantum capillus-veneris | Ledum glandulosum |
| Adiantum jordanii | Ledum groenlandicum |
| Adiantum pedatum | Leonurus cardiaca |
| Adoxa moschatellina | Leonurus sibirica |
| Aesculus californica | Lepechinia calycina |
| Aesculus glabra | Lepidium montanum |
| Aesculus hippocastanum | Lespedeza violacea |
| Aesculus pavia | Leucophyllum frutescens |
| Agastache urticifolia | Levisticum ligusticum |
| Agave chisoensis | Lewisia rediviva |
| Agave parryi | Liatris punctata |
| Agrimonia gryposepala | Liatris squarrosa |
| Agrimonia striata | Ligusticum filicinum |
| Agropyron repens | Ligusticum grayi |
| Alchemilla mollis | Ligusticum porteri |
| Alchemilla vulgaris | Lilium grayi |
| Aletris farinosa | Lilium philadelphicum |
| Alhagi camelorum | Linaria canadensis |
| Allium cernuum | Linaria dalmatica |
| Allium geyeri | Linaria vulgaris |
| Allium schoenoprasum | Linnaea borealis |
| Alnus incana | Linum lewisii |
| Aloe spp. | Linum medium |
| Aloe vera | Linum usitatissimum |
| Althea officinalis | Liquidambar orientalis |
| Amaranthus hybridus | Liquidamber styraciflua |
| Ambrosia ambrosioides | Lithospermum arvense |
| Ambrosia artemisiifolia | Lithospermum multiflorum |
| Ambrosia trifida | Lithospermum ruderale |
| Amelanchier alnifolia | Lobelia cardinalis |
| Amsinckia intermedia | Lobelia cardinalis, |
| Amsonia hirtella | Lobelia cardinalis, |
| Amygdalus persica | Lobelia inflata |
| Anaphalis margaritacea | Lobelia kalmii |
| Anemone deltoidea | Lobelia siphilitica |
| Anemone globosa | Lomatium cous |
| Anemone halleri | Lomatium dissectum |
| Anemone occidentalis | Lophocereus (Pachycereus) |
| Anemone patens | Lycium fremontii |
| Anemone patens, | Lycium pallidum |
| Anemone quinquefolia | Lycopodium clavatum |
| Anemone tuberosa | Lycopus americanus |
| Anemopsis californica | Lycopus asper |
| Anethum graveolens | Lycopus uniflorus |
| Angelica sp. | Lycopus virginicus |
| Angelica archangelica | Lysichitum americanum |
| Angelica arguta | Lythrum salicaria |
| Angelica dawsonii | Macromeria viridiflora |
| Angelica genuflexa | Magnolia grandiflora |
| Angelica grayi | Mahonia aquifolia |
| Angelica hendersonii | Mahonia fremontii |
| Angelica lineariloba | Mahonia haematocarpa |
| Angelica pinnata | Mahonia nervosa |
| Angelica venenosa | Mahonia repens |
| Antennaria howellii | Mahonia trifoliata |
| Antennaria rosea | Mahonia wilcoxii |
| Apocynum androsaemifolium | Malus sylvestris |
| Apocynum cannabinum | Malva neglecta |
| Apocynum medium | Mammillaria arizonica |
| Aquilegia caerulea | Marah gilensis |
| Aquilegia chrysantha | Marrubium vulgare |
| Aralia californica | Matricaria chamomilla |
| Aralia nudicaulis | Matricaria matricarioides |
| Aralia racemosa | Medicago sativa |
| Aralia spinosa | Melampyrum lineare |
| Arbutus menziesii | Melilotus albus |
| Arctium minus | Menispermum canadense |
| Arctostaphylos pungens | Mentha arvensis |
| Arctostaphylos uva-ursi | Mentha pulegium |
| Argemone corymbosa | Mentha spicata |
| Argemone mexicana | Menyanthes trifoliata |
| Argemone platyceras | Mertensia ciliata |
| Argemone polyanthemos | Mimulus guttatus |
| Arisaema atrorubens | Mirabilis longiflora |
| Arisaema dracontium | Mirabilis multiflorum |
| Arisaema stewardsonii | Mitchella repens |
| Arisaema triphyllum | Monarda citriodora |
| Aristolochia californica | Monarda didyma |
| Aristolochia serpentaria | Monarda fistulosa |
| Aristolochia watsonii | Monarda media |
| Arnica angustifolium | Monarda menthaefolia |
| Arnica cordifolia | Monarda mollis |
| Arnica latifolia | Monarda pectinata |
| Arnica mollis | Monarda punctata |
| Arnica montana | Monardella villosa |
| Artemisia douglasiana | Moneses uniflora |
| Artemisia filifolia | Monotropa hypopitys |
| Artemisia franserioides | Monotropa uniflora |
| Artemisia frigida, | Mortonia scabrella |
| Artemisia frigida | Myrica californica |
| Artemisia ludoviciana | Myrica cerifera |
| Artemisia tridentata | Myristica fragrans |
| Artemisia vulgaris | Nelumbo lutea |
| Asarum canadense | Nepeta cataria |
| Asarum caudatum | Nicotiana attenuata |
| Asclepias albicans | Nicotiana glauca |
| Asclepias asperula | Nicotiana repanda |
| Asclepias brachystephana | Nicotiana tabacum |
| Asclepias erosa | Nicotiana trigonophylla |
| Asclepias fascicularis | Nuphar luteum |
| Asclepias speciosa | Nymphaea odorata |
| Asclepias subulata | Ocimum basilicum |
| Asclepias syriaca | Oenothera biennis |
| Asclepias texana | Oenothera hookeri |
| Asclepias tuberosa | Oplopanax horridum |
| Asclepas viridis | Opuntia erinacea |
| Asclepias viridis | Opuntia phaeacantha |
| Asparagus officinale | Orobanche fasciculata |
| Aspidium filix-mas | Orobanche ludoviciana |
| Astragalus gummifer | Orobanche uniflora |
| Astragalus americanus | Osmorhiza obtusa |
| Astragalus membranaceus | Osmorrhiza longistylis |
| Atriplex canescens | Osmorrhiza occidentalis |
| Avena fatua | Ourouparia gambir |
| Avena sativa | Oxalis cymosa |
| Balsamorhiza sagittata | Oxalis oregana |
| Baptisia australis | Oxalis metcalfei |
| Baptisia leucantha | Paeonia brownii |
| Baptisia leucophaea | Paeonia californica |
| Baptisia sphaerocarpa | Panax quinquefolium |
| Baptisia tinctoria | Panax trifolium |
| Buddleya sp. | Papaver rhoeas |
| Berberis fendleri | Papaver somniferum |
| Berberis vulgaris | Parthenium incanum |
| Berberis - | Parthenocissus inserta |
| Besseya wyomingensis | Parthenocissus quinquefolia |
| Bidens frondosa | Passiflora foetida |
| Bidens pilosa | Passiflora incarnata |
| Bignonia capreolata | Passiflora lutea |
| Bouvardia ternifolia | Passiflora sanguinea |
| Brassica arvensis | Paullinia cupana |
| Brickellia amplexicaulis | Pedicularis bracteosa |
| Brickellia californica | Pedicularis canadensis |
| Brickellia grandiflora | Pedicularis contorta |
| Brugmansia sp. | Pedicularis densiflora |
| Bryonia alba | Pedicularis grayii |
| Bupleurum americanum | Pedicularis groenlandica |
| Bursera microphylla | Pedicularis lanceolata |
| Bursera odorata | Pedicularis parryi |
| Cacalia decomposita | Pedicularis racemosa |
| Caesalpinia gilliessii | Peganum harmala |
| Caesalpinia pulcherrima | Peniocereus greggii |
| Caffea arabica | Penstemon cobaea |
| Calendula officinalis | Penstemon eatoni |
| Callirhoe involucrata | Penstemon lyallii |
| Caltha biflora | Perezia nana |
| Caltha leptosepala | Perezia wrightii |
| Caltha palustris | Perideridia gairdneri |
| Calypso bulbosa | Perilla frutescens |
| Camassia quamash | Petasites frigidus |
| Camissonia (Oenothera) | Petasites frigidus, |
| Campsis radicans | Petasites sagittatus |
| Cannabis sativa | Philadelphus lewisii |
| Capsella bursa-pastoris | Phoradendron flavescens |
| Capsicum annuum | Phoradendron juniperinum |
| Capsicum frutescens | Physalis crassifolia |
| Cardamine cordifolia | Physocarpus monogynus |
| Carnegia gigantea | Physostigma venenosum |
| Cassia angustifolia | Phytolacca americana |
| Cassia covesii | Picea engelmanni |
| Cassia fasciculata | Pinus contorta |
| Cassia fistula | Pinus edulis |
| Cassia leptocarpa | Pinus palustris |
| Cassia marilandica | Pinus ponderosa |
| Cassia senna | Pinus strobus |
| Cassia wislizenii | Pinus taeda |
| Castanopsis chrysophylla | Piper sp |
| Castela emoryi | Piper cubeba |
| Castilleja sp. | Plantago lanceolata |
| Castilleja miniata | Plantago major |
| Caulophyllum thalictrioides | Plantago patagonica |
| Ceanothus americanus | Plantago rugeli |
| Ceanothus cuneatus | Pluchea camphorata |
| Ceanothus fendleri | Podophyllum peltatum |
| Ceanothus greggii | Polygala alba |
| Ceanothus herbaceum | Polygala lutea |
| Ceanothus spinosus | Polygala obscura |
| Ceanothus velutinus | Polygala paucifolia |
| Celastrus scandens | Polygala senega |
| Celtis occidentalis | Polygonatum biflorum |
| Centaurium venustum | Polygonatum canaliculatum |
| Cephaelis ipecacuanha | Polygonum bistortioides |
| Cephalanthus occidentalis | Polymnia spp |
| Cerastium arvense | Polymnia canadensis |
| Cercis occidentalis | Polypodium glycyrriza |
| Cercocarpus sp. | Polystichum munitum |
| Cetraria islandica | Populus balsamifera |
| Chamaelirium luteum | Populus fremontii |
| Chelidonium majus | Populus tremulioides |
| Chelone glabra | Portulaca oleracea |
| Chelone lyoni | Potentilla diversifolia |
| Chenopodium ambrosioides | Potentilla fruticosa |
| Chilopsis linearis | Potentilla palustris |
| Chimaphila umbellata | Potentilla strigosa |
| Chimaphila umbellata, | Potentilla tridentata |
| Chionanthus virginiana | Proboscidea parviflora |
| Chlorogalum pomeridianum | Prosopis juliflora |
| Chondrus crispus | Prunella vulgaris |
| Choisya arizonica | Prunus americana |
| Chrysanthemum leucanthemum | Prunus avium |
| Chrysanthemum parthenium | Prunus laurocereus |
| Cichorium intybus | Prunus serotina |
| Cicuta douglasii | Prunus virginiana |
| Cimicifuga arizonica | Pseudotsuga menziesii |
| Cimicifuga elata | Psoralea esculenta |
| Cimicifuga racemosa | Ptelea pallida |
| Cinchona succirubra | Ptelea trifoliata |
| Cinnamomum camphora | Pulsatilla ludoviciana |
| Cirsium undulatum | Punica granatum |
| Citrullus colocynthis | Purshia tridentata |
| Citrus sinensis | Pyrola asarifolia |
| Claviceps purpurea | Pyrola minor |
| Claytonia lanceolata | Pyrola rotundifolia |
| Clematis columbiana | Pyrola secunda |
| Clematis hirsutissima | Prola virens |
| Clematis ligusticifolia | Quercus alba |
| Clematis pseudoalpina | Quercus gambelii |
| Clematis viorna | Quillaja saponaria |
| Clematis virginiana | Ratibida columnaris |
| Cleome serrulata | Rhamnus alnifolia |
| Cocculus sp. | Rhamnus betulifolia |
| Cola nitida | Rhamnus californica |
| Colchicum autumnale | Rhamnus frangula |
| Collinsonia canadensis | Rhamnus purshiana |
| Commandra umbellata | Rheum officinale |
| Conium maculatum | Rhus choriophylla |
| Conopholis alpina | Rhus glabra |
| Conopholis americana | Rhus microphylla |
| Convallaria majus | Rhus (Toxicodendron) |
| Convolvulus arvensis | Rhus trilobata |
| Convolvulus scammonia | Ribes aureum |
| Conyza canadense | Ricinus communis |
| Copaiba langsdorffii | Romneya coulteri |
| Coptis groenlandica | Rosa acicularis |
| Coptis laciniata | Rosa humilis |
| Coptis occidentalis | Rosa virginiana |
| Corallorhiza maculata | Rosa woodsii |
| Corallorrhiza striata | Rubus idaeus |
| Cordia boissieri | Rubus odoratus |
| Cornus canadensis | Rubus parviflorus |
| Cornus florida | Rudbeckia hirta |
| Cornus stolonifera | Rudbeckia laciniata |
| Corydalis aureus | Ruellia ciliosa |
| Corydalis sempervirens | Rumex acetosella |
| Crataegus spp. | Rumex crispus |
| Crataegus columbiana | Rumex hymenosepalus |
| Crataegus douglasii | Ruta graveolens |
| Crataegus mollis | Sabal texana |
| Crataegus rivularis | Sabatia angularis |
| Crataegus succulenta | Sabatia campestris |
| Cucurbita foetidissima | Sabatia stellaris |
| Cupressus arizonica | Sagittaria cuneata |
| Cupressus macrocarpa | Sagittaria latifolia |
| Curcuma sp. | salix sp. |
| Cuscuta gronovi | Salix discolor |
| Cymopterus fendleri | Salvia apiana |
| Cynanchum nigrum | Salvia azurea |
| Cynara sp. | Salvia clevelandii |
| Cynoglossum officinale | Salvia columbariae |
| Cypripedium sp. | Salvia greggii |
| Cypripedium acaule | Salvia henryi |
| Cypripedium arietinum | Salvia lemmonii |
| Cypripedium calceolus | Salvia leucophylla |
| Cypripedium montanum | Salvia mellifera |
| Cypripedium parviflorum | Salvia regla |
| Cypripedium reginae | Salvia reflexa |
| Cytisus scoparius | Salvia spathaceae |
| Dalea formosa | Sambucus canadensis |
| Darlingtonia californica | Sambucus mexicana |
| Datura ferox | Sambucus racemosa |
| Datura metelioides | Sanguinaria canadensis |
| Datura wrightii | Sanguisorba canadensis |
| Daucus carota | Sanicula marilandica |
| Delphinium barbeyi | Santalum album |
| Delphinium elongatum | Sanvitalia abertii |
| Dendromecon rigida | Sapindus saponaria |
| Dicentra canadensis | Saponaria officinalis |
| Dicentra cucullaria | Sarracenia psittacina |
| Dicentra formosa | Sarracenia purpurea |
| Dicentra spectabilis | Sarracenia rubra |
| Digitalis purpurea | Sassafras IL |
| Dionaea muscipula | Satureja douglasii |
| Dioscorea villosa | Saururus cernuus |
| Dipsacus sylvestris | Scopola carniolica |
| Dipsacus fullonum | Scrophularia californica |
| Dodecathion pulchellum | Scrophularia lanceolata |
| Dracocephalum moldavica | Scutellaria brittonii |
| Dracocephalum parviflorum | Scutellaria californica |
| Drosera linearis | Scutellaria drummondii |
| Drosera rotundifolia | Scutellaria epilobiifolia |
| Dyssodia papposa | Scuteliaria galericulata |
| Ecballium elaterium | Scutellaria incana |
| Echevaria rusbyi | Scutellaria integrifolia |
| Echinacea angustifolia | Scutellaria latiflora |
| Echinacea pallida | Scutellaria resinosa |
| Echinacea purpurea | Scutellaria serrata |
| Echinacea tennessiensis | Scutellaria tesselata |
| Elettaria carmamomum | Scutellaria wrightii |
| Encelia farinosa | Sedum rhodanthum |
| Ephedra californica | Sedum roseum |
| Ephedra nevadensis | Selenicereus spp. |
| Ephedra torreyana | Senecio aureus |
| Ephedra trifurca | Senecio cineraria |
| Ephedra viridis | Sequoia sempervirens |
| Epifagus virginianum | Serenoa repens |
| Epigaea repens | Shephardia argentea |
| Epilobium angustifolium | Shephardia canadensis |
| Epilobium hirsutum | Sida hederacea |
| Epipactis gigantea | Sidalcea neomexicana |
| Epipactis helleborine | Sidalcea malvaeflora |
| Equisetum arvense | Silphium laciniata |
| Equisetum pratense | Silphium perfoliatum |
| Eremocarpus setigerus | Silphium terebinthinaceum |
| Eriodictyon angustifolia | Silybum marianum |
| Eriodictyon californica | Simmondsia chinensis |
| Eriodictyon crassifolium | Smilacina racemosa |
| Eriodictyon glutinosa | Smilacina stellata |
| Eriogonum leptophyllum | Smilacina trifolia |
| Eriogonum umbellata | Smilax spp. |
| Eriogonum wrightii | Smilax californica |
| Erodium cicutarium | Smilax glauca |
| Eryngium leavenworthii | Smilax herbacea |
| Eryngium lemmonii | Smilax rotundifolia |
| Eryngium yuccafolium | Solanum carolinense |
| Erysimum capitatum | Solanum dulcamara |
| Erythronium grandiflorum | Solanum eleagnifolium |
| Erythronium montanum | Solanum nodiflorum |
| Erythroxylon coca | Solidago canadensis |
| Eschscholtzia californica | Sophora secundiflora |
| Eschscholtzia mexicana | Sorbus scopulina |
| Eschscholtzia minutiflora | Spartium junceum |
| Eucalyptus sp. | Sphaeralcea ambigua |
| Euonymus occidentalis | Sphaeralcea angustifolia |
| Eupatorium coelestinum | Sphaeralcea coccinea |
| Eupatorium greggii | Sphaeralcea fendleri |
| Eupatorium herbaceum | Sphaeralcea parviflora |
| Eupatorium maculatum | Sphenosciadium capitellatum |
| Eupatorium perfoliatum | Spigelia marilandica |
| Eupatorium purpureum | Spiraea alba |
| Eupatorium rugosum | Spiraea tomentosa |
| Eustoma grandiflorum | Stachys albens |
| Eysenhardtia polystachya | Stachys palustris |
| Fallugia paradoxa | Stachys rigida |
| Ferula foetida | Stellaria media |
| Ferula galbaniflua | Stenocereus thurberi |
| Flourensia cernua | Sticta PH |
| Fouquieria splendens | Stillingia sylvatica |
| Fragaria glauca | Streptopus amplexifolius |
| Fragaria ovalis | Strychnos nux-vomica |
| Fragaria virginiana | Swertia radiata |
| Frankenia grandiflora | Symphytum officinalis |
| Frankenia palmeri | Symplocarpus foetidus |
| Fraxinus ornus | Tanacetum huronense |
| Fremontia californica | Tanacetum parthenium |
| Fritillaria atropurpurea | Tanacetum vulgare |
| Fritillaria pudica | Taraxacum sp. |
| Fucus vesiculosus | Taxus brevifolia |
| Fumaria officinalis | Tecoma stans |
| Gaillardia pinnatifida | Teucrium laciniatum |
| Galium aparine | Thalictrum fendleri |
| Galium borealis | Thamnosma texana |
| Garcinia hanburyi | Thamnosma montana |
| Garrya spp. | Thelesperma gracile |
| Garrya elliptica | Tephrosia virginiana |
| Garrya flavescens | Thermopsis montana |
| Garrya wrightii | Thuja plicata |
| Gaultheria procumbens | Thymus vulgaris |
| Gaultheria shallon | Tillandsia recurvata |
| Gaura lindheimeri | Tillandsia usnioides |
| Gaura parviflora | Toluifera balsamum |
| Gaylussacia brachycera | Toluifera pereirae |
| Gelsemium sempervirens | Toxicodendron radicans |
| Gentiana affinis | Toxicodendron vernix |
| Gentiana algida | Tradescantia occidentalis |
| Gentiana andrewsi | Tragopogon dubius |
| Gentiana calycosa | Trautvettaria carolinensis |
| Gentiana crinata | Tribulus terrestrus |
| Gentiana heterosepala | Trichostema lanatum |
| Gentiana parryi | Trifolium pratense |
| Gentiana saponaria | Trillium erectum |
| Gentiana simplex | Trillium grandiflorum |
| Gentiana thermalis | Trillium ovatum |
| Gentianella (Gentian) | Trillium sessile |
| Geranium maculatum | Trillium undulatum |
| Geranium richardsonii | Trollius laxus |
| Geranium viscosissimum | Tsuga mertensiana |
| Geum rivale | Turnera diffusa |
| Geum triflorum | Umbellularia californica |
| Gigartina mamillosa | Urginea maritima |
| Gillenia trifoliata | Urtica dioica |
| Glecoma hederacea | Usnea barbata |
| Glycyrriza glabra | Usnea hirsutissima |
| Glycyrrhiza lepidota | Vaccinium corymbosum |
| Gnaphallium sp. | Vaccinium myrtillus |
| Goodyera spp. | Vaccinium ovatum |
| Gossypium thurberi | Vaccinium oxycoccos |
| Grindelia aphanactis | Vaccinium parvifolium |
| Grindelia squarrosa | Vaccinium scoparium |
| Guaiacum angustifolium | Vaccinium tenellum |
| Guaiacum coulteri | Vaccinium uliginosum |
| Guaiacum sanctum | Vaccinium vitis-idaea |
| Gutierrezia sarothrae | Valeriana acutiloba |
| Habenaria blephariglottis | Valeriana arizonica |
| Habeneria fimbriata | Valeriana edulus |
| Habenaria (Plantanthera) | Valeriana officinalis |
| Hagenia abyssinica | Valeriana occidentalis |
| Hamamelis virginiana | Valeriana sitchensis |
| Haplopappus laricifolius | Vancouveria hexandra |
| Hedeoma hyssopifolium | Veratrum californicum |
| Hedeoma oblongifolia | Veratrum viride |
| Hedysarum alpinum | Verbascum blattaria |
| Helenium (Dugaldia) | Verbascum thapsus |
| Heliotropium convolvulaceum | Verbena bipinnatifida |
| Heracleum lanatum | Verbena bracteata |
| Heterotheca grandiflora | Verbena canadensis |
| Heterotheca psammophylla | Verbena ciliata |
| Heterotheca subaxillaris | Verbena gooddingii |
| Heuchera americanus | Verbena hastata |
| Heuchera micrantha | Verbena macdougalii |
| Heuchera parvifolia | Verbena stricta |
| Heuchera sanguinea | Verbena wrightii |
| Hibiscus moscheutos | Verbesina encelioides |
| Hibiscus oculiroseus | Veronica americana |
| Hierochloe odorata | Veronica chamaedrys |
| Holodiscus dumosus | Veronicastrum IM |
| Humulus americanus | Viburnum acerifolium |
| Humulus lupulus | Viburnum americanum |
| Hydrastis canadensis | Viburnum cassinoides |
| Hydrocotyle bonariensis | Viburnum edule |
| Hydrophyllum capitatum | Viburnum ellipticum |
| Hyocyamus niger | Viburnum opulus |
| Hypericum ascyron | Viburnum prunifolium |
| Hypericum aureum | Viburnum rufidulum |
| Hypericum formosum | Vigueria dentata |
| Hypericum perforatum | Vinca major |
| Hyptis emoryi | Viola sp |
| Hyssopus officinalis | Viola canadensis |
| Ilex vomitoria | Viola pedata |
| Impatiens biflora | Viola tricolor |
| Impatiens capensis | Vitex agnus-castus |
| Impatiens pallida | Xanthium spinosum |
| Indigofera sphaerocarpa | Xanthium strumarium |
| Inula helenium | Xerophyllum tenax |
| Ipomea arborescens | Yucca baccata |
| Ipomea jalapa | Yucca baileyi |
| Ipomea leptophylla | Yucca elata |
| Iris missouriensis | Yucca schottii |
| Iris prismatica | Zanthoxylum fagaria |
| Iris versicolor | Zauschneria latifolia |
| Jateorhiza palmata | Zigadenus elegans |
| Jatropha cardiophylla | Zigadenus venenosus |
| Jatropha dioica | Zingiber sp. |
| Jatropha macrorhiza | Zizia aptera |
| Jeffersonia diphylla | |

The dwarf phenotype may be created using the cDNAs of the present invention in conjunction with a wide variety of plant virus expression vectors. The plant virus selected may depend on the plant system chosen and its known susceptibility to viral infection. Preferred embodiments of the plant virus expression vectors include, but are not limited to those in Table 3.

TABLE 3

| Plant Viruses | Plant Viruses |
|---|---|
| Abelia latent tymovirus | Lucerne transient streak |
| Abutilon mosaic bigeminivirus | Lychnis ringspot hordeivirus |
| Ahlum waterborne carmovirus | Maclura mosaic macluravirus |
| Alfalfa 1 alphacryptovirus | Maize dwarf mosaic potyvirus |
| Alfalfa 2 betacryptovirus | Maize streak monogeminivirus |
| Alfalfa mosaic alfamovirus | Maracuja mosaic tobamovirus |
| Alsike clover vein mosaic virus | Marigold mottle potyvirus |
| Alstroemeria ilarvirus | Melandrium yellow fleck |
| Alstroemeria mosaic potyvirus | Melilotus mosaic potyvirus |
| Alstroemeria streak potyvirus | Melon Ourmia ourmiavirus |
| Amaranthus leaf mottle potyvirus | Melothria mottle potyvirus |
| Amaryllis alphacryptovirus | Milk vetch dwarf nanavirus |
| Amazon lily mosaic potyvirus | Mulberry latent carlavirus |
| Apple mosaic ilarvirus | Muskmelon vein necrosis carlavirus |
| Apple stem grooving capillovirus | Myrobalan latent ringspot nepovirus |
| Arabis mosaic nepovirus | Nandina mosaic potexvirus |
| Arracacha A nepovirus | Narcissus late season yellows |
| Arracacha A nepovirus | Narcissus latent macluravirus |
| Arracacha B nepovirus | Narcissus mosaic potexvirus |
| Arracacha Y potyvirus | Narcissus tip necrosis carmovirus |
| Artichoke Italian latent nepovirus | Narcissus tip necrosis carmovirus |
| Artichoke latent potyvirus | Narcissus yellow stripe potyvirus |
| Artichoke latent S carlavirus | Neckar River tombusvirus |
| Artichoke mottled crinkle | Nerine potyvirus |
| Artichoke vein banding nepovirus | Nicotiana velutina mosaic furovirus |
| Artichoke yellow ringspot | Oat blue dwarf marafivirus |
| Asparagus 1 potyvirus | Oat blue dwarf marafivirus |
| Asparagus 2 ilarvirus | Oat golden stripe furovirus |
| Asparagus 3 potexvirus | Odontoglossum ringspot |
| Aster chlorotic stunt carlavirus | Okra leaf-curl bigeminivirus |
| Asystasia gangetica mottle | Okra mosaic tymovirus |
| Aucuba ringspot badnavirus | Olive latent 1 sobemovirus |
| Barley stripe mosaic hordeivirus | Olive latent 2 ourmiavirus |
| Barley stripe mosaic hordeivirus | Onion mite-borne latent potexvirus |
| Barley yellow dwarf luteovirus | Onion yellow dwarf potyvirus |
| Barley yellow streak mosaic virus | Orchid fleck rhabdovirus |
| Bean calico mosaic bigeminivirus | Panicum mosaic sobemovirus |
| Bean common mosaic potyvirus | Papaya mosaic potexvirus |
| Bean distortion dwarf | Papaya ringspot potyvirus |
| Bean leaf roll luteovirus | Paprika mild mottle tobamovirus |
| Bean pod mottle comovirus | Parietaria mottle ilarvirus |
| Bean yellow mosaic potyvirus | Parsnip leafcurl virus |
| Beet curly top hybrigeminivirus | Parsnip mosaic potyvirus |
| Beet leaf curl rhabdovirus | Parsnip yellow fleck sequivirus |
| Beet mild yellowing luteovirus | Passiflora ringspot potyvirus |
| Beet mosaic potyvirus | Passionfruit woodiness potyvirus |
| Beet necrotic yellow vein furovirus | Patchouli mosaic potyvirus |
| Beet pseudo-yellows closterovirus | Pea early browning tobravirus |
| Beet soil-borne furovirus | Pea enation mosaic enamovirus |
| Beet western yellows leuteovirus | Pea mild mosaic comovirus |
| Beet yellows closterovirus | Pea mosaic potyvirus |
| Belladonna mottle tymovirus | Pea seed-borne mosaic potyvirus |
| Bidens mosaic potyvirus | Pea streak carlavirus |
| Black raspberry necrosis virus | Peach enation nepovirus |
| Blueberry leaf mottle nepovirus | Peach rosette mosaic nepovirus |
| Blueberry necrotic shock ilarvirus | Peanut chlorotic streak caulimovirus |
| Bramble yellow mosaic potyvirus | Peanut clump furovirus |
| Broad bean mottle bromovirus | Peanut mottle potyvirus |
| Broad bean necrosis furovirus | Peanut stunt cucumovirus |
| Broad bean stain comovirus | Peanut yellow spot tospovirus |
| Broad bean true mosaic comovirus | Pelargonium flower break |
| Broad bean wilt fabavirus | Pelargonium line pattern |
| Brome mosaic bromovirus | Pelargonium vein clearing |
| Burdock yellow mosaic potexvirus | Pelargonium zonate spot |
| Cacao necrosis nepovirus | Pepino mosaic potexvirus |
| Cacao swollen shoot badnavirus | Pepper Indian mottle potyvirus |
| Cacao yellow mosaic tymovirus | Pepper mild mosaic potyvirus |
| Cactus 2 carlavirus | Pepper mild mottle tobamovirus |
| Cactus X potexvirus | Pepper Moroccan tombusvirus |
| Canavalia maritima mosaic | Pepper mottle potyvirus |
| Caper latent carlavirus | Pepper ringspot tobravirus |
| Caraway latent nepovirus | Pepper severe mosaic potyvirus |
| Carnation rhabdovirus | Pepper Texas bigeminivirus |
| Carnation rhabdovirus | Pepper veinal mottle potyvirus |
| Carnation 1 alphacryptovirus | Petunia asteroid mosaic |
| Carnation 2 alphacryptovirus | Physalis mild chlorosis luteovirus |
| Carnation etched ring caulimovirus | Physalis mosaic tymovirus |
| Carnation Italian ringspot | Pineapple chlorotic leaf streak |
| Carnation latent carlavirus | Pineapple wilt-associated |
| Carnation mottle carmovirus | Pittosporum vein yellowing |
| Carnation mottle carmovirus | Plantain 6 carmovirus |
| Carnation necrotic fleck | Plantain 7 potyvirus |
| Carnation ringspot dianthovirus | Plantain X potexvirus |
| Carnation vein mottle potyvirus | Plum American line pattern ilarvirus |
| Carnation yellow stripe necrovirus | Plum pox potyvirus |
| Carrot mosaic potyvirus | Poinsettia mosaic tymovirus |
| Carrot mottle mimic umbravirus | Poplar mosaic carlavirus |
| Carrot mottle umbravirus | Poplar vein yellowing |
| Carrot yellow leaf closterovirus | Potato 14R tobamovirus |
| Cassava African mosaic | Potato A potyvirus |
| Cassava brown streak potyvirus | Potato Andean latent tymovirus |
| Cassava brown streak-associated | Potato Andean mottle comovirus |
| Cassava Caribbean mosaic | Potato aucuba mosaic potexvirus |
| Cassava Colombian symptomless | Potato black ringspot nepovirus |
| Cassava common mosaic | Potato leafroll luteovirus |
| Cassava green mottle nepovirus | Potato M carlavirus |
| Cassava Indian mosaic | Potato mop-top furovirus |
| Cassava Ivorian bacilliform | Potato mop-top furovirus |
| Cassava Ivorian bacilliform | Potato T trichovirus |
| Cassava X potexvirus | Potato U nepovirus |
| Cassia mild mosaic carlavirus | Potato V potyvirus |
| Cassia severe mosaic closterovirus | Potato X potexvirus |
| Celery latent potyvirus | Potato Y potyvirus |
| celery mosaic potyvirus | Potato yellow dwarf |
| Cherry leaf roll nepovirus | Primula mosaic potyvirus |
| Chickpea bushy dwarf potyvirus | Primula mottle potyvirus |
| Chickpea chlorotic dwarf | Prune dwarf ilarvirus |
| Chickpea distortion mosaic | Prunus necrotic ringspot ilarvirus |
| Chicory yellow mottle nepovirus | Radish mosaic comovirus |
| Chilli veinal mottle potyvirus | Raspberry ringspot nepovirus |
| Chino del tomat, bigeminivirus | Red clover mottle comovirus |
| Citrus leaf rugose ilarvirus | Red clover necrotic mosaic |
| Citrus ringspot virus | Red clover vein mosaic carlavirus |
| Clover mild mosaic virus | Rhynchosia mosaic bigeminivirus |
| Clover wound tumor phytoreovirus | Ribgrass mosaic tobamovirus |
| Clover wound tumor phytoreovirus | Rice hoja blanca tenuivirus |
| Clover yellow mosaic potexvirus | Rice stripe necrosis furovirus |
| Clover yellow vein potyvirus | Rice stripe tenuivirus |
| Colocasia bobone disease | Rose tobamovirus |
| Commelina X potexvirus | Rubus Chinese seed-borne |
| Cowpea chlorotic mottle | saguaro cactus carmovirus |
| Cowpea mild mottle carlavirus | Scrophularia mottle tymovirus |
| Cowpea mosaic comovirus | Shallot latent carlavirus |
| Cowpea mosaic comovirus | Shallot mite-borne latent potexvirus |
| Cowpea mottle carmovirus | Shallot yellow stripe potyvirus |
| Cowpea severe mosaic comovirus | Silene X potexvirus |
| Cowpea severe mosaic comovirus | Sint-Jan's onion latent carlavirus |
| Croton yellow vein mosaic | Sitke waterborne tombusvirus |
| Cucumber green mottle mosaic | Solanum apical leaf curling |
| Cucumber leaf spot carmovirus | Solanum nodiflorum mottle |
| Cucumber mosaic cucumovirus | Solanum nodiflorum mottle |
| Cucumber mosaic cucumovirus | Sonchus cytorhabdovirus |
| Cucumber necrosis tombusvirus | Sonchus yellow net |
| Cycas necrotic stunt nepovirus | Sorghum mosaic potyvirus |
| Cymbidium ringspot tombusvirus | Sowbane mosaic sobemovirus |
| Cynara nucleorhabdovirus | Soybean crinkle leaf bigeminivirus |
| Dahlia mosaic caulimovirus | Soybean dwarf luteovirus |
| Dandelion yellow mosaic | Soybean mild mosaic virus |
| sequivirus | Soybean mosaic potyvirus |
| Daphne Y potyvirus | Spinach latent ilarvirus |
| Dasheen bacilliform badnavirus | Spinach temperate alphacryptovirus |
| Dasheen mosaic potyvirus | Spring beauty latent bromovirus |
| Datura Colombian potyvirus | Statice Y potyvirus |
| Datura distortion mosaic potyvirus | Strawberry latent ringspot |
| Datura innoxia Hungarian mosaic | Subterranean clover red leaf |
| Datura mosaic potyvirus | Sugarcane mosaic potyvirus |
| Datura necrosis potyvirus | Sunflower ringspot ilarvirus |
| Datura shoestring potyvirus | Sunn-hemp mosaic tobamovirus |
| Datura yellow vein | Sweet clover latent |
| Desmodium mosaic potyvirus | Sweet clover necrotic mosaic |
| Dioscorea green banding mosaic | Sweet potato feathery mottle |
| Dioscorea latent potexvirus | Sweet potato latent potyvirus |
| Dogwood mosaic nepovirus | Sweet potato mild mottle |
| Dulcamara mottle tymovirus | Sweet potato ringspot nepovirus |
| Eggplant green mosaic potyvirus | Sweet potato sunken vein |
| Eggplant mild mottle carlavirus | Tamarillo mosaic potyvirus |
| Eggplant mottled crinkle | Tamus latent potexvirus |
| Eggplant mottled dwarf | Telfairia mosaic potyvirus |
| Eggplant severe mottle potyvirus | Tobacco etch potyvirus |
| Elderberry carlavirus | Tobacco leaf curl bigeminivirus |
| Elderberry latent carmovirus | Tobacco mild green mosaic |
| Elm mottle ilarvirus | Tobacco mosaic satellivirus |
| Epirus cherry ourmiavirus | Tobacco mosaic tobamovirus |
| Erysimum latent tymovirus | Tobacco mottle umbravirus |
| Eucharis mottle nepovirus | Tobacco necrosis necrovirus |
| Euphorbia mosaic bigeminivirus | Tobacco necrosis satellivirus |
| Foxtail mosaic potexvirus | Tobacco necrotic dwarf luteovirus |
| Foxtail mosaic potexvirus | Tobacco rattle tobravirus |
| Foxtail mosaic potexvirus | Tobacco ringspot nepovirus |
| Frangipani mosaic tobamovirus | Tobacco streak ilarvirus |
| Furcraea necrotic streak | Tobacco stunt varicosavirus |
| Galinsoga mosaic carmovirus | Tobacco vein mottling potyvirus |
| Garlic common latent carlavirus | Tobacco vein-distorting luteovirus |
| Glycine mottle carmovirus | Tobacco wilt potyvirus |
| Grapevine A trichovirus | Tobacco yellow dwarf |
| Grapevine ajinashika disease | Tobacco yellow net luteovirus |
| Grapevine Algerian latent | Tobacco yellow vein umbravirus |
| Grapevine B trichovirus | Tobacco yellow vein assistor |
| Grapevine Bulgarian latent | Tomato aspermy cucumovirus |
| Grapevine chrome mosaic | Tomato Australian leafcurl |
| Grapevine chrome mosaic | Tomato black ring nepovirus |
| Grapevine corky bark-associated | Tomato black ring nepovirus |
| Grapevine fanleaf nepovirus | Tomato bushy stunt tombusvirus |
| Grapevine fleck virus | Tomato golden mosaic |
| Grapevine leafroll-associated | Tomato mild mottle potyvirus |
| Grapevine line pattern ilarvirus | Tomato mosaic tobamovirus |
| Grapevine stem pitting associated | Tomato mottle bigeminivirus |
| Grapevine stunt virus | Tomato Peru potyvirus |
| Groundnut chlorotic spot | Tomato ringspot nepovirus |
| Groundnut rosette umbravirus | Tomato spotted wilt tospovirus |
| Guar top necrosis virus | Tomato top necrosis nepovirus |
| Habenaria mosaic potyvirus | Tomato yellow leaf curl |
| Helenium S carlavirus | Tropaeolum 1 potyvirus |
| Henbane mosaic potyvirus | Tropaeolum 2 potyvirus |
| Heracleum latent trichovirus | Tulare apple mosaic ilarvirus |
| Hibiscus latent ringspot nepovirus | Tulip chlorotic blotch potyvirus |
| Hippeastrum mosaic potyvirus | Tulip halo necrosis virus |
| Honeysuckle latent carlavirus | Tulip X potexvirus |
| Hop American latent carlavirus | Turnip crinkle carmovirus |
| Hop latent carlavirus | Turnip mosaic potyvirus |
| Humulus japonicus ilarvirus | Turnip rosette sobemovirus |
| Hydrangea mosaic ilarvirus | Turnip yellow mosaic tymovirus |
| Impatiens latent potexvirus | Ullucus mild mottle tobamovirus |
| Impatiens necrotic spot tospovirus | Ullucus mosaic potyvirus |
| Iris fulva mosaic potyvirus | Vallota mosaic potyvirus |
| Ivy vein clearing cytorhabdovirus | Vanilla necrosis potyvirus |
| Johnsongrass mosaic potyvirus | Viola mottle potexvirus |
| Kalanchoe isometric virus | Viola mottle potexvirus |
| Konjak mosaic potyvirus | Watercress yellow spot virus |
| Kyuri green mottle mosaic | Watermelon mosaic 1 potyvirus |
| Lamium mild mottle fabavirus | Watermelon mosaic 2 potyvirus |
| Lato River tombusvirus | Weddel waterborne carmovirus |
| Leek yellow stripe potyvirus | Welsh onion yellow stripe |
| Lettuce big-vein varicosavirus | Wheat soil-borne mosaic furovirus |
| Lettuce infectious yellows | Wheat streak mosaic rymovirus |
| Lettuce mosaic potyvirus | White clover mosaic potexvirus |
| Lettuce necrotic yellows | Wild cucumber mosaic tymovirus |
| Lettuce speckles mottle umbravirus | Wild potato mosaic potyvirus |
| Lilac chlorotic leafspot capillovirus | Wild potato mosaic potyvirus |
| Lilac ring mottle ilarvirus | Wineberry latent virus |
| Lily X potexvirus | Wisteria vein mosaic potyvirus |
| Lisianthus necrosis necrovirus | Yam mosaic potyvirus |
| Lucerne Australian latent | Zygocactus Montana X potexvirus |
| nepovirus | |
| Lucerne Australian symptomless | |
| Lucerne enation nucleorhabdovirus | |

A further listing of plants and plant viruses that may used with the methods of the invention is shown in Table 4. Additional examples of virus infections of plant species can be found at: http://image.fs.uidaho.edu/vide/. Additional virus accessions can be retrieved at: http://www.atcc.org.

TABLE 4

| Plant or Virus Name | Plant or Virus Name |
|---|---|
| Cryptomeria japonica | Tulip band-breaking |
| Eucalyptus grandis | potyvirus |
| Eucalyptus nitens | Tulip breaking potyvirus |
| Eucalyptus urophylla | Tulip chlorotic blotch |
| Picea abies | potyvirus |
| Picea glauca | Tulip halo necrosis (?) virus |
| Pinus albicaulis | Tulip X potexvirus |
| Pinus aristata | Linum usitatissimum |
| Pinus armandii | Synonyms: |
| Pinus attenuata | Linum crepitans; Linum |
| Pinus ayacahuite | humile; Linum usitatissimum ssp. |
| Pinus balfouriana | transitorium; Linum usitatissimum |
| Pinus brutia | var. humile |
| Pinus bungeana | Common names: |
| Pinus canariensis | Flax; Linseed; Lino |
| Pinus cembroides | Susceptible to: |
| Pinus contorta | Alfalfa mosaic alfamovirus |
| Pinus culminicola | Beet curly top |
| Pinus durangensis | hybrigeminivirus |
| Pinus echinata | Beet pseudo-yellows (?) |
| Pinus edulis | closterovirus |
| Pinus elliottii | Oat blue dwarf marafivirus |
| Pinus engelmannii | Tobacco rattle tobravirus |
| Pinus flexilis | Hibiscus |
| Pinus gerardiana | Susceptible to: |
| Pinus griffithii | Abutilon mosaic |
| Pinus halepensis | bigeminivirus |
| Pinus hartwegii | Cotton leaf crumple |
| Pinus jefferyi | bigeminivirus |
| Pinus koraiensis | Hibiscus yellow mosaic (?) |
| Pinus lambertiana | tobamovirus |
| Pinus lumholtzii | Hibiscus cannabinus |
| Pinus massoniana | Common names: |
| Pinus monticola | Deccan-hemp; Indian-hemp; |
| Pinus mugo | Kenaf |
| Pinus palustris | Susceptible to: |
| Pinus pinaster | Cotton anthocyanosis (?) |
| Pinus pinceana | luteovirus |
| Pinus ponderosa | Cotton leaf crumple |
| Pinus pungens | bigeminivirus |
| Pinus radiata | Cotton leaf curl |
| Pinus resinosa | bigeminivirus |
| Pinus roxburghii | Hibiscus chlorotic ringspot |
| Pinus sabiniana | carmovirus |
| Pinus serotina | Hibiscus latent ringspot |
| Pinus strobus | nepovirus |
| Pinus sylvestris | Kenaf vein-clearing (?) |
| Pinus tabulaeformis | rhabdovirus |
| Pinus taeda | Malva vein clearing |
| Pinus thunbergii | potyvirus |
| Pinus torreyana | Okra mosaic tymovirus |
| Pinus virginiana | Ficus carica |
| Pinus wangii | Common names: |
| Pinus yunnanensis | Fig; Higo |
| Populus deltoides | Susceptible to: |
| Populus tremuloides | Fig (?) potyvirus |
| Cryptomeria japonica | Fig S carlavirus |
| Eucalyptus grandis | Morus alba |
| Eucalyptus nitens | Synonyms: |
| Eucalyptus urophylla | Morus alba f. tatarica; |
| Picea abies | Morus alba var. |
| Picea glauca | constantinopolitana; Morus alba |
| Pinus albicaulis | var. multicaulis; Morus indica; |
| Pinus aristata | Morus multicaulis |
| Pinus armandii | Common names: |
| Pinus attenuata | White mulberry; Mora |
| Pinus ayacahuite | Susceptible to: |
| Pinus balfouriana | Citrus enation- woody gall |
| Pinus brutia | (?) luteovirus |
| Pinus bungeana | Mulberry latent carlavirus |
| Pinus canariensis | Mulberry ringspot |
| Pinus cembroides | nepovirus |
| Pinus contorta | Mirabilis jalapa |
| Pinus culminicola | Common names: |
| Pinus durangensis | Common four-o'clock |
| Pinus echinata | Susceptible to: |
| Pinus edulis | Mirabilis mosaic |
| Pinus elliottii | caulimovirus |
| Pinus engelmannii | Fraxinus excelsior |
| Pinus flexilis | Synonyms: |
| Pinus gerardiana | Fraxinus excelsior var. |
| Pinus griffithii | pendula |
| Pinus halepensis | Common names: |
| Pinus hartwegii | European ash |
| Pinus jefferyi | Susceptible to: |
| Pinus koraiensis | Arabis mosaic nepovirus |
| Pinus lambertiana | Jasminum officinale |
| Pinus lumholtzii | Common names: |
| Pinus massoniana | Poet's jasmine; Common |
| Pinus monticola | jasmine; Jessamine |
| Pinus mugo | Susceptible to: |
| Pinus palustris | Arabis mosaic nepovirus |
| Pinus pinaster | Ligustrum vulgare |
| Pinus pinceana | Synonyms: |
| Pinus ponderosa | Ligustrum insulare; |
| Pinus pungens | Ligustrum insulense |
| Pinus radiata | Common names: |
| Pinus resinosa | Common privet |
| Pinus roxburghii | Susceptible to: |
| Pinus sabiniana | Arabis mosaic nepovirus |
| Pinus serotina | Petunia asteroid mosaic |
| Pinus strobus | tombusvirus |
| Pinus sylvestris | Olea europaea |
| Pinus tabulaeformis | Common names: |
| Pinus taeda | Olive; Aceituna |
| Pinus thunbergii | Susceptible to: |
| Pinus torreyana | Cherry leaf roll nepovirus |
| Pinus virginiana | Olive latent ringspot |
| Pinus wangii | nepovirus |
| Pinus yunnanensis | Olive latent 1 (?) |
| Populus deltoides | sobemovirus |
| Populus tremuloides | Olive latent 2 (?) |
| Populus trichocarpa | ourmavirus |
| Pseudotsuga menziesii | Oenothera biennis |
| Taxus brevifolia | Synonyms: |
| Ulmus parvifolia | Oenothera biennis ssp. |
| Chamaecyparis lawsoniana | sulfurea; Oenothera chicagoensis; |
| Common names: | Oenothera muricata; Oenothera |
| Port Orford-cedar; Ginger- | suaveolens; Onagra biennis |
| pine; Oregon-cedar; Lawson's | Common names: |
| cypress | Common evening-primrose; |
| Susceptible to: | German rampion |
| Arabis mosaic nepovirus | Insusceptible to: |
| Eucalyptus cloeziana | Carnation vein mottle |
| Common names: | potyvirus |
| Cloeziana gum; Gympie | Cymbidium |
| messmate | Susceptible to: |
| Populus balsamifera | Cymbidium mosaic |
| Susceptible to: | potexvirus |
| Poplar mosaic carlavirus | Cymbidium ringspot |
| Poplar vein yellowing (?) | tombusvirus |
| nucleorhabdovirus | Cymbidium alexanderi |
| Populus candicans | Susceptible to: |
| Synonyms: | Odontoglossum ringspot |
| Populus balsamifera ssp. | tobamovirus |
| balsamifera; Populus tacamahacca | Odontoglossum grande |
| Common names: | Synonyms: |
| Balsam poplar; Tacamahac | Rossioglossum grande |
| poplar; Balm of Gilead | Susceptible to: |
| Susceptible to: | Odontoglossum ringspot |
| Poplar mosaic carlavirus | tobamovirus |
| Populus deltoides subspecies | Cocos nucifera |
| angulata, monilifera, | Common names: |
| missouriensis | Coconut; Coconut palm; |
| Susceptible to: | Copra; Khopra; Nariyal; Coco |
| Poplar mosaic carlavirus | Susceptible to: |
| Ulmus americana | Coconut foliar decay |
| Common names: | nanavirus |
| American elm | Papaver nudicaule |
| Susceptible to: | Synonyms: |
| Cherry leaf roll nepovirus | Papaver miyabeanum |
| Ulmus glabra | Common names: |
| Synonyms: | Iceland poppy; Arctic poppy |
| Ulmus montana; Ulmus | Susceptible to: |
| scabra | Beet curly top |
| Common names: | hybrigeminivirus |
| Scotch elm; Wych elm | Tobacco mosaic |
| Susceptible to: | tobamovirus |
| Elm mottle ilarvirus | Tomato spotted wilt |
| Ulmus minor | tospovirus |
| Synonyms: | Turnip mosaic potyvirus |
| Ulmus campestris; Ulmus | Papaver somniferum |
| carpinifolia; Ulmus carpinifolia | Common names: |
| var. suberosa; Ulmus foliacea | Opium poppy |
| Ulmus foliacea var. suberosa; | Susceptible to: |
| Ulmus glabra var. | Bean yellow mosaic |
| suberosa; Ulmus nitens; | potyvirus |
| Ulmus suberosa | Papaver rhoeas |
| Susceptible to: | Common names: |
| Elm mottle ilarvirus | Corn poppy; Shirley poppy; |
| Subject: turf | Field poppy |
| Agropyron cristatum | Susceptible to: |
| Festuca arizonica | Beet western yellows |
| Agropyron cristatum x | clostreovirus |
| desertorum | Sesamum indicum |
| Festuca arundinacea | Synonyms: |
| Agropyron dasystachyum | Sesamum orientale |
| Festuca duriuscula | Common names: |
| Agropyron desertorum | Sesame; Benne seed |
| Festuca eliator | Susceptible to: |
| Agropyron elongatum | Abelia latent tymovirus |
| Festuca eliator | Apple stem pitting virus |
| arundinacea | Arracacha A nepovirus |
| Agropyron inerme | Asparagus 3 potexvirus |
| Festuca idahoensis | Asystasia gangetica mottle (?) |
| Agropyron intermedium | potyvirus |
| Festuca longifolia | Blackgram mottle (?) |
| Agropyron riparium | carmovirus |
| Festuca megalura | Cassia yellow spot |
| Agropyron sibericum | potyvirus |
| Festuca ovina | Cherry leaf roll nepovirus |
| Agropyron smithii | Citrus ringspot virus |
| Festuca rubra | Lisianthus necrosis (?) |
| Agropyron spicatum | necrovirus |
| Festuca rubra var. | Malva veinal necrosis (?) |
| commutata | potexvirus |
| Agropyron spicatum x | Melothria mottle (?) |
| repens | potyvirus |
| Festuca rubra var. rubra | Mulberry latent carlavirus |
| Agropyron trachycaulum | Mulberry ringspot |
| Hordeum brachyantherum | nepovirus |
| Agropyron trichophorum | Okra mosaic tymovirus |
| Koeleria cristata | Patchouli mottle (?) |
| Agrostis alba | potyvirus |
| Lolium multiflorum | Pea stem necrosis virus |
| Agrostis palustris | Peach enation (?) nepovirus |
| Lolium perenne | Peanut green mosaic |
| Agrostis tenuis | potyvirus |
| Oryzopsis hymenoides | Peanut mottle potyvirus |
| Alopecurus arundinaceus | Peanut stunt cucumovirus |
| Phalaris arundinacea | Satsuma dwarf (?) |
| Alopecurus pratensis | nepovirus |
| Phleum alpinum | Soybean mild mosaic virus |
| Arcatagrostis latifolia | Sweet potato yellow dwarf |
| Phleum pratense | (?) ipomovirus |
| Beckmannia syzigachne | Tobacco ringspot nepovirus |
| Phragmites australis | Watermelon mosaic 2 |
| Bromus biebersteinii | potyvirus |
| Poa alpina | Phytolacca americana |
| Bromus carinatus | Synonyms: |
| Poa ampla | Phytolacca decandra |
| Bromus catharticus | Common names: |
| Poa bulbosa | Pokeweed; Poke; |
| Bromus inermis | Pigeonberry |
| Poa canbyi | Susceptible to: |
| Bromus marginatus | Alfalfa mosaic alfamovirus |
| Poa compressa | Bean yellow mosaic |
| Bromus mollis | potyvirus |
| Poa glauca | Beet curly top |
| Dactylis glomerata | hybrigeminivirus |
| Poa palustris | Beet mosaic potyvirus |
| Deschampsia caespitosa | Carnation mottle |
| Poa pratensis | carmovirus |
| Viruses for Graminae: | Carnation ringspot |
| Maize streak monogeminivirus | dianthovirus |
| Wheat streak mosaic rymovirus | Cucumber mosaic |
| Barley yellow dwarf luteovirus | cucumovirus |
| Barley stripe mosaic hordeivirus | Cymbidium ringspot |
| Sugarcane mosaic potyvirus | tombusvirus |
| Beet western yellows luteovirus | Pepper veinal mottle |
| Maize dwarf mosaic potyvirus | potyvirus |
| Foxtail mosaic potexvirus | Pokeweed mosaic potyvirus |
| Johnsongrass mosaic potyvirus | Red clover necrotic mosaic |
| Panicum mosaic (?) sobemovirus | dianthovirus |
| Rice stripe tenuivirus | Tobacco rattle tobravirus |
| Rice hoja blanca tenuivirus | Tobacco ringspot nepovirus |
| Wheat yellow leaf closterovirus | Tomato black ring |
| Brome mosaic bromovirus | nepovirus |
| Ribgrass mosaic tobamovirus | Turnip mosaic potyvirus |
| Wheat soil-borne mosaic furovirus | Plantago major |
| Deschampsia caespitosa (L.) | Common names: |
| Beauv. ssp. Beringensis | Common plantain; |
| Poa sandbergii | Broadleaf plantain; Great plantain |
| Elymus angustus | Susceptible to: |
| Poa trivialis | Carnation vein mottle |
| Elymus canadensis | potyvirus |
| Puccinellia distans | Cherry rasp leaf nepovirus |
| Elymus cinereus | Plantago 4 (?) caulimovirus |
| Secale cereale | Plantago mottle tymovirus |
| Elymus dahuricus | Ribgrass mosaic |
| Sitanion hystrix | tobamovirus |
| Elymus glaucus | Phlox drummondii |
| Stipa comata | Common names: |
| Elymus junceus | Drummond phlox; Annual |
| Stipa viridula | phlox |
| Elymus triticoides | Susceptible to: |
| Triticum aestivum, spp. | Apple mosaic ilarvirus |
| WARM SEASON GRASSES | Arabis mosaic nepovirus |
| Andropogon geradii | Beet curly top |
| Distichlis stricta | hybrigeminivirus |
| Andropogon hallii | Beet western yellows |
| Panicum virgatum | luteovirus |
| Bouteloua curtipendula | Carnation ringspot |
| Schizachyrium scoparium | dianthovirus |
| Bouteloua gracillis | Cherry leaf roll nepovirus |
| Sorghastrum nutans | Cymbidium ringspot |
| Buchloe dactyloides | tombusvirus |
| Sporobolus airoides | Dogwood mosaic (?) |
| Calamovilfa longifolia | nepovirus |
| Sporobolus crypatandrus | Elm mottle ilarvirus |
| Cynodon dactylon | Melon Ourmia ourmiavirus |
| LEGUMES | Okra mosaic tymovirus |
| Astragalus cicer | Poplar mosaic carlavirus |
| Onobrychis viciaefolia | Prune dwarf ilarvirus |
| Coronilla varia | Ribgrass mosaic |
| Trifolium hybridum | tobamovirus |
| Hedysarum boreale | Spinach latent ilarvirus |
| Trifolium pratense | Strawberry latent ringspot |
| Lotus corniculatus | (?) nepovirus |
| Trifolium repens | Sweet potato mild mottle |
| Lupinus spp. | ipomovirus |
| Trifolium repens L. | Tobacco ringspot nepovirus |
| Medicago sativa | Tobacco streak ilarvirus |
| Vicia villosa | Tomato spotted wilt |
| Melilotus officinalis | tospovirus |
| Tritolium ambigium | Polypodium vulgare |
| Astragalus glycyphyllos | Susceptible to: |
| Common names: | Fern (?) potyvirus |
| Liquorice milk-vetch | rimula malacoides |
| Susceptible to: | Susceptible to: |
| Alfalfa mosaic alfamovirus | Carnation mottle |
| Astragalus sinicus | carmovirus |
| Susceptible to: | Hydrangea ringspot |
| Bean leaf roll luteovirus | potexvirus |
| Milk vetch dwarf nanavirus | Primula mottle (?) potyvirus |
| Soybean dwarf luteovirus | Sweet potato mild mottle |
| Subterranean clover red leaf | ipomovirus |
| luteovirus | Viola mottle potexvirus |
| Subterranean clover stunt | Pteris ‘Childsii’ |
| nanavirus | Susceptible to: |
| Watermelon mosaic 2 | Harts tongue fern (?) |
| potyvirus | tobravirus |
| Coronilla varia | Ranunculus repens |
| Synonyms: | Common names: |
| Securigera varia | Creeping buttercup |
| Common names: | Susceptible to: |
| Crown-vetch; Trailing | Arabis mosaic nepovirus |
| crown-vetch | Ranunculus repens |
| Susceptible to: | symptomless (?) rhabdovirus |
| Peanut stunt cucumovirus | Malus domestica |
| Trifolium hybridum | Synonyms: |
| Common names: | Malus malus; Pyrus malus |
| Alsike clover; Swedish | Common names: |
| clover; Trefle-hybride; Trefle- | Apple; Common apple |
| batard; Schwedenklee; | Susceptible to: |
| Bastardklee; Trevo-hibrido; | Apple mosaic ilarvirus |
| Trebol-hibrido | Insusceptible to: |
| Susceptible to: | Plum pox potyvirus |
| Alfalfa mosaic alfamovirus | Malus platycarpa |
| Alsike clover vein mosaic | Susceptible to: |
| virus | Apple chlorotic leaf spot |
| Bean leaf roll luteovirus | trichovirus |
| Bean yellow mosaic | Apple stem pitting virus |
| potyvirus | Malus sylvestris |
| Beet curly top | Common names: |
| hybrigeminivirus | Crab apple; Wild apple |
| Beet yellows closterovirus | Susceptible to: |
| Broad bean mottle | Apple chlorotic leaf spot |
| bromovirus | trichovirus |
| Broad bean stain comovirus | Apple stem grooving |
| Clover mild mosaic virus | capillovirus |
| Clover yellow mosaic | Apple stem pitting virus |
| potexvirus | Cherry rasp leaf nepovirus |
| Clover yellow vein | Horseradish latent |
| potyvirus | caulimovirus |
| Cucumber mosaic | Tomato ringspot nepovirus |
| cucumovirus | Tulare apple mosaic |
| Muskmelon vein necrosis | ilarvirus |
| carlavirus | Prunus avium |
| Pea early browning | Synonyms: |
| tobravirus | Cerasus avium var. |
| Pea enation mosaic | aspleniifolia; Prunus avium var. |
| enamovirus | aspleniifolia; Prunus cerasus var. |
| Pea streak carlavirus | avium |
| Peanut stunt cucumovirus | Common names: |
| Red clover mottle | Mazzard cherry; Sweet |
| comovirus | cherry |
| Red clover vein mosaic | Susceptible to: |
| carlavirus | Arabis mosaic nepovirus |
| Soybean dwarf luteovirus | Cherry leaf roll nepovirus |
| Subterranean clover red leaf | Cherry mottle leaf (?) |
| luteovirus | trichovirus |
| Tomato ringspot nepovirus | Cherry rasp leaf nepovirus |
| Turnip mosaic potyvirus | Epirus cherry ourmiavirus |
| White clover mosaic | Myrobalan latent ringspot |
| potexvirus | nepovirus |
| Lotus corniculatus | Petunia asteroid mosaic |
| Synonyms: | tombusvirus |
| Lotus corniculatus ssp. | Prunus domestica |
| major; Lotus corniculatus var. | Common names: |
| major; Lotus major | Plum |
| Common names: | Susceptible to: |
| Bird's-foot trefoil | Apple chlorotic leaf spot |
| Susceptible to: | trichovirus |
| Cucumber mosaic | Arabis mosaic nepovirus |
| cucumovirus | Citrus enation-woody gall |
| Lupinus albus | (?) luteovirus |
| Common names: | Petunia asteroid mosaic |
| White lupine; Egyptian | tombusvirus |
| lupine | Plum American line pattern |
| Susceptible to: | ilarvirus |
| Alfalfa mosaic alfamovirus | Plum pox potyvirus |
| Amaranthus leaf mottle | Prune dwarf ilarvirus |
| potyvirus | Sowbane mosaic |
| Bean common mosaic | sobemovirus |
| potyvirus | Strawberry latent ringspot |
| Bean yellow mosaic | (?) nepovirus |
| potyvirus | Prunus persica |
| Beet western yellows | Synonyms: |
| luteovirus | Amygdalus persica; |
| Bidens mosaic potyvirus | Amygdalus persica var. |
| Broad bean mottle | camelliiflora; Amygdalus persica |
| bromovirus | var. densa; Persica vulgaris; |
| Broad bean true mosaic | Prunus persica var. camelliiflora; |
| comovirus | Prunus persica var. densa |
| Carnation yellow stripe (?) | Common names: |
| necrovirus | Peach; Melocotonero; |
| Cassia mild mosaic (?) | Abridor; Durazno |
| carlavirus | Susceptible to: |
| Chicory yellow mottle | Apple chlorotic leaf spot |
| nepovirus | trichovirus |
| Cowpea chlorotic mottle | Arabis mosaic nepovirus |
| bromovirus | Cherry leaf roll nepovirus |
| Cucumber mosaic | Cherry mottle leaf (?) |
| cucumovirus | trichovirus |
| Dogwood mosaic (?) | Cherry rasp leaf nepovirus |
| nepovirus | Myrobalan latent ringspot |
| Epirus cherry ourmiavirus | nepovirus |
| Glycine mottle (?) | Peach enation (?) nepovirus |
| carmovirus | Peach rosette mosaic |
| Lucerne Australian latent | nepovirus |
| nepovirus | Peach yellow leaf (?) |
| Lucerne transient streak | closterovirus |
| sobemovirus | Plum American line pattern |
| Pea enation mosaic | ilarvirus |
| enamovirus | Plum pox potyvirus |
| Pea streak carlavirus | Prune dwarf ilarvirus |
| Peanut mottle potyvirus | Prunus necrotic ringspot |
| Peanut stunt cucumovirus | ilarvirus |
| Pepper Moroccan | Strawberry latent ringspot |
| tombusvirus | (?) nepovirus |
| Plum pox potyvirus | Tomato ringspot nepovirus |
| Prunus necrotic ringspot | Pyrus communis |
| ilarvirus | Synonyms: |
| Ribgrass mosaic | Pyrus asiae-mediae; Pyrus |
| tobamovirus | balansae; Pyrus bourgaeana; |
| Soybean dwarf luteovirus | Pyrus domestica; Pyrus elata; |
| Soybean mild mosaic virus | Pyrus medvedevii |
| Soybean mosaic potyvirus | Common names: |
| Subterranean clover red leaf | Pear; Pera |
| luteovirus | Susceptible to: |
| Turnip mosaic potyvirus | Apple chlorotic leaf spot |
| Watermelon mosaic 2 | trichovirus |
| potyvirus | Apple stem pitting virus |
| Wisteria vein mosaic | Rosa |
| potyvirus | Susceptible to: |
| Medicago sativa | Apple mosaic ilarvirus |
| Synonyms: | Arabis mosaic nepovirus |
| Medicago caerulea var. | Citrus enation - woody gall |
| pauciflora; Medicago | (?) luteovirus |
| karatschaica; Medicago lavrenkoi; | Prunus necrotic ringspot |
| Medicago pauciflora; Medicago | ilarvirus |
| sativa var. pilifera | Rose (?) tobamovirus |
| Susceptible to: | Strawberry latent ringspot |
| Alfalfa 1 alphacryptovirus | (?) nepovirus |
| Alfalfa 2 (?) betacryptovirus | Rubus fruticosus |
| Alfalfa mosaic alfamovirus | Synonyms: |
| Bean leaf roll luteovirus | Rubus plicatus; Rubus |
| Bean yellow mosaic | affinis |
| potyvirus | Common names: |
| Beet curly top | Blackberry; Bramble; |
| hybrigeminivirus | European blackberry |
| Broad bean mottle | Susceptible to: |
| bromovirus | Black raspberry necrosis |
| Carnation mottle | virus |
| carmovirus | Raspberry leaf curl (?) |
| Carrot mosaic (?) potyvirus | luteovirus |
| Cassia mild mosaic (?) | Strawberry latent ringspot |
| carlavirus | (?) nepovirus |
| Chickpea distortion mosaic | Rubus idaeus |
| potyvirus | Synonyms: |
| Clover yellow mosaic | Rubus buschii; Rubus |
| potexvirus | idaeus var. vulgatus; Rubus |
| Clover yellow vein | vulgatus var. buschii |
| potyvirus | Common names: |
| Cucumber mosaic | European red raspberry; |
| cucumovirus | Red raspberry |
| Lucerne Australian latent | Susceptible to |
| nepovirus | Arabis mosaic nepovirus |
| Lucerne Australian | Black raspberry necrosis |
| symptomless (?) nepovirus | virus |
| Lucerne enation (?) | Cherry leaf roll nepovirus |
| nucleorhabdovirus | Cole latent (?) carlavirus |
| Lucerne transient streak | Raspberry bushy dwarf |
| sobemovirus | idaeovirus |
| Milk vetch dwarf nanavirus | Raspberry leaf curl (?) |
| Narcissus mosaic potexvirus | luteovirus |
| Pea enation mosaic | Raspberry ringspot |
| enamovirus | nepovirus |
| Pea seed-borne mosaic | Raspberry vein chlorosis (?) |
| potyvirus | nucleorhabdovirus |
| Pea streak carlavirus | Rubus yellow net (?) |
| Peanut stunt cucumovirus | badnavirus |
| Red clover mottle | Strawberry latent ringspot |
| comovirus | (?) nepovirus |
| Red clover necrotic mosaic | Thimbleberry ringspot virus |
| dianthovirus | Tomato ringspot nepovirus |
| Red clover vein mosaic | Citrus limon |
| carlavirus | Synonyms: |
| Subterranean clover stunt | Citrus limonum; Citrus |
| nanavirus | medica var. limon |
| Tobacco ringspot nepovirus | Common names: |
| Tobacco streak ilarvirus | Lemon; Limonero; |
| Tobacco yellow dwarf | Limoniere; Citronnier; |
| monogeminivirus | Zitronenbaum |
| Watermelon mosaic 2 | Susceptible to: |
| potyvirus | Citrus enation - woody gall |
| White clover mosaic | (?) luteovirus |
| potexvirus | Citrus leaf rugose ilarvirus |
| Melilotus albus | Citrus ringspot virus |
| Synonyms: | Citrus tatter leaf capillovirus |
| Melilotus albus var. annuus; | Citrus tristeza closterovirus |
| Melilotus leucanthus | Citrus variegation ilarvirus |
| Common names: | Citrus paradisi |
| White sweet-clover; White | Common names: |
| melilot; Hubam | Grapefruit; Pomelo; Toronja |
| Susceptible to: | Susceptible to: |
| Alfalfa mosaic alfamovirus | Citrus enation - woody gall |
| Apple mosaic ilarvirus | (?) luteovirus |
| Bean common mosaic | Citrus leaf rugose ilarvirus |
| potyvirus | Citrus ringspot virus |
| Bean yellow mosaic | Citrus tristeza closterovirus |
| potyvirus | Pepper veinal mottle |
| Beet curly top | potyvirus |
| hybrigeminivirus | Citrus sinensis |
| Broad bean mottle | Synonyms: |
| bromovirus | Citrus aurantium var. |
| Broad bean necrosis | sinensis; Citrus macracantha |
| furovirus | Common names: |
| Broad bean stain comovirus | Sweet orange; Naranja |
| Broad bean true mosaic | Susceptible to: |
| comovirus | Citrus enation - woody gall |
| Clover yellow mosaic | (?) luteovirus |
| potexvirus | Citrus leaf rugose ilarvirus |
| Clover yellow vein | Citrus leprosis (?) |
| potyvirus | rhabdovirus |
| Cucumber mosaic | Citrus ringspot virus |
| cucumovirus | Citrus tatter leaf capillovirus |
| Galinsoga mosaic | Citrus tristeza closterovirus |
| carmovirus | Sambucus canadensis |
| Milk vetch dwarf nanavirus | Common names: |
| Muskmelon vein necrosis | American elder; American |
| carlavirus | elderberry; Sweet elder |
| Pea enation mosaic | Susceptible to: |
| enamovirus | Elderberry carlavirus |
| Pea mild mosaic comovirus | Elderberry latent (?) |
| Pea streak carlavirus | carmovirus |
| Peanut clump furovirus | Dodonaea viscosa |
| Peanut stunt cucumovirus | Common names: |
| Plum pox potyvirus | Hop shrub |
| Prune dwarf ilarvirus | Susceptible to: |
| Prunus necrotic ringspot | Dodonaea yellows- |
| ilarvirus | associated virus |
| Red clover mottle | Antirrhinum majus |
| comovirus | Common names: |
| Red clover vein mosaic | Snapdragon |
| carlavirus | Susceptible to: |
| Subterranean clover stunt | Alfalfa mosaic alfamovirus |
| nanavirus | Arabis mosaic nepovirus |
| Sweet clover latent (?) | Asystasia gangetica mottle |
| nucleorhabdovirus | (?) potyvirus |
| Sweet clover necrotic | Broad bean wilt fabavirus |
| mosaic dianthovirus | Carnation mottle |
| Tobacco etch potyvirus | carmovirus |
| Tobacco rattle tobravirus | Carnation ringspot |
| Tobacco ringspot nepovirus | dianthovirus |
| Tobacco streak ilarvirus | Cherry leaf roll nepovirus |
| Turnip mosaic potyvirus | Clover yellow vein |
| Watermelon mosaic 2 | potyvirus |
| potyvirus | Cowpea mosaic comovirus |
| White clover mosaic | Cucumber mosaic |
| potexvirus | cucumovirus |
| Trifolium dubium | Cymbidium ringspot |
| Synonyms: | tombusvirus |
| Trifolium filiforme var. | Dogwood mosaic (?) |
| dubium; Trifolium minus; | nepovirus |
| Trifolium parviflorum; Trifolium | Elm mottle ilarvirus |
| procumbens | Groundnut eyespot |
| Common names: | potyvirus |
| Small hop clover; Suckling | Maracuja mosaic (?) |
| clover; Lesser yellow trefoil; Low | tobamovirus |
| hop clover; Yellow clover; | Marigold mottle potyvirus |
| Shamrock | Papaya mosaic potexvirus |
| Susceptible to: | Pea streak carlavirus |
| Alfalfa mosaic alfamovirus | Peanut clump furovirus |
| Bean leaf roll luteovirus | Pepper Moroccan |
| Peanut stunt cucumovirus | tombusvirus |
| Soybean dwarf luteovirus | Plantago mottle tymovirus |
| Subterranean clover stunt | Poplar mosaic carlavirus |
| nanavirus | Prune dwarf ilarvirus |
| WETLAND - RIPARIAN | Prunus necrotic ringspot |
| Agrostis alba | ilarvirus |
| Glyceria occidentalis | Red clover necrotic mosaic |
| Alopecurus arundinaceus | dianthovirus |
| Glyceria striata | Red clover vein mosaic |
| Alopecurus pratensis | carlavirus |
| Hordeum brachyantherum | Rubus Chinese seed-borne |
| Beckmannia syzigachne | (?) nepovirus |
| Phalaris arundinacea | Scrophularia mottle |
| Deschampsia caespitosa | tymovirus |
| Poa palustris | Soybean mild mosaic virus |
| WILDFLOWERS AND | Soybean mosaic potyvirus |
| FORBES | Spinach latent ilarvirus |
| Achillea millefolium | Strawberry latent ringspot |
| Lupinus albicalus | (?) nepovirus |
| Cheiranthus allionii | Tamus latent (?) potexvirus |
| Lupinus perennis | Tobacco necrosis necrovirus |
| Coreopsis lanceolata | Tobacco rattle tobravirus |
| Papaver rhoeas | Tobacco ringspot nepovirus |
| Echinacea purpurea | Tobacco streak ilarvirus |
| Ratibida columnaris | Tomato black ring |
| Eschscholtzia californica | nepovirus |
| Rudbeckia hirta | Tomato bushy stunt |
| Linum lewisii | tombusvirus |
| Lupinus luteus | Viola mottle potexvirus |
| Common names: | White clover mosaic |
| European yellow lupine; | potexvirus |
| Yellow lupine | Scrophularia nodosa |
| Susceptible to: | Common names: |
| Bean yellow mosaic | Figwort; Figwort herb |
| potyvirus | Susceptible to: |
| Clover yellow vein | Scrophularia mottle |
| potyvirus | tymovirus |
| Dogwood mosaic (?) | Capsicum annuum |
| nepovirus | Synonyms: |
| Peanut stunt cucumovirus | Capsicum cordiforme |
| Cheiranthus cheiri | Common names: |
| Synonyms: | Pimiento; Bell pepper; |
| Erysimum cheiri | Cayenne pepper; Chili pepper; |
| Common names: | Common garden pepper; Green |
| Wallflower | pepper; Mango pepper; Paprika |
| Susceptible to: | pepper |
| Alfalfa mosaic alfamovirus | Susceptible to: |
| Beet western yellows | Alfalfa mosaic alfamovirus |
| luteovirus | Bean distortion dwarf (?) |
| Chicory yellow mottle | bigeminivirus |
| nepovirus | Beet western yellows |
| Cucumber mosaic | luteovirus |
| cucumovirus | Cassia mild mosaic (?) |
| Tobacco rattle tobravirus | carlavirus |
| Tobacco ringspot nepovirus | Celery latent (?) potyvirus |
| Tomato spotted wilt | Chilli veinal mottle (?) |
| tospovirus | potyvirus |
| Turnip crinkle carmovirus | Chino del tomat, |
| Turnip mosaic potyvirus | bigeminivirus |
| Turnip yellow mosaic | Cucumber mosaic |
| tymovirus | cucumovirus |
| Coreopsis lanceolata | Datura distortion mosaic |
| Susceptible to: | potyvirus |
| Bidens mosaic potyvirus | Eggplant mosaic tymovirus |
| Papaver rhoeas | Eggplant mottled dwarf |
| Common names: | nucleorhabdovirus |
| Corn poppy; Shirley poppy; | Eggplant severe mottle (?) |
| Field poppy | potyvirus |
| Susceptible to: | Henbane mosaic potyvirus |
| Beet western yellows | Marigold mottle potyvirus |
| clostrovirus | Melon Ourmia ourmiavirus |
| Linum grandiflorum | Paprika mild mottle |
| Synonyms: | tobamovirus |
| Linum rubrum | Peanut stunt cucumovirus |
| Common names: | Pelargonium vein clearing (?) |
| Flowering flax | cytorhabdovirus |
| Susceptible to: | Pepper hausteco |
| Beet pseudo-yellows (?) | bigeminivirus |
| closterovirus | Pepper Indian mottle |
| Oat blue dwarf marafivirus | potyvirus |
| Linum usitatissimum | Pepper mild mosaic (?) |
| Synonyms: | potyvirus |
| Linum crepitans; Linum | Pepper mild mottle |
| humile; Linum usitatissimum ssp. | tobamovirus |
| transitorium; Linum usitatissimum | Pepper mild tigr, (?) |
| var. humile | bigeminivirus |
| Common names: | Pepper Moroccan |
| Flax; Linseed; Lino | tombusvirus |
| Susceptible to: | Pepper mottle potyvirus |
| Alfalfa mosaic alfamovirus | Pepper ringspot tobravirus |
| Beet curly top | Pepper severe mosaic |
| hybrigeminivirus | potyvirus |
| Beet pseudo-yellows (?) | Pepper Texas bigeminivirus |
| closterovirus | Pepper veinal mottle |
| Oat blue dwarf marafivirus | Potyvirus |
| Tobacco rattle tobravirus | Physalis mosaic tymovirus |
| ORNAMENTAL GRASSES | Pittosporum vein yellowing |
| Acorus Gramineus | nucleorhabdovirus |
| Acorus Calamus | Potato aucuba mosaic |
| Acorus Gramineus | potexvirus |
| Alopecurus Pratensis | Potato mop-top furovirus |
| Andropogon Scoparius | Potato Y potyvirus |
| Andropogon Gerardii | Red pepper 1 (?) |
| Arrhenatherum Elatius | alphacryptovirus |
| Arundo Formosana | Red pepper 2 (?) |
| Briza Media | alphacryptovirus |
| Calamagrostis Acutiflora | Ribgrass mosaic |
| Calamagrostis Arundinacea | tobamovirus |
| Calamagrostis Acutiflora | Serrano golden mosaic |
| Calamagrostis Acutiflora | bigeminivirus |
| Carex Glauca | Sweet potato ringspot (?) |
| Carex Siderostica | nepovirus |
| Carex Albula | Tobacco etch potyvirus |
| Carex Nigra | Tobacco leaf curl |
| Carex Muskingumensis | bigeminivirus |
| Carex Riparia | Tobacco mild green mosaic |
| Carex Evergold | tobamovirus |
| Carex Comans | Tobacco mosaic satellivirus |
| Cortaderia Selloana | Tobacco rattle tobravirus |
| Cortaderia Selloana Rosea | Tobacco streak ilarvirus |
| Deschampsia Cespitosa | Tomato bushy stunt |
| Elymus Arenarius | tombusvirus |
| Erianthus Ravennae | Tomato mosaic tobamovirus |
| Ovina Gigantea | Tomato Peru potyvirus |
| Ovina Glauca | Tomato spotted wilt |
| Glyceria Maxima | tospovirus |
| Hakonechloa Macra | Lycopersicon esculentum |
| Hakonechloa Macra | Common names: |
| Helictotrichon Sempervirens | Tomato; Tomate |
| Holcus Variegated | Susceptible to: |
| Hystrix Patula | Abelia latent tymovirus |
| Imperata Red Baron | Abutilon mosaic |
| Juncus Effusus | bigeminivirus |
| Juncus Ensifolius | Alfalfa mosaic alfamovirus |
| Juncus Filiformis | Arabis mosaic nepovirus |
| Juncus Inflexus | Arracacha A nepovirus |
| Koeleria Cristata | Arracacha B (?) nepovirus |
| Koeleria Glauca | Beet curly top |
| Luzula Sylvatica | hybrigeminivirus |
| Melica Ciliata | Beet western yellows |
| Melica Nutans | luteovirus |
| Miscanthus Sinensis | Blueberry leaf mottle |
| Molinia Caerulea | nepovirus |
| Virgatum Rotstrahlbusch | Brinjal mild mosaic (?) |
| Pennisetum Alopecuroides | potyvirus |
| Pennisetum Ruppelianum | Carnation mottle |
| Pennisetum Alopecuroides | carmovirus |
| Pennisetum Alopecuroides | Carrot mosaic (?) potyvirus |
| Pennisetum Alopecuroides | Cassava green mottle |
| Pennisetum Setaceum | nepovirus |
| Pennisetum Setaceum | Cassia mild mosaic (?) |
| Pennisetum Cassian | carlavirus |
| Phalaris Arundinacea | Chickpea chlorotic dwarf (?) |
| Phalaris Arundinacea | monogeminivirus |
| Phalaris Arundinacea | Chino del tomat, |
| Sesleria Autumnalis | bigeminivirus |
| Sesleria Caerulea | Clover wound tumor |
| Sporobolus Helerolepsis | phytoreovirus |
| Stipa Capillata | Commelina X potexvirus |
| Stipa Extremiorientalis | Cowpea mild mottle (?) |
| Stipa Gigantea | carlavirus |
| Stipa Tenuissima | Croton yellow vein mosaic |
| Stipa Grandis | bigeminivirus |
| Stipa Pennata | Cucumber mosaic |
| Stipa Ucrainica | cucumovirus |
| Impatiens | Cymbidium ringspot |
| Impatiens necrotic spot tospovirus | tombusvirus |
| Carnation mottle carmovirus | Datura distortion mosaic |
| Helenium S carlavirus | potyvirus |
| Impatiens latent (?) potexvirus | Datura innoxia Hungarian |
| Aster chlorotic stunt (?) carlavirus | mosaic (?) potyvirus |
| Dasheen mosaic potyvirus | Datura mosaic (?) potyvirus |
| Aglaonema | Datura necrosis potyvirus |
| Alocasia | Datura yellow vein |
| Amorphophallus | nucleorhabdovirus |
| Arisaema | Dogwood mosaic (?) |
| Caladium hortulanum | nepovirus |
| Chenopodium amaranticolor | Dulcamara mottle |
| Chenopodium ambrosioides | tymovirus |
| Chenopodium quinoa | Eggplant green mosaic |
| Colocasia esculenta | potyvirus |
| Cryptocoryne | Eggplant mosaic tymovirus |
| Cyrtosperma | Eggplant mottled dwarf |
| Dieffenbachia picta | nucleorhabdovirus |
| Nicotiana benthamiana | Eggplant severe mottle (?) |
| Philodendron selloum | potyvirus |
| Philodendron verrucosum | Elderberry latent (?) |
| Richardia | carmovirus |
| Saponaria vaccaria | Elm mottle ilarvirus |
| Spathiphyllum | Epirus cherry ourmiavirus |
| Tetragonia tetragonioides | Foxtail mosaic potexvirus |
| Xanthosoma caracu | Groundnut eyespot |
| Zantedeschia (no species name | potyvirus |
| provided) | Henbane mosaic potyvirus |
| Zantedeschia elliottiana | Lettuce necrotic yellows |
| Colocasia bobone disease (?) | cytorhabdovirus |
| rhabdovirus | Maracuja mosaic (?) |
| Dasheen bacilliform (?) | tobamovirus |
| badnavirus | Marigold mottle potyvirus |
| Dasheen mosaic potyvirus | Melilotus mosaic (?) |
| Colocasia esculenta | potyvirus |
| Konjak mosaic (?) potyvirus | Melon Ourmia ourmiavirus |
| Philodendron | Nerine X potexvirus |
| oxycardium | Okra leaf-curl bigeminivirus |
| Philodendron selloum | Ononis yellow mosaic |
| Abelia latent tymovirus | tymovirus |
| Abelia grandiflora | Parietaria mottle ilarvirus |
| Abelmoschus esculentus | Parsnip yellow fleck |
| Acer palmatum | sequivirus |
| Amaranthus caudatus | Pea streak carlavirus |
| Atropa belladonna | Peanut clump furovirus |
| Brassica campestris ssp. | Peanut stunt cucumovirus |
| pekinensis | Pelargonium line pattern (?) |
| Catharanthus roseus | carmovirus |
| Celosia argentea | Pelargonium zonate spot |
| Chenopodium amaranticolor | ourmiavirus |
| Chenopodium murale | Pepino mosaic potexvirus |
| Chenopodium quinoa | Pepper Indian mottle |
| Datura metel | potyvirus |
| Datura stramonium | Pepper mild tigr, (?) |
| Glycine max | bigeminivirus |
| Gomphrena globosa | Pepper Moroccan |
| Gossypium hirsutum | tombusvirus |
| Hordeum vulgare | Pepper mottle potyvirus |
| Lobelia erinus | Pepper ringspot tobravirus |
| Lycopersicon esculentum | Pepper severe mosaic |
| Momordica balsamina | potyvirus |
| Nicotiana clevelandii | Pepper Texas bigeminivirus |
| Nicotiana glutinosa | Pepper veinal mottle |
| Nicotiana rustica | potyvirus |
| Petunia x hybrida | Physalis mosaic tymovirus |
| Physalis peruviana | Pittosporum vein yellowing |
| Sesamum indicum | nucleorhabdovirus |
| Solanum melongena | Plantain X potexvirus |
| Solanum tuberosum | Plum pox potyvirus |
| Tetragonia tetragonioides | Potato 14R (?) tobamovirus |
| Tithonia speciosa | Potato Andean latent |
| Torenia fournieri | tymovirus |
| Vicia faba | Potato Andean mottle |
| Allium | comovirus |
| Susceptible to: | Potato aucuba mosaic |
| Onion yellow dwarf | potexvirus |
| potyvirus | Potato black ringspot |
| Allium ampeloprasum var. | nepovirus |
| holmense | Potato leafroll luteovirus |
| Garlic common latent (?) | Potato M carlavirus |
| carlavirus | Potato mop-top furovirus |
| Allium ampeloprasum var. | Potato U nepovirus |
| sectivum | Potato V potyvirus |
| Susceptible to: | Potato Y potyvirus |
| Sint-Jan's onion latent (?) | Potato yellow mosaic |
| carlavirus | bigeminivirus |
| Allium cepa | Raspberry ringspot |
| Synonyms: | nepovirus |
| Allium ascalonicum; Allium | Red clover necrotic mosaic |
| cepa var. aggregatum; Allium | dianthovirus |
| cepa var. solaninum | Ribgrass mosaic |
| Common names: | tobamovirus |
| Onion; Shallot; Tama-negi; | Rose (?) tobamovirus |
| Eschalot; Potato onion; Multiplier | Rubus Chinese seed-borne (?) |
| onion; Cebolla; Spanish onion | nepovirus |
| Susceptible to: | Serrano golden mosaic |
| Leek yellow stripe potyvirus | bigeminivirus |
| Onion mite-borne latent (?) | Solanum apical leaf curling (?) |
| potexvirus | bigeminivirus |
| Onion yellow dwarf | Soybean crinkle leaf (?) |
| potyvirus | bigeminivirus |
| Pepper venial mottle | Soybean mild mosaic virus |
| potyvirus | Strawberry latent ringspot (?) |
| Shallot latent carlavirus | nepovirus |
| Shallot mite-borne latent (?) | Sunflower ringspot (?) |
| potexvirus | ilarvirus |
| Shallot yellow stripe (?) | Sweet potato mild mottle |
| potyvirus | ipomovirus |
| Sint-Jan's onion latent (?) | Tamarillo mosaic potyvirus |
| carlavirus | Tamus latent (?) potexvirus |
| Tobacco rattle tobravirus | Tobacco etch potyvirus |
| Welsh onion yellow stripe (?) | Tobacco leaf curl |
| potyvirus | bigeminivirus |
| Amaranthaceae | Tobacco mild green mosaic |
| Susceptible to: | tobamovirus |
| Apple stem grooving | Tobacco mosaic satellivirus |
| capillovirus | Tobacco mosaic |
| Insusceptible to: | tobamovirus |
| Voandzeia necrotic mosaic | Tobacco mottle umbravirus |
| tymovirus | Tobacco necrosis necrovirus |
| Amaranthus bicolor | Tobacco necrotic dwarf |
| Insusceptible to: | luteovirus |
| Onion mite-borne latent (?) | Tobacco rattle tobravirus |
| potexvirus | Tobacco ringspot nepovirus |
| Amaranthus caudatus | Tobacco streak ilarvirus |
| Synonyms: | Tobacco stunt varicosavirus |
| Amaranthus caudatus ssp. | Tobacco vein-distorting (?) |
| mantegazzianus; Amaranthus | luteovirus |
| caudatus var. alopecurus; | Tobacco vein mottling |
| Amaranthus dussii; Amaranthus | potyvirus |
| edulis; Amaranthus | Tobacco yellow dwarf |
| mantegazzianus | monogeminivirus |
| Common names: | Tobacco yellow net (?) |
| Inca wheat; Love-lies- | luteovirus |
| bleeding; Tassel-flower; Kiwichi; | Tobacco yellow vein |
| Coimi | assistor (?) luteovirus |
| Susceptible to: | Tobacco yellow vein (?) |
| Abelia latent tymovirus | umbravirus |
| Alfalfa mosaic alfamovirus | Tomato aspermy |
| Amaranthus leaf mottle | cucumovirus |
| potyvirus | Tomato Australian leafcurl |
| Amaranthus mosaic (?) | bigeminivirus |
| potyvirus | Tomato black ring |
| Arracacha A nepovirus | nepovirus |
| Arracacha B (?) nepovirus | Tomato bushy stunt |
| Bean yellow mosaic | tombusvirus |
| potyvirus | Tomato chlorotic spot (?) |
| Beet curly top | tospovirus |
| hybrigeminivirus | Tomato golden mosaic |
| Beet mosaic potyvirus | bigeminivirus |
| Cactus X potexvirus | Tomato infectious chlorosis (?) |
| Carnation mottle | closterovirus |
| carmovirus | Tomato mild mottle (?) |
| Carnation ringspot | potyvirus |
| dianthovirus | Tomato mosaic tobamovirus |
| Carnation vein mottle | Tomato mottle |
| potyvirus | bigeminivirus |
| Celery latent (?) potyvirus | Tomato Peru potyvirus |
| Chicory yellow mottle | Tomato pseudo curly top (?) |
| nepovirus | hybrigeminivirus |
| Clover yellow mosaic | Tomato ringspot nepovirus |
| potexvirus | Tomato spotted wilt |
| Clover yellow vein | tospovirus |
| potyvirus | Tomato top necrosis (?) |
| Cucumber mosaic | nepovirus |
| cucumovirus | Tomato vein clearing |
| Cymbidium ringspot | nucleorhabdovirus |
| tombusvirus | Tomato yellow leaf curl |
| Dahlia mosaic caulimovirus | bigeminivirus |
| Elderberry carlavirus | Tomato yellow mosaic |
| Grapevine fanleaf nepovirus | bigeminivirus |
| Heracleum latent trichovirus | Tulip chlorotic blotch |
| Humulus japonicus ilarvirus | potyvirus |
| Iris fulva mosaic potyvirus | Tulip X potexvirus |
| Lamium mild mottle | Turnip crinkle carmovirus |
| fabavirus | Ullucus mild mottle |
| Lettuce mosaic potyvirus | tobamovirus |
| Maclura mosaic | White clover mosaic |
| macluravirus | potexvirus |
| Marigold mottle potyvirus | Wild potato mosaic |
| Peanut stunt cucumovirus | potyvirus |
| Plantain X potexvirus | Wineberry latent virus |
| Potato 14R (?) tobamovirus | Nicotiana benthamiana |
| Potato Andean latent | Susceptible to: |
| tymovirus | Ahlum waterborne (?) |
| Potato black ringspot | carmovirus |
| nepovirus | Alstroemeria (?) ilarvirus |
| Potato leafroll luteovirus | Alstroemeria mosaic |
| Red clover necrotic mosaic | potyvirus |
| dianthovirus | Alstroemeria streak (?) |
| Ribgrass mosaic | potyvirus |
| tobamovirus | Amazon lily mosaic (?) |
| Telfairia mosaic potyvirus | potyvirus |
| Tobacco etch potyvirus | Apple mosaic ilarvirus |
| Tobacco necrosis necrovirus | Arracacha Y potyvirus |
| Tobacco rattle tobravirus | Artichoke latent potyvirus |
| Tobacco ringspot nepovirus | Artichoke latent S (?) |
| Tobacco streak ilarvirus | carlavirus |
| Tomato black ring | Artichoke mottled crinkle |
| nepovirus | tombusvirus |
| Tomato spotted wilt | Artichoke vein banding (?) |
| tospovirus | nepovirus |
| Turnip mosaic potyvirus | Asparagus 3 potexvirus |
| Ullucus mild mottle | Asystasia gangetica mottle |
| tobamovirus | (?) potyvirus |
| Viola mottle potexvirus | Barley yellow streak mosaic |
| Watermelon mosaic 2 | virus |
| potyvirus | Bean calico mosaic |
| Zygocactus Montana X (?) | bigeminivirus |
| potexvirus | Bean common mosaic |
| Amaranthus tricolor | potyvirus |
| Synonyms: | Beet curly top |
| Amaranthus gangeticus; | hybrigeminivirus |
| Amaranthus gangeticus var. | Blueberry leaf mottle |
| melancholicus; Amaranthus | nepovirus |
| mangostanus; Amaranthus | Blueberry necrotic shock |
| polygamus; Amaranthus | ilarvirus |
| tricolor ssp. mangostanus; | Caper latent carlavirus |
| Amaranthus tricolor ssp. tristis | Caraway latent (?) |
| Common names: | nepovirus |
| Chinese amaranth; | Carrot mottle mimic |
| Tampala; Ganges amaranth | umbravirus |
| Susceptible to: | Carrot mottle umbravirus |
| Amaranthus leaf mottle | Carrot yellow leaf (?) |
| potyvirus | closterovirus |
| Amaranthus mosaic (?) | Cassava African mosaic |
| potyvirus | bigeminivirus |
| Apple mosaic ilarvirus | Cassava brown streak- |
| Amaryllis | associated (?) carlavirus |
| Susceptible to: | Cassava brown streak |
| Amaryllis (?) | potyvirus |
| alphacryptovirus | Cassava Caribbean mosaic (?) |
| Narcissus | potexvirus |
| Susceptible to: | Cassava Colombian |
| Narcissus yellow stripe | symptomless (?) potexvirus |
| potyvirus | Cassava common mosaic (?) |
| Insusceptible to: | potexvirus |
| Silene X (?) potexvirus | Cassava green mottle |
| Narcissus jonquilla | nepovirus |
| Common names: | Cassava Indian mosaic |
| Jonquil | bigeminivirus |
| Susceptible to: | Cassava Ivorian bacilliform |
| Strawberry latent ringspot | ourmiavirus |
| (?) nepovirus | Cassava X potexvirus |
| Insusceptible to: | Cherry leaf roll nepovirus |
| Ornithogalum mosaic | Chickpea bushy dwarf |
| potyvirus | potyvirus |
| Narcissus poeticus | Chickpea chlorotic dwarf (?) |
| Common names: | monogeminivirus |
| Narcissus; Pheasant's-eye; | Chickpea distortion mosaic |
| Poet's narcissus | potyvirus |
| Susceptible to: | Chicory yellow mottle |
| Narcissus tip necrosis (?) | nepovirus |
| carmovirus | Chino del tomat, |
| Narcissus pseudonarcissus | bigeminivirus |
| Common names: | Citrus ringspot virus |
| Daffodil; Common daffodil | Cowpea chlorotic mottle |
| Susceptible to: | bromovirus |
| Arabis mosaic nepovirus | Croton yellow vein mosaic |
| Narcissus late season | bigeminivirus |
| yellows (?) potyvirus | Cucumber necrosis |
| Narcissus latent | tombusvirus |
| macluravirus | Cymbidium ringspot |
| Narcissus mosaic potexvirus | tombusvirus |
| Narcissus tip necrosis (?) | Cynara (?) |
| carmovirus | nucleorhabdovirus |
| Raspberry ringspot | Dandelion yellow mosaic |
| nepovirus | sequivirus |
| Tobacco rattle tobravirus | Dasheen mosaic potyvirus |
| Tomato black ring | Desmodium mosaic |
| nepovirus | potyvirus |
| Yucca | Dioscorea green banding |
| Susceptible to: | mosaic potyvirus |
| Furcraea necrotic streak (?) | Dioscorea latent (?) |
| dianthovirus | potexvirus |
| Chlorophytum comosum | Dogwood mosaic (?) |
| Common names: | nepovirus |
| Spider plant; Spider-ivy; | Eggplant green mosaic |
| Ribbon plant | potyvirus |
| Insusceptible to: | Eggplant mottled dwarf |
| Onion mite-borne latent (?) | nucleorhabdovirus |
| potexvirus | Eggplant severe mottle (?) |
| Shallot mite-borne latent (?) | potyvirus |
| potexvirus | Elderberry latent (?) |
| Sint-Jan's onion latent (?) | carmovirus |
| carlavirus | Epirus cherry ourmiavirus |
| Tradescantia-Zebrina | Euphorbia mosaic |
| potyvirus | bigeminivirus |
| Catharanthus roseus | Grapevine A (?) trichovirus |
| Synonyms: | Grapevine Algerian latent |
| Ammocallis rosea; | tombusvirus |
| Lochnera rosea; Vinca rosea | Grapevine Bulgarian latent |
| Common names: | nepovirus |
| Bright-eyes; Madagascar | Grapevine chrome mosaic |
| periwinkle; Old-maid; Rose | nepovirus |
| periwinkle; Rosy periwinkle | Grapevine fanleaf nepovirus |
| Susceptible to: | Groundnut chlorotic spot (?) |
| Abelia latent tymovirus | potexvirus |
| Alfalfa mosaic alfamovirus | Groundnut rosette |
| Apple mosaic ilarvirus | umbravirus |
| Bean pod mottle comovirus | Hibiscus latent ringspot |
| Beet curly top | nepovirus |
| hybrigeminivirus | Hydrangea mosaic ilarvirus |
| Belladonna mottle | Ivy vein clearing (?) |
| tymovirus | cytorhabdovirus |
| Cacao yellow mosaic | Kalanchoe isometric virus |
| tymovirus | Lato River tombusvirus |
| Carnation mottle | Lettuce big-vein |
| carmovirus | varicosavirus |
| Cassava green mottle | Lettuce mosaic potyvirus |
| nepovirus | Lilac chlorotic leafspot |
| Cherry leaf roll nepovirus | capillovirus |
| Citrus leaf rugose ilarvirus | Lily X potexvirus |
| Citrus ringspot virus | Lucerne Australian |
| Clover wound tumor | symptomless (?) nepovirus |
| phytoreovirus | Maracuja mosaic (?) |
| Clover yellow mosaic | tobamovirus |
| potexvirus | Melon Ourmia ourmiavirus |
| Cowpea severe mosaic | Melothria mottle (?) |
| comovirus | potyvirus |
| Cucumber mosaic | Nandina mosaic (?) |
| cucumovirus | potexvirus |
| Dogwood mosaic (?) | Narcissus latent |
| nepovirus | macluravirus |
| Dulcamara mottle | Narcissus tip necrosis (?) |
| tymovirus | carmovirus |
| Elm mottle ilarvirus | Neckar River tombusvirus |
| Erysimum latent tymovirus | Nerine potyvirus |
| Foxtail mosaic potexvirus | Nicotiana velutina mosaic (?) |
| Humulus japonicus ilarvirus | furovirus |
| Lilac ring mottle ilarvirus | Oat golden stripe furovirus |
| Nandina mosaic (?) | Okra mosaic tymovirus |
| potexvirus | Olive latent 1 (?) |
| Narcissus mosaic potexvirus | sobemovirus |
| Okra mosaic tymovirus | Olive latent 2 (?) |
| Pea seed-borne mosaic | ourmiavirus |
| potyvirus | Paprika mild mottle |
| Peach enation (?) nepovirus | tobamovirus |
| Peanut stunt cucumovirus | Parsnip yellow fleck |
| Pepper ringspot tobravirus | sequivirus |
| Pepper veinal mottle | Passiflora ringspot |
| potyvirus | potyvirus |
| Plum American line pattern | Peanut chlorotic streak |
| ilarvirus | caulimovirus |
| Poplar mosaic carlavirus | Peanut clump furovirus |
| Potato 14R (?) tobamovirus | Peanut green mosaic |
| Potato black ringspot | potyvirus |
| nepovirus | Peanut yellow spot |
| Potato T trichovirus | tospovirus |
| Prune dwarf ilarvirus | Pelargonium vein clearing (?) |
| Prunus necrotic ringspot | cytorhabdovirus |
| ilarvirus | Pepper Moroccan |
| Scrophularia mottle | tombusvirus |
| tymovirus | Pepper mottle potyvirus |
| Spring beauty latent | Pepper ringspot tobravirus |
| bromovirus | Pepper Texas bigeminivirus |
| Tobacco mosaic satellivirus | Pepper veinal mottle |
| Tobacco necrosis necrovirus | potyvirus |
| Tobacco rattle tobravirus | Physalis mosaic tymovirus |
| Tobacco ringspot nepovirus | Pittosporum vein yellowing |
| Tobacco streak ilarvirus | nucleorhabdovirus |
| Tobacco stunt varicosavirus | Plantain 6 (?) carmovirus |
| Tomato spotted wilt | Plantain 7 (?) potyvirus |
| tospovirus | Plantain X potexvirus |
| Tulare apple mosaic | Plum American line pattern |
| ilarvirus | ilarvirus |
| Turnip crinkle carmovirus | Plum pox potyvirus |
| Watermelon mosaic 2 | Poinsettia mosaic (?) |
| potyvirus | tymovirus |
| Wild cucumber mosaic | Potato 14R (?) tobamovirus |
| tymovirus | Potato Andean latent |
| Hedera helix | tymovirus |
| Common names: | Potato Andean mottle |
| English ivy | comovirus |
| Susceptible to: | Potato black ringspot |
| Ivy vein clearing (?) | nepovirus |
| cytorhabdovirus | Potato mop-top furovirus |
| sparagus officinalis | Potato T trichovirus |
| Synonyms: | Prune dwarf ilarvirus |
| Asparagus longifolius | Prunus necrotic ringspot |
| Common names: | ilarvirus |
| Garden asparagus; | Red clover necrotic mosaic |
| Asparagus; Esparrag | dianthovirus |
| Susceptible to: | Rice stripe necrosis (?) |
| Arabis mosaic nepovirus | furovirus |
| Asparagus 1 potyvirus | Rubus Chinese seed-borne (?) |
| Asparagus 2 ilarvirus | nepovirus |
| Strawberry latent ringspot (?) | Silene X (?) potexvirus |
| nepovirus | Sitke waterborne (?) |
| Tobacco streak ilarvirus | tombusvirus |
| Dryopteris filix-mas | Solanum apical leaf curling (?) |
| Common names: | bigeminivirus |
| Male fern | Solanum nodiflorum mottle |
| Susceptible to: | sobemovirus |
| Fern (?) potyvirus | Sonchus yellow net |
| Polystichum falcatum | nucleorhabdovirus |
| Susceptible to: | Soybean mosaic potyvirus |
| Harts tongue fern (?) | Sweet potato feathery |
| tobravirus | mottle potyvirus |
| Phyllitis scolopendrium | Sweet potato latent (?) |
| Synonyms: | potyvirus |
| Asplenium scolopendrium | Sweet potato mild mottle |
| Common names: | ipomovirus |
| Hart's-tongue fern | Sweet potato ringspot (?) |
| Susceptible to: | nepovirus |
| Harts tongue fern (?) | Sweet potato sunken vein (?) |
| tobravirus | closterovirus |
| Aucuba japonica | Tamus latent (?) potexvirus |
| Synonyms: | Telfairia mosaic potyvirus |
| Aucuba japonica var. | Tobacco mosaic satellivirus |
| variegata | Tobacco mosaic |
| Common names: | tobamovirus |
| Spotted-laurel; Japanese- | Tobacco rattle tobravirus |
| laurel | Tobacco streak ilarvirus |
| Susceptible to: | Tobacco stunt varicosavirus |
| Aucuba ringspot (?) | Tomato Australian leafcurl |
| badnavirus | bigeminivirus |
| Cycas necrotic stunt | Tomato bushy stunt |
| nepovirus | tombusvirus |
| Begonia elatior | Tomato golden mosaic |
| Susceptible to: | bigeminivirus |
| Carnation mottle | Tomato mild mottle (?) |
| carmovirus | potyvirus |
| Begonia x tuberhybrida | Tomato mosaic tobamovirus |
| Common names: | Tomato mottle |
| Hybris tuberous begonia | bigeminivirus |
| Insusceptible to: | Tomato ringspot nepovirus |
| Aster chlorotic stunt (?) | Tomato yellow leaf curl |
| carlavirus | bigeminivirus |
| Catalpa bignonioides | Tomato yellow mosaic |
| Synonyms: | bigeminivirus |
| Catalpa bignonioides f. | Tropaeolum 1 potyvirus |
| aurea | Tropaeolum 2 potyvirus |
| Common names: | Tulip chlorotic blotch |
| Catawba; Common catalpa; | potyvirus |
| Indian-bean; Southern catalpa; | Tulip halo necrosis (?) virus |
| Cigartree; Smoking-bean | Tulip X potexvirus |
| Susceptible to: | Ullucus mild mottle |
| Scrophularia mottle | tobamovirus |
| tymovirus | Ullucus mosaic potyvirus |
| Acer palmatum | Vanilla necrosis potyvirus |
| Abelia latent tymovirus | Watercress yellow spot |
| Betula | virus |
| Susceptible to: | Watermelon mosaic 2 |
| Cherry leaf roll nepovirus | potyvirus |
| Ceiba pentandra | Weddel waterborne (?) |
| Synonyms: | carmovirus |
| Bombax pentandrum; Ceiba | Wild potato mosaic |
| casearia; Eriodendron | potyvirus |
| anfractuosum | Yam mosaic potyvirus |
| Common names: | Nicotiana tabacum |
| Ceiba; Kapok; Silk-cotton- | Synonyms: |
| tree; White silk-cotton-tree; | Nicotiana chinensis; |
| Kapokbaum; Kapokier; Arbe- | Nicotiana tabacum var. |
| kapok | macrophylla |
| Susceptible to: | Common names: |
| Cacao swollen shoot | Tobacco |
| badnavirus | Susceptible to: |
| Cacao yellow mosaic | Abutilon mosaic |
| tymovirus | bigeminivirus |
| Okra mosaic tymovirus | Alfalfa mosaic alfamovirus |
| Myosotis sylvatica | Alstroemeria (?) ilarvirus |
| Synonyms: | Alstroemeria mosaic |
| Myosotis alpestris; | potyvirus |
| Myosotis oblongata | Amaranthus leaf mottle |
| Common names: | potyvirus |
| Garden forget-me-not; | Arabis mosaic nepovirus |
| Wood forget-me-not | Arracacha A nepovirus |
| Susceptible to: | Arracacha B (?) nepovirus |
| Arabis mosaic nepovirus | Arracacha Y potyvirus |
| Carnation ringspot | Artichoke Italian latent |
| dianthovirus | nepovirus |
| Cymbidium ringspot | Artichoke yellow ringspot |
| tombusvirus | nepovirus |
| Tobacco rattle tobravirus | Asparagus 2 ilarvirus |
| Tobacco ringspot nepovirus | Asparagus 3 potexvirus |
| Tomato black ring | Asystasia gangetica mottle |
| nepovirus | (?) potyvirus |
| Ananas comosus | Barley stripe mosaic |
| Synonyms: | hordeivirus |
| Ananas duckei; Ananas | Bean distortion dwarf (?) |
| sativus; Ananas sativus var. | bigeminivirus |
| duckei; Bromelia ananas; | Bean yellow mosaic |
| Bromelia comosa | potyvirus |
| Common names: | Beet curly top |
| Pineapple; Pina | hybrigeminivirus |
| Susceptible to: | Beet pseudo-yellows (?) |
| Pineapple chlorotic leaf | closterovirus |
| streak (?) nucleorhabdovirus | Belladonna mottle |
| Pineapple wilt-associated | tymovirus |
| (?) closterovirus | Bidens mosaic potyvirus |
| Tomato spotted wilt | Blueberry leaf mottle |
| tospovirus | nepovirus |
| Buxus sempervirens | Blueberry necrotic shock |
| Synonyms: | ilarvirus |
| Buxus colchica | Bramble yellow mosaic (?) |
| Common names: | potyvirus |
| Boxwood; Common | Broad bean wilt fabavirus |
| boxwood; Turkish boxwood | Burdock yellow mosaic (?) |
| Susceptible to: | potexvirus |
| Arabis mosaic nepovirus | Cacao necrosis nepovirus |
| Cactaceae family | Cacao yellow mosaic |
| Including: | tymovirus |
| Austrocylindropuntia cylindrica | Carnation ringspot |
| Cactaceae | dianthovirus |
| Carnegiea gigantea (syn. Cereus | Cassava African mosaic |
| giganteus) | bigeminivirus |
| Saguaro; Giant cactus | Cassava green mottle |
| Cereus | nepovirus |
| Chamaecereus sylvestrii | Cassava Indian mosaic |
| Echinocereus procumbens | bigeminivirus |
| Echinopsis | Cassava Ivorian bacilliform |
| Epiphyllum | ourmiavirus |
| Ferocactus acanthodes (syn. | Cassia mild mosaic (?) |
| Echinocactus acanthodes) | carlavirus |
| Opuntia engelmannii | Cassia severe mosaic (?) |
| Opuntia vulgaris (syn. Cactus | closterovirus |
| monacanthos; Opuntia | Celery latent (?) potyvirus |
| monacantha) | Cherry leaf roll nepovirus |
| Prickly-pear cactus; Tuna; | Chickpea chlorotic dwarf (?) |
| Prickly-pear; Drooping prickly- | monogeminivirus |
| pear | Chicory yellow mottle |
| Pereskia saccharosa | nepovirus |
| Schlumbergera bridgesii | Chilli veinal mottle (?) |
| Zygocactus | potyvirus |
| Zygocactus truncatus | Chino del tomat, |
| Zygocactus x Schlumbergera | bigeminivirus |
| Susceptible to: | Citrus ringspot virus |
| Cactus X potexvirus | Clover wound tumor |
| Cactus 2 carlavirus | phytoreovirus |
| Lobelia erinus | Clover yellow vein |
| Common names: | potyvirus |
| Edging lobelia | Commelina X potexvirus |
| Susceptible to: | Cowpea chlorotic mottle |
| Abelia latent tymovirus | bromovirus |
| Arabis mosaic nepovirus | Cowpea mosaic comovirus |
| Carnation ringspot | Cowpea mottle (?) |
| dianthovirus | carmovirus |
| Cherry leaf roll nepovirus | Cowpea severe mosaic |
| Elm mottle ilarvirus | comovirus |
| Peanut stunt cucumovirus | Croton yellow vein mosaic |
| Strawberry latent ringspot | bigeminivirus |
| (?) nepovirus | Cucumber green mottle |
| Tobacco rattle tobravirus | mosaic tobamovirus |
| Tomato black ring | Cucumber mosaic |
| nepovirus | cucumovirus |
| Humulus japonicus | Cucumber necrosis |
| Synonyms: | tombusvirus |
| Humulus scandens | Cymbidium ringspot |
| Common names: | tombusvirus |
| Japanese hop | Datura Colombian potyvirus |
| Susceptible to: | Datura distortion mosaic |
| Hop latent carlavirus | potyvirus |
| Humulus japonicus ilarvirus | Datura innoxia Hungarian |
| Lonicera | mosaic (?) potyvirus |
| Susceptible to: | Datura mosaic (?) potyvirus |
| Eggplant mottled dwarf | Datura necrosis potyvirus |
| nucleorhabdovirus | Datura shoestring potyvirus |
| Pittosporum vein yellowing | Datura yellow vein |
| nucleorhabdovirus | nucleorhabdovirus |
| Insusceptible to: | Dioscorea latent (?) |
| Tomato yellow leaf curl | potexvirus |
| bigeminivirus | Dogwood mosaic (?) |
| Lonicera americana | nepovirus |
| Susceptible to: | Eggplant green mosaic |
| Honeysuckle latent | potyvirus |
| carlavirus | Eggplant mild mottle (?) |
| Carica papaya | carlavirus |
| Synonyms: | Eggplant mottled crinkle |
| Carica peltata; Carica | tombusvirus |
| posoposa; Papaya carica | Eggplant mottled dwarf |
| Common names: | nucleorhabdovirus |
| Papaya; Pawpaw | Eggplant severe mottle (?) |
| Susceptible to: | potyvirus |
| Croton yellow vein mosaic | Elderberry latent (?) |
| bigeminivirus | carmovirus |
| Papaya mosaic potexvirus | Elm mottle ilarvirus |
| Papaya ringspot potyvirus | Epirus cherry ourmiavirus |
| Watermelon mosaic 1 | Eucharis mottle (?) |
| potyvirus | nepovirus |
| Dianthus barbatus | Foxtail mosaic potexvirus |
| Common names: | Frangipani mosaic |
| Sweet William | tobamovirus |
| Susceptible to: | Galinsoga mosaic |
| Alfalfa mosaic alfamovirus | carmovirus |
| Arabis mosaic nepovirus | Grapevine Bulgarian latent |
| Beet curly top | nepovirus |
| hybrigeminivirus | Grapevine chrome mosaic |
| Beet mosaic potyvirus | nepovirus |
| Carnation latent carlavirus | Grapevine fanleaf nepovirus |
| Carnation mottle | Guar top necrosis virus |
| carmovirus | Henbane mosaic potyvirus |
| Carnation necrotic fleck | Hibiscus latent ringspot |
| closterovirus | nepovirus |
| Carnation (?) rhabdovirus | Hippeastrum mosaic |
| Carnation ringspot | potyvirus |
| dianthovirus | Hop American latent |
| Carnation vein mottle | carlavirus |
| potyvirus | Humulus japonicus ilarvirus |
| Carnation yellow stripe (?) | Ivy vein clearing (?) |
| necrovirus | cytorhabdovirus |
| Clover wound tumor | Kalanchoe isometric virus |
| phytoreovirus | Kyuri green mottle mosaic |
| Melon Ourmia ourmiavirus | tobamovirus |
| Okra mosaic tymovirus | Lamium mild mottle |
| Peanut stunt cucumovirus | fabavirus |
| Pelargonium line pattern (?) | Lilac chlorotic leafspot |
| carmovirus | capillovirus |
| Potato black ringspot | Lilac ring mottle ilarvirus |
| nepovirus | Lisianthus necrosis (?) |
| Potato M carlavirus | necrovirus |
| Silene X (?) potexvirus | Lucerne Australian latent |
| Strawberry latent ringspot (?) | nepovirus |
| (?) nepovirus | Lucerne Australian |
| Tobacco ringspot nepovirus | symptomless (?) nepovirus |
| Tomato bushy stunt | Lucerne transient streak |
| tombusvirus | sobemovirus |
| Viola mottle potexvirus | Lychnis ringspot |
| Dianthus caryophyllus | hordeivirus |
| Common names: | Maclura mosaic |
| Carnation; Clavel | macluravirus |
| Susceptible to: | Maracuja mosaic (?) |
| Alfalfa mosaic alfamovirus | tobamovirus |
| Arabis mosaic nepovirus | Marigold mottle potyvirus |
| Beet curly top | Melandrium yellow fleck |
| hybrigeminivirus | bromovirus |
| Carnation 1 | Melilotus mosaic (?) |
| alphacryptovirus | potyvirus |
| Carnation 2 (?) | Melon Ourmia ourmiavirus |
| alphacryptovirus | Milk vetch dwarf nanavirus |
| Carnation etched ring | Myrobalan latent ringspot |
| caulimovirus | nepovirus |
| Carnation Italian ringspot | Narcissus latent |
| tombusvirus | macluravirus |
| Carnation latent carlavirus | Neckar River tombusvirus |
| Carnation mottle | Nerine potyvirus |
| carmovirus | Nicotiana velutina mosaic (?) |
| Carnation necrotic fleck | furovirus |
| Closterovirus | Odontoglossum ringspot |
| Carnation (?) rhabdovirus | tobamovirus |
| Carnation ringspot | Okra leaf-curl bigeminivirus |
| dianthovirus | Olive latent 1 (?) |
| Carnation vein mottle | sobemovirus |
| Potyvirus | Olive latent 2 (?) |
| Carnation yellow stripe (?) | ourmiavirus |
| necrovirus | Orchid fleck (?) rhabdovirus |
| Lettuce infectious yellows | Paprika mild mottle |
| (?) closterovirus | tobamovirus |
| Melandrium yellow fleck | Parietaria mottle ilarvirus |
| bromovirus | Parsnip yellow fleck |
| Potato M carlavirus | sequivirus |
| Tobacco stunt varicosavirus | Passionfruit woodiness |
| Gypsophila elegans | potyvirus |
| Common names: | Patchouli mosaic potyvirus |
| Baby's-breath | Pea early browning |
| Susceptible to: | tobravirus |
| Belladonna mottle | Pea mosaic potyvirus |
| tymovirus | Pea streak carlavirus |
| Lychnis ringspot | Peach enation (?) nepovirus |
| hordeivirus | Peach rosette mosaic |
| Tobacco etch potyvirus | nepovirus |
| Tobacco necrosis necrovirus | Peanut chlorotic streak |
| Tobacco rattle tobravirus | caulimovirus |
| Tobacco ringspot nepovirus | Peanut clump furovirus |
| Tomato bushy stunt | Peanut stunt cucumovirus |
| tombusvirus | Pelargonium line pattern (?) |
| Euonymus europaeus | carmovirus |
| Synonyms: | Pelargonium vein clearing |
| Euonymus vulgaris | (?) cytorhabdovirus |
| Common names: | Pelargonium zonate spot |
| European spindletree; | ourmiavirus |
| Spindletree | Pepino mosaic potexvirus |
| Susceptible to: | Pepper Indian mottle |
| Arabis mosaic nepovirus | potyvirus |
| Strawberry latent ringspot (?) | Pepper mild mosaic (?) |
| nepovirus | potyvirus |
| Euonymus japonica | Pepper mild mottle |
| Susceptible to: | tobamovirus |
| Euonymus fasciation (?) | Pepper Moroccan |
| rhabdovirus | tombusvirus |
| Euonymus (?) rhabdovirus | Pepper mottle potyvirus |
| Beta vulgaris | Pepper ringspot tobravirus |
| Common names: | Pepper severe mosaic |
| Beet | potyvirus |
| Susceptible to: | Pepper Texas bigeminivirus |
| Alfalfa mosaic alfamovirus | Pepper veinal mottle |
| Arabis mosaic nepovirus | potyvirus |
| Arracacha A nepovirus | Physalis mosaic tymovirus |
| Asparagus 2 ilarvirus | Pittosporum vein yellowing |
| Asparagus 3 potexvirus | nucleorhabdovirus |
| Barley stripe mosaic | Plantain X potexvirus |
| hordeivirus | Plum American line pattern |
| Beet 1 alphacryptovirus | ilarvirus |
| Beet 2 alphacryptovirus | Plum pox potyvirus |
| Beet 3 alphacryptovirus | Poinsettia mosaic (?) |
| Beet curly top | tymovirus |
| hybrigeminivirus | Poplar mosaic carlavirus |
| Beet distortion mosaic virus | Potato 14R (?) tobamovirus |
| Beet leaf curl (?) | Potato A potyvirus |
| rhabdovirus | Potato Andean mottle |
| Beet mild yellowing | comovirus |
| luteovirus | Potato aucuba mosaic |
| Beet mosaic potyvirus | potexvirus |
| Beet necrotic yellow vein | Potato black ringspot |
| furovirus | nepovirus |
| Beet pseudo-yellows (?) | Potato mop-top furovirus |
| closterovirus | Potato T trichovirus |
| Beet soil-borne furovirus | Potato U nepovirus |
| Beet western yellows | Potato V potyvirus |
| luteovirus | Potato X potexvirus |
| Beet yellow net (?) | Potato Y potyvirus |
| luteovirus | Potato yellow dwarf |
| Beet yellow stunt | nucleorhabdovirus |
| closterovirus | Primula mosaic potyvirus |
| Beet yellows closterovirus | Primula mottle (?) potyvirus |
| Broad bean wilt fabavirus | Prune dwarf ilarvirus |
| Butterbur mosaic (?) | Radish mosaic comovirus |
| carlavirus | Raspberry ringspot |
| Cacao necrosis nepovirus | nepovirus |
| Cacao yellow mosaic | Red clover necrotic mosaic |
| tymovirus | dianthovirus |
| Cactus X potexvirus | Red clover vein mosaic |
| Caraway latent (?) | carlavirus |
| nepovirus | Rhynchosia mosaic |
| Carnation latent carlavirus | bigeminivirus |
| Carnation mottle | Ribgrass mosaic |
| carmovirus | tobamovirus |
| Carnation vein mottle | Rose (?) tobamovirus |
| potyvirus | Rubus Chinese seed-borne |
| Celery latent (?) potyvirus | (?) nepovirus |
| Cherry leaf roll nepovirus | Silene X (?) potexvirus |
| Chickpea chlorotic dwarf | Solanum nodiflorum mottle |
| (?) monogeminivirus | sobemovirus |
| Chicory yellow blotch (?) | Sonchus cytorhabdovirus |
| carlavirus | Sowbane mosaic |
| Clover yellow mosaic | sobemovirus |
| potexvirus | Soybean crinkle leaf (?) |
| Clover yellow vein | bigeminivirus |
| potyvirus | Soybean mild mosaic virus |
| Cowpea chlorotic mottle | Soybean mosaic potyvirus |
| bromovirus | Spinach latent ilarvirus |
| Cowpea mild mottle (?) | Strawberry latent ringspot (?) |
| carlavirus | nepovirus |
| Croton yellow vein mosaic | Sunn-hemp mosaic |
| bigeminivirus | tobamovirus |
| Cucumber mosaic | Sweet clover necrotic |
| cucumovirus | mosaic dianthovirus |
| Cucumber soil-borne | Sweet potato latent (?) |
| carmovirus | potyvirus |
| Cycas necrotic stunt | Sweet potato mild mottle |
| nepovirus | ipomovirus |
| Cymbidium ringspot | Sweet potato ringspot (?) |
| tombusvirus | nepovirus |
| Dogwood mosaic (?) | Tamarillo mosaic potyvirus |
| nepovirus | Telfairia mosaic potyvirus |
| Elderberry carlavirus | Tobacco etch potyvirus |
| Elderberry latent (?) | Tobacco leaf curl |
| carmovirus | bigeminivirus |
| Elm mottle ilarvirus | Tobacco mild green mosaic |
| Epirus cherry ourmiavirus | tobamovirus |
| Foxtail mosaic potexvirus | Tobacco mosaic satellivirus |
| Grapevine Bulgarian latent | Tobacco mosaic |
| nepovirus | tobamovirus |
| Grapevine fanleaf nepovirus | Tobacco mottle umbravirus |
| Groundnut eyespot | Tobacco necrosis necrovirus |
| potyvirus | Tobacco necrosis |
| Helenium S carlavirus | satellivirus |
| Heracleum latent trichovirus | Tobacco necrotic dwarf |
| Humulus japonicus ilarvirus | luteovirus |
| Impatiens latent (?) | Tobacco rattle tobravirus |
| potexvirus | Tobacco ringspot nepovirus |
| Lettuce infectious yellows | Tobacco streak ilarvirus |
| (?) closterovirus | Tobacco stunt varicosavirus |
| Lettuce mosaic potyvirus | Tobacco vein-distorting (?) |
| Lettuce speckles mottle | luteovirus |
| umbravirus | Tobacco vein mottling |
| Lilac chlorotic leafspot | potyvirus |
| capillovirus | Tobacco wilt potyvirus |
| Marigold mottle potyvirus | Tobacco yellow dwarf |
| Mulberry latent carlavirus | monogeminivirus |
| Odontoglossum ringspot | Tobacco yellow net (?) |
| tobamovirus | luteovirus |
| Parsnip leafcurl virus | Tobacco yellow vein |
| Parsnip yellow fleck | assistor (?) luteovirus |
| sequivirus | Tobacco yellow vein (?) |
| Pea seed-borne mosaic | umbravirus |
| potyvirus | Tomato aspermy |
| Peanut clump furovirus | cucumovirus |
| Peanut stunt cucumovirus | Tomato Australian leafcurl |
| Pelargonium line pattern (?) | bigeminivirus |
| carmovirus | Tomato black ring |
| Pepper ringspot tobravirus | nepovirus |
| Physalis mild chlorosis (?) | Tomato bushy stunt |
| luteovirus | tombusvirus |
| Potato 14R (?) tobamovirus | Tomato golden mosaic |
| Potato black ringspot | bigeminivirus |
| nepovirus | Tomato mild mottle (?) |
| Potato M carlavirus | potyvirus |
| Potato mop-top furovirus | Tomato mosaic tobamovirus |
| Potato T trichovirus | Tomato mottle |
| Potato U nepovirus | bigeminivirus |
| Radish mosaic comovirus | Tomato Peru potyvirus |
| Raspberry ringspot | Tomato ringspot nepovirus |
| nepovirus | Tomato spotted wilt |
| Red clover necrotic mosaic | tospovirus |
| dianthovirus | Tomato top necrosis (?) |
| Ribgrass mosaic | nepovirus |
| tobamovirus | Tomato yellow leaf curl |
| Rubus Chinese seed-borne | bigeminivirus |
| (?) nepovirus | Tomato yellow mosaic |
| Sowbane mosaic | bigeminivirus |
| sobemovirus | Tulare apple mosaic |
| Soybean dwarf luteovirus | ilarvirus |
| Spinach latent ilarvirus | Tulip chlorotic blotch |
| Strawberry latent ringspot | potyvirus |
| (?) nepovirus | Tulip halo necrosis (?) virus |
| Subterranean clover red leaf | Turnip mosaic potyvirus |
| luteovirus | Turnip rosette sobemovirus |
| Sunn-hemp mosaic | Ullucus mild mottle |
| tobamovirus | tobamovirus |
| Sweet potato mild mottle | Ullucus mosaic potyvirus |
| ipomovirus | Watermelon mosaic 2 |
| Tobacco etch potyvirus | potyvirus |
| Tobacco mosaic | Wild potato mosaic |
| tobamovirus | potyvirus |
| Tobacco necrosis necrovirus | Wisteria vein mosaic |
| Tobacco rattle tobravirus | potyvirus |
| Tobacco ringspot nepovirus | Petunia x hybrida |
| Tobacco streak ilarvirus | Common names: |
| Tobacco stunt varicosavirus | Common garden petunia; |
| Tobacco yellow dwarf | Garden petunia |
| monogeminivirus | Susceptible to: |
| Tomato black ring | Abelia latent tymovirus |
| nepovirus | Alfalfa mosaic alfamovirus |
| Tulip halo necrosis (?) virus | Alstroemeria (?) ilarvirus |
| Tulip X potexvirus | Alstroemeria mosaic |
| Turnip mosaic potyvirus | potyvirus |
| Viola mottle potexvirus | Amaranthus leaf mottle |
| Spinacia oleracea | potyvirus |
| Common names: | Amaranthus mosaic (?) |
| Spinach | potyvirus |
| Susceptible to: | Aquilegia (?) potyvirus |
| Alfalfa mosaic alfamovirus | Arabis mosaic nepovirus |
| Amaranthus leaf mottle | Arracacha A nepovirus |
| potyvirus | Arracacha B (?) nepovirus |
| Arabis mosaic nepovirus | Artichoke latent potyvirus |
| Asparagus 3 potexvirus | Artichoke vein banding (?) |
| Barley stripe mosaic | nepovirus |
| hordeivirus | Artichoke yellow ringspot |
| Bean yellow mosaic | nepovirus |
| potyvirus | Asparagus 2 ilarvirus |
| Beet curly top | Bean yellow mosaic |
| hybrigeminivirus | potyvirus |
| Beet leaf curl (?) | Beet curly top |
| rhabdovirus | hybrigeminivirus |
| Beet mild yellowing | Beet western yellows |
| luteovirus | luteovirus |
| Beet mosaic potyvirus | Bidens mottle potyvirus |
| Beet necrotic yellow vein | Black raspberry necrosis |
| furovirus | virus |
| Beet pseudo-yellows (?) | Brinjal mild mosaic (?) |
| closterovirus | potyvirus |
| Beet soil-borne furovirus | Broad bean V (?) potyvirus |
| Beet western yellows | Broad bean wilt fabavirus |
| luteovirus | Butterbur mosaic (?) |
| Beet yellows closterovirus | carlavirus |
| Black raspberry necrosis | Cacao necrosis nepovirus |
| virus | Caper latent carlavirus |
| Broad bean wilt fabavirus | Carnation mottle |
| Canavalia maritima mosaic | carmovirus |
| (?) potyvirus | Cassava green mottle |
| Carnation mottle | nepovirus |
| carmovirus | Cassava Indian mosaic |
| Carnation ringspot | bigeminivirus |
| dianthovirus | Cassava Ivorian bacilliform |
| Carnation vein mottle | ourmiavirus |
| potyvirus | Celery latent (?) potyvirus |
| Celery latent (?) potyvirus | Cherry leaf roll nepovirus |
| Cherry leaf roll nepovirus | Chicory yellow mottle |
| Clover yellow mosaic | nepovirus |
| potexvirus | Chrysanthemum B |
| Clover yellow vein | carlavirus |
| potyvirus | Citrus ringspot virus |
| Cowpea mild mottle (?) | Cowpea chlorotic mottle |
| Carlavirus | bromovirus |
| Cowpea mosaic comovirus | Cowpea mosaic comovirus |
| Croton yellow vein mosaic | Cowpea severe mosaic |
| bigeminivirus | comovirus |
| Cumcumber leaf spot | Croton yellow vein mosaic |
| carmovirus | bigeminivirus |
| Cucumber mosaic | Cucumber leaf spot |
| cucumovirus | carmovirus |
| Cycas necrotic stunt | Cymbidium ringspot |
| nepovirus | tombusvirus |
| Cymbidium ringspot | Datura distortion mosaic |
| tombusvirus | potyvirus |
| Dandelion yellow mosaic | Datura innoxia Hungarian |
| sequivirus | mosaic (?) potyvirus |
| Daphne Y potyvirus | Datura mosaic (?) potyvirus |
| Dogwood mosaic (?) | Dogwood mosaic (?) |
| nepovirus | nepovirus |
| Elderberry latent (?) | Eggplant green mosaic |
| carmovirus | potyvirus |
| Elm mottle ilarvirus | Eggplant mosaic tymovirus |
| Epirus cherry ourmiavirus | Eggplant mottled dwarf |
| Foxtail mosaic potexvirus | nucleorhabdovirus |
| Galinsoga mosaic | Elderberry latent (?) |
| carmovirus | carmovirus |
| Habenaria mosaic (?) | Elm mottle ilarvirus |
| potyvirus | Epirus cherry ourmiavirus |
| Heracleum latent trichovirus | Galinsoga mosaic |
| Lettuce infectious yellows | carmovirus |
| (?) closterovirus | Grapevine chrome mosaic |
| Lettuce mosaic potyvirus | nepovirus |
| Lettuce necrotic yellows | Grapevine fanleaf nepovirus |
| cytorhabdovirus | Groundnut eyespot |
| Lettuce speckles mottle | potyvirus |
| umbravirus | Guar top necrosis virus |
| Lucerne Australian latent | Henbane mosaic potyvirus |
| nepovirus | Hibiscus latent ringspot |
| Lucerne Australian | nepovirus |
| symptomless (?) nepovirus | Hibiscus yellow mosaic (?) |
| Lucerne transient streak | tobamovirus |
| sobemovirus | Hippeastrum mosaic |
| Lychnis ringspot | potyvirus |
| hordeivirus | Honeysuckle latent |
| Melon Ourmia ourmiavirus | carlavirus |
| Melothria mottle (?) | Humulus japonicus ilarvirus |
| potyvirus | Kyuri green mottle mosaic |
| Milk vetch dwarf nanavirus | tobamovirus |
| Mulberry latent carlavirus | Lamium mild mottle |
| Nandina mosaic (?) | fabavirus |
| potexvirus | Lettuce infectious yellows |
| Nicotiana velutina mosaic | (?) closterovirus |
| (?) furovirus | Lettuce necrotic yellows |
| Oat blue dwarf marafivirus | cytorhabdovirus |
| Okra mosaic tymovirus | Lilac chlorotic leafspot |
| Parietaria mottle ilarvirus | capillovirus |
| Parsnip leafcurl virus | Lilac mottle carlavirus |
| Parsnip mosaic potyvirus | Lisianthus necrosis (?) |
| Parsnip yellow fleck | necrovirus |
| sequivirus | Lucerne Australian |
| Patchouli mosaic potyvirus | symptomless (?) nepovirus |
| Pea early browning | Lucerne transient streak |
| tobravirus | sobemovirus |
| Pea streak carlavirus | Lychnis ringspot |
| Peanut chlorotic streak | hordeivirus |
| caulimovirus | Marigold mottle potyvirus |
| Peanut clump furovirus | Melandrium yellow fleck |
| Peanut mottle potyvirus | bromovirus |
| Peanut stunt cucumovirus | Melilotus mosaic (?) |
| Pelargonium flower break | potyvirus |
| carmovirus | Melon Ourmia ourmiavirus |
| Pelagonium line pattern (?) | Narcissus mosaic potexvirus |
| carmovirus | Neckar River tombusvirus |
| Pepper Moroccan | Olive latent ringspot |
| tombusvirus | nepovirus |
| Pepper ringspot tobravirus | Olive latent 2 (?) |
| Petunia asteroid mosaic | ourmiavirus |
| tombusvirus | Paprika mild mottle |
| Physalis mild chlorosis (?) | tobamovirus |
| luteovirus | Parietaria mottle ilarvirus |
| Potato 14R (?) tobamovirus | Parsnip yellow fleck |
| Potato T trichovirus | sequivirus |
| Potato U nepovirus | Passionfruit Sri Lankan |
| Radish mosaic comovirus | mottle (?) potyvirus |
| Raspberry ringspot | Passionfruit woodiness |
| neprovirus | potyvirus |
| Red clover necrotic mosaic | Pea early browning |
| dianthovirus | tobravirus |
| Ribgrass mosaic | Pea seed-borne mosaic |
| tubamovirus | potyvirus |
| Rose (?) tobamovirus | Peach enation (?) nepovirus |
| Sowbane mosaic | Peanut chlorotic streak |
| sobemovirus | caulimovirus |
| Soybean mild mosaic virus | Peanut clump furovirus |
| Spinach latent ilarvirus | Peanut green mosaic |
| Spinach temperate | potyvirus |
| alphacryptovirus | Peanut stunt cucumovirus |
| Statice Y potyvirus | Peanut yellow spot |
| Strawberry latent ringspot | tospovirus |
| (?) nepovirus | Pelargonium line pattern (?) |
| Sunflower ringspot (?) | carmovirus |
| ilarvirus | Pelargonium vein clearing (?) |
| Sunn-hemp mosaic | cytorhabdovirus |
| tobamovirus | Pepper mild mottle |
| Sweet potato mild mottle | tobamovirus |
| ipomovirus | Pepper Moroccan |
| Tobacco necrosis necrovirus | tombusvirus |
| Tobacco necrotic dwarf | Pepper ringspot tobravirus |
| luteovirus | Pepper severe mosaic |
| Tobacco rattle tobravirus | potyvirus |
| Tobacco ringspot nepovirus | Pepper veinal mottle |
| Tobacco streak ilarvirus | potyvirus |
| Tobacco stunt varicosavirus | Petunia asteroid mosaic |
| Tomato black ring | tombusvirus |
| nepovirus | Petunia vein clearing (?) |
| Tomato bushy stunt | caulimovirus |
| tombusvirus | Physalis mosaic tymovirus |
| Tomato spotted wilt | Pittosporum vein yellowing |
| tospovirus | nucleorhabdovirus |
| Tulip halo necrosis (?) virus | Plantago mottle tymovirus |
| Tulip X potexvirus | Plantain X potexvirus |
| Turnip mosaic potyvirus | Plum American line pattern |
| Vallota mosaic potyvirus | ilarvirus |
| Viola mottle potexvirus | Plum pox potyvirus |
| Watermelon mosaic 2 | Poplar mosaic carlavirus |
| potyvirus | Potato 14R (?) tobamovirus |
| Wineberry latent virus | Potato Andean latent |
| Wisteria vein mosaic | tymovirus |
| potyvirus | Potato aucuba mosaic |
| Cleome spinosa | potexvirus |
| Synonyms: | Potato black ringspot |
| Cleome hassleriana; Cleome | nepovirus |
| arborea; Cleome pungens | Potato mop-top furovirus |
| Common names: | Potato U nepovirus |
| Spider-flower | Potato yellow mosaic |
| Susceptible to: | bigeminivirus |
| Turnip yellow mosaic | Primula mosaic potyvirus |
| tymovirus | Prune dwarf ilarvirus |
| Gloriosa rothschildiana | Prunus necrotic ringspot |
| Synonyms: | ilarvirus |
| Gloriosa superba; Gloriosa | Raspberry ringspot |
| abyssinica; Gloriosa homblei; | nepovirus |
| Gloriosa hybrid; Gloriosa simplex; | Ribgrass mosaic |
| Gloriosa speciosa; Gloriosa | tobamovirus |
| virescens | Rose (?) tobamovirus |
| Common names: | Rubus Chinese seed-borne |
| Flame lily; Glory lily; | (?) nepovirus |
| Climbing lily; Creeping lily | Solanum nodiflorum mottle |
| Susceptible to: | sobemovirus |
| Gloriosa fleck (?) | Sonchus cytorhabdovirus |
| nucleorhabdovirus | Soybean crinkle leaf (?) |
| Tradescantia zebrina | bigeminivirus |
| Synonyms: | Soybean mild mosaic virus |
| Tradescantia pendula; | Soybean mosaic potyvirus |
| Zebrina pendula | Spinach latent ilarvirus |
| Common names: | Sunflower ringspot (?) |
| Wandering-jew | ilarvirus |
| Susceptible to: | Sunn-hemp mosaic |
| Tradescantia-Zebrina | tobamovirus |
| potyvirus | Sweet potato mild mottle |
| Chrysanthemum morifolium | ipomovirus |
| Synonyms: | Tamarillo mosaic potyvirus |
| Dendranthema x | Tobacco etch potyvirus |
| grandiflorum; Anthemis | Tobacco leaf curl |
| grandiflorum; Anthemis | bigeminivirus |
| stipulacea; Chrysanthemum | Tobacco mild green mosaic |
| sinense; Chrysanthemum | tobamovirus |
| stipulaceum; | Tobacco rattle tobravirus |
| Dendranthema x | Tobacco ringspot nepovirus |
| morifolium; Matricaria morifolia | Tobacco streak ilarvirus |
| Common names: | Tobacco stunt varicosavirus |
| Florist's chrysanthemum; | Tobacco yellow vein (?) |
| Mum; Chrisanthemum | umbravirus |
| Susceptible to: | Tomato black ring |
| Chrysanthemum B | nepovirus |
| carlavirus | Tomato bushy stunt |
| Cucumber mosaic | tombusvirus |
| cucumovirus | Tomato golden mosaic |
| Oat blue dwarf marafivirus | bigeminivirus |
| Tomato aspermy | Tomato infectious chlorosis (?) |
| cucumovirus | closterovirus |
| Helianthus annuus | Tomato mosaic tobamovirus |
| Synonyms: | Tomato mottle |
| Helianthus annuus var. | bigeminivirus |
| macrocarpus; Helianthus | Tomato Peru potyvirus |
| lenticularis | Tomato ringspot nepovirus |
| Common names: | Tomato spotted wilt |
| Common annual sunflower; | tospovirus |
| Sunflower; Hopi sunflower; | Tomato top necrosis (?) |
| Common sunflower; Girasol | nepovirus |
| Susceptible to: | Tomato vein clearing |
| Alfalfa mosaic alfamovirus | nucleorhabdovirus |
| Artichoke curly dwarf (?) | Tomato yellow mosaic |
| potexvirus | bigeminivirus |
| Artichoke latent potyvirus | Tulip chlorotic blotch |
| Beet western yellows | potyvirus |
| luteovirus | Tulip halo necrosis (?) virus |
| Bidens mosaic potyvirus | Turnip mosaic potyvirus |
| Bidens mottle potyvirus | Ullucus mild mottle |
| Cassia mild mosaic (?) | tobamovirus |
| carlavirus | Ullucus mosaic potyvirus |
| Cherry leaf roll nepovirus | White clover mosaic |
| Citrus ringspot virus | potexvirus |
| Clover yellow mosaic | Wisteria vein mosaic |
| potexvirus | potyvirus |
| Clover yellow vein | Theobroma cacao |
| potyvirus | Synonyms: |
| Cucumber mosaic | Theobroma sativa |
| cucumovirus | Common names: |
| Cymbidium ringspot | Cacao; Chocolate-tree |
| tombusvirus | Susceptible to: |
| Elm mottle ilarvirus | Cacao necrosis nepovirus |
| Galinsoga mosaic | Cacao swollen shoot |
| carmovirus | badnavirus |
| Humulus japonicus ilarvirus | Cacao yellow mosaic |
| Lettuce infectious yellows | tymovirus |
| (?) closterovirus | Cowpea mild mottle (?) |
| Maracuja mosaic (?) | carlavirus |
| tobamovirus | Okra mosaic tymovirus |
| Melandrium yellow fleck | Tetragonia tetragonioides |
| bromovirus | Susceptible to: |
| Patchouli mosaic potyvirus | Abelia latent tymovirus |
| Peanut stunt cucumovirus | Alfalfa mosaic alfamovirus |
| Pepper veinal mottle | Alstroemeria (?) ilarvirus |
| potyvirus | Alstroemeria mosaic |
| Physalis mosaic tymovirus | potyvirus |
| Prune dwarf ilarvirus | Alstroemeria streak (?) |
| Prunus necrotic ringspot | potyvirus |
| ilarvirus | Amaranthus leaf mottle |
| Red clover necrotic mosaic | potyvirus |
| dianthovirus | Apple stem pitting virus |
| Sunflower crinkle (?) | Arabis mosaic nepovirus |
| umbravirus | Arracacha A nepovirus |
| Sunflower mosaic (?) | Arracacha B (?) nepovirus |
| potyvirus | Arracacha latent (?) |
| Sunflower ringspot (?) | carlavirus |
| ilarvirus | Arracacha Y potyvirus |
| Sunflower yellow blotch (?) | Asparagus 1 potyvirus |
| umbravirus | Asparagus 3 potexvirus |
| Tobacco necrosis necrovirus | Asystasia gangetica mottle |
| Tobacco rattle tobravirus | (?) potyvirus |
| Tobacco streak ilarvirus | Bean common mosaic |
| Tomato black ring | potyvirus |
| nepovirus | Bean yellow mosaic |
| Tomato spotted wilt | potyvirus |
| tospovirus | Beet leaf curl (?) |
| Tropaeolum 2 potyvirus | rhabdovirus |
| Convolvulus arvensis | Beet mild yellowing |
| Common names: | luteovirus |
| Field bindweed | Beet mosaic potyvirus |
| Insusceptible to: | Beet necrotic yellow vein |
| Carnation vein mottle | furovirus |
| potyvirus | Beet western yellows |
| Cornus florida | luteovirus |
| Common names: | Beet yellows closterovirus |
| Flowering dogwood; | Broad bean necrosis |
| American-boxwood | furovirus |
| Susceptible to: | Cacao necrosis nepovirus |
| Cherry leaf roll nepovirus | Cacao yellow mosaic |
| Dogwood mosaic (?) | tymovirus |
| nepovirus | Carnation mottle |
| Synonyms: | carmovirus |
| Corylus avellana f. aurea; | Carnation ringspot |
| Corylus avellana f. contorta; | dianthovirus |
| Corylus avellana f. fusco-rubra; | Carnation vein mottle |
| Corylus avellana f. heterophylla; | potyvirus |
| Corylus avellana f. | Cassava green mottle |
| pendula; Corylus avellana | nepovirus |
| var. aurea; Corylus avellana var. | Cassava Ivorian bacilliform |
| contorta; Corylus avellana var. | ourmiavirus |
| fusco-rubra; Corylus avellana var. | Cassia mild mosaic (?) |
| heterophylla; | carlavirus |
| Corylus avellana var. | Celery latent (?) potyvirus |
| pendula; Corylus heterophylla | Chickpea distortion mosaic |
| Common names: | potyvirus |
| European filbert; European | Chrysanthemum B |
| hazel; Avellana; Hazelnut | carlavirus |
| Susceptible to: | Clover wound tumor |
| Tulare apple mosaic | phytoreovirus |
| ilarvirus | Clover yellow vein |
| Kalanchoe blossfeldiana | potyvirus |
| Synonyms: | Commelina X potexvirus |
| Kalanchoe globulifera var. | Cowpea mild mottle (?) |
| coccinea | carlavirus |
| Susceptible to: | Cucumber mosaic |
| Kalanchoe latent carlavirus | cucumovirus |
| Kalanchoe mosaic (?) | Cycas necrotic stunt |
| potyvirus | nepovirus |
| Kalanchoe top-spotting | Cymbidium ringspot |
| badnavirus | tombusvirus |
| Brassica napus var. napus | Dasheen mosaic potyvirus |
| Synonyms: | Dioscorea latent (?) |
| Brassica campestris f. | potexvirus |
| annua; Brassica campestris f. | Dogwood mosaic (?) |
| biennis; Brassica napus f. annua; | nepovirus |
| Brassica napus f. biennis; Brassica | Eucharis mottle (?) |
| napus ssp. oleifera; | nepovirus |
| Brassica napus var. annua; | Foxtail mosaic potexvirus |
| Brassica napus var. biennis; | Groundnut eyespot |
| Brassica napus var. oleifera | potyvirus |
| Common names: | Habenaria mosaic (?) |
| Rape; Colza; Bird rape; | potyvirus |
| Canola | Helenium S carlavirus |
| Susceptible to: | Heracleum latent trichovirus |
| Watercress yellow spot | Hibiscus latent ringspot |
| virus | nepovirus |
| Brassica nigra | Hypochoeris mosaic (?) |
| Synonyms: | furovirus |
| Brassica nigra var. | Impatiens latent (?) |
| abyssinica; Sinapis nigra | potexvirus |
| Common names: | Iris mild mosaic potyvirus |
| Black mustard | Kalanchoe isometric virus |
| Susceptible to: | Kalanchoe latent carlavirus |
| Beet western yellows | Lamium mild mottle |
| luteovirus | fabavirus |
| Ribgrass mosaic | Lettuce big-vein |
| tobamovirus | varicosavirus |
| Turnip mosaic potyvirus | Lettuce mosaic potyvirus |
| Turnip yellow mosaic | Lilac chlorotic leafspot |
| tymovirus | capillovirus |
| Citrullus vulgaris | Lily X potexvirus |
| Synonyms: | Lisianthus necrosis (?) |
| Citrullus lanatus var. | necrovirus |
| lanatus; Citrullus aedulis; Citrullus | Lucerne Australian latent |
| lanatus var. caffer; Colocynthis | nepovirus |
| citrullus; Cucurbita citrullus | Lychnis ringspot |
| Common names: | hordeivirus |
| Watermelon | Maclura mosaic |
| Susceptible to: | macluravirus |
| Cucumber green mottle | Malva veinal necrosis (?) |
| mosaic tobamovirus | potexvirus |
| Cucumber vein yellowing | Marigold mottle potyvirus |
| virus | Melandrium yellow fleck |
| Telfairia mosaic potyvirus | bromovirus |
| Watermelon chlorotic stunt | Melilotus mosaic (?) |
| bigeminivirus | potyvirus |
| Wild cucumber mosaic | Melon Ourmia ourmiavirus |
| tymovirus | Narcissus latent |
| Cucurbita maxima | macluravirus |
| Common names: | Narcissus mosaic potexvirus |
| Squash; Pumpkin | Narcissus tip necrosis (?) |
| Susceptible to: | carmovirus |
| Apple mosaic ilarvirus | Nerine potyvirus |
| Bean yellow mosaic | Nerine X potexvirus |
| potyvirus | Odontoglossum ringspot |
| Beet curly top | tobamovirus |
| hybrigeminivirus | Okra mosaic tymovirus |
| Cherry leaf roll nepovirus | Ornithogalum mosaic |
| Clover yellow mosaic | potyvirus |
| potexvirus | Parietaria mottle ilarvirus |
| Cucumber leaf spot | Parsnip leafcurl virus |
| carmovirus | Parsnip yellow fleck |
| Cucumber mosaic | sequivirus |
| cucumovirus | Patchouli mottle (?) |
| Daphne X potexvirus | potyvirus |
| Elm mottle ilarvirus | Pea early browning |
| Eucharis mottle (?) | tobravirus |
| nepovirus | Pea mosaic potyvirus |
| Grapevine fanleaf nepovirus | Pea seed-borne mosaic potyvirus |
| Humulus japonicus ilarvirus | Peach enation (?) nepovirus |
| Kyuri green mottle mosaic | Peanut clump furovirus |
| tobamovirus | Peanut green mosaic |
| Lettuce infectious yellows | potyvirus |
| (?) closterovirus | Peanut stunt cucumovirus |
| Lisianthus necrosis (?) | Pelargonium flower break |
| necrovirus | carmovirus |
| Maracuja mosaic (?) | Pelargonium line pattern (?) |
| tobamovirus | carmovirus |
| Melandrium yellow fleck | Pepino mosaic potexvirus |
| bromovirus | Pepper ringspot tobravirus |
| Melon leaf curl | Plantago mottle tymovirus |
| bigeminivirus | Poplar mosaic carlavirus |
| Melothria mottle (?) | Potato 14R (?) tobamovirus |
| potyvirus | Potato black ringspot |
| Papaya ringspot potyvirus | nepovirus |
| Pea seed-borne mosaic | Potato mop-top furovirus |
| potyvirus | Potato U nepovirus |
| Peanut stunt cucumovirus | Primula mosaic potyvirus |
| Poplar mosaic carlavirus | Red clover necrotic mosaic |
| Prune dwarf ilarvirus | dianthovirus |
| Prunus necrotic ringspot | Ribgrass mosaic |
| ilarvirus | tobamovirus |
| Radish mosaic comovirus | Solanum nodiflorum mottle |
| Sowbane mosaic | sobemovirus |
| sobemovirus | Soybean dwarf luteovirus |
| Squash leaf curl | Spinach latent ilarvirus |
| bigeminivirus | Strawberry latent ringspot |
| Squash mosaic comovirus | (?) nepovirus |
| Strawberry latent ringspot | Sweet clover necrotic |
| (?) nepovirus | mosaic dianthovirus |
| Sunflower ringspot (?) | Sweet potato mild mottle |
| ilarvirus | ipomovirus |
| Tobacco necrosis necrovirus | Sweet potato ringspot (?) |
| Tobacco ringspot nepovirus | nepovirus |
| Tobacco streak ilarvirus | Tamus latent (?) potexvirus |
| Tomato bushy stunt | Telfairia mosaic potyvirus |
| tombusvirus | Tobacco etch potyvirus |
| Watermelon curly mottle | Tobacco necrosis necrovirus |
| bigeminivirus | Tobacco ringspot nepovirus |
| Watermelon mosaic 1 | Tobacco stunt varicosavirus |
| potyvirus | Tomato black ring |
| Watermelon mosaic 2 | nepovirus |
| potyvirus | Tomato bushy stunt |
| Wild cucumber mosaic | tombusvirus |
| tymovirus | Tomato vein clearing |
| Zucchini yellow fleck | nucleorhabdovirus |
| potyvirus | Tulip chlorotic blotch |
| Zucchini yellow mosaic | potyvirus |
| potyvirus | Tulip halo necrosis (?) virus |
| Cycas revoluta | Tulip X potexvirus |
| Common names: | Turnip crinkle carmovirus |
| Sago cycas; Sotesu-nut | Turnip mosaic potyvirus |
| Susceptible to: | Ullucus C comovirus |
| Cycas necrotic stunt | Ullucus mild mottle |
| nepovirus | tobamovirus |
| Dioscorea alata | Ullucus mosaic potyvirus |
| Synonyms: | Vallota mosaic potyvirus |
| Dioscorea rubella | Viola mottle potexvirus |
| Common names: | Watermelon mosaic 2 |
| Yam; Greater yam; Water | potyvirus |
| yam; Winged yam; White yam; | Wineberry latent virus |
| Guyana arrowroot; Ten-months | Wisteria vein mosaic |
| yam; Name-de-Agna | potyvirus |
| Susceptible to: | Camellia japonica |
| Dioscorea alata potyvirus | Synonyms: |
| Dioscorea trifida (?) | Camellia japonica var. |
| potyvirus | hortensis; Camellia japonica var. |
| Yam internal brown spot (?) | hozanensis; Camellia japonica var. |
| badnavirus | spontanea; Thea japonica |
| Yam mosaic potyvirus | Common names: |
| Vaccinium corymbosum | Common camellia |
| Synonyms: | Susceptible to: |
| Vaccinium constablaei | Camellia yellow mottle (?) |
| Common names: | varicosavirus |
| Highbush blueberry; | Thunbergia alata |
| Blueberry; American blueberry; | Common names: |
| Swamp blueberry | Black-eyed-Susan-vine; |
| Susceptible to: | Ojitos-negros |
| Blueberry leaf mottle | Susceptible to: |
| nepovirus | Datura yellow vein |
| Blueberry necrotic shock | nucleorhabdovirus |
| ilarvirus | Prune dwarf ilarvirus |
| Blueberry red ringspot | Daphne cneorum |
| caulimovirus | Common names: |
| Blueberry scorch carlavirus | Rose daphne; Garland |
| Blueberry shoestring | flower |
| sobemovirus | Susceptible to: |
| Croton bonplandianus | Daphne S (?) carlavirus |
| Synonyms: | Daphne X potexvirus |
| Croton sparsiflorus | Daphne Y potyvirus |
| Susceptible to: | Corchorus olitorius |
| Croton yellow vein mosaic | Common names: |
| bigeminivirus | Nalta jute; Tossa jute; Tussa |
| Euphorbia marginata | jute |
| Synonyms: | Susceptible to: |
| Euphorbia variegata | Okra mosaic tymovirus |
| Common names: | Tropaeolum majus |
| Snow-on-the-mountain | Common names: |
| Susceptible to: | Garden nasturtium; Indian- |
| Beet curly top | cress; Mastuerzo |
| hybrigeminivirus | Susceptible to: |
| Dulcamara mottle | Alfalfa mosaic alfamovirus |
| tymovirus | Apple mosaic ilarvirus |
| Poinsettia mosaic (?) | Arabis mosaic nepovirus |
| tymovirus | Beet curly top |
| Watermelon mosaic 2 | hybrigeminivirus |
| potyvirus | Beet western yellows |
| Quercus velutina | luteovirus |
| Common names: | Broad bean wilt fabavirus |
| Black oak | Cherry leaf roll nepovirus |
| Susceptible to: | Clover mild mosaic virus |
| Oak ringspot virus | Cucumber mosaic |
| Eustoma russellianum | cucumovirus |
| Synonyms: | Cymbidium mosaic |
| Bilamista grandiflora; | potexvirus |
| Eustoma grandiflorum; | Cymbidium ringspot |
| Lisianthius russellianus | tombusvirus |
| Common names: | Lamium mild mottle |
| Bluebells; Prairie-gentian | fabavirus |
| Susceptible to: | Lettuce infectious yellows |
| Bean yellow mosaic | (?) closterovirus |
| potyvirus | Melandrium yellow fleck |
| Lisianthus necrosis (?) | bromovirus |
| necrovirus | Nasturtium mosaic (?) |
| Pelargonium peltatum | potyvirus |
| Synonyms: | Okra mosaic tymovirus |
| Geranium peltatum | Pea early browning |
| Common names: | tobravirus |
| Ivy geranium; Hanging | Poplar mosaic carlavirus |
| geranium | Red clover necrotic mosaic |
| Susceptible to: | dianthovirus |
| Pelargonium flower break | Ribgrass mosaic |
| carmovirus | tobamovirus |
| Pelargonium line pattern (?) | Strawberry latent ringspot |
| carmovirus | (?) nepovirus |
| Pelargonium vein clearing | Sunn-hemp mosaic |
| (?) cytorhabdovirus | tobamovirus |
| Pelargonium x domesticum | Tobacco rattle tobravirus |
| Insusceptible to: | Tobacco ringspot nepovirus |
| Aster chlorotic stunt (?) | Tomato black ring |
| carlavirus | nepovirus |
| Carnation vein mottle | Tomato spotted wilt |
| potyvirus | tospovirus |
| Chrysanthemum B | Tropaeolum 2 potyvirus |
| carlavirus | White clover mosaic |
| Saintpaulia ionantha | potexvirus |
| Common names: | Anethum graveolens |
| African violet; Usambara | Synonyms: |
| violet | Anethum sowa; |
| Susceptible to: | Peucedanum graveolens |
| Carnation ringspot | Common names: |
| dianthovirus | Dill; Dill seed; Garden dill; |
| Saintpaulia leaf necrosis (?) | Eneldo; Aneto; Fenouil-batard; |
| rhabdovirus | Endro |
| Ribes nigrum | Susceptible to: |
| Common names: | Artichoke yellow ringspot |
| Black currant; Cassis | nepovirus |
| Susceptible to: | Carrot mottle umbravirus |
| Strawberry latent ringspot (?) | Carrot red leaf luteovirus |
| nepovirus | Celery mosaic potyvirus |
| Hypericum perforatum | Heracleum latent trichovirus |
| Common names: | Parsnip yellow fleck |
| Common St. John's-wort; | sequivirus |
| Klamathweed; St. John's-wort; | Foeniculum vulgare |
| Goatweed | Common names: |
| Insusceptible to: | Fennel; Florence fennel; |
| Carnation ringspot | Finocchio; Hinojo |
| dianthovirus | Susceptible to: |
| Hyacinthus orientalis | Coriander feathery red vein |
| Common names: | nucleorhabdovirus |
| Common hyacinth | Insusceptible to: |
| Susceptible to: | Celery yellow spot (?) |
| Hyacinth mosaic potyvirus | luteovirus |
| Crocus vernus | Heracleum latent trichovirus |
| Susceptible to: | Parsnip yellow fleck |
| Iris severe mosaic potyvirus | sequivirus |
| Freesia refracta | Valeriana officinalis |
| Synonyms: | Common names: |
| Freesia leichtlinii; Gladiolus | Common valeriana; Garden- |
| refractus | heliotrope |
| Susceptible to: | Susceptible to: |
| Freesia leaf necrosis | Watermelon mosaic 2 |
| varicosavirus | potyvirus |
| Freesia mosaic potyvirus | Verbena hybrida |
| Gladiolus | Common names: |
| Susceptible to: | Garden verbena; Florist's |
| Artichoke Italian latent | verbena |
| nepovirus | Susceptible to: |
| Bean yellow mosaic | Carnation ringspot |
| potyvirus | dianthovirus |
| Cycas necrotic stunt | Melilotus mosaic (?) |
| nepovirus | potyvirus |
| Narcissus latent | Viola odorata |
| macluravirus | Common names: |
| Iris | English violet; Sweet violet; |
| Susceptible to: | Garden violet |
| Iris mild mosaic potyvirus | Susceptible to: |
| Iris severe mosaic potyvirus | Tulip X potexvirus |
| Juglans regia | Viola mottle potexvirus |
| Synonyms: | Vitis vinifera |
| Juglans duclouxiana; | Common names: |
| Juglans fallax; Juglans kamaonica; | European grape; Wine |
| Juglans orientis; Juglans regia ssp. | grape; Vid |
| kamaonica; Juglans regia var. | Susceptible to: |
| orientis; Juglans | Arabis mosaic nepovirus |
| regia var. sinensis; Juglans | Artichoke Italian latent |
| sinensis | nepovirus |
| Common names: | Grapevine A (?) trichovirus |
| English walnut; Persian | Grapevine ajinashika |
| walnut; Nogal | disease (?) luteovirus |
| susceptible to: | Grapevine Algerian latent |
| Cherry leaf roll nepovirus | tombusvirus |
| Leguminosae | Grapevine B (?) trichovirus |
| Insusceptible to: | Grapevine Bulgarian latent |
| Voandzeia necrotic mosaic | nepovirus |
| tymovirus | Grapevine chrome mosaic |
| Mimosa pudica | nepovirus |
| Common names: | Grapevine corky bark- |
| Sensitive-plant; Touch-me- | associated (?) closterovirus |
| not; Shame plant | Grapevine fanleaf nepovirus |
| Insusceptible to: | Grapevine fleck virus |
| Mimosa mosaic virus | Grapevine leafroll- |
| Soybean mosaic potyvirus | associated (?) closteroviruses |
| Lilium | Grapevine line pattern (?) |
| Susceptible to: | ilarvirus |
| Lily mottle potyvirus | Grapevine stem pitting |
| Tomato aspermy | associated closterovirus |
| cucumovirus | Grapevine stunt virus |
| Tulip breaking potyvirus | Petunia asteroid mosaic |
| Tulipa | tombusvirus |
| Susceptible to: | Strawberry latent ringspot |
| Arabis mosaic nepovirus | (?) nepovirus |
| Tobacco rattle tobravirus | Zingiber officinale |
| Tomato black ring | Synonyms: |
| nepovirus | Amomum zingiber |
| Tomato bushy stunt | Common names: |
| tombusvirus | Ginger; Jengibre |
| | Susceptible to: |
| | Ginger chlorotic fleck (?) |
| | sobemovirus |

Overview of Bioinformatics Methods

A. Phred, Phrap and Consed

Phred, Phrap and Consed are a set of programs which read DNA sequencer traces, make base calls, assemble the shotgun DNA sequence data and analyze the sequence regions that are likely to contribute to errors. Phred is the initial program used to read the sequencer trace data, call the bases and assign quality values to the bases. Phred uses a Fourier-based method to examine the base traces generated by the sequencer. The output files from Phred are written in FASTA, phd or scf format. Phrap is used to assemble contiguous sequences from only the highest quality portion of the sequence data output by Phred. Phrap is amenable to high-throughput data collection. Finally, Consed is used as a “finishing tool” to assign error probabilities to the sequence data. Detailed description of the Phred, Phrap and Consed software and its use can be found in the following references which are hereby incorporated herein by reference: Ewing, B., Hillier, L., Wendl, M. C. and Green, P. (1998) “Base-calling of automated sequencer traces using Phred. I. Accuracy assessment.” Genome Res. 8: 175-178; Ewing, B. and Green, P. (1998) “Base-calling of automated sequencer traces using Phred. II. Error probabilities.” Genome Res. 8:186-194; Gordon, D., Abajian, C. and Green, P. (1998) “Consed: a graphical tool for sequence finishing.” Genome Res. 8: 195-202.

B. BLAST

The BLAST (“Basic Local Alignment Search Tool”) set of programs may be used to compare the large numbers of sequences and obtain homologies to known protein families. These homologies provide information regarding the function of newly sequenced genes. Detailed description of the BLAST software and its uses can be found in the following references which are hereby incorporated herein by reference: Altschul, S. F., Gish, W., Miller, W., Myers, E. W. and Lipman, D. J. (1990) “Basic Local Alignment Search Tool.” J. Mol. Biol. 215: 403-410; Altschul, S. F. (1991) “Amino acid subsitution matrices from an informatics theoretic perspective.” J. Mol. Biol. 219: 555-565.

Generally, BLAST performs sequence similarity searching and is divided into 5 basic programs: (1) BLASTP compares an amino acid sequence to a protein sequence database; (2) BLASTN compares a nucleotide sequence to a nucleic acid sequence database; (3) BLASTX compares translated protein sequences done in 6 frames to a protein sequence database; (4) TBLASTN compares a protein sequence to a nucleotide sequence database that is translated into all 6 reading frames; (5) TBLASTX compares the 6 frame translated protein sequence to the 6-frame translation of a nucleotide sequence database. Programs (3)-(5) may be used to identify weak similarities in nucleic acid sequence.

The BLAST program is based on the High Segment Pair (HSP), two sequence fragments of arbitrary but equal length whose alignment is locally maximized and whose alignment meets or exceeds a cutoff threshold. BLAST determines multiple HSP sets statistically using “sum” statistics. The score of the HSP is then related to its expected chance of frequency of occurrence, E. The value, E, is dependent on several factors such as the scoring system, residue composition of sequences, length of query sequence and total length of database. In the output file will be listed these E values, these are typically in a histogram format, and are useful in determining levels of statistical significance at the user's predefined expectation threshold. Finally, the Smallest Sum Probability, P(N) is the probability of observing the shown matched sequences by chance alone and is typically in the range of 0-1.

BLAST measures sequence similarity using a matrix of similarity scores for all possible pairs of residues and these specify scores for aligning pairs of amino acids. The matrix of choice for a specific use depends on several factors: the length of the query sequence and whether or not a close or distant relationship between sequences is suspected. Several matrices are available including PAM40, PAM120, PAM250, BLOSUM 62 and BLOSUM 50. Altschul et al. (1990) found PAM120 to be the most broadly sensitive matrix (i.e. point accepted mutation matrix per 100 residues). However, in some cases the PAM120 matrix may not find short but strong or long but weak similarities between sequences. In these cases, pairs of PAM matrices may be used, such as PAM40 and PAM 250, and the results compared. Typically, PAM 40 is used for database searching with a query of 9-21 residues long, while PAM 250 is used for lengths of 47-123.

The BLOSUM (Blocks Substitution Matrix) series of matrices are constructed based on percent identity between two sequence segments of interest. Thus, the BLOSUM62 matrix is based on a matrix of sequence segments in which the members are less than 62% identical. BLOSUM62 shows very good performance for BLAST searching. However, other BLOSUM matrices, like the PAM matrices, may be useful in other applications. For example, BLOSUM45 is particularly strong in profile searching.

C. FASTA

The FASTA suite of programs permits the evaluation of DNA and protein similarity based on local sequence alignment. The FASTA search algorithm utilizes Smith/Waterman- and Needleman/Wunsch-based optimization methods. These algorithms consider all of the alignment possibilities between the query sequence and the library in the highest-scoring sequence regions. The search algorithm proceeds in four basic steps:

1). The identities or pairs of identities between the two DNA or protein sequences are determined. The ktup parameter, as set by the user, is operative and determines how many consecutive sequence identities are required to indicate a match.

2). The regions identified in step 1 are re-scored using a PAM or BLOSUM matrix. This allows conservative replacements and runs of identities shorter than that specified by ktup to contribute to the similarity score.

3). The region with the single best scoring initial region is used to characterize pairwise similarity and these scores are used to rank the library sequences.

4). The highest scoring library sequences are aligned using the Smith-Waterman algorithm. This final comparison takes into account the possible alignments of the query and library sequence in the highest scoring region.

Further detailed description of the FASTA software and its use can be found in the following reference which is hereby incorporated herein by reference: Pearson, W. R. and Lipman, D. J. (1988) “Improved tools for biological sequence comparison.” Proc.Natl.Acad. Sci. 85: 2444-2448.

D. Pfam

Despite the large number of different protein sequences determined through genomics-based approaches, relatively few structural and functional domains are known. Pfam is a computational method that utilizes a collection of multiple alignments and profile hidden Markov models of protein domain families to classify existing and newly found protein sequences into structural families. Detailed description of the Pfam software and its uses can be found in the following references which are hereby incorporated herein by reference: Sonhammer, E. L. L., Eddy, S. R. and Durbin, R. (1997) “Pfam: a comprehensive database of protein domain families based on seed alignments.” Proteins: Structure, Function and Genetics 28: 405-420; Sonhammer, E. L. L., Eddy, S. R. Bimey, E., Bateman, A. and Durbin, R. (1998) “Pfam: multiple sequence alignments and HMM-profiles of protein domains.” Nucleic Acids Res. 26: 320-322; Bateman, A., Birney, E., Durbin, R., Eddy, S. R. Finn, R. D. and Sonhammer, E. L. L. (1999) Nucleic Acids Res. 27: 260-262.

Pfam 3.1, the latest version, includes 54% of proteins in SWISS_PROT and SP-TrEMBL-5 as a match to the database and includes expectation values for matches. Pfam consists of parts A and B. Pfam-A, contains a hidden Markov model and includes curated families. Pfam-B, uses the Domainer program to cluster sequence segments not included in Pfam-A. Domainer uses pairwise homology data from Blastp to construct aligned families.

Alternative protein family databases that may be used include PRINTS and BLOCKS, which both are based on a set of ungapped blocks of aligned residues. However, these programs typically contain short conserved regions whereas Pfam represents a library of complete domains that facilitates automated annotation. Comparisons of Pfam profiles may also be performed using genomic and EST data with the programs, Genewise and ESTwise, respectively. Both of these programs allow for introns and frameshifting errors.

E. BLOCKS

The determination of sequence relationships between unknown sequences and those that have been categorized can be problematic because background noise increases with the number of sequences, especially at a low level of similarity detection. One recent approach to this problem has been tested that efficiently detects and confirms weak or distant relationships among protein sequences based on a database of blocks. The BLOCKS database provides multiple alignments of sequences and contains blocks or protein motifs found in known families of proteins.

Other programs such as PRINTS and Prodom also provide alignments, however, the BLOCKS database differs in the manner in which the database was constructed. Construction of the BLOCKS database proceeds as follows: one starts with a group of sequences that presumably have one or more motifs in common, such as those from the PROSITE database. The PROTOMAT program then uses a motif finding program to scan sequences for similarity looking for spaced triplets of amino acids. The located blocks are then entered into the MOTOMAT program for block assembly. Weights are computed for all sequences. Following construction of a BLOCKS database one can use BLIMPS to perform searches of the BLOCKS database. Detailed description of the construction and use of a BLOCKS database can be found in the following references which are hereby incorporated herein by reference: Henikoff, S. and Henikoff, J. G. (1994) “Protein family classification based on searching a database of blocks.” Genomics 19: 97-10; Henikoff, J. G. and Henikoff, S. (1996) “The BLOCKS database and its applications.” Meth. Enz. 266: 88-105.

F. PRINTS

The PRINTS database of protein family fingerprints can be used in addition to BLOCKS and PROSITE. These databases are considered to be secondary databases because they diagnose the relationship between sequences that yield function information. Presently, however, it is not recommended that these databases be used alone. Rather, it is strongly suggested that these pattern databases be used in conjunction with each other so that a direct comparison of results can be made to analyze their robustness.

Generally, these programs utilize pattern recognition to discover motifs within protein sequences. However, PRINTS goes one step further, it takes into account not simply single motifs but several motifs simultaneously that might characterize a family signature. Other programs, such as PROSITE, rely on pattern recognition but are limited by the fact that query sequences must match them exactly. Thus, sequences that vary slightly will be missed. In contrast, the PRINTS database fingerprinting approach is capable of identifying distant relatives due to its reliance on the fact that sequences do not have match the query exactly. Instead they are scored according to how well they fit each motif in the signature. Another advantage of PRINTS is that it allows the user to search both PRINTS and PROSITE simultaneously. A detailed description of the use of PRINTS can be found in the following references which are hereby incorporated herein by reference:Attwood, T. K., Beck, M. E., Bleasly, A. J., Degtyarenko, K., Michie, A. D. and Parry-Smith, D. J. (1997) Nucleic Acids Res. 25: 212-216.

Related, Variant, Altered and Extended Nucleic Acid Sequences

In one embodiment, the invention provides a polypeptide comprising the amino acid sequence encoded by a cDNA identified by a polynucleotide sequence chosen from the group consisting of SEQ ID NO: 1-122. The invention also encompasses variant polypeptides which retain the functional activity of causing a dwarf phenotype in a plant. A preferred variant is one having at least 80%, more preferably 90%, and most preferably 95% amino acid sequence identity to the original polypeptide sequence.

It will be appreciated by those skilled in the art that as a result of the degeneracy of the genetic code, a multitude of nucleotide sequences encoding the same polypeptide, some bearing minimal homology to the nucleotide sequences of any known and naturally occurring gene, may be produced. Thus, the invention contemplates each and every possible variation of nucleotide sequence that could be made by selecting combinations based on possible codon choices. These combinations are made in accordance with the standard triplet genetic code as applied to the nucleotide sequence, and all such variations are to be considered as being specifically disclosed.

It may be advantageous to produce nucleotide sequences encoding polypeptide or its derivatives possessing a substantially different codon usage. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding a polypeptide and its derivatives without altering the encoded amino acid sequences include the production of RNA transcripts having more desirable properties, such as a greater half-life, than transcripts produced from the naturally occurring sequence.

The invention also encompasses production of DNA sequences having the function of causing a dwarf phenotype in a plant, or portions thereof, entirely by synthetic chemistry. After production, the synthetic sequence may be inserted into any of the many available expression vectors and cell systems using reagents that are well known in the art. Moreover, synthetic chemistry may be used to introduce mutations into such a sequence or any portion thereof.

Also encompassed by the invention are polynucleotide sequences that are capable of hybridizing to the polynucleotide sequences shown in SEQ ID NO: 1-122, under various conditions of stringency. Hybridization conditions are based on the melting temperature (Tₘ) of the nucleic acid binding complex or probe, as taught in Wahl, G. M. and S. L. Berger (1987; Methods Enzymol. 152:399-407) and Kimmel, A. R. (1987; Methods Enzymol. 152:507-511), and may be used at a defined stringency.

Altered nucleic acid sequences causing a dwarf phenotype in a plant which are encompassed by the invention include deletions, insertions, or substitutions of different nucleotides resulting in a polynucleotide that is functionally equivalent. The encoded polypeptide may also contain deletions, insertions, or substitutions of amino acid residues which produce a silent change and consequently remains functionally equivalent. Deliberate amino acid substitutions may be made on the basis of similarity in polarity, charge, solubility, hydrophobicity, hydrophilicity, and/or the amphipathic nature of the residues as long as the functional activity is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; phenylalanine and tyrosine.

Also included within the scope of the present invention are alleles of the genes encoded by cDNAs identified by the polynucleotide sequences SEQ ID NO: 1-122. As used herein, an “allele” or “allelic sequence” is an alternative form of the gene which may result from at least one mutation in the nucleic acid sequence. Alleles may result in altered mRNAs or polypeptides whose structure or function may or may not be altered. Any given gene may have none, one, or many allelic forms. Common mutational changes which give rise to alleles are generally ascribed to natural deletions, additions, or substitutions of nucleotides. Each of these types of changes may occur alone, or in combination with the others, one or more times in a given sequence.

Methods for DNA sequencing which are well known and generally available in the art may be used to practice any embodiments of the invention. The methods may employ such enzymes as the Klenow fragment of DNA polymerase I, SEQUENASE® (US Biochemical Corporation, Cleveland, Ohio), TAQ® polymerase (U.S. Biochemical Corporation, Cleveland, Ohio), thermostable T7 polymerase (Amersham Pharmacia Biotech, Chicago, Ill.), or combinations of recombinant polymerases and proofreading exonucleases such as the ELONGASE® amplification system (Life Technologies, Rockville, Md.). Preferably, the process is automated with machines such as the MICROLAB® 2200 (Hamilton Company, Reno, Nev.), PTC200 DNA Engine thermal cycler (MJ Research, Watertown, Mass.) and the ABI 377™ DNA sequencer (Perkin Elmer).

The nucleic acid sequences of the invention may be extended utilizing a partial nucleotide sequence and employing various methods known in the art to detect upstream sequences such as promoters and regulatory elements. For example, one method which may be employed, “restriction-site” PCR, uses universal primers to retrieve unknown sequence adjacent to a known locus (Sarkar, G. (1993) PCR Methods Applic. 2:318-322). In particular, genomic DNA is first amplified in the presence of primer to linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region (Triglia, T. et al. (1988) Nucleic Acids Res. 16:8186). The primers may be designed using OLIGO 4.06 primer analysis software (National Biosciences Inc., Plymouth, Minn.), or another appropriate program, to be 22-30 nucleotides in length, to have a GC content of 50% or more, and to anneal to the target sequence at temperatures about 68-72° C. The method uses several restriction enzymes to generate a suitable fragment in the known region of a gene. The fragment is then circularized by intramolecular ligation and used as a PCR template.

Another method which may be used is capture PCR which involves PCR amplification of DNA fragments adjacent to a known sequence in human and yeast artificial chromosome DNA (Lagerstrom, M. et al. (1991) PCR Methods Applic. 1:111-119). In this method, multiple restriction enzyme digestions and ligations may also be used to place an engineered double-stranded sequence into an unknown portion of the DNA molecule before performing PCR.

Another method which may be used to retrieve unknown sequences is that of Parker, J. D. et al. (1991; Nucleic Acids Res. 19:3055-3060). Additionally, one may use PCR, nested primers, and PROMOTERFINDER™ DNA Walking Kits libraries (Clontech, Palo Alto, Calif.) to walk in genomic DNA. This process avoids the need to screen libraries and is useful in finding intron/exon junctions.

When screening for full-length cDNAs, it is preferable to use libraries that have been size-selected to include larger cDNAs. Also, random-primed libraries are preferable, in that they will contain more sequences which contain the 5′ regions of genes. Use of a randomly primed library may be especially preferable for situations in which an oligo d(T) library does not yield a full-length cDNA. Genomic libraries may be useful for extension of sequence into the 5′ and 3′ non-transcribed regulatory regions.

Capillary electrophoresis systems which are commercially available (e.g. from PE Biosystems, Inc., Foster City, Calif.) may be used to analyze the size or confirm the nucleotide sequence of sequencing or PCR products. In particular, capillary sequencing may employ flowable polymers for electrophoretic separation, four different fluorescent dyes (one for each nucleotide) which are laser activated, and detection of the emitted wavelengths by a charge coupled devise camera. Output/light intensity may be converted to electrical signal using appropriate software (e.g. GENOTYPER® and SEQUENCE NAVIGATOR® from PE Biosystems, Foster City, Calif.) and the entire process from loading of samples to computer analysis and electronic data display may be computer controlled. Capillary electrophoresis is especially preferable for the sequencing of small pieces of DNA which might be present in limited amounts in a particular sample.

Vectors, Engineering, and Expression of Sequences

In another embodiment of the invention, cDNA sequences or fragments thereof which have the function of causing a dwarf phenotype in a plant, or functional equivalents thereof, may be used in recombinant DNA molecules to direct expression of polypeptides in appropriate host cells. Due to the inherent degeneracy of the genetic code, other polynucleotide sequences which encode substantially the same or a functionally equivalent polypeptide also may be produced and these sequences may be used to clone and express the polypeptide of interest.

As will be understood by those of skill in the art, it may be advantageous to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

The polynucleotide sequences of the present invention can be engineered using methods generally known in the art in order to alter their polypeptide encoding sequences for a variety of reasons, including but not limited to, introducing alterations which modify the cloning, processing, and/or expression of the gene product. DNA shuffling by random fragmentation and PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, or introduce mutations, and so forth.

In another embodiment of the invention, natural, modified, or recombinant polynucleotide sequences having the function of causing a dwarf phenotype in a plant may be ligated to a heterologous sequence to encode a fusion protein. For example, to screen peptide libraries for inhibitors of the dwarf phenotype, it may be useful to encode a chimeric protein that can be recognized by a commercially available antibody. A fusion protein may also be engineered to contain a cleavage site located between the wild-type coding sequence and the heterologous protein sequence, so that the wild-type polypeptide may be cleaved and purified away from the heterologous moiety.

In another embodiment, polynucleotide sequences having the function of causing a dwarf phenotype in a plant may be synthesized, in whole or in part, using chemical methods well known in the art (see Caruthers, M. H. et al. (1980) Nucl. Acids Res. Symp. Ser. 215-223, Horn, T. et al. (1980) Nucl. Acids Res. Symp. Ser. 225-232). Alternatively, the polypeptide product may be produced using chemical methods to synthesize the amino acid sequence. For example, peptide synthesis can be performed using various solid-phase techniques (Roberge, J. Y. et al. (1995) Science 269:202-204) and automated synthesis may be achieved, for example, using the ABI 431A™ peptide synthesizer (PE Corporation, Norwalk, Conn.).

The newly synthesized peptide may be substantially purified by preparative high performance liquid chromatography (see, e.g., Creighton, T. (1983) Proteins, Structures and Molecular Principles, WH Freeman and Co., New York, N.Y.). The composition of the synthetic peptides may be confirmed by amino acid analysis or sequencing (e.g., the Edman degradation procedure; or Creighton, supra). Additionally, the amino acid sequence, or any part thereof, may be altered during direct synthesis and/or combined using chemical methods with sequences from other proteins, or any part thereof, to produce a variant polypeptide.

In order to express a biologically active polypeptide, the encoding nucleotide sequences or their functional equivalents, may be inserted into appropriate expression vector, i.e., a vector which contains the necessary elements for the transcription and translation of the inserted coding sequence.

Methods which are well known to those skilled in the art may be used to construct expression vectors containing nucleic acid sequences and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York, N.Y, both of which are hereby incorporated by reference herein.

A variety of expression vector/host systems may be utilized to contain and express sequences having the function of causing a dwarf phenotype in a plant. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV; brome mosaic virus) or with bacterial expression vectors (e.g., Ti or pBR322 plasmids); or animal cell systems.

The “control elements” or “regulatory sequences” are those non-translated regions of the vector—enhancers, promoters, 5′ and 3′ translated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the BLUESCRIPT® phagemid (Stratagene, La Jolla, Calif.) or PSPORT1™ plasmid (Life Technologies, Inc., Rockville, Md.) and the like may be used. The baculovirus polyhedrin promoter may be used in insect cells. Promoters or enhancers derived from the genomes of plant cells (e.g., heat shock, RUBISCO; and storage protein genes) or from plant viruses (e.g., viral promoters or leader sequences) may be cloned into the vector. In mammalian cell systems, promoters from mammalian genes or from mammalian viruses are preferable. If it is necessary to generate a cell line that contains multiple copies of the sequence, vectors based on SV40 or EBV may be used with an appropriate selectable marker.

In bacterial systems, a number of expression vectors may be selected depending upon the use intended for the resulting gene product. For example, when large quantities of gene product are needed for the induction of antibodies, vectors which direct high level expression of fusion proteins that are readily purified may be used. Such vectors include, but are not limited to, the multifinctional E.coli cloning and expression vectors such as BLUESCRIPT® phagemid (Stratagene, La Jolla, Calif.), in which a sequence may be ligated into the vector in frame with sequences for the amino-terminal Met and the subsequent 7 residues of β-galactosidase so that a hybrid protein is produced; pIN vectors (Van Heeke, G. and S. M. Schuster (1989) J. Biol. Chem. 264:5503-5509); and the like. pGEMX™ vectors (Promega Corporation, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

In cases where plant expression vectors are used, the expression of sequences having the function of causing a dwarf phenotype in a plant may be driven by any of a number of promoters. In a preferred embodiment, plant vectors are created using a recombinant plant virus containing a recombinant plant viral nucleic acid, as described in PCT publication WO 96/40867 which is hereby incorporated herein by reference. Subsequently, the recombinant plant viral nucleic acid which contains one or more non-native nucleic acid sequences may be transcribed or expressed in the infected tissues of the plant host and the product of the coding sequences may be recovered from the plant, as described in WO 99/36516, which is hereby incorporated herein by reference.

An important feature of this embodiment is the use of recombinant plant viral nucleic acids which contain one or more non-native subgenomic promoters capable of transcribing or expressing adjacent nucleic acid sequences in the plant host and which result in replication and local and/or systemic spread in a compatible plant host. The recombinant plant viral nucleic acids have substantial sequence homology to plant viral nucleotide sequences and may be derived from an RNA, DNA, cDNA or a chemically synthesized RNA or DNA. A partial listing of suitable viruses is described below.

The first step in producing recombinant plant viral nucleic acids according to this particular embodiment is to modify the nucleotide sequences of the plant viral nucleotide sequence by known conventional techniques such that one or more non-native subgenomic promoters are inserted into the plant viral nucleic acid without destroying the biological function of the plant viral nucleic acid. The native coat protein coding sequence may be deleted in some embodiments, placed under the control of a non-native subgenomic promoter in other embodiments, or retained in a further embodiment. If it is deleted or otherwise inactivated, a non-native coat protein gene is inserted under control of one of the non-native subgenomic promoters, or optionally under control of the native coat protein gene subgenomic promoter. The non-native coat protein is capable of encapsidating the recombinant plant viral nucleic acid to produce a recombinant plant virus. Thus, the recombinant plant viral nucleic acid contains a coat protein coding sequence, which may be native or a nonnative coat protein coding sequence, under control of one of the native or non-native subgenomic promoters. The coat protein is involved in the systemic infection of the plant host.

Some of the viruses which meet this requirement include viruses from the tobamovirus group such as Tobacco Mosaic virus (TMV), Ribgrass Mosaic Virus (RGM), Cowpea Mosaic virus (CMV), Alfalfa Mosaic virus (AMV), Cucumber Green Mottle Mosaic virus watermelon strain (CGMMV-W) and Oat Mosaic virus (OMV) and viruses from the brome mosaic virus group such as Brome Mosaic virus (BMV), broad bean mottle virus and cowpea chlorotic mottle virus. Additional suitable viruses include Rice Necrosis virus (RNV), and geminiviruses such as tomato golden mosaic virus (TGMV), Cassava latent virus (CLV) and maize streak virus (MSV). However, the invention should not be construed as limited to using these particular viruses, but rather the method of the present invention is contemplated to include all plant viruses at a minimum.

Other embodiments of plant vectors used for the expression of sequences having the function of stunting a plant include, for example, viral promoters such as the 35S and 19S promoters of CaMVused alone or in combination with the omega leader sequence from TMV (Takamatsu, N. (1987) EMBO J. 6:307-311). Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196.

An insect system may be used to express the polypeptides of the invention. For example, in one such system, Autographa californica nuclear polyhedrosis virus (AcNPV) is used as a vector to express foreign genes in Spodoptera frugiperda cells or in Trichoplusia larvae. The sequences encoding the gene product may be cloned into a non-essential region of the virus, such as the polyhedrin gene, and placed under control of the polyhedrin promoter. Successful insertion of the sequence will render the polyhedrin gene inactive and produce recombinant virus lacking coat protein. The recombinant viruses may then be used to infect, for example, S. frugiperda cells or Trichoplusia larvae in which the gene product may be expressed (Engelhard, E. K. et al. (1994) Proc. Nat. Acad. Sci. 91:3224-3227).

In mammalian host cells, a number of viral-based expression systems may be utilized. In cases where an adenovirus is used as an expression vector, the nucleic acid sequences of the invention may be ligated into an adenovirus transcription/translation complex consisting of the late promoter and tripartite leader sequence. Insertion in a non-essential E1 or E3 region of the viral genome may be used to obtain a viable virus which is capable of expressing the relevant gene product in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of the nucleic acid sequences of the invention. Such signals include the ATG initiation codon and adjacent sequences. In cases where a sequence, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and WI38, which have specific cellular machinery and characteristic mechanisms for such post-translational activities, may be chosen to ensure the correct modification and processing of the foreign protein.

For long-term, high-yield production of recombinant proteins, stable expression is preferred. For example, cell lines which stably express a specific gene product may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1980) Cell 22:817-23) genes which can be employed in tk⁻ or aprt⁻ cells, respectively. Also, antimetabolite, antibiotic, or herbicide resistance can be used as the basis for selection; for example, dhfr, which confers resistance to methotrexate (Wigler, M. et al. (1980) Proc. Natl. Acad. Sci. 77:3567-70); npt, which confers resistance to the aminoglycosides neomycin and G-418 (Colbere-Garapin, F. et al (1981) J. Mol. Biol. 150: 1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). Recently, the use of visible markers has gained popularity with such markers as anthocyanins, β-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if a nucleic acid sequence of the invention is inserted within a marker gene sequence, recombinant cells containing that specific sequence can be identified by the absence of marker gene function. Alternatively, a marker gene can be placed in tandem with a sequence of the invention under the control of a single promoter. Expression of the marker gene in response to induction or selection usually indicates expression of the tandem gene as well.

Alternatively, host cells which contain a nucleic acid sequence of the invention and which express its gene product may be identified by a variety of procedures known to those of skill in the art. These procedures include, but are not limited to, DNA-DNA or DNA-RNA hybridizations and protein bioassay or immunoassay techniques which include membrane, solution, or chip based technologies for the detection and/or quantification of nucleic acid or protein.

The presence of polynucleotide sequences of the invention can be detected by DNA-DNA or DNA-RNA hybridization or amplification using probes or portions or fragments of polynucleotide sequence of interest. Nucleic acid amplification based assays involve the use of oligonucleotides or oligomers based on the sequences of interest to detect transformants containing the relevant DNA or RNA. As used herein “oligonucleotides” or “oligomers” refer to a nucleic acid sequence of at least about 10 nucleotides and as many as about 60 nucleotides, preferably about 15 to 30 nucleotides, and more preferably about 20-25 nucleotides, which can be used as a probe or amplimer.

A variety of protocols for detecting and measuring the expression of a cDNA, using either polyclonal or monoclonal antibodies specific for the protein are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on the protein is preferred, but a competitive binding assay may be employed. These and other assays are described, among other places, in Hampton, R. et al. (1990; Serological Methods, a Laboratory Manual, APS Press, St Paul, Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to the polynucleotide sequences of the invention include oligonucleotide labeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits from Pharmacia & Upjohn (Kalamazoo, Mich.), Promega Corporation (Madison, Wis.) and U.S. Biochemical Corp. (Cleveland, Ohio). Suitable reporter molecules or labels, which may be used, include radionuclides, enzymes, fluorescent, chemiluminescent, or chromogenic agents as well as substrates, cofactors, inhibitors, magnetic particles, and the like.

Host cells transformed with a polynucleotide sequence of the invention may be cultured under conditions suitable for the expression and recovery of protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of its corresponding polypeptide through a prokaryotic or eukaryotic cell membrane. Other recombinant constructions may be used to join polynucleotide sequences of the invention to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS™ extension/affinity purification system (Immunex Corp., Seattle, Wash.). The inclusion of cleavable linker sequences such as those specific for Factor XA or enterokinase (available from Invitrogen, San Diego, Calif.) between the purification domain and polypeptide of interest may be used to facilitate purification. One such expression vector provides for expression of a fusion protein comprising a nucleic acid encoding 6 histidine residues preceding a thioredoxin or an enterokinase cleavage site. The histidine residues facilitate purification on IMIAC (immobilized metal ion affinity chromatography) as described in Porath, J. et al. (1992, Prot. Exp. Purif 3: 263-281,) while the enterokinase cleavage site provides a means for purifying polypeptide of interest from the fusion protein. A discussion of vectors which contain fusion proteins is provided in Kroll, D. J. et al. (1993; DNA Cell Biol. 12:441-453).

In addition to recombinant production, a fragment of a polypeptide of the invention may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using the Applied Biosystems 431A peptide synthesizer (Perkin Elmer). Various peptide fragments may be chemically synthesized separately and combined using chemical methods to produce the full length molecule.

In additional embodiments, the nucleotide and amino acid sequences of the present invention may be incorporated into any molecular biology techniques yet to be developed, provided these new techniques rely on properties of nucleotide and amino acid sequences that are currently known, including, but not limited to, such properties as the triplet genetic code and specific base pair interactions.

The following examples further illustrate the present invention. These examples are intended merely to be illustrative of the present invention and are not to be construed as being limiting. The examples are intended specifically to illustrate the various methods used to identify and characterize the cDNAs of the present invention and the method by which they can be used to cause a dwarf phenotype in a plant.

### EXAMPLES

I. Construction and Characterization of a Normalized Arabidopsis cDNA library in GENEWARE® Vectors

A. Plant Tissue Generation:

Arabidopsis thaliana ecotype Columbia (0) seeds were sown and grown on PEAT LITE MIX (Speedling Inc., Sun City, Fla.) supplemented with NUTRICOTE fertilizer (Plantco Inc., Ontario, Canada). Plants were grown under a 16-hour light/8-hour dark cycle in an environmental controlled growth chamber. The temperature was set at 22° C. for daytime and 18° C. for nighttime. The entire plant, root, leaves and all aerial parts were collected 4 weeks post sowing. Tissue was washed in deionized water and frozen in liquid nitrogen.

B. RNA Extraction:

High quality total RNA is isolated using a hot borate method. All solutions were made in DEPC-treated, double-deionized water and autoclaved. All glassware, mortars, pestles, spatulas, and glass rods were baked at 400° C. for four hours. All plasticware was DEPC-treated for at least three hours and then autoclaved.

Thirty-five milliliters of XT buffer (0.2 M Na borate decahydrate, 30 mM EGTA, 1% SDS (w/v), 1% deoxycholate, sodium) per 10 grams of tissue was dispensed into 50 milliliter Falcon tubes. PVP-40, 000 was added to a final concentration of 2% (w/v). NP-40 was added to a final concentration of 1% (w/v). Tubes were placed in an 80° C. water bath. The mortar and pestles were then pre-cooled in liquid nitrogen. Proteinase K (0.5 mg/ml XT buffer) was dispensed into 250 ml centrifuge bottles and the bottles were then placed on ice.

The tissue was added to the pre-chilled mortar and pestle and ground to a fine powder. Working as quickly as possible, the tissue was transferred to a glass beaker using a spatula chilled in liquid nitrogen. DTT (1.54 mg/ml XT buffer) was added to the XT buffer/PVP/NP-40 buffer and was immediately added to the ground tissue. The tissue was homogenized using a polytron at level 5 for one minute. The homogenate was decanted into the 250 ml centrifuge bottle containing the proteinase K. The homogenate was incubated at 42° C., 100 rpm for 1.5 hours. Eighty microliters of 2M KCl/ml of XT buffer was added to the homogenate and gently swirled until mixed. The samples were then incubated on ice for one hour. The samples were centrifuged at 12,000× G in a BECKAN® JA-14 rotor (Beckman Instruments, Inc., Fullerton, Calif.) for 20 minutes at 4° C. to remove debris. The supernatant was then filtered through a funnel lined with sterile miracloth into a sterile 250 ml centrifuge bottle. Eight molar LiCl was added to a final concentration of 2M LiCl and the samples were incubated on ice overnight.

Precipitated RNA was pelleted by centrifugation at 12,000× G in a BECKMAN® JA-14 rotor for 20 minutes (Beckman Instruments, Inc., Fullerton, Calif.) and the supernatant was discarded. The RNA pellet was washed in 5 milliliters of cold 2M LiCl in 30 ml centrifuge tubes. Glass rods and gentle vortexing were used to break and disperse the RNA pellet. The pellets were centrifuged in a Beckman JA-20 rotor for 10 krpm at 4° C. for 10 minutes. The supernatant was decanted. This wash step was repeated 3 times until the supernatant was relatively colorless. The RNA pellet was resuspended in 5 milliliters of 10 Tris-Cl (pH 7.5). The insoluble material was pelleted in a JA-17 at 10 k rpm for 10 minutes at 4° C. The supernatant was transferred to another 30 ml centrifuge tube and 0.1× volume of 2M K-acetate (pH 5.5) was added. The samples were incubated on ice for 15 minutes and centrifuged in a BECKMAN® JA-17 rotor (Beckman Instruments, Inc., Fullerton, Calif.) at 10 k rpm, 4° C., for 10 minutes to remove polysaccharides and insoluble material. The supernatant was transferred to a sterile 30 ml centrifuge tube and RNA was precipitated by adding 2.5× volumes of 100% ethanol. The RNA was precipitated overnight at −20° C. The precipitated RNA was pelleted by centrifugation at 9 krpm, 4° C. for 30 minutes in a JA-17 rotor. The RNA pellet was washed with 5 milliliters of cold 70% ethanol and centrifuged in a JA-17 rotor at 9 k rpm, 4° C. for 10 minutes. The residual ethanol was removed using a BECKMAN® speed vac (Beckman Instruments, Inc., Fullerton, Calif.). The RNA pellet was resuspended in 3 milliliters of DEPC-ddH₂O+1 mM EDTA. The RNA was precipitated with 0.1× volumes of 3M Na-acetate pH 6.0 and 2× volumes of cold 100% ethanol. The RNA was put at -80° C. for storage. A BECKMAN® spectrophotometer (Beckman Instruments, Inc., Fullerton, Calif.) was used to measure absorbance (A) at A₂₆₀ and A₂₈₀. The A₂₆₀ was used to determine concentration (40 μg RNA/ml=1 A₂₆₀ absorbance unit) and the A₂₆₀/A₂₈₀ ratio was used to determine the initial quality of the RNA (1.8 to 2.0 is good).

The yield of total RNA from 60 g of tissue is ˜15 mg. Then, mRNA was isolated from total RNA using oligo (dT)₂₅ DYNABEADS® (Dynal, Inc., Lake Success, N.Y.). Typically, 1% of total RNA population can be recovered as mRNA in Arabidopsis thaliana whole plant and from 5 μg of poly A⁺ RNA, approximate 4.5 μg of single strand cDNA and 6.7 μg of double strand cDNA was synthesized.

C. cDNA Synthesis:

Poly A⁺ RNA was purified from total RNA using the oligo (dT)₂₅ DYNABEADS® kit (Dynal, Inc., Lake Success, N.Y.) according to manufacturer's instructions. Briefly, DYNABEADS® was resuspended by mixing on a roller and transfer 600 μl to an RNase free tube. The beads were further equilibriated with 2× binding buffer (20 mM Tris-HCl, pH 7.5, 1M LiCl, 2 mM EDTA) twice and resuspended in 200 μl of 2× binding buffer. Total RNA 1 mg (200 μl) was heated at 70° C. for 5 minutes and incubated with the above oligo (dT)₂₅ DYNABEADS® for 10 min at RT. The supernatant containing unbound rRNA and tRNA was subsequently removed by magnetic stand and washed twice with 1× wash buffer (10 mM Tris-HCl, pH 7.5, 0.15M LiCl, 1 mM EDTA). The mRNA was eluted from the DYNABEADS® in ddH₂O and used as the starting material for double strand cDNA synthesis.

Double strand cDNA was synthesized either with NotI-(dT)₂₅ primer or on oligo (dT)₂₅ DYNABEADS® based on the manufacturer's instruction (Gibco-BRL superscript system). Typically, 5 μg of poly A⁺ RNA was annealed and reverse transcribed at 37° C. with SUPERSCRIPT II reverse transcriptase (Stratagene, La Jolla, Calif.). For the non-normalized cDNA library, double stranded cDNAs were ligated to a 500 to 1000-fold molar excess SalI adaptor, restriction enzyme NotI digested and size-selected by column fractionation. Those cDNAs were then cloned directionally into the XhoI-NotI sites of the TMV expression vector, 1057 N/P.

D. Normalization Procedure:

For the normalized cDNA preparation, the supernatant was removed from the DYNABEADS® and the cDNA containing beads were washed twice with 1× TE buffer. To carry out the normalization process, the second strand cDNA were eluted from the beads. 100 μl of TE buffer was added to the beads and heated at 95° C. for 5 min and the supernatant was then collected on magnetic stand. The above procedure was repeated once to ensure complete elution. The yield of second strand cDNA was quantitated using a UV spectrophotometer.

First strand cDNA beads is combined with second strand cDNA in 4× SSC, 5× Denhardt's and 0.5% SDS for multiple rounds of short hybridization. Since the second strand cDNA was synthesized using the first strand cDNA as the template, approximately the same amount of first and second strand cDNAs were present in the hybridization reaction. Nine μg of second strand cDNA in 200 μl of 1× TE buffer was added to the cDNA driver (first strand cDNA on beads) in a screw cap tube. The reaction was heated at 95° C. for 5 min, then 60 μl of 20× SSC, 30 μl of 50× Denhardt's (1% of Ficoll, 1% of polyvinylpyrrolidone and 1% of bovine serum albumin) and 15μl of 10% SDS were added and the reaction was brought to 65° C. for 8 hours.

The beads and supernatant were separated at 65° C. by magnet. The supernatant was transferred to a fresh tube and kept at 65° C. The beads were regenerated by adding 200 μl of ddH₂O and heated at 95° C. for 5 min. We collected the beads for the next round of hybridization and kept the solution containing the bound second strand cDNA for further analysis. The partially normalized second strand cDNA solution was added back to the regenerated beads and a return to another round of hybridization of 8 hours. This procedure was repeated 4-5 times.

E. Slot Blot Analysis:

To follow the process of cDNA normalization a rapid slot blot procedure was developed. Following sequencing of 960 cDNAs, 46 cDNAs were selected to follow the representation of various classes of cDNAs through the normalization procedure. Based on their frequency of appearance in the sequence, these clones represent transcripts of different expression levels (high, moderate and low). Ten nanograms of each cDNA were deposited onto a HYBOND™-N⁺ membrane (Amersham Pharmacia Biotech, Chicago, Ill.) along with control vector (pBS) and water controls. DNA was denatured, neutralized, and subsequently crosslinked into the membrane using UV-STRATALINKER™ 2400 (Stratagene, La Jolla, Calif.).

cDNAs from either the non-normalized or normalized pool were labelled with ³²P and hybridized on the slot blot membrane overnight at 65° C. in 1% bovine serum albumin, 1 mM ethylenediaminetetraacetic acid (EDTA), 0.5 M sodium phosphate (pH 7.2), and 7% sodium dodecyl sulfate (SDS). Then, blots were washed once in 1× SSC/0.2% SDS for 20 min at room temperature followed by two washes in 0.2× SSC/0.2% SDS for 20 min. at 65° C. The resulting membranes were then developed using a PHOSPHORIMAGER™ (Amersham Pharmacia Biotech, Chicago, Ill.) and quantitated using available software.

F. Conversion of Single-Stranded Normalized cDNAs to Double-Stranded Form:

Second strand normalized cDNA in hybridization solution was purified by QIAQUICK™ column (QIAGEN GmbH, Hilden, Germany) and eluted in 88 μl of ddH₂O (total 1.2 μg of DNA is recovered). One μl (3 μg) of NotI-oligo dT primer was added and heated at 95° C. for 5 min followed by cool down to 37° C. The first strand cDNA was extended with T7 DNA polymerase (Amersham Pharmacia Biotech, Chicago, Ill.) in the presence of dNTP in 120 μl reaction at 37° C. for 1 hour. T4 DNA polymerase (NEB) was then used to polish the ends following the extension reaction for 5 min at 16° C. The resulting double strand cDNA was ethanol precipitated and ligated with 500- to 1 000-fold molar excess of SalI adaptor followed by NotI digestion. The resulting cDNAs were size-fractionated using a Clontech spin column 400 and the first two fractions that contained the cDNAs were pooled and used for the subsequent cloning process.

G. Construction of cDNA Libraries in GENEWARE® Vectors:

(+) Sense cDNA clones were prepared as follows. The Tobacco Mosaic Virus expression vector, 1 056GTN-AT9 was linearized with NotI and XhoI and a 900 bp stuffer DNA was removed. The presence of the stuffer DNA in between those two sites is to ensure the complete digestion by restriction enzymes and thus achieve the high cloning efficiency. The digested vector was gel purified and then used to set up ligation reaction with normalized cDNA SalI-NotI fragments to generate (+) sense cDNA clones.

(−) Sense cDNA clones were prepared as follows. The Tobacco Mosaic Virus expression vector 1057 NP also linearized with NotI and XhoI and a stuffer DNA fragment was removed. The digested vector was gel purified and used to set up ligation reaction to generate (−) sense strand library.

Each ligation was transformed into chemically competent E. coli cells, DH5 α according to manufacturer's instruction (Life Technologies, Rockville, Md.). Preliminary analysis of cloning efficiency was measured by plating of a small portion of the transformation, while archiving the majority for future applications. Vector-only ligations gave ˜2×10⁴ cfu/μg vector and ligations with cDNA insertions gave ˜5×10⁵ cfu/μg.

H. Analysis of Normalized cDNA Populations:

With each successive round of kinetic re-association, the total cDNA population is depleted thereby confirming the removal of a population of the cDNA from the mixture at each step. To further understand the consequences of this depletion and measure the relative normalization in cDNA representation following various stages of the kinetic re-association method, slot blots of 46 genes of varying representations were hybridized with probes made from non-normalized and normalized cDNA preparations. The resulting blots were then analyzed for representation by PHOSPHORIMAGER® analysis. The hybridization pattern of non-normalized cDNA to the gene array reveals a quite asymmetric representation with some genes hybridizing with great intensity while others showing no hybridization at all. The variance among hybridization intensities for each spot within the filter was measured by standard deviation and found to be 649. In order to analyze the cDNA fraction depleted from the mixture, the first strand magnetic bead matrix was eluted, a radioactive probe was generated and hybridized to a replica of the slot blot described above. The resulting hybridization intensities indicated that primarily those cDNAs of higher copy number were bound and removed from the normalized cDNA population, confirming that the depletion phenomenon correlated with removal of primarily high copy number cDNAs. The cDNA population not bound to first strand magnetic beads after 5 serial passages was collected, radioactive probe was generated and hybridized to a replica slot blot of known gene set described above. The resulting hybridization pattern (i.e. the relative intensity of the slots on the blot) was in striking contrast to that of the non-normalized cDNA and to that of the bound cDNA fraction. Assuming that the majority of the hybridization signal to the slot blot for the non-normalized cDNA blot results from hybridization to high abundance genes, an initial comparison can be made between the number of bound counts on the normalized versus non-normalized slot blots. This comparison is possible since each probe added to the blots was derived from the same quantity of cDNA material and an equal number of probe counts were applied to the blots. The non-normalized blot contained 17,898 counts while the normalized blot contained only 1494 counts. This represents a 12-fold reduction in overall signal indicating a significant reduction in high gene copy number in the normalized cDNA population.

When the hybridization intensity of the non-normalized cDNA probe to each gene is plotted against the relative number of counts (following subtraction of the pBS vector control intensity from each sample), there is almost a 4-log difference in sequence representation in the cDNA population and an overall variance in standard deviation of 649-fold. In contrast, the hybridization of normalized cDNA probe to each gene revealed an average of only 32-fold difference. This represents both a reduction in high copy cDNAs and an increased representation in low copy cDNAs by >3 logs. The variance between the most highly represented cDNA and lowest represented cDNA within the normalized cDNA population was ˜1.5 logs. The above values characterizing the degree of library normalization are equivalent to those achieved by Soares, et al. (1994).

I. Analysis of GENEWARE® Clones:

To ascertain the cloning efficiency of normalized cDNA into each vector and the average insert size, 96 random colonies were picked and grown by standard methods. DNA was isolated from bacteria using a BIOROBOT™ 9600 (QIAGEN GmbH, Hilden, Germany). DNA was digested with Not I and BsiWI restriction endonucleases (recognition sites flank the cDNA insertion). The digestions were separated on agarose gels and visualized by ethidium bromide staining. The digestions revealed a vector religation background of ˜4%. Ligations giving >75% insertions were passed as to quality control and more colonies were picked. Approximently 600 independent clones were analyzed by restriction digestion as described above. Interestingly, a similar percentage of vector background was detected ˜4% and the average insert size in the vector was ˜1 kb, with many inserts with 2 kb or greater sized inserts. Following analysis of DNA by restriction mapping, DNA was subjected to sequencing and further analysis.

J. Sequence Analysis of the Normalized Arabidopisis Library in GENEWARE®:

Initial analysis of non-normalized Arabidopsis cDNA library required the sequencing of 1709 independent clones. Three 96-well plates of randomly picked normalized Arabidopsis library in GENEWARE® [(−) sense] were initially sequenced by primer TP6 to yield 262 5′ sequences and passed sequence quality control. Initially, internal cluster analysis was performed to identify identical sequences in this sequence subset. Analysis using BLASTN algorithm showed that of the 262 sequences analyzed, 252 were unique and only 10 were found to cluster into five two-member clusters. We then identified the redundancy of the sequences against the larger public databases. For cluster analysis, we used a very low BLASTX score criteria (e=10⁻⁶) and compared all sequences against the GENBANK® nr database (United States Department of Health and Human Services). In this manner, we could derive the most information concerning the redundancy, gene type found and open reading frame status of all clones simultaneously. The low BLASTX score was used to allow all possible protein homologues to be identified. The clustering analysis revealed that of the 262 sequences there were 252 single member sequence clusters and five two-gene clusters. This represents 96% singletons from this sample size. The genes appearing more than once in the library varied from two different chlorophyll a/b binding proteins, lipid transport proteins to ferrodoxin-thioredoxin reductases. This result compares quite favorably to the 4 redundant clones (of one gene type) identified by Soares, et al. (1994) from 187 randomly picked clones from one normalized library.

Further analysis of the sequences from the GENEWARE® normalized cDNA library revealed that of the 262 sequences subjected to BLASTX search of the GENBANK® nr database, 29% of the sequences failed to show significant homology to any characterized protein or open reading frame (ORF). Of the 252 singletons in the library, 179 showed single hit to an identified ORF, while 73 showed no hit. These results suggest that, in spite of the well characterized nature of the sequence database quality libraries can still contain a high proportion of new expressed sequences.

The excellent representation and extremely low redundancy observed in these initial plates of normalized Arabidopsis cDNAs in GENEWARE® prompted us to sequence additional clones. This was important because there is often a significant bias in small sample sizes with regard to representation. A total of 1,151 sequences passed sequence quality control. Internal cluster analysis showed that ˜260 multi-sequence clusters were present, with the highest representation at 6 members and the majority with only 2 members (˜150). About 600 unique clusters were identified from the total of 856 clusters from the 1151 sequences. Therefore, from the 1151 sequences analyzed, 1,010 unique genes were identified, or a 87.7% gene discovery rate. In contrast, internal cluster analysis of the non-normalized Arabidopsis cDNA sequences revealed ˜840 multi-gene clusters with the highest represented cluster containing 27 members. Cluster analysis of the 1709 non-normalized Arabidopsis cDNAs revealed clusters of 27 members and many other highly populated clusters, a dramatic difference from the normalized cDNAs.

Further comparison of 1,151 randomly chosen non-normalized sequences for redundancy with the results from the 1,151 normalized population clearly indicated the positive effects of normalization and the greater number of unique genes identified from this normalized population. Many genes that have representations of >12 in the non-normalized library have been reduced to 1-4 members in the normalized population. One chlorophyll a/b binding protein gene exhibited a reduction from 15 members in the non-normalized population to 1 in the normalized library, whereas a gene encoding a distinct chlorophyll a/b binding protein showed less reduction in the normalized gene population. This observation is consistent with the conclusion that certain genes do not undergo the same degree of normalization compared with other genes.

Additional sequences from the normalized Arabidopsis library were obtained by sequence analysis. BLASTN analysis of the 1,343 normalized sequences revealed that 858 were represented in the Arabidopsis EST database, while the remaining 485 sequences were apparently unique, with no obvious homologue in the database. Of those sequences showing BLASTN hits, 43.6% showed coverage of the first through tenth base in the longest EST in the database. Furthermore, 242 of the 858 (28%) showed 5′ sequences that were at the first base of the longest EST or longer. These data show that the cDNAs cloned into GENEWARE® are of significant quality and represent, in many cases, the longest 5′ sequences obtained to date. To further ascertain the proportion of cDNAs containing full-length protein open reading frames, we employed the ORF finder program used to analyze the ABRC library for sense clones. This algorithm checks for ATG sequences in the first 70 bases of a sequence and then scans for sequences lacking an in-frame stop codon for at least 300 nt downstream in the same frame. To understand the number of quality ORFs in a library, we used the ABRC library as a benchmark. Analysis of 11,957 sequences within the ABRC library with the ORF finder program revealed 3,207 hits (26.8%) with putative open reading frames. From the 1,343 sequences of the normalized Arabidopsis cDNA library in GENEWARE®, 907 (67.5%) were hits using the ORF finder program. Coupling the number of cDNAs that represent near the 5′ end of the known RNA sequence (43.6%) with the number of clones that contain putative intact ORFs (67.5%) testifies to the quality and integrity of the cDNAs in the GENEWARE® vector. These data clearly indicate a high proportion of full-length clones.

K. Quantity of Normalized Arabidopsis cDNAs Cloned into GENEWARE® Vectors:

As previously described, the normalized Arabidopsis cDNA population was cloned into GENEWARE® vectors in both the positive (+) and negative (−) sense direction to allow for both overexpression and gene knockout analysis. The total number of clones in the 1057 PN vector in negative orientation was 20,160. These were arrayed into 210 96-well glycerol stock plates. Likewise, 20,160 clones from the ligation of normalized Arabidopsis cDNA in sense orientation into 1056 GTN vector have been arrayed in 210 96-well glycerol stock plates. These numbers clearly show that the GENEWARE® vectors can be used as primary cloning vectors and that very complex libraries can be obtained in two orientations from a single pool on non-amplified normalized cDNA.

II. Construction of Tissue-Specific N. benthamiana cDNA Libraries

A. mRNA Isolation:

Leaf, root, flower, meristem, and pathogen-challenged leaf cDNA libraries were constructed. Total RNA samples from 10-5 μg of the above tissues were isolated by TRIZOL reagent (Life Technologies, Rockville, Md.). The typical yield of total RNA was 1 mg. PolyA+RNA was purified from total RNA by DYNABEADS® oligo (T)₂₅. Purified mRNA was quantified by UV absorbance at OD₂₆₀. The typical yield of mRNA was 2% of total RNA. The purity was also determined by the ratio of OD₂₆₀/OD₂₈₀. The integrity of the samples has OD values of 1.8-2.0.

B. cDNA Synthesis:

cDNA was synthesized from mRNA using the SUPERSCRIPT® plasmid system (Life Technologies, Rockville, Md.) with cloning sites of NotI at the 3′ end and SalI at the 5′ end. After fractionation through a gel column to eliminate adapter fragments and short sequences, cDNA was cloned into both GENEWARE® vector p1057 NP and phagemid vector PSPORT™ in the multiple cloning region between NotI and XhoI sites. Over 20,000 recombinants were obtained for all of the tissue-specific libraries.

C. Library Analysis:

The quality of the libraries was evaluated by checking the insert size and percentage from representative 24 clones. Overall, the average insert size was above 1 kb, and the recombinant percentage was >95%.

III. Construction of Normalized N. benthamiana cDNA Library in GENEWARE® Vectors

A. cDNA Synthesis.

A pooled RNA source from the tissues described above was used to construct a normalized cDNA library. Total RNA samples were pooled in equal amounts first, then polyA+RNA was isolated by DYNABEADS® oligo (dT)25. The first strand cDNA was synthesized by the Smart III system (Clontech, Palo Alto, Calif.). During the synthesis, adapter sequences with Sfi1a and Sfi1b sites were introduced by the polyA priming at the 3′ end, and 5′ end by the template switch mechanism (Clontech, Palo Alto, Calif.). Eight μg first strand cDNA was synthesized from 24 μg mRNA. The yield and size were confirmed by UV absorbance and agarose gel electrophoresis.

B. Construction of Genomic DNA Driver.

Genomic DNA driver was constructed by immobilizing biotinylated DNA fragments onto streptavidin-coated magnetic beads. Fifty μg genomic DNA was digested by EcoR1 and BamH1 followed by fill-in reaction using biotin-21-dUTP. The biotinylated fragments were denatured by boiling and immobilized onto DYNABEADS® by the conjugation of streptavidin and biotin.

C. Normalization Procedure.

Six μg of the first strand cDNA was hybridized to 1 μg of genomic DNA driver in 100 μl of hybridization buffer (6× SSC, 0.1% SDS, 1× Denhardt's buffer) for 48 hours at 65° C. with constant rotation. After hybridization, the cDNA bound on genomic DNA beads was washed 3 times by 20 μl 1× SSC/0.1% SDS at 65° C. for 15 min and one time by 0.1× SSC at room temperature. The bounded cDNA on the beads was then eluted in 10μl of fresh-made 0.1N NaOH from the beads and purified by using a QIAGEN DNA purification column (QIAGEN GmbH, Hilden, Germany), which yielded 110 ng of normalized cDNA fragments. The normalized first strand cDNA was converted to double strand cDNA in 4 cycles of PCR with Smart primers annealed to the 3′ and 5′end adapter sequences.

D. Evaluation of Normalization Efficiency.

Ninety-six non-redundant cDNA clones selected from a randomly sequenced pool of 500 clones of a previously constructed whole seedling library were used to construct a nylon array. One hundred ng of the normalized cDNA fragments vs. the non-normalized fragments were radioactively labeled by ³²P and hybridized to DNA array nylon filters. Hybridization images and intensity data were acquired by a PHOSPHORIMAGER® (Amersham Pharmacia Biotech, Chicago, Ill.). Since the 96 clones on the nylon arrays represent different abundance classes of genes, the variance of hybridization intensity among these genes on the filter were measured by standard deviation before and after normalization. These results indicated that by using this type of normalization approach, we could achieve a 1 000-fold reduction in variance among this set of genes.

E. Cloning of Normalized cDNA into GENEWARE® Vector.

The normalized cDNA fragments were digested by Sfi1 endonuclease, which recognizes 8-bp sites with variable sequences in the middle 4 nucleotides. After size fractionation, the cDNA was ligated into GENEWARE® vector p1057 NP in antisense orientation and transformed into DH5α cells. Over 50,000 recombinants were obtained for this normalized library. The percentage of insert and size were evaluated by Sfi digestion of randomly picked 96 clones followed by electrophoresis on 1% of agarose gel. The average insert size was 1.5 kb, and the percentage of insert was 98% with vector only insertions of >2%.

F. Sequence Analysis of Normalized cDNA Library.

As of the date of this report, 2 plates of 96 randomly picked clones have been sequenced from the 5′ end of cDNA inserts. One hundred ninety-two quality sequences were obtained after trimming of vector sequences and other standard quality checking and filtering procedure, and subjected to BLASTX search in DNA and protein databases. Over 40% of these sequences had no hit in the databases. Clustering analysis was conducted based on accession numbers of BLASTX matches among the 112 sequences that had hits in the databases. Only three genes (tumor-related protein, citrin, and rubit) appeared twice. All other members in this group appeared only once. This was a strong indication that this library is well-normalized. Sequence analysis also revealed that 68% of these 192 sequences had putative open reading frames using the ORF finder program (as described above), indicating possible full-length cDNA.

IV. DNA Preparation

A. High Throughput Clone Preparation.

Arraying of the ABRC library into GENEWARE® vectors occurred as previously discussed to obtain ˜5,000 antisense and ˜3,000 sense clones with minimal redundancy. The ligations were between highly purified and quality controlled GENEWARE® cloning vector plasmids and the corresponding fragments from each individual pool of ABRC clones. Cloning efficiencies were in the range of 1×10⁵ to 5×10⁵ per μg of plasmid. Colonies were picked using a Flexys Colony Picker (The Sanger Centre, England) and manual methods. Colonies were applied to deep-well cell growth blocks (DWBs) and grown from 18-26 hours at 37° C. at ˜500 rpm in the presence of ampicillin concentrations of 500 μg/ml. From the almost 9,000 colonies picked by the Flexys, >97% of the cultures successfully grew. DNA was prepared using the QIAGEN BIOROBOT 9600 DNA robots and QIAGEN 96-well manifolds (manual preparation) at a rate of 2,000 DNA preparations per day. The final throughput, during campaign production, estimated for each system was ˜20 plates of 96 samples per day, per production line—robotic or manual. Such throughput could be sustained to generate 20-40,000 samples in a matter of one to two weeks of effort. During one ten day period, one hundred four (140) 96-well plates of DNA were produced.

B. Quality Control Methods:

DNA samples were subjected to quality control (QC) analysis by at least one of two methods: 1) restriction endonuclease digestion and analysis by agarose gel electrophoresis (all plates) or 2) UV spectroscopy to determine DNA quantitation for all 96 samples of a plate (statistical sampling of each days output). For UV analysis, an aliquot of the DNA samples from each plate was taken and measured using a Molecular Dynamics UV spectrometer in 96-well format (Molecular Dynamics, Sunnyvale, Calif.). DNA concentrations of 0.05-0.2 μl with OD 260/280 ratios of 1.7+0.2 are expected. For DNA sequencing purposes (a downstream method to be used to analyze all “hit” samples), DNA quantity of 0.04-0.2 μg/μl is desired. In general, plates that contain >25% of samples not conforming to this metric are rejected and new DNA for the plate must be generated once again. For conformation of the presence of insertions and full-length GENEWARE® vector, agarose gel electrophoresis of restriction endonuclease fragments was used. Aliquots of sixteen samples from each 96-well DNA plate were targeted for restriction digestion using Nco I and BstE II restriction endonucleases. Samples were separated on 1% agarose gels. Generally, plates that showed >25% of samples that were not full length or did not contain insertions were rejected. From a total of 140 96-well DNA plates prepared, 112 passed QC and were made available for generation of infectious units.

V. High-Throughput DNA Sequencing and Sequence Analysis Protocols

A. Generation of Raw Sequence Data and Filtering Protocols:

High-throughput sequencing was carried out using the PCT200® and TETRAD® PCR machines (MJ Research, Watertown, Mass.) in 96-well plate format in combination with two ABI 377™ automated DNA sequencers (PE Corporation, Norwalk, CT). The throughput at present is six 96-well plates per day.

The electropherogram generated from sequencer by ABI Sequencing Analysis (version 3.3) was used to generate sequence in the text format using “Phred,” which also gives a confidence score for each base call that reflect the error probability and the quality for that base. Cross_match was used to mask the vector sequence. The low quality portion of the sequence (i.e. phred score lower than 20) was removed. The vector and the polyA or polyT were also removed from the raw sequence. The high quality, processed sequences with the processing information were stored in the database. Sequences were used for further bioinformatic analysis.

B. Sequence Data Analysis and Bioinformatics:

Once the filtering and the vector sequence removal steps are completed, the resulting sequences are subjected to database search. First, low sensitivity methods such as BLASTN and BLASTX can be used. For those sequences that have no hit, more sensitive methods, such as Blimps and Pfam can be used. To speed up the analysis process, appropriate filters may be used. For example, for EST sequences from a given cDNA library sequenced from the 5′ end, an ATG filter can be used to make sure that only full-length cDNA will be analyzed. The filtered sequence can be translated in one frame rather than six frames for Pfam analysis.

The results from the database search are stored in the relational database and can be used for further analysis. For example, all the BLAST results can be stored in a relational table that contains Query, Score, pValue, Hit, Length, Annotation, Frame, Identity, Homology, Query Length, Subject Length, Database Queried and Method used to analyze. Any result can be queried and analyzed by the fields mentioned. A database link between the analysis result database and the laboratory information management system (LIMS) has been created so that the analysis result can be related to the experimental data.

C. Metabolic Pathway Analysis:

Many metabolic pathway databases have been constructed that group proteins based on their roles in a metabolic pathway. The basic identifiers for these proteins are E.C. numbers; therefore, the position of a given enzyme in a metabolic pathway may be determined based on its E.C. number. The E.C. number of a protein can be obtained by its Genbank ID. This approach can be used to assign the corresponding E.C. number to the hits found for each cDNA sequence. By querying the metabolic pathway using the E.C. number of a hit, a potential link between this cDNA sequence and the metabolic pathway may be established. Each link can be used as a building block for a plant metabolic pathway. This potential link between cDNA sequence and metabolic pathway provides a starting point to analyze the gene's role in a metabolic pathway.

In addition, we have created an interactive, queriable relational prokaryotic and eukaryotic metabolic pathway database. This metabolic pathway database was created by accessing all public sequences that have associated E.C. numbers, running HMMs (hidden Markov models) and other proprietary LSBC algorithms against these sequences, and classifying these sequences into protein families based on conserved domains (Pfam database assignments). Pfam is a database of multiple alignments of protein domains or conserved protein regions. It is assumed that they represent some evolutionary conserved structure which has implications for the protein's function. Pfam is actually formed in two separate ways. Pfam-A are accurate human crafted multiple alignments whereas Pfam-B is an automatic clustering of the rest of SWISSPROT and TrEMBL derived from the Prodom (http://www.toulouse.inra.fr/prodom.html) database. Each protein family has the following data: 1). A seed alignment which is a hand edited multiple alignment representing the domain; 2). A Hidden Markov Model (HMM) derived from the seed alignment which can be used to find new members of the domain and also take a set of sequences to realign them to the model; 3). A full alignment which is a automatic alignment of all the examples of the domain using the HMM to find and then align the sequences; and 4). An annotation file which contains a brief description of the domain, some parameters for Pfam methods, and links to other databases.

We have run HMMs and other LSBC algorithms against the LSBC Sequence Database and classified these sequences into protein families based on conserved domains, and relate these sequences back to public sequences for E.C. mapping to metabolic pathways. We have run HMMs and other LSBC algorithms against all sequenced microbial genomes and classified these sequences into protein families based on conserved domains, and relate these sequences back to public sequences for E.C. mapping to metabolic pathways. We further related the Arabidopsis, N. benthamiana, and Oryza clones to specific sites on metabolic pathways.

D. Sequence Analysis of Library Created from GENEWARE® Vectors:

Five hundred sixty-eight (568) independent clones were sequenced from the virus expression library and the clones from this library were analyzed by vector, N filters and BLAST analysis. Of the 568 initial sequences submitted for analysis, 131 were eliminated by the N-filter indicating that ˜15% of the sequence were undetermined Ns. The remaining 437 sequences were then subjected to analysis for duplication within each set of submitted plates. Fifty-five (55) sequences were removed due to this duplication filter. These sequences were BLASTN searched against 539 sequences from the AtwpLNLH library in Lambda Zap II. Thirty percent (30%) of the sequences (i.e., 132 sequences) found a match in both libraries. From the original set of GENEWARE® clones, 305 were found to be unique with respect to the Lambda Zap II library. These sequences were then BLASTX-searched against non-redundant GENBANK . From the 305 submitted sequences, 173 sequences found solid hits in protein coding sequence as determined by hit criteria and 132 were found to be unique. Further BLASTN analysis showed a range of sequence homology, but many represented hits to BAC or chromosomal sequences. A wide range of sequences were found including, ribosomal proteins, photosystem reaction center proteins, fumarase and other general metabolism proteins, transcription factors, kinase homologs, omega-6 fatty acid desaturase and various hypothetical proteins. These results strongly suggest that little or no bias is introduced during the construction of cDNA libraries in GENEWARE®.

VI. Preparation of Infectious Units

DNA plates that pass QC testing were then moved to the next stage of the cycle, the generation of infectious units. In vitro RNA transcriptions have been optimized to produce maximal amounts of RNA in smaller volumes to reduce costs and increase the lifetime of a DNA preparation. A transcription mixture containing a 6-to-1 RNA cap structure-to-rGTP ratio, Ambion mMessage Machine buffer and enzyme mix (Ambion, Inc., Austin, Tex.) is delivered to a 96-well plate by the TECAN liquid handling robot (TECAN, Research Triangle Park, N.C.). To this reaction mix, the Robbins Scientific HYDRA 96-sample pipeting robot (Robbins Scientific, Sunnyvale, Calif.) delivers 2 μl of DNA solution. This final transcription reaction is incubated at 37° C. for 1.5 hours. Following incubation, the TECAN robot delivers 95 μl of a 100 mM Na/K PO₄ buffer containing TMV coat protein (devoid of all infectious RNA) to the transcription plate and it is incubated overnight. This incubation generates encapsidated transcripts, which are very stable at room temperature or 4° C. and amplified with regard to number of infectious units per μg of RNA transcript. The generation of infectious materials is measured by inoculation of GFP-expressing virus to systemic host or Nicotiana tabacum NN lines, incubation at permissive temperatures and counting of developing local lesions on inoculated leaves. Before addition of the TMV coat protein mixture, 0.5 μl from 8 wells of each transcription plate is removed and analyzed by agarose gel electrophoresis. The presence of an RNA band of ˜1.6 to 3.5 kb is strong evidence for a successful transcription. If >25% contain only lower molecular weight RNA bands, or if the band is diffuse <500 bp of dsDNA marker, the transcription plate is considered to have failed and removed from the stream of plates prepared for inoculation. During a two week period, 112 plates were transcribed and 108 plates were passed for plant inoculation in growth rooms and in the field.

VII. Plant Inoculation with Encapsidated RNA Transcripts

In order to prepare for plant inoculation, 90 μl of each encapsidated RNA transcript sample and 90 μl of FES transcript inoculation buffer (0.1 M glycine, 0.06 M K₂HPO₄, 1% sodium pyrophosphate, 1% diatomaceous earth and 1% silicon carbide) were combined in the wells of a new 96-well plate. The 96 well plate was then placed on ice.

Nicotiana benthamiana plants 14 days post sowing were removed from the greenhouse and brought into the laboratory. Humidity domes were placed over the plants to retain moisture. The RNA transcript sample was mixed by pipetting the solution prior to application to ensure that the silicon carbide and the diatomaceous earth were resuspended. The entire sample, 180 μl, was drawn up and pipetted in equal aliquots (approximately 30 μl), onto the first two true leaves of three separate Nicotiana benthamiana plants. The mixture was spread across the leaf surface using a Texwipe™ Cleanfoam™ swab (The Texwipe Co, Upper Saddle River, N.J.). The wiping action caused by the swab together with the silicon carbide in the buffer sufficiently abrades the leaves so as to allow the encapsidated RNA transcript to enter the plant cell structure. Other methods used for inoculation have included pipeting of encapsidation-FES mixture onto leaves and rubbing by hand, cotton swab or nylon inoculation wand. Alternatively, nylon inoculation wands may be incubated in the transcript-FES mixture for ˜30 min to soak up ˜15 μl and then rubbed directly onto the leaves.

Once an entire 32 plant flat was inoculated, the plants were misted with deionized water and the humidity domes were replaced over them. The inoculated plants were retained in the laboratory for 6 hours and then returned to the greenhouse. Once in the greenhouse, the humidity domes were removed and the plants were misted a second time with deionized water.

VIII. Inoculated Plant Growth

Plants inoculated with encapsidated virus were grown in a greenhouse. Day length was set to 16 hours and shade curtains (33% transmittance) were used to reduce solar intensity. Whenever ambient light fell below 250 μmol m²s⁻¹, a 50:50 mixture of metal halide and sodium halide lamps (Sylvania), delivering an irradiance of approximately 250 μmol m²s⁻¹, were used to provide supplemental lighting. Evaporative cooling and steam heat were used to regulate temperature, with a daytime set point of 27° C. and a nighttime set point of 22° C. The plants were irrigated with Hogland's fertilizer mix as required. Drainage water was collected and treated with 0.5% sodium hypochlorite for 10 minutes before discharging into the municipal sewer.

To allow space for increased plant size, the inoculated N. benthamiana were repositioned at seven days post-inoculation (dpi) so that they occupied twice their original area. At 13 dpi, the plants were examined visually for symptoms of TMV infection and were assigned a numerical score to indicate the extent of viral infection (0=no infection, 1=possible infection, 2=limited/late infection, 3=typical infection, 4=severe infection). At the same time, the plants were assigned a fate for harvest (typically the highest quality plant in each triplicate was assigned to metabolic screens and the second highest quality plant was assigned to focused screens). In cases where plant symptoms deviated substantially from those of plants inoculated with control vectors, a description of plant phenotype was recorded (as described below). At 14 dpi infected plants were harvested.

IX. Infectivity Analysis

The method to measure the infectivity of the transcript encapsidations was to inoculate a set of 96-well plates from both positive and negative sense clones and look for systemic virus movement and phenotype development. Of the 8,352 plants inoculated with unique encapsidated transcriptions, 6,266 became systemically infected for an infection rate of 76%. Overall, the majority of plates generated showed very good infection rates. As shown in a graph of the number of systemically infectious constructs per each individual plate plotted against plate number. The majority of plates had systemic rates >70% with one at 100%. Approximately 25 plates had infection rates ranging between 40 and 70% while only 6% (>5 plates) showed infection rates <45%.

A population of constructs did not show systemic infection on Nicotiana benthamiana plants. Analysis using the LIMS revealed a substantial correlation between a subset of inoculators and the transcription plates showing poor infection rates. These results strongly suggest that inoculation technique is critical for good infectivity although other possible causes could include poor DNA or transcription quality, or simply inoculation error. In some cases the constructs may be restricted to inoculated leaves by way of adverse influence of the gene insertion on virus replication and movement. For example, one observed healthy inoculated Nicotiana benthamiana plant exhibited clear chlorotic spots on inoculated leaves, yet no systemic symptoms. Other plants, not scored as infected in our LIMS, were observed to have subliminal infections in source tissues. It was clear that the properties of the genetic insertion had differing effects on virus phenotypic symptoms. Eighty-two of those constructs exhibiting poor systemic infection were re-inoculated into Nicotiana tobacum NN plants to test for local lesions. The presence of local lesions indicated infectious viral vectors. From this data, a statistical calculation can be made to determine the percentage of non-systemic infective constructs that are locally infectious. Plants were scored 6 days post-inoculation for the presence of localized necrotic lesions resulting from infection and localized movement of virus vectors on the inoculated leaves of the plants. Of the 82 constructs analyzed, 50 showed local lesions indicating the presence of infectious viral vectors. Based on the infection rate observed in Nicotiana benthamiana and NN tobacco plants, we estimate that 1,181 (˜61%) of the constructs not showing systemic infection on Nicotiana benthamiania plants were still infectious and amenable to biochemical analysis.

X. Phenotypic Evaluation

At 13 dpi a visual examination was made to identify plants whose phenotype deviates substantially from plants infected with a GENEWARE® control. The phenotypically different plants were divided into regions (for example: shoot apical region, infected phloem source leaves, stem) and descriptive terms were applied to each region to document the visual observation. Additionally, a confirmation was made as to whether or not the operator considered the plant to be a “hit” and a numerical score was applied to document the phytotoxic/herbicide effect of the RNA insert (1=possible effect, 2=mild, 3=moderate, 4=severe).

A matrix-style phenotypic database was created using the LIMS software. The LIMS software allows all descriptive terms to be used for any major part of the plant and the capacity of sub-parts to be described. Notable phenotypic events are captured by description of individual plant parts. The matrix is configured in a Web-based page that allows one to score infection and phenotyping using a graphic replicated of the physical arrangement of plants in the growth room. This approach is rapid, allowing 96 plants to be described in detail as being infected, not infected with a detailed phenotype in ˜15 min. Editing of output files can occur rapidly in MS Excel if desired. The output file is then loaded as CSV files into the LIMS where it is immediately available to Boolean query as to phenotype descriptors with “and, or, not” statements. Images of infected plants are linked to the SeqIDs in the database so that the plant tray bar code (for infection), well position, SeqID, phenotype and picture all link together when a query is made. This is linked back to the sequence database for sequence annotation data. Using this system, 8,352 phenotypic observations were made in the period of two days and entered into the LIMS. Hundreds of interesting visual phenotypes were observed.

XI. Field-Scale Genomics

The effects of gene overexpression and gene silencing in plants may have dramatic differences when grown under different conditions. The Kentucky field test plots available to Biosource provides an opportunity to subject plants to substantially different growth conditions and thereby broaden the chances of detecting various types of “hits” in a genomics screen. To compare the ability of virus vectors to be applied under field conditions and under controlled growth room conditions, we inoculated, in duplicate, 960 positive-sense constructs on Nicotiana benthamiana plants grown in the field test plot in Owensboro, Ky. This activity was concurrent with inoculations and screens performed in Vacaville, Calif. Complete encapsidated transcription reactions were prepared at Large Scale Biology Corporation in Vacaville, Calif. and following incubation with TMV coat protein, FES buffer was added to each well. All samples in column 12 of each plate contained encapsidated transcripts of 1057 vector containing the GFP gene. The mixture was then overnight-mailed to Owensboro, Ky. where it was inoculated onto 4-5 week post-sowing plants by rubbing cotton swabs, pre-wetted by incubation with encapsidated transcript-FES mixture, on plant leaves. Plants were inoculated in duplicate. Plants were allowed to remain in the field for 4 weeks post-inoculation and then subjected to phenotypic analysis. Photographic documentation of the plants both pre- and post-inoculation was prepared. Plants were scored by visual evaluation as to number of infected plants compared with total number of plants inoculated. Of the 1920 plants inoculated, 1,712 (88%) showed systemic infections. More than 100 new phenotypes were noted in the field. Each was compared with the phenotype of the same construct inoculated into plants in Vacaville, Calif. growth rooms. Two new phenotypes are particularly noteworthy: two independent plants showed survival phenotypes under anaerobic conditions, whereas all neighbors had succumbed to root rot in a low spot in the field.

In order to evaluate the effect of gene silencing in Nicotiana tabacum plants, mRNA from Arabidopsis thaliana whole plants was subjected to fragment normalization such that small cDNA fragments were produced. The cDNA population showed high degree of normalization by hybridizations with known genes of variable expression and by comparison with non-normalized cDNA fragments. The average size of the normalized fragments in the GENEWARE® vectors was between 400-500 bp allowing facile movement of the recombinant viruses systemically in field Nicotiana tabacum c.v. MD609 plants. A total of 11 plates of DNA constructs (1056) were prepared, transcribed and encapsidated with GFP constructs integrated at every 12^{th} position. These were mixed with FES and overnight-mailed to Owensboro, Ky. These 1056 constructs were inoculated in duplicate (2112 total) on MD609 tobacco plants 11 weeks post-sowing. One set of the replicates (1056 plants) were scored by visual evaluation as to number of infected plants compared with total number of plants inoculated. Of the 1056 plants inoculated, 808 showed systemic infections, or 76.5% infection rate. “Hits” were determined by unusual visual symptoms and corresponding constructs will be characterized by DNA sequencing.

An uncharacterized GENEWARE® library comprised of 20,000 Arabidopsis thaliana normalized fragment cDNAs and 10,000 of Nicotiana benthamiana genomic DNA fragments was prepared and sprayed as a population on Nicotiana tabacum c.v. MD609 plants. The Arabidopsis cDNA library, ˜10,000, was constructed by ligation into prepared GENEWARE® vectors and purified from pooled bacterial transformants and followed by pooled transcription. The remaining 10,000 cDNA fragments were individual clones prepared and transcribed independently and then mixed in a pooled encapsidation. The Nicotiana library was a prototype cell-free cloning library from restriction endonuclease fragmented gDNA of <500 bp in size. The number of clones corresponds to an approximation of the amount of DNA undergoing complete ligation. Transcriptions from each non-encapsidated library were inoculated separately into Nicotiana tabacum protoplasts and allowed to incubate for three days. Cells were lysed and libraries combined. The pool of cell lysates and encapsidated transcriptions containing viral libraries were shipped to Owensboro, KY where they were inoculated onto Nicotiana tabacum c.v. MD609 plants at 1, 1/10, 1/100 and 1/000 dilution of the mixed virion preparation (using 60 ml, 6 mls, 0.6 mls and 0.06 mls of the library respectively). Eight hundred (800) plants were spray-inoculated with each library virion dilution. Plants were visually scored and of the 3,200 plants inoculated, 1,304 showed visual symptoms 3 weeks post-infection. The infectivity rate varied from ˜60% for the most concentrated inoculum to ˜20% for the most dilute as would be expected due to dilution. Analysis will continue to define “Hits” by unusual visual symptoms and PCR amplification and DNA sequencing will characterize corresponding construct.

XII. GC/MS Metabolite Analysis

A. Harvest and Preparation of Tissues for Metabolic Screening

Fourteen dpi infected plants to be harvested were moved from the greenhouse to the laboratory. Plants were scanned and identified by a bar-code that linked the infected plant to the tissue sample. The infected tissue was cut off of the plant and placed in a corresponding centrifuge tube. A tungsten carbide ball was placed on top of the infected tissue sample. The tungsten carbide ball facilitates pulverization of plant tissue. The tubes and sample were stored on dry ice during the harvesting procedure. The samples were then stored at −70° C. Before conducting a metabolic screen, the tissue samples must be pulverized. The sample tubes were loaded into a KLECO pulverizer and pulverized to create a fine powder of the tissue sample. The tissue sample powder was then weighed out into a metabolic extraction vial.

B. FAME Analysis Procedure for FAME Screen.

Nicotiana benthamiana plants expressing genes of interest in RNA vectors were grown for 14 dpi as described above. Three leaf disks (0.5 cm in diameter) were placed in cell wells of a borosilicate 96-deepwell plate (Zinsser). 500 μl of heptane was added to each well using a Biomek 2000 Laboratory Automation Workstation. The heptane/tissue samples were stirred on a Bodine magnetic stirrer. After 30 minutes, 50 μl of 0.5N sodium methoxide in methanol was added to each well using the Biomek 2000. After 30 minutes of stirring, 10 μl of water was added to each well. Injections were made directly from the 96-deepwell plate into a Hewlett Packard gas chromatograph (GC) using a LEAP auto injector. The GC method involved a 2 μl injection into a split/splitless injection port using a DB 23 narrow bore column (15 M, 0.25 I.D.). The oven temperature was isothermic at 170° C. The injector temperature was 230° C. and the detector (flame ionization) temperature was 240° C. The run time was 5 minutes, with an equilibration time of 0.5 minutes. The split ratio was 20:1 and the helium flow rate was held at a constant pressure of 19 psi. This GC method allowed for separation and quantification of fatty acid methyl esters which included C16:0,C16:1,C18:0,C18:1,C18:2,and C18:3. Using a dual column GC, four 96-well plates could be sampled in less than 24 hours.

The following sequences exhibited a positive FAME result (had altered levels of the fatty acids assayed): SEQ ID NOs: 7, 53, and 92. The result of the FAME analysis for SEQ ID NO:92 is shown in Table 5. Table 5 shows the relative percent amounts of fatty acids found in plants transfected with a viral vector comprising SEQ ID NO: 92. An increase in 16:0 fatty acids was observed in 3 of the 5 samples assayed. Table 6 shows the relative percent amounts of fatty acids found in plants transfected with SEQ ID NOs: 7 and 53.

TABLE 5
FAME Profile

| Sample | 16:0 | 16:1 | unk | 16:3 | unk | 18:0 | 18:1 | 18:2 | 18:3 | unk |
|---|---|---|---|---|---|---|---|---|---|---|
| 1 | 24.7 | 3.4 | 1.1 | 3.2 | 2.6 | 2.6 | 3.3 | 9.2 | 47.8 | 2.0 |
| 2 | 20.1 | 2.9 | 0.8 | 4.6 | 2.9 | 3.5 | 7.1 | 9.2 | 46.7 | 2.3 |
| 3 | 17.6 | 1.8 | 1.0 | 3.5 | 2.9 | 2.2 | 6.0 | 11.8 | 50.4 | 2.7 |
| 4 | 23.3 | 1.9 | 1.0 | 3.1 | 4.6 | 3.8 | 8.9 | 10.6 | 37.6 | 5.3 |
| 5 | 23.0 | 2.6 | 0.7 | 3.5 | 1.6 | 2.3 | 3.8 | 8.1 | 52.9 | 1.6 |
| control | 19.6 | 2.8 | 1.1 | 3.3 | 1.8 | 1.8 | 3.1 | 12.0 | 53.6 | 1.0 |
| control | 18.4 | 2.7 | 1.1 | 3.3 | 1.7 | 1.7 | 3.1 | 11.3 | 55.4 | 1.3 |

TABLE 6
FAME Profile

| Sample | 16:0 | 16:1 | unk | 16:3 | unk | 18:0 | 18:1 | 18:2 | 18:3 | unk |
|---|---|---|---|---|---|---|---|---|---|---|
| SEQ ID | 23.0 | 3.5 | 1.9 | 2.6 | 1.7 | 2 | 3.3 | 11.7 | 49.1 | 1.3 |
| NO: 53 | | | | | | | | | | |
| SEQ ID | 25.7 | 3.4 | 1.3 | 1.8 | 0.8 | 2.3 | 2.1 | 8 | 54.7 | 0 |
| NO: 7 | | | | | | | | | | |
| control | 18.7 | 2.8 | 1.2 | 3.8 | 1.4 | 1.5 | 4.2 | 10.7 | 55 | 0.6 |

C. Insect Control Bioassays.

Nicotiana benthamiana plants expressing genes of interest in RNA viral vectors were grown for 14 dpi as described previously. Fresh leaf tissue (sample size ˜2.5 cm diameter) was excised from the base of infected leaves using a scalpel and placed in insect-rearing tray (Bio RT32, C-D International) wells containing 3 ml of 2% agar. Using a small paintbrush to handle insects, 2 first-instar larvae of tobacco hornworm (Manduca sexta) were placed in each well and trays were sealed using vented covers. Trays were then incubated at 28 C with 48% humidity for 72 hours with a 12-hour photoperiod. Following incubation, samples were scored for mortality and leaf damage according to the following criteria: mortality, 0=0 dead/2 alive; 1=1 dead/1 alive; 2=2 dead/0 alive; leaf damage, 0=0 to 20% leaf consumed; 1=21 to 40% leaf consumed; 2=41 to 60% leaf consumed; 3=61 to 80% leaf consumed; and 4=81 to 100% leaf consumed. Following scoring, insects were weighed on an analytical balance and photographed using a digital camera.

The following sequences exhibited a positive insect control phenotype: SEQ ID NOs: 3, 5, 7, 27, 32, 37, 59, 80, 92, 103, 106, 108, 109, 110, and 111.

D. Carbohydrate Screen.

The dry residue was transferred from the extracting cartridge (10-20 mg) into a 100×13 mm glass tube containing 0.5 ml of 0.5 N HCI in methanol and 0.12 ml of methyl acetate and then sealed (Teflon coated screw cap) under nitrogen and heated for 16 hours at 80° C. The liquid phase was then transferred using an 8-channel pipetter (Matrix) to a glass insert supported by a 96 well aluminum block plate (Modem Metal Craft) and evaporated to dryness (Concentrator Evaparray). The methyl-glycosides and methyl-glycoside methyl esters were silylated in 0.1 ml pyridine and 0.1 ml BSTFA+1% TMCS at room temperature for one hour. The sample generated was analyzed on a DB 1 capillary column (15 meters) with an 11 minute program temperature (from 160° C. to 190° C. at 5° C./min and 190° C. to 298° C. at 36° C./minute and hold 2 minutes) and 3 minutes equilibration time. The following components of the plant cell wall were identified in the tobacco sample: arabinose, rhamnose, xylose, galactose, galacturonic acid, mannose, glucuronic acid and glucose.

E. GC/MS Metabolite Analysis:

A 3 mm tungsten carbide ball bearing was placed into each well of a 96-well deep well block and 300 μl of grinding buffer (2 mM NaOH, 1 mM PMSF, 10 mM beta-mercaptoethanol, and deuterium-labeled compounds) was added to each well. A 13 mm circle (˜20 mg) leaf disc plug from ˜4 week old Nicotiana benthamiana (2 week post-inoculation) apical leaves were placed into the 96-well microtiter deepwell plate. The plate was tightly sealed and placed on a mechanical shaker (paint mixer, up to four at a time) for 2 min, then rotated 180° and shaken for an additional 2 min. Subsequently, the samples were spun for 10 min at 3200 RPM in a refrigerated (15° C.) centrifuge equipped for microtiter plates. Following centrifugation, the 96-well plate containing the homogenized samples was placed on a TECAN GENESIS RSP 200 (TECAN, Research Triangle Park, N.C.) liquid handler/robotics system. Both Logic and Gemini software were used to control the TECAN liquid handler. Approximately 200 μl was transferred to a pre-conditioned (1 ml MeOH followed by 1 ml of distilled deionized H₂O) Waters 96-well Oasis HLB solid phase extraction (SPE) plate by the TECAN liquid handler for metabolite analysis by GC/MS. The Waters Extraction Plate Manifold Kit and a vacuum not greater than 5 mm Hg was used to aspirate plant samples from SPE plate into a waste reservoir. The SPE plate was then washed with 1 ml of 5% MeOH in H₂O by aspirating into waste reservoir and compounds eluted from SP resin with 350 μl of MeOH into a 96-well collection plate. Samples were then transferred to GC autosampler vials, capped and stored in the freezer at 80° C. for metabolite analysis.

An internal standard solution was prepared by making a stock solution at a concentration of 1 μl (using compound density). Grinding buffer (2 mM NaOH above) with the internal standard was prepared at a concentration of 10 ng/μl for each (3,000 ng/300 μl) to yield a concentration equivalent of approximately 150 ng/mg wet weight of plant tissue. Following extraction of plant material, this solution was transferred to the SPE plate by the TECAN liquid handler and extracted with 350 μl of MeOH. Approximately 20 μl of the sample will be injected onto a 30 m×0.32 mm DB-WAX (1 μm film thickness) GC column with a large volume injector during the preliminary study. The GC column oven was temperature held at 35 C for 5 min, then programmed at 2.5° C./min to 250° C. and held for 15 min.

Samples that contained peaks that were present in altered levels relative to control samples as identified from chromatograms were further analysis using mass spectroscopy. Samples that were transfected with the following nucleic acid sequences were found to have altered metabolic profiles: SEQ ID NO: 43, 50, 81, 85, and 92. Table 7 shows the retention time and % change in peaks relative to controls for several sequences. Table 7 also shows the identity of the peaks as determined by mass spectroscopy.

TABLE 7
Metabolic Profiles

| SEQ ID NO | RT (MIN) | % Change | Compound |
|---|---|---|---|
| 43 | 10.68 | +130 | Malic Acid |
| 43 | 11.63 | +250 | Ribonic Acid; Gamma- |
| | | | lactone |
| 43 | 12.93 | +260 | Quinic Acid |
| 43 | 14.12 | +120 | Inositol |
| 81 | 10.67 | +300 | Malic Acid |
| 81 | 10.87 | +150 | L-Aspartic Acid |
| 81 | 10.92 | +80 | 5-Oxo-L-Proline |
| | | | (pyroglutamic) |
| 81 | 12.48 | +100 | Ribonic Acid |
| 81 | 12.64 | +800 | Citric Acid |
| 81 | 16.44 | +60 | Sucrose |
| 92 FA | 9.31 | −95 | Dodecanoic Acid (12:0) |
| 92 FA | 10.28 | −90 | Myristic Acid (14:0) |
| 92 FA | 11.20 | +500 | Hexadecenoic Acid (16:1) |
| 92 FA | 11.96 | +200 | Oleic Acid (18:1) |
| 92 | 10.68 | +700 | Malic Acid |
| 92 | 11.63 | +300 | Ribonic Acid; Gamma- |
| | | | lactone |
| 92 | 12.33 | +300 | Phosphoric Acid |
| 92 | 12.65 | −1400 | Citric Acid |
| 92 | 12.93 | +500 | Quinic Aci |
| 92 | 14.12 | +800 | Inositol |
| 50 | 11.0 | New | |
| 50 | 11.7 | New | |

A 3 mm tungsten carbide ball bearing was placed into each well of a 96-well deep well block and 300 μl of grinding buffer (2 mM NaOH, 1 mM PMSF, 10 mM beta-mercaptoethanol, and deuterium-labeled compounds) was added to each well. A 13 mm circle (˜20 mg) leaf disc plug from ˜4 week old Nicotiana benthamiana (2 week post-inoculation) apical leaves were placed into the 96-well microtiter deepwell plate. The plate was tightly sealed and placed on a mechanical shaker (paint mixer, up to four at a time) for 2 min, then rotated 180° and shaken for an additional 2 min. Subsequently, the samples were spun for 10 min at 3200 RPM in a refrigerated (15° C.) centrifuge equipped for microtiter plates. Following centrifugation, the 96-well plate containing the homogenized samples was placed on a TECAN GENESIS RSP 200 (TECAN, Research Triangle Park, N.C.) liquid handler/robotics system. Both Logic and Gemini software were used to control the TECAN liquid handler. Approximately 200 μl was transferred to a pre-conditioned (1 ml MeOH followed by 1 ml of distilled deionized H₂O) Waters 96-well Oasis HLB solid phase extraction (SPE) plate by the TECAN liquid handler for metabolite analysis by GC/MS. The Waters Extraction Plate Manifold Kit and a vacuum not greater than 5 mm Hg was used to aspirate plant samples from SPE plate into a waste reservoir. The SPE plate was then washed with 1 ml of 5% MeOH in H₂O by aspirating into waste reservoir and compounds eluted from SP resin with 350 μl of MeOH into a 96-well collection plate. Samples were then transferred to GC autosampler vials, capped and stored in the freezer at −80° C. for metabolite analysis.

XIII. Protein Profiling by MALDI-TOF

Approximately 14 days post-inoculation, 960 different N. benthamiana leaf plugs transfected with encapsidated virion from a GENEWARE® expression library from growth rooms and 38 from N. benthamiana infected in Owensboro, Ky. were collected and the soluble proteins extracted with a high throughput micro-extraction technique described below. An aliquot of this solution was automatically diluted with matrix by a liquid handler in preparation for analysis by MALDI-TOF mass spectrometry for proteins.

A. Sample Preparation by High Throughput Micro-Extraction:

A 3 mm tungsten carbide ball bearing was placed into each well of a 96-well deep well block and 300 μl of grinding buffer (2 mM NaOH, 1 mM PMSF, 10 mM beta-mercaptoethanol, and deuterium-labeled compounds-GC/MS analysis) was added to each well. A 13 mm circle (˜20 mg) leaf disc plug from ˜4 week old Nicotiana benthamiana (2 week post-inoculation) apical leaves were placed into the 96-well microtiter deepwell plate. The plate was tightly sealed and placed on a mechanical shaker (paint mixer, up to four at a time) for 2 min, then rotated 180° and shaken for an additional 2 min. Subsequently, the samples were spun for 10 min at 3200 RPM in a refrigerated (15° C.) centrifuge equipped for microtiter plates. Following centrifugation, the 96-well plate containing the homogenized samples was placed on a TECAN GENESIS RSP 200 (TECAN, Research Triangle Park, N.C.) liquid handler/robotics system. Both Logic and Gemini software were used to control the TECAN liquid handler. Samples were diluted by the TECAN liquid handler in a round bottom 96-well plate for MALDI-TOF analysis by adding 18 μl of sinapinic acid matrix and 2 μl of plant extract to each well. Samples were mixed well by aspirating/dispensing 10 μl volumes five times. A 2 μl aliquot of each sample was spotted onto a 100 sample MALDI plate. In addition, a 5.0 μl aliquot of each sample was transferred to a 96-well microtiter plate for PCR and/or MALDI backup analysis and stored at −80° C. Two plant trays containing 96 individually infected each were extracted each day for 5 days.

B. MALDI-TOF Mass Spectrometry Analysis:

An aliquot of the homogenized plant samples were diluted 1 :10 with sinapinic acid (Aldrich, Milwaukee, Wis.) matrix, 2 μl applied to a stainless steel MALDI plate surface and allowed to air dry for analysis. The sinapinic acid was prepared at a concentration of 10 mg/ml in 0.1% TFA/acetonitrile (70/30) by volume. MALDI-TOF mass spectra were obtained with a PerSeptive Biosystems Voyager DE-PRO operated in the linear mode. A pulsed nitrogen laser operating at 337 nm was used in the delayed extraction mode for ionization. An acceleration voltage of 25 kV with a 90% grid voltage and a 0.1% guide wire voltage was used. Approximately 150 scans were acquired and averaged over the mass range of 2000-156,000 Da. with a low mass gate of 2000. Ion source and mirror pressures were approximately 2.2×10⁻⁷ and 8×10⁻⁸ Torr, respectively. All spectra were mass calibrated with a single-point fit using horse apomyoglobin (16,952 Da).

C. Results:

This study describes a method that was developed using the high-throughout capabilities of MALDI-TOF MS to detect changes in total protein profiles of crude plant extracts derived from a GENEWARE® cDNA library. As many as 192 samples per day were extracted and analyzed for protein profiling using MALDI-TOF mass spectrometry. In addition, the method has been optimized in house for detection of a wide range of protein masses from one MALDI-TOF scan. More than 50 proteins were routinely detected in a MALDI profile spectrum ranging from approx. 3,000 to 110,000 Da. In addition to the coat protein (˜17,500 Da), both small (˜14,500 Da) and large (˜52,750 Da) subunits of RuDP carboxylase were routinely detected in the plant samples. Several other proteins were common to most of the plants analyzed. The most abundant proteins were observed at around 3,386, 3,970, 4,408, 5,230, 7,280 (doubly charged ion for small sub-unit of RuDP carboxylase), 8,334, 9,350, 10,450 (most abundant protein overall), 14,020, 18,006, 19,628, 20,286, 21,173, 24,014, 25,124 and 29,140 (dimer of small sub-unit) daltons. A series of less abundant proteins were also detected. Up-regulated or novel proteins were detected in 17.3% of the 960 spectra that were analyzed. This data was entered into the LIMS database.

XIV. ABRC Library Construction in GENEWARE Expression Vectors

Expressed sequence tag (EST) clones were obtained from the Arabidopsis Biological Resource Center (ABRC; The Ohio State University, Columbus, Ohio 43210). These clones originated from Michigan State University (from the labs of Dr. Thomas Newman of the DOE Plant Research Laboratory and Dr. Chris Somerville, Carnegie Institution of Washington) and from the Centre National de la Recherche Scientifique Project (CNRS project; donated by the Groupement De Recherche 1003, Centre National de la Recherche Scientifique, Dr. Bernard Lescure and colleagues). The clones were derived from cDNA libraries isolated from various tissues of Arabidopsis thaliana var Columbia. A clone set of 11,982 clones was received as glycerol stocks arrayed in 96 well plates, each with an ABRC identifier and associated EST sequence.

An ORF finding algorithm was performed on the EST clone set to find potential full-length genes. Approximately 3,200 full-length genes were found and used to make GENEWARE constructs in the sense orientation. Five thousand of the remaining clones (not full-length) were used to make GENEWARE constructs in the antisense orientation.

Full-length clones used to make constructs in the sense orientation were grown and DNA was isolated using Qiagen (Qiagen Inc., Valencia, Calif. 91355) mini-preps. Each clone was digested with NotI and Sse 8387 eight base pair enzymes. The resultant fragments were individually isolated and then combined. The combined fragments were ligated into pGTN P/N vector (with polylinker extending from PstI to NotI −5′ to 3′). For each set of 96 original clones approximately 192 colonies were picked from the pooled GENEWARE ligations, grown until confluent in deep-well 96-well plates, DNA prepped and sequenced. The ESTs matching the ABRC data was bioinformatically checked by BLAST and a list of missing clones was generated. Pools of clones found to be missing were prepared and subjected to the same process. The entire process resulted in greater than 3,000 full-length sense clones.

The negative sense clones were processed in the same manner, but ligated into pGTN N/P vector (with polylinker extending from NotI to PstI −5′ to 3′). For each set of 96 original clones approximately 192 colonies were picked from the pooled geneware ligations and DNA prepped. The DNA from the GENEWARE ligations was subjected to RFLP analysis using TaqI 4 base cutter. Novel patterns were identified for each set. The RFLP method was applied and only applicable for comparison within a single ABRC plate. This procedure resulted in greater than 6,000 negative sense clones.

The identified clones were re-arrayed, transcribed, encapsidated and used to inoculate plants.

XV. Inoculation of Plants

A. Plant Growth.

N. benthamiana seeds were sown in 6.5 cm pots filled with Redi-earth medium (Scotts) that had been pre-wetted with fertilizer solution (prepared by mixing 147 kg Peters Excel 15-5-15 Cal-Mag (The Scotts Company, Marysville Ohio), 68 kg Peters Excel 15-0-0 Cal-Lite (15% Ca), and 45 kg Peters Excel 10-0-0 MagNitrate (10% Mg) in hot tap water to 596 liters total volume and then injecting this concentrate into irrigation water using an injection system (H. E. Anderson, Muskogee Okla.), at a ratio of 200:1). Seeded pots were placed in the greenhouse for 1 d, transferred to a germination chamber, set to 27° C., for 2 d (Carolina Greenhouses, Kinston, N.C.), and then returned to the greenhouse. Shade curtains (33% transmittance) were used to reduce solar intensity in the greenhouse and artificial lighting, a 1:1 mixture of metal halide and high pressure sodium lamps (Sylvania) that delivered an irradiance of approximately 220 μmol m²s⁻¹, was used to extend day length to 16 h and to supplement solar radiation on overcast days. Evaporative cooling and steam heat were used to regulate greenhouse temperature, maintaining a daytime set point of 27° C. and a nighttime set point of 22° C. At approximately 7 days post sowing (dps), seedlings were thinned to one seedling per pot and at 17 to 21 dps, the pots were spaced farther apart to accommodate plant growth. Plants were watered with Hoagland nutrient solution as required. Following inoculation, waste irrigation water was collected and treated with 0.5% sodium hypochlorite for 10 minutes to neutralize any viral contamination before discharging into the municipal sewer.

B. Innoculation.

For each GENEWARE™ clone, 180 μL of inoculum was prepared by combining equal volumes of encapsidated RNA transcript and FES buffer (0.1M glycine, 0.06 M K₂HPO₄, 1% sodium pyrophosphate, 1% diatomaceous earth (Sigma), and either 1% silicon carbide (Aldrich), or 1% Bentonite (Sigma)). The inoculum was applied to three greenhouse-grown Nicotiana benthamiana plants at 14 or 17 days post sowing (dps) by distributing it onto the upper surface of one pair of leaves of each plant (30 μL per leaf). Either the first pair of leaves or the second pair of leaves above the cotyledons was inoculated on 14 or 17 dps plants, respectively. The inoculum was spread across the leaf surface using one of two different procedures. The first procedure utilized a Cleanfoam swab (Texwipe Co, N.J.) to spread the inoculm across the surface of the leaf while the leaf was supported with a plastic pot label (¾×5 2M/RL, White Thermal Pot Label, United Label). The second implemented a 3″ cotton tipped applicator (Calapro Swab, Fisher Scientific) to spread the inoculum and a gloved finger to support the leaf. Following inoculation the plants were misted with deionized water.

C. Infection.

At 13 days post inoculation (dpi), the plants were examined visually and a numerical score was assigned to each plant to indicate the extent of viral infection symptoms. 0=no infection, 1=possible infection, 2=infection symptoms limited to leaves<50-75% fully expanded, 3=typical infection, 4=atypically severe infection, often accompanied by moderate to severe wilting and/or necrosis.

XVI: Phenotypic Evaluation

At 13 dpi plants were examined and in cases where a plant's visual phenotype deviated substantially from the phenotypes of control plants, a controlled vocabulary utilizing a five-part phrase was used to describe the plants. Phrase: plant region/sub-part/modifier (optional)/symptom/severity. Plant regions: sink leaves (the upper region of the plant considered to be primarily phloem sink tissue at the time of evaluation), source leaves (expanded, fully-infected leaves considered to be phloem source tissue at the time of evaluation), bypassed leaves (leaves [three and four] that display little or no infection symptoms), inoculated leaves (leaves one and two), stem. Subparts: blade, entire, flower, foci, intervein, leaf, lower, major vein, margin, minor vein, node, petiole, shoot apex, upper, vein, viral path. Modifiers: apical, associated, banded, basal, blotchy, bright, central, crinkled, dark, epinastic, flecked, glossy, gray, hyponastic, increased, intermittent, large-spotted, light, light-colored, light-green, mottled, narrowed, orange, patchy, patterned, radial, reduced, ringspot, small-spotted, smooth, spotted, streaked, subtending, uniform, unusual, white. Symptoms: bleaching, chlorosis, color, contortion, corrugation, curling, dark green, elongation, etching, hyperbranching, mild symptoms, necrosis, patterning, recovery, stunting, texture, trichomes, wilting. Severity: 1—extremely mild/trace, 2—mild symptom (<30% of subpart affected), 3—moderate symptom (30%-70% of subpart affected), 4—severe symptom (>70% of subpart affected). Based on the symptoms a phenotypic hit value (PHV) and a herbicide hit value (HHV) were assigned to each plant phenotyped. Phenotype Hit Value: 1—no predicted value; do not request for repeat analysis, 2—of uncertain value, 3—of potential value; strong phenotype, 4—highly unusual phenotype. Herbicide Hit Value: 1—no predicted value; do not request for repeat analysis, 2—of uncertain value, 3—moderate chlorosis (especially in apical region) or necrosis, 4—Severe phytotoxicity/herbicide mode of action. Comments were added if additional information was required to complete the plant characterization. Results are presented in Table 8.

TABLE 8

| SEQ ID NO | Library | Summary of Visual Phenotype |
|---|---|---|
| SEQ ID NO:12 | ABRC | Stunting |
| SEQ ID NO:27 | ABRC | Stunting |
| SEQ ID NO:48 | ABRC | Stunting |
| SEQ ID NO:49 | ABRC | Stunting |
| SEQ ID NO:59 | ABRC | Stunting |
| SEQ ID NO:60 | ABRC | Stunting |
| SEQ ID NO:71 | ARAB | Stunting |
| SEQ ID NO:84 | ABRC | Stunting |
| SEQ ID NO:99 | ABRC | Stunting |
| SEQ ID NO:100 | ABRC | Stunting |
| SEQ ID NO:102 | ABRC | Stunting |
| SEQ ID NO:103 | ABRC | Stunting |
| SEQ ID NO:105 | ABRC | Stunting |
| SEQ ID NO:106 | ABRC | Stunting |
| SEQ ID NO:107 | ABRC | Stunting |
| SEQ ID NO:108 | ABRC | Stunting |
| SEQ ID NO:109 | ABRC | Stunting |
| SEQ ID NO:110 | ABRC | Stunting |

XVII: Metabolic Screens

A. Sample Generation.

Individual dwarf tobacco nicotiana benthamiana, (Nb) plants were manually transfected with an unique DNA sequence at 14 or 17 days post sowing using the GENEWARETM viral vector technology (1). Plants were grown and maintained under greenhouse conditions. At 13 days after infection, an infection rating of 0, 1, 2, 3, or 4 was assigned to each plant. The infection rating documents the degree of infection based on a visual observation. A score of 0 indicates no visual infection. Scores of 1 and 2 indicate varying degrees of partial infection. A score of 4 indicates a plant with a massive overload of infection, the plant is either dead or near death. A score of 3 indicates optimum spread of systemic infection.

Samples were grouped into sets of up to 96 samples per set for inoculation, harvesting and analysis. Each sample set (SDG) included 8 negative control (reference samples), up to 80 unknown (test) samples, and 8 quality control samples.

B. Harvesting.

At 14 days after infection, infected leaf tissue, excluding stems and petioles, was harvested from plants with an infection score of 3. Infected tissue was placed in a labeled, 50-milliliter (mL), plastic centrifuge tube containing a tungsten carbide ball approximately 1 cm in diameter. The tube was immediately capped, and dipped in liquid nitrogen for approximately 20 seconds to freeze the sample as quickly as possible to minimize degradation of the sample due to biological processes triggered by the harvesting process. Harvested samples were maintained at −80 C between harvest and analysis. Each sample was assigned a unique identifier, which was used to correlate the plant tissue to the DNA sequence that the plant was transfected with. Each sample set was assigned a unique identifier, which is referred to as the harvest or meta rack ID.

C. Extraction.

Prior to analysis, the frozen sample was homogenized by placing the centrifuge tube on a mechanical shaker. The action of the tungsten carbide ball during approximately 30 seconds of vigorous shaking reduced the frozen whole leaf tissue to a finely homogenized frozen powder. Approximately 1 gram of the frozen powder was extracted with 7.5 mL of a solution of isopropanol (IPA):water 70:30 (v:v) by shaking at room temperature for 30 minutes.

D. Fractionation.

A 1200 microliter (μL) aliquot of the IPA:water extract was partitioned with 1200 μL of hexane. The hexane layer was removed to a clean glass container. This hexane extract is referred to as fraction 1 (F1). A 90 μL aliquot of the hexane extracted IPA:water extract was removed to a clean glass container. This aliquot is referred to as fraction 4 (F4). The remaining hexane extracted IPA:water extract is referred to as fraction 3 (F3). A 200 μL aliquot of the IPA:water extract was transferred to a clean glass container and referred to as fraction 2 (F2). Each fraction for each sample was assigned a unique aliquot ID (sample name).

E. Sample Preparation & Data Generation

Fraction 1:

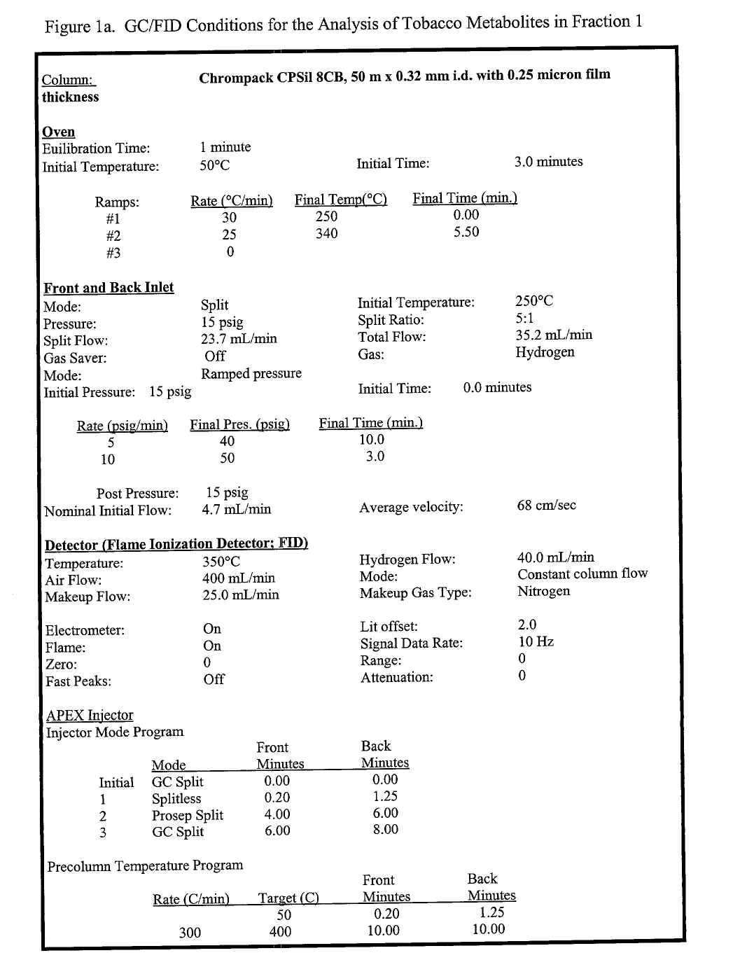

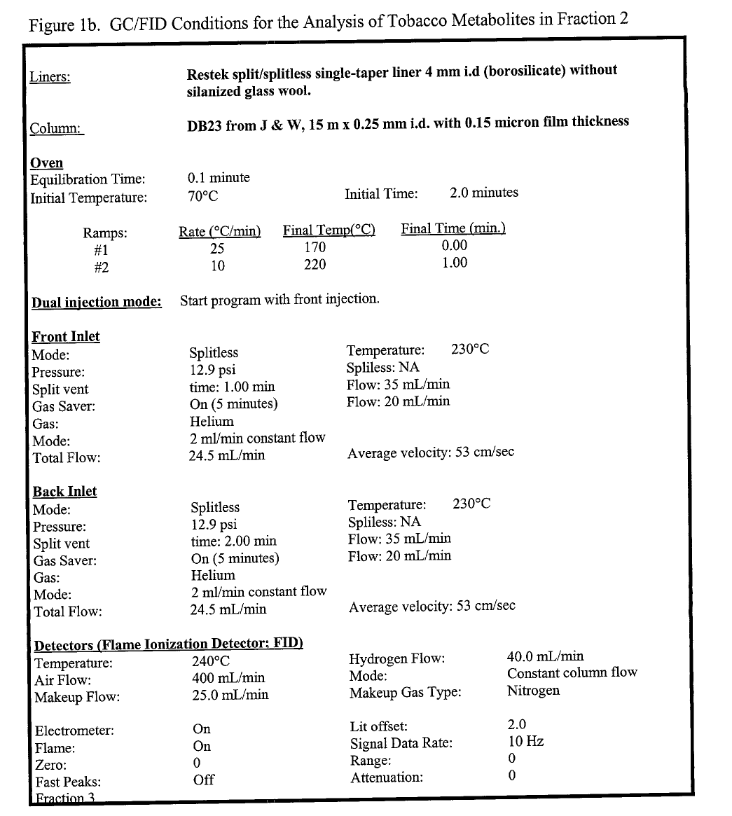

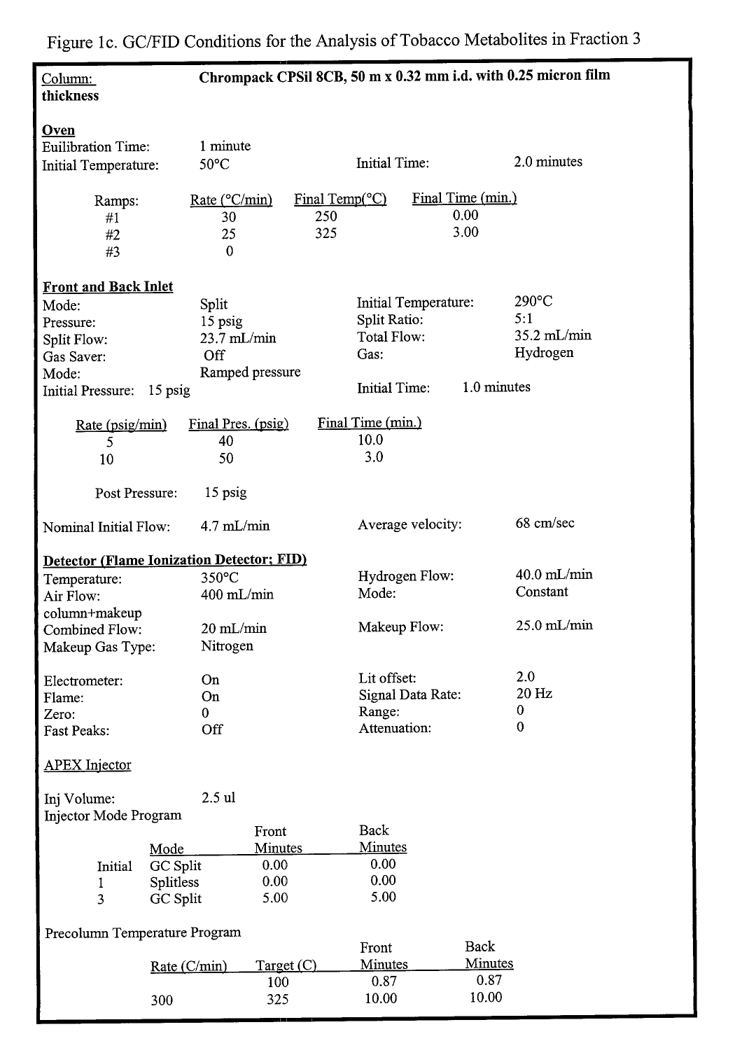

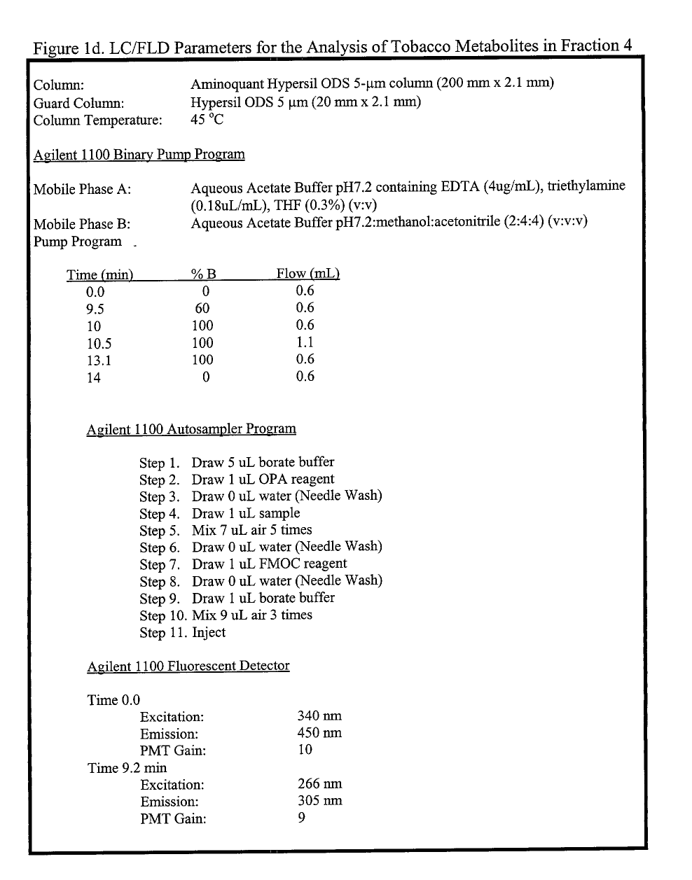

The hexane extract was evaporated to dryness under nitrogen at room temperature. The sample containers were sealed and stored at 4 C prior to analysis, if storage was required. Immediately prior to capillary gas chromatographic analysis using flame ionization detection (GC/FID), the F1 residue was reconstituted with 120 μL of hexane containing pentacosane and hexatriacontane which were used as internal standards for the F1 analyses. The chromatographic data files generated following GC separation and flame ionization detection were named with the fraction 1 aliquot ID for each sample and stored in a folder named after the harvest rack (sample set) ID. FIG. 1 a summarizes the GC/FID parameters used to analyze fraction 1 samples.

Fraction 2:

The F2 aliquot was evaporated to dryness under nitrogen at room temperature and reconstituted in heptane containing 2 internal standards, C11:0 and C24:0. In general, fraction 2 is designed to analyze esterified fatty acids, such as phospholipids, triacylglycerides, and thioesters. In order to analyze these compounds by GC/FID, they were transmethylated to their respective methyl esters by addition of sodium methoxide in methanol and heat. Excess reagent was quenched by the addition of a small amount of water, which results in phase separation. The fatty acid methyl esters (FAMEs) were contained in the organic phase. FIG. 1b summarizes the GC/FID parameters used to analyze fraction 1 samples.

Fraction 3:

The F3 aliquot was evaporated to dryness under nitrogen at 40 C. In general, the metabolites in this fraction are highly polar and water-soluble. In order to analyze these compounds by GC/FID, the polar functional groups on these compounds were silylated through a 2-step derivatization process. Initially, the residue was reconstituted with 400 μL of pyridine containing hydroxylamine hydrochloride (25 mg/ml) and the internal standard, n-octyl-β-D-glucopyranoside (OXIME solution). The derivatization was completed by the addition of 400 μL of the commercially available reagent (N,O-bis[Trimethylsily] trifluoroacetamide)+1% Trimethylchlorosilane (BSTFA+1% TMCS). The chromatographic data files generated following GC separation and flame ionization detection were named with the fraction 3 aliquot ID for each sample and stored in a folder named after the harvest rack (sample set) ID. FIG. 1c summarizes the GC/FID parameters used to analyze fraction 1 samples.

Fraction 4:

The F4 aliquot was diluted with 90 μL of distilled water and 20 μL of an 0.1 N hydrochloric acid solution containing norvaline and sarcosine, which are amino acids that are used as internal standards for the amino acids analysis. Immediately prior to high performance liquid chromatographic analysis using fluorescence detection (HPLC/FLD), the amino acids in F4 are mixed in the HPLC injector at room temperature with buffered orthophtaldehyde solution, which derivatizes primary amino acids, followed by fluorenyl methyl chloroformate, which derivatizes secondary amino acids. Following HPLC separation and fluorescence detection, chromatographic data files were generated for each sample, named with a sequential number which can be tracked back to the F4 aliquot ID, and stored in a folder named after the harvest rack (sample set) ID. FIG. 1d summarizes the GC/FID parameters used to analyze fraction 1 samples.

F. Data Analysis & Hit Detection.

Two complementary methods were used to identify modifications in the metabolic profile of test samples from reference samples. These data analysis methods are called automated data analysis (ADA) and quantitative data analysis. Each fraction from each sample was analyzed by one or both of these methods to identify hits. If either method identified a fraction as a hit, the sample was called a hit for that fraction. Therefore a sample could be a hit for 1 through 4 fractions.

ADA employs a qualitative pattern recognition approach using ABNORM (U.S. Pat. No. 5,592,402), which is a proprietary software utility of the Dow Chemical Company. ADA was performed on chromatograms from all 4 fractions. The ADA process developed a statistical model from chromatograms that ideally depict unaltered (reference) metabolic profiles. This model was then used to identify test sample chromatograms that contain statistically significant differences from the normal (control) chromatograms. Updated models for each fraction were generated for each sample set. Chromatograms identified as hits by ADA, were manually reviewed and the data quality visually verified.

Quantitative data analysis is based on individual peak areas. Quantitative data analysis was applied to specific compounds of interest in fraction 2, fatty acids, and fraction 4, amino acids. The peak areas corresponding to these compounds in these fractions were generated. For fraction 2, the relative percent of the peak areas for the compounds in Table 9 were calculated for each sample. The average (\overline{x}) and standard deviation (STD) of the relative % of the peak areas for the individual compounds were calculated from the reference sample chromatograms analyzed within the sample set. The average and STD were used to calculate a range for each compound. Depending on the compound, this range was typically \overline{x}+/−3 or 5 STDs. If the relative percent of the peak area from an unknown was outside this range, the compound was considered to be significantly different from the ‘normal’ level and the sample was identified as a hit for F2. For fraction 4, the concentration, in micrograms/gram was calculated for each of the amino acids listed in Table 9, from calibration standards analyzed at the same time as the test samples. The amino acid concentrations from reference samples were used to calculate the acceptable range from the \overline{x} and STD for each amino acid. If the amino acid concentration for an unknown falls outside this range, the amino acid was considered to be different from normal and sample was identified as a hit for F4.\

TABLE 9
Tobacco Metabolites Monitored in Fractions 2 and 4 by
Quantitative Analysis

| | | Fraction 4 | |
|---|---|---|---|
| Fraction 2 (Fatty Acids) | | (Amino Acids) | |
| undecanoic acid methyl ester* | C11:0 | Aspartic Acid | ASP |
| Pentadecanoic acid methyl ester** | C15:0 | Glutamic | GLU |
| | | Acid | |
| Pentadecanoic acid ethyl ester** | C15:0 | Serine | SER |
| palmitic acid methyl ester | C16:0 | Histidine | HIS |
| palmitoleic acid methyl ester | C16:1 | Glycine | GLY |
| iso methylpentadecanoic acid methyl | C16:0:Me | Threonine | THR |
| ester | | | |
| palmitoleic acid methyl ester | C16:2 | Alanine | ALA |
| palmitolenic acid methyl ester | C16:3 | Arginine | ARG |
| iso methylhexadecanoic acid methyl | C17:0Me | Tyrosine | TYR |
| ester | | | |
| Stearic acid methyl ester | C18:0 | Cystine | CY2 |
| Oleic acid methyl ester | C18:1 | Valine | VAL |
| Linoleic acid methyl ester | C18:2 | Methionine | MET |
| Linolenic acid methyl ester | C18:3 | Norvaline* | NVA |
| Arachidic acid methyl ester | C20:0 | Tryptohane | TRP |
| Lignoceric acid methyl ester* | C24:0 | Phenylalanine | PHE |
| | | Isoleucine | ILE |
| | | Leucine | LEU |
| | | Lysine | LYS |
| | | Sarcosine* | SAR |
| | | Proline | PRO |

*Internal Standard
**Surrogate Standard

Shipping Hits.

Any F1, F2, or F3 fractions identified as hits by ADA or quantitative analysis, and the most typical null for each fraction for each sample set as identified by ADA, were sent to the Function Discovery Laboratory (see Example 20) for structural characterization of the specific compounds identified. Samples were sealed, packaged on dry ice and shipped for overnight delivery.

XVIII: Identification of Metabolic Changes

This Example describes the identification of the chemical nature of genetic modifications made in tobacco plants using GENEWARE viral vector technology. The protocols involved the use of gas chromatography/mass spectrometry (GC/MS) for the analyses of three primary fractions obtained from extraction and fractionation processes.

A. Methods.

Major instruments and accessories used included Bioinformatics computer programs, mass spectral libraries, Biotech databases, Nautilus LIMS system (BLIMS; Dow), Biotech Database (eBRAD; Dow), HP Model 6890 capillary Gas Chromatograph (GC; Agilent Technologies), HP Model 5973 Mass Selective Detector (MSD; Agilent Technologies), Auto Sampler and Sample Preparation Station (Leap Technologies), Large Volume Injector system (APEX), Ultra Freezer (Revco), and model LS1006 Barcode Reader (Symbol Technologies).

Samples and corresponding References (also referred to as controls or nulls) were shipped via overnight mail. Samples were removed from the shipping container, inspected for damage, and then placed in a freezer until analysis by GC/MS.

Samples were received in vials or in titer plates with a bar-coded titer plate (TP) number, also referred to as a Rack Identification number that is used to track the sample in the BLIMS system. The barcode number is used by the FDL to extract from BLIMS pertinent information from ADA (Automated chromatographic pattern recognition Data Analysis) HIT reports and/or QUANT (a quantitative data analysis approach that makes use of individual peak areas of select peaks corresponding to specific compounds of interest in the fatty acid Fraction 2) HIT reports generated by the Metabolic Screening Laboratory. The information in these reports includes the well position of the respective HITs (Samples), the corresponding well position of the Reference, and other pertinent information, such as, aliquot identification. This information is used to generate ChemStation and Leap sequences for FDL analyses.

Samples were sequenced for analysis in the following order:

TABLE 10
Analysis Order

Solvent Blank
Instrument Performance Standard
Samples and Associated Reference
.
.
.
Performance Standard
Solvent Blank

Samples were analyzed on GC/MS systems using the following procedures. Fraction 1 samples were shipped dry and required a hexane reconstitution step. Fraction 2 and Fraction 3 samples were analyzed as received. Internal standards were added to the samples prior to analysis.

B. Fraction 1 Analysis.

The name of the GC/MS method used is BIONEUTx (where x is a revision number of the core GC/MS method). The method is retention-time locked to the retention time of pentacosane, an internal standard, using the ChemStation RT Locking algorithm.

| | |
|---|---|
| Internal Standard(s) | |
| Pentacosane | |
| Hexatriacontane | |
| Chromatography | |
| Column: | J & W DB-5MS |
| | 50 M × 0.320 mm × 0.25 μm film |
| Mode: constant flow | |
| | Flow: 2.0 mL/min |
| | Detector: MSD |
| | Outlet psi: vacuum |
| Oven: | 40° C. for 2.0 min |
| | 20° C./min to 350° C., hold 15.0 min |
| | Equilibration time: 1 min |
| Inlet: | Mode: split |
| | Inj Temp: 250° C. |
| | Split ratio: 50:1 |
| | Gas Type: Helium |
| LEAP Injector: | |
| Injector: | Inj volume: optimized to pentacosane peak intensity |
| | (typically 20 μL) |
| | Sample pumps: 2 |
| | Wash solvent A: Hexane |
| | Wash solvent B: Acetone |
| | Preinj Solvent A washes: 2 |
| | Preinj Solvent B washes: 2 |
| | Postinj Solvent A washes: 2 |
| | Postinj Solvent B washes: 2 |
| APEX Injector | |
| Method Name: | BIONEUTx (where x is a revision number of the |
| | core APEX method). |
| Modes: | Initial: Standby (GC Split) |
| | Splitless: (Purge Off) 0.5 min |
| | GC Split: (Standby) 4 min |
| | ProSep Split: (Flow Select) 23 min |
| Temps: 50° C. for | |
| 0.0 min. | |
| | 300° C./min to 350° C., hold for 31.5 min |
| Mass Spectrometer | |
| Scan: 35-800 | Da at sampling rate 2 (1.96 scans/sec) |
| | Solvent delay: 4.0 min |
| Detector: | EM absolute: False |
| | EM offset: 0 |
| Temps: | Transfer line: 280° C. |
| | Ion source: 150° C. |
| | MS Source: 230° C. |

C. Fraction 2 Analysis:

The name of the GC/MS method used is BIOFAMEx (where x is a revision number of the core GC/MS method). The method is retention-time locked to RT of undecanoic acid, methyl ester, an internal standard, using the ChemStation RT Locking algorithm.

| | |
|---|---|
| Internal Standard(s) | |
| Undecanoic acid, methyl ester | |
| Tetracosanoic acid, methyl ester | |
| Chromatography | |
| Column: | J & W DB-23 FAME |
| | 60 M × 0.250 mm × 0.15 μm film |
| | Mode: constant flow |
| | Flow: 2.0 mL/min |
| | Detector: MSD |
| | Outlet psi: vacuum |
| Oven: | 50° C. for 2.0 min |
| | 20° C./min to 240° C., hold 10.0 min |
| | Equilibration time: 1 min |
| Inlet: | Mode: split |
| | Inj Temp: 240° C. |
| | Split ratio: 50:1 |
| | Gas Type: Helium |
| LEAP Injector: | |
| Injector: | Inj volume: optimized to undecanoic |
| | acid, methyl ester peak intensity |
| | (Typically 10 μL) |
| | Sample pumps: 2 |
| | Wash solvent A: Methanol |
| | Wash solvent B: Methanol |
| | Preinj Solvent A washes: 2 |
| | Preinj Solvent B washes: 2 |
| | Postinj Solvent A washes: 2 |
| | Postinj Solvent B washes: 2 |
| APEX Injector | |
| Method Name: | BIOFAMEx (where x is a revision |
| | number of the core APEX method). |
| Modes: | Initial: GC Split |
| | Splitless: 0.5 min |
| | GC Split: 4 min |
| ProSep Split: | 21 min |
| Temps: | 60° C. for 0.5 min. |
| | 300° C./min to 250° C., hold for 20 min |
| | 300° C./min to 260° C., hold for 5 min |
| Mass Spectrometer | |
| Scan: 35-800 | Da at sampling rate 2 (1.96 scans/sec) |
| | Solvent delay: 4.5 min |
| Detector: | EM absolute: False |
| EM offset: 0 | |
| Temps: | Transfer line: 200° C. |
| | Ion source: 150° C. |
| | MS Source: 230° C. |

D. Fraction 3 Analysis.

The name of the GC/MS method used is BIOAQUAx (where x is a revision number of the core GC/MS method). Method is retention-time locked to the RT of n-Octyl-β-D-Glucopyranoside, an internal standard, using the ChemStation RT Locking algorithm.

| | |
|---|---|
| Internal Standard(s) | |
| n-Octyl-β-D-Glucopyranoside | |
| Chromatography | |
| Column: | Chrompack 7454 CP-SIL 8 |
| | 60 M × 0.320 mm × 0.25 μm film |
| | Mode: constant flow |
| | Flow: 2.0 mL/min |
| | Detector: MSD |
| | Outlet psi: vacuum |
| Oven: | 40° C. for 2.0 min |
| | 20° C./min to 350° C., hold 10.0 min |
| | Equilibration time: 1 min |
| Inlet: Mode: split | |
| | Inj Temp: 250° C. |
| | Split ratio: 50:1 |
| | Gas Type: Helium |
| LEAP Injector: | |
| Injector: | Inj volume: Optimized to n-Octyl-β-D- |
| | Glucopyranoside peak intensity |
| (Typically 2.5 μL) | |
| | Sample pumps: 2 |
| | Wash solvent A: Hexane |
| | Wash solvent B: Acetone |
| | Preinj Solvent A washes: 2 |
| | Preinj Solvent B washes: 2 |
| | Postinj Solvent A washes: 2 |
| | Postinj Solvent B washes: 2 |
| APEX Injector | |
| Method Name: | BIQAQUAx (where x is a revision |
| | number of the core APEX method). |
| Modes: | Initial: GC Split |
| | Splitless: 0.5 min |
| | GC Split: 4 min |
| | ProSep Split: 20 min |
| Temps: | 60° C. for 0.5 min. |
| | 300° C./min to 350° C., hold for 21.1 min |
| Mass Spectrometer | |
| Scan: 35-800 | Da at sampling rate 2 (1.96 scans/sec) |
| | Solvent delay: 4.0 min |
| Detector: | EM absolute: False |
| EM offset: 0 | |
| Temps: | Transfer line: 280° C. |
| | Ion source: 150° C. |
| | MS Source: 230° C. |

E. Performance Standard:

Two mixtures were used as instrument performance standards. One standard was run with Fraction 1 and 3 samples and the second was run with Fraction 2 samples. Below is the composition of the standards as well as approximate retention time values observed when run under the GC/MS conditions previously described. These retention time values are subject to change depending upon specific instrument and chromatographic conditions.

TABLE 11
Fraction 1 and 3 Performance Standard

| Time | Compound |
|---|---|
| 6.25 | dimethyl malonate |
| 7.25 | dimethyl succinate |
| 8.15 | dimethyl glutarate |
| 8.98 | dimethyl adipate |
| 11.06 | dimethyl azelate |
| 11.42 | hexadecane |
| 11.70 | dimethyl sebacate |
| 13.57 | eicosane |
| 15.36 | tetracosane |
| 16.88 | octacosane |
| 18.26 | dotriacontane |
| 19.95 | hexatriacontane |

TABLE 12
Fraction 2 Performance Standard

| Time | Compound |
|---|---|
| 8.82 | undecanoic acid, methyl ester |
| 9.32 | dodecanoic acid, methyl ester |
| 10.24 | tetradecanoic acid, methyl ester |
| 11.07 | hexadecanoic acid, methyl ester |
| 11.84 | octadecanoic acid, methyl ester |
| 11.90 | oleic acid, methyl ester |
| 12.14 | linoleic acid, methyl ester |
| 12.39 | linolenic acid, methyl ester |
| 12.60 | eicosanoic acid, methyl ester |
| 13.42 | docosanoic acid, methyl ester |

F. Data Analysis.

Sample and Reference data sets were processed using the Bioinformatics computer program Maxwell. The principal elements of the program are 1) Data Reduction, 2) two-dimensional Peak Matching, 3) Quantitative Peak Differentiation (Determination of Relative Quantitative Change), 4) Peak Identification, 5) Data Sorting, and 6) Customized Reporting.

The program queries the user for the filenames of the Reference data set and Sample data set(s) to compare against the Reference. A complete listing of user inputs with example input is shown below.

TABLE 13
Bioinformatics Analysis

| USER QUERY | EXAMPLE USER INPUT |
|---|---|
| Operator Name | M. Maxwell |
| Total number of data files to process | 5 |
| Which Fraction | 3 |
| Reference (Control) File Name | AAPR0020.D |
| Process a specific RT Range | Y |
| Specific RT range | 6.5-23 |
| Internal Standard Retention Time | 14.902 |
| +/− variation in Internal Std. RT | .004 |
| Variation in peak RI, ChemStation | .005 |
| Percent variation in peak RI, Biotech | .010 |
| Database | |
| Threshold for determining Area % change | 60 |
| Spectral Matching Value (Threshold MS- | .95 |
| XCR for peaks to be a match) | |
| Percent to determine LOP-PM* Value | 1 |
| Percent to determine LOP-SRT** Value | 3 |
| Quality Level for Library (Library match) | 80 |
| Subtract Background | Y |
| Time Range for Background | 21.5-22.6 |
| SHORT SUMMARY (y/n, y = no | Y |
| chromatograms) | |

*LOP-PM - Limit of Processing for Peak Matching
**LOP-SRT - Limit of Processing for Sorting

The program integrates the Total Ion Chromatogram (TIC) of the data sets using Agilent Technologies HP ChemStation integrator parameters determined by the analyst. The corresponding raw peak areas are then normalized to the respective Internal Standard peak area. It should be noted that before the normalization is performed, the program chromatographically and spectrally identifies the Internal Standard peak. Should the identification of the Internal Standard not meet established criteria for a given Fraction, then the data set will not be further processed and it will be flagged for analyst intervention.

Peak tables from the Reference and each Sample were generated. The peak tables are comprised of retention time (RT), retention index (RI)—the retention time relative to the Internal Standard RT, raw peak areas, peak areas normalized to the Internal Standard, and other pertinent information.

The first of two filtering criteria, established by the analyst was then invoked and must be met before a peak is further processed. The criterion is based upon a peak's normalized area. All normalized peaks having values below the Limit of Processing for Peak Matching (LOP-PM), were considered to be “background”. These “peaks” were not carried forth for any type of mathematical calculation or spectral comparison.

In the initial peak-matching step, the Sample peak table was compared to the Reference peak table and peaks between the two were paired based upon their respective RI values matching one another (within a given variable window). The next step in the peak matching routine utilized mass spectral data. Sample and Reference peaks that have been chromatographically matched were then compared spectrally. The spectral matching was performed using a mass spectral cross-correlation algorithm within the Agilent Technologies HP ChemStation software. The cross-correlation algorithm generates an equivalence value based upon spectral “fit” that was used to determine whether the chromatographically matched peaks are spectrally similar or not. This equivalence value is referred to as the MS-XCR value and must meet or exceed a predetermined value for a pair of peaks to be “MATCHED,” which means they appear to be the same compound in both the Reference and the Sample. The MS-XCR value can also be used to judge peak purity. This two-dimensional peak matching process was repeated until all potential peak matches were processed. At the end of the process, peaks are categorized into two categories, MATCHED and UNMATCHED.

A second filtering criterion was next invoked, again based upon the normalized area of the MATCHED or UNMATCHED peak. For a peak to be reported and further processed, its normalized area must meet or exceed the predetermined Limit of Processing for Sorting (LOP-SRT).

Peaks that are UNMATCHED are immediately flagged as different. UNMATCHED peaks are of two types. There are those that are reported in the Reference but appear to be absent in the Sample (based upon criteria for quantitation and reporting). These peaks were designated in the Analyst Report with a percent change of “−100 percent” and the description “UNMATCHED IN SAMPLE.” The second types of peaks are those that were not reported in the Reference (again, based upon criteria for quantitation and reporting) but were reported in the Sample, thus appearing to be “new” peaks. These peaks were designated in the Analyst Report with a percent change of “100 percent” and the description “NEW PEAK UNMATCHED IN NULL.”

MATCHED peaks were processed further for relative quantitative differentiation. This quantitative differentiation is expressed as a percent change of the Sample peak area relative to the area of the Reference peak. A predetermined threshold for change must be observed for the change to be determined biochemical and statistically significant. The change threshold is based upon previously observed biological and analytical variability factors. Only changes above the threshold for change were reported.

Peaks were then processed through the peak identification process as follows. The mass spectra of the peaks were first searched against mass spectral plant metabolite libraries. The equivalence value assigned to the library match was used as an indication of a proper identification.

To provide additional confirmation to the identity of a peak, or to suggest other possibilities, library hits were searched further against a Biotechnology database. The Biotechnology database is based on the Access database program from Accelrys (formerly Synopsis) and utilizes Accord for Access (also available from Accelrys) to incorporate chemical structures into the database.

The Chemical Abstract Services (CAS) number of the compound from the library was searched against those contained in the database. If a match was found, the CAS number in the database was then correlated to the data acquisition method for that record. If the method was matched, the program then compared the retention index (RI), in the Peak Table, of the component against the value contained in the database for that given method. Should the RI's match (within a given window of variability) then the peak identity was given a high degree of certainty. Components in the Sample that are not identified by this process were assigned a unique identifier based upon Fraction Number and RI (example: F1-U0.555). The unique identifier was used to track unknown components. The program then sorts the data and generates an Analyst Report.

An Analyst Report is an interim report consisting of PBM algorithm match quality value (equivalence value), RT, Normalized Peak Area, RI (Sample), RI (database) Peak Identification status [peak identity of high certainty (peaks were identified by the program based on the pre-established criteria) or criteria not met (program did not positively identify the component)], Component Name, CAS Number, Mass Spectral Library (containing spectrum most closely matched to that of the component), Unknown ID (unique identifier used to track unidentified components), MS-XCR value, Relative % Change, Notes (MATCHED UNMATCHED), and other miscellaneous information. The Analyst Report was reviewed manually by the analyst who determined what further analysis was necessary. The analyst also generated a modified report, for further processing by the program, by editing the Analyst Report accordingly.

For Fractions 2 and 3, derivatization procedures were performed prior to analysis to make the certain components more amenable to gas chromatography. Thus, the compound names in the modified analyst report (MAR) were those of the derivatives. To accurately reflect the true components of these fractions, the MAR was further processed using information contained in an additional database. This database cross-references the observed derivatized compound to that of the original, underivatized “parent” compound by way of their respective CAS numbers and replaces derivatives with parent names and information for the final report. In addition, any unidentified components were assigned a “999999-99-9” CAS number.

The Modified Analyst Report also contains a HIT Score of 0, 1, or 2. The value is assigned by the analyst to the data set of the Sample aliquot based on the following criteria:

0 No FDL data on Sample

1 FDL data collected; Sample not FDL HIT

2 FDL data collected; Sample is FDL HIT

An FDL HIT is defined as a reportable percent change (modification) observed in a Sample relative to Reference in a component of biochemical significance.

An electronic copy of the final report is entered into the Nautilus LIMS system (BLIMS) and subsequently into eBRAD (Biotech database). The program also generated a hardcopy of the pinpointed TIC and the respective mass spectrum of each component that was reported to have changed.

“NQ” and “NEW” are two terms used in the final report. Both terms refer to UNMATCHED peaks whose percent changes cannot be reported in a numerically quantitative fashion. These terms are defined as follows:

“NQ” is used in the case where there was a peak reported in the Reference for which there was no match in the Sample (either because there was no peak in the Sample or, if there was, the area of the peak did not satisfy the Limit of Processing for Peak Matching). The percent change designation of “−100%” used in the Analyst report is replaced with “NQ”.

“NEW” is used in those situations where a peak was reported in the Sample but for which there was no corresponding match in the Reference (either because there was no peak in the Reference or, if there was, the area of the peak did not satisfy the Limit of Processing for Peak Matching). For these situations, the percent change designation of “100%” used in the Analyst Report is replaced with “NEW”. The designation of “NEW” in the final report to a component that is present in the Sample but not in the Reference was necessary to eliminate any ambiguity with the appearance of “100%” for MATCHED peaks. A “100%” designation in the final report exclusively refers to a component with modification that doubled in the Sample relative to the Reference.

G. Results.

The results of the metabolic screening revealed that transfection with 55 of the inserts resulted in measurable metabolic changes.

1. A method of creating a transfected or transgenic plant chosen from the group consisting of ornamental, horticultural, forestry, medicinal or Nicotiana sp. plants, exhibiting a dwarf phenotype comprising: expressing in the plant the DNA identified by a polynucleotide sequence chosen from the group consisting of SEQ. ID NO: 1-122 or the mRNA encoded by the DNA identified by a polynucleotide sequence chosen from the group consisting of SEQ. ID NO: 1-122:

2. A method of creating a transfected or transgenic plant chosen from the group consisting of ornamental, horticultural, forestry, medicinal or Nicotiana sp. plants, exhibiting a dwarf phenotype comprising the steps of:
(a) providing a viral inoculum capable of infecting a plant comprising the DNA identified by a polynucleotide sequence chosen from the group of SEQ. ID NO: 1-122 or the mRNA encoded by the DNA identified by a polynucleotide sequence chosen from the group of SEQ. ID NO: 1-122;
(b) applying said viral inoculum to a plant;
whereby the plant is infected and the DNA or the mRNA is expressed in the plant.

3. The method of claims 1 or 2 wherein the plant is turfgrass.

4. The method of claims 1 or 2 wherein the plant is fir tree.

5. A transfected or transgenic plant chosen from the group consisting of ornamental, horticultural, forestry, medicinal or Nicotiana sp. plants, exhibiting a dwarf phenotype made by the method comprising: expressing in the plant the DNA identified by a polynucleotide sequence chosen from the group consisting of SEQ. ID NO: 1-122 or the mRNA encoded by the DNA identified by a polynucleotide sequence chosen from the group consisting of SEQ. ID NO: 1-122.

6. The transfected or transgenic plant of claim 5 wherein the plant is turfgrass.

7. The transfected or transgenic plant of claim 5 wherein the plant is fir tree.

8. A transfected or transgenic plant chosen from the group consisting of ornamental, horticultural, forestry, medicinal or Nicotiana sp. plants, exhibiting a dwarf phenotype made by the method comprising the steps of:
(a) providing a viral inoculum capable of infecting a plant comprising the DNA identified by a polynucleotide sequence chosen from the group of SEQ. ID NO: 1-122 or the mRNA encoded by the DNA identified by a polynucleotide sequence chosen from the group of SEQ. ID NO: 1-122;
(b) applying said viral inoculum to a plant;
whereby the plant is infected and the DNA or the mRNA is expressed in the plant.

9. The transfected or transgenic plant of claim 8 wherein the plant is turfgrass.

10. The transfected or transgenic plant of claim 8 wherein the plant is fir tree.

11. A method of producing multiple crops of the plant of claims 5-10 comprising the steps of:
(a) planting a reproductive unit of the plant;
(b) growing the planted reproductive unit under natural light conditions;
(c) harvesting the plant; and
(d) repeating steps (a) through (c) at least once in the year.

12. A method of manufacturing a biopharmaceutical comprising:
(a) providing a plant that expresses a biopharmaceutical in the plant;
(b) providing a viral inoculum capable of infecting a plant comprising the DNA identified by a polynucleotide sequence chosen from the group of SEQ. ID NO: 1-122 or the mRNA encoded by the DNA identified by a polynucleotide sequence chosen from the group of SEQ. ID NO: 1-122;
(c) applying said viral inoculum to the plant;
whereby the plant is infected, exhibits a dwarf phenotype, and expresses the biopharmaceutical.


SEQUENCE LISTING (122 sequences)

SEQ ID NO: 1 - DNA, 817 bp, Nicotiana benthamiana
agcaatcttaactccgccttctacctcgatctcccaacagaggcaacccttgcccaacttctctgaaacacgcatcaccattgcacctctctgctctccgtactcacttgggcagtatgagaagcaatatgtttacggggttcacgaggctttgcaagggtcttgcggttgtgcttgtgggcggtcacattgttgtccagattcttccttctgctctttcctatcttgctctcatccctgtcaagatgaggacgttgcatggagctggagggatggatttctcccacctgatgatcatcatctttaatattctcaatttgtctacgaggagaggatgatatttcattagcccagccttcattttccgcattggatattcgactctcctcattactatattgcgggtaactgcataataagtgaaccggggaggtaccatgcaacggtgtggccggtctgctagagacaggggtgttgtttcccgatagccggccgtgtttcacccgctttttcgctgtgttgtgccagcttctaagagctgttgccacattatcaccaaagattataggtttcattgatgttcccatctgcaaatttcaccaaatctctcagcactaatttatctttataagagtttttgttgtgaaaagggaagactagtttagttatagagtacctgtgtgaccaaggcataaagagggagagtcacatagctgcaatggacctgtatgatcaccctgaaacaagaaacaaaaactatcaatatagaaggaattaaaatatgcatctttaattgttcgaacaaaaaaaaaaaaaa

SEQ ID NO: 2 - DNA, 813 bp, Nicotiana benthamiana
tgctgattttgggtatacaactgaaatgtttgagaaggacatggagctttggcaacgaagggttgaacattactggaatcttttaagtccaaagatctcttcagacagtctgagaaacatcatggatatgaaggccaatttggggtcatttgctgctgctttgaaggacaaagatgtttgggtcatgaatgttgtatccaaagatggacctaacactctcaagattgtatatgaccgtggtttgatcggcacaactcatgactggtgtgaagcattttcgacatatcctaggacctatgatttggtccatgcgtggagtgttttctctgacattgaaaagaaaggttgcagcggtgaggatctgttactcgagatagatcgcatactaaggcctagtggttttgttatcttcaacgacaaacaacatgttattgactttgtaaagaagtatttatcggcattgcactgggaagcagtagctgatccaacttcagatccagaccaagaaggagatgacattgtttttatcatccaaaagaaaatgtggctgacaagtgaaagcatcagagatacagagtaaataaagtttgccactaagtacacttcttgattcattttccccttccttttgggattaagaaatacacacccctaaaggtttgggagatatcagtttgattttgtagtatttatgatatttatttcttccttttcttcattaacttaatttcaacttgttgtttcttttaattgataaacaaactcatagactatatatgcatttataggctattctcgaaaaaaaaaaaaaaa

SEQ ID NO: 3 - DNA, 945 bp, Arabidopsis thaliana
gaagcacgaacggcgtcgggttagtccgacggaggaaccatgtcctcgtctcttcttctctccggttctactgtatcttcttcgtttatcgctccatctaagccttctctcgtacgaaattccagtaagacatcactgttaccatttcgtaatgtttcgagaagcttcaaaaccgtcaagtgcaccgttgattcttcatatggaggcaatgttcccacgttccctcggacgagagtttgggacccgtacaaacgtctaggagttagtccatatgcttccgaggaagaaatctgggcctctcgtaactttcttttacagcagtacgctggacatgaaagaagcgaagagtctatagaaggagcctttgagaagcttctcatgtctagttttatcagaaggaagaagactaaaatcaatcttaaatcaaagttgaagaagaaagttgaggaatctcctccgtggctcaaagctcttctcgatttcgttgaaatgcctcccatggacactattttcagaagacttttcctctttgccttcatgggtggttggagtatcatgaactctgcagaaggcggtcctgcgtttcaggtggcggtatcattggctgcgtgcgtatattttctgaatgagaagacaaagagcttggggagagcttgcttaatcggaattggagctttagttgccgggtggttctgcggttcgttaatcattcccatgattccgacgtttctcattcagcctacatggacactcgagctcctaacatcactggtcgcttatgtgtttttgtttctttcttgtactttcctcaagtaagttacgttgtggttttatccaaactctttttgttcttttcgcccagacatttacagaacctttcggaaaaattagtgaaagttgttaagtgaaaaaaaaaaaaaaagggcggccgcaccctaggccagt

SEQ ID NO: 4 - DNA, 945 bp, Arabidopsis thaliana; identical to SEQ ID NO: 3

SEQ ID NO: 5 - DNA, 934 bp, Arabidopsis thaliana
ctcaatggagtacaaacatttcagccatccacacactctaaaactccaacagattcagccacataaaagctcagattcttcagtaatctgctcaggttgtgaatcagccatctctgaatccgaaaccgcgtatatctgttcaacatgtgacttcaatcttcatgagcaatgtggtaacgcagtgcgtgggatgcaacatccttctcacgctggtctccaccacttgactctagtcccttacacaacttacagcgctggtaccttcctctgcagagcctgtggctgcactggaggtaaagggttctcttactgttgtcctttgtgtgactttgaccttcatgttcaatgcgctcacctgcctcaggtcttggttcatgagtctcatcctatgcatagtcttcttcttgtctacaacagtactcctcctatgtcttttactcagtttggtttcgggaatcagcttgtttgcaatctttgtaatatgactatggatggtaggttttggtcttacaactgttatgcttgtaactatcatattcatgcttcatgtgctgtgaataagcccaatccagtggctgcttctgctgagaactgtggggcgagtgatgaaggaaagacaccgactgctgaatctgttcctgttcagggtttggagactgagcagacggaacaagtagctgcaataacagagcaagtggaagatccagttttgaggcaacagcttgagcttcagaagcttcagcttgagctagatatgagttctgctctcgcaaacatgattggttccttcaatctcagttctttcgtttgaagtgtctttgtgtttcagtttgtttgattttatgcatttacatgtgttgaattgtctctgttcttgtgttccctaatgtgcttctgatttgaataaatatatcctatctatttggtttaaaaa

SEQ ID NO: 6 - DNA, 761 bp, Arabidopsis thaliana
aaaggatttgctctgagggctgggctcgggggtcccagttccgaacccgtcggctgtcagcggactgctcgagctgcttccgcggcgagagcgggtcgccgcgtgccggccgggggacggactgggaacggctctctcgggagctttccccgggcgtcgaacagtcagctcagaactggtacggacaaggggaatccgactgtttaattaaaacaaagcattgcgatggtccctgcggatgctaacgcaatgtgatttctgcccagtgctctgaatgtcaaagtgaagaaattcaaccaagcgcgggtaaacggcgggagtaactatgactctcttaaggtagccaaatgcctcgtcatctaattagtgacgcgcatgaatggattaacgagattcccactgtccctgtctactatccagcgaaaccacagccaagggaacgggcttggcagaatcagcggggaaagaagaccctgttgagcttgactctagtccgactttgtgaaatgacttgagaggtgtaggataagtgggagcttcggcgcaagtgaaataccactacttttaacgttattttacttactccgtgaatcggaggcggggtacaacccctgtttttggtcccaaggctcgcttcggcgggtcgatccgggcggaggacattgtcaggtggggagtttggctggggcggcacatctgttaaaagataacgcaggtgtcctaagatgagctcaacgagaacagaaatctcgtgtggaaca

SEQ ID NO: 7 - DNA, 727 bp, Arabidopsis thaliana
ctctttccttctctcaccgcgagagtaaccgagagacatgattctgataaactctaattctccgacgctaatctcagccgttagattcgtgggctcatctccgttcaccactcgggggctttctcagtccactgtctcaatctctagaaacaaaagcttcttcttccacttcaccgagacgaaggagaagaacgcaagaagagattatttgagagtatcaatcgtgtgtgacgcaggagggatgtttccggtggatccatgggctccaaccattgattcacagagcatagcatcacaactcttcgctgtatctctgtttccttacattggctttctctatttcctcactaaatccaaatcagctccaaaactcacacttttcggtttctacttcttgcttgccttcgttggagctacaattccagctgggatttatgctaaggtgcattatggaacatcgttgtcgaatgttgattggttacacggaggagctgaatcacttcttgctcttaccaatttgtttatcgtgttgggtcttagacaagctctgaggaagtctcaagatgatgatgatgataaacttggtaatgatgatgaagttccaacaactcaagaacaagggaaatcttcagtgtagtaaaacaaatgtaaattttttaattatggagtttcacttgttttttaattagattatatatagtcgacgcccatctaattcccattttag

SEQ ID NO: 8 - DNA, 288 bp, Arabidopsis thaliana
tgactgattactactacttgtactaactctaatacatttacaaaacaagtcctccttttccccaagtatacagataaagatttaccagaaccggttttccgccttcatctcacatggaaatcgtaaggagaagacgcatacacttgatctggaaccactagtggtaacttctcaatgtacataaacaatcgtttctggttctctctagcgattgcagtgagattcactgtatcgttttggtccaaaaacatccagagatcacctgaatctactcttttaaggctgtct

SEQ ID NO: 9 - DNA, 452 bp, Arabidopsis thaliana
acctccagccctgatgatggtgtatggaataccggaatcagccaagtattgctcagcctttctcttccagaccagaatgttagcattgccaatactattgagagggtgattaatgtttgttcctcccatcgacccaaccaaaacaatctgcttaactcctgcagagacagccttaaaagagtagattcaggtgatctctggatgtttttggaccaaaacgatacagtgaatctcactgcaatcgctagagagaaccagaaacgattgtttatgtacattgagaagttaccactagtggttccagatcaagtgtatgcgtcttctccttacgatttccatgtgagatgaaggcggaaaaccggttctggtaaatctttatctgtatacttggggaaaaggaggacttgttttgtaaatgtattagagttagtacaagtagtagtaatcagtca

SEQ ID NO: 10 - DNA, 552 bp, Arabidopsis thaliana
aaaagccctccatgtcccaccaggacttgcaccatcaaaatgggatacttgcagtgatgtcgtgagtgaacactggaatgactctccttcctcggttctaaacatttaccacgagcttatagctgctgggcttcgtatctgggttttcagtggggacgcagatgccgttgtaccagtcacatcaacccggtacagtatcgatgcactaaaccttcgtcctttgagtgcctatggtccttggtacttagatggacaggtgggagggtggagtcagcagtatgctggtctgaactttgtgacagtgagaggtgcaggccatgaagttcctttgcacagaccgaagcaagctcttgcgctcttcaaggcttttatatctggaactccattgtccacacatgagaacagcatcagccgcgacatgtctgaactcgttagtgactcataatgagttctgatttgatgtaatgtgtgatttggattctcaatcaaaaactttccacataggccgttgaaataagaagagggaaagagaataaatcagtgttttaagtg

SEQ ID NO: 11 - DNA, 391 bp, Arabidopsis thaliana
ttttgaatgaataaaagtcttataattatgatgtgtgtacaactacaaagttttccttggagtatagtttgaggatttatccagaagtagcagaagaagcagctacagactcggagagttcttccatgagttccttttgctccaaagcagcacaagcctgcactgcgtcctctaaagcaccgtcaagaaatgttgtaagcgcaaagttcatctttagcctatgatcagtcactctactgtccttataattgtatgttcttatcttttctgaacgagctccagtcccaacctgagatttcctttcattccttatcttctcttgttgttcccttacttttatttcatacagttttgctcgcagaagctggaaagcacgcgccttattcctaat

SEQ ID NO: 12 - DNA, 200 bp, Arabidopsis thaliana
ctgcagctgtgtgctccttagctaaggtggcaatggcagacgatgagccaaagagaggaacagaagctgccaagaagaagtatgctccagtctgtgtcacaatgcctaccgccaagatatgccgtaactgagtttgctatttaaccagcaactgtatctatgtcgtataactattctcagtgtggtttgtaaggatcata

SEQ ID NO: 13 - DNA, 1063 bp, Arabidopsis thaliana
ttttccagtctgcaacatatggaaaggttttggttttggatggagtgattcaactcactgagagagatgaatgtgcgtatcaagaaatgatcactcatcttcctttgtgctctatctccaaccccaaaaaggtactggtgattggaggaggagatggaggagtcctgagggaagtggcacgtcatagttctgttgagcagattgacatttgtgaaatagataaaatggtggttgatgtggctaagcagtatttccctaatgtagcagttggatacgaggatcctcgtgtcaacctcatcattggcgatggtgttgctttcttgaagaacgctgctgaaggaacctatgatgcagttattgttgattcatctgatccaatcggtccagcaaaagagctatttgagaaacctttctttgagtcagtgaatagagctcttcgtcctggtggagttgtgtgcacacaagctgaaagcttgtggcttcacatggatatcattgaagacattgtttctaattgccgtgacatctttaaaggatctgttaactacgctggttctctgagattagtcctatgtggccaggagaagcacattctctcaaggtagagaagattctattccaagggaaatcagattaccaggatgttattgttggaccagtgttccaacttacccgagtggagtcattggattcatgctttgttcatctgaaggaccacaagtcgatttcaagaagccagtgagtctaatcgatactgatgaaagctctatcaaatcacactgtcccttgaagtattacaacgctgagattcactcagctgctttctgcttgccctcttttgctaagaaggtgattgattcgaaagccaactagaaaagagaagagaaatcatttgctttagagaaacttcatgtggaagtgataatatgatgatacaatgatcctttggaaaaaaataaagaagttttaatttttagaatgtaatgttctttcacctgcaatgttatgtgactgcactgagctatcaatctctttttataagcattacacatatttcaaaaaa

SEQ ID NO: 14 - DNA, 1173 bp, Arabidopsis thaliana
aaatccccaaattttcaacaaggataagagccggaagctcatcgccggtgaacggaactagggtttcattcatccccaaattgataacaagaaaatggctcatgcttgcgtctctacatcggcttcttctctcagattcacagctggattcgtctccgctagtcccaatggctcctctttcgattctcccaagctttctcttcctttcgagcctctccgttcaaggaagacgaataagttagttagcgatagaaagaattggaagaattcaactccgaaagctgtatattccggcaatctctggacaccggagattccgtctcctcaaggagtttggtccattagagatgatttacaagtcccttcttcgccgtattttcctgcttatgctcaaggacaaggaccacctcctatggtgcaagaacgtttccagagtatcattagtcagctcttccaatataggattattcgctgtggtggtgctgtggatgacgatatggcaaacataattgtagctcaactcctgtatcttgatgctgttgatcctactaaggatattgtcatgtatgttaattctcctggtggatcagttacagctggcatggctatattcgatactatgaggcacatccggcctgatgtgtccactgtttgtgttggtctagctgctagtatgggagcttttctgcttagtgctggaaccaaaggaaaaagatacagtctaccaaactcaaggataatgatccatcagccgcttggtggagctcaaggtggccaaaccgacattgacattcaggcaaatgaaatgctgcatcacaaggcaaacctaaacggttacctcgcataccacactggtcaaagcctggagaagataaaccaggacacagaccgtgatttcttcatgagtgccaaagaagcaaaagagtatggacttatcgacggtgttatcatgaaccctcttaaagctctccagccacttgcagcagcttaatcgcctaaaggtagtggttcagctttagcacttgttcttttttgggcctttgatgaactgagattttccatgaaatatgtttctattctacaaggaaaatcagatttgtttgggatcaaactctgtagttgatacatacatgaagaccaaagtaaagtttcttactgtgctgaaaaaaaaaa

SEQ ID NO: 15 - DNA, 959 bp, Arabidopsis thaliana
agaaacgatgagttctcagatttcggagattgaacaagagcagctgatcgagaagcttgagatcttcaagatccatggcagagacaaacgtggccgtaagatccttcgtattatcggaaaattcttcccagctcgatttctgtcactggatgtgttgaagaagtatctagaggagaagatatttcctcgattaggtagaaaaccattcgccgtactctacgtccacaccggcgtacagagaagcgagaacttcccaggtatctcagctctacgagcgatctacgacgcaattccggtaaacgtcagagacaatcttcaggaggtttacttcctccatccaggtcttcaatcacgtctcttcctcgccacctgcggccgatttctattttccggcgggttgtacgggaagctgaggtacataagcagagttgattatctgtgggaacatgtgaggaggaatgagatagagatgccggagtttgtatacgatcacgatgatgatctggagtatcgtccgatgatggattacggtcaagaaagcgatcacgcgagggttttcgccggagccgccgtggattcatcagtctcaagtttctccatgaggtgtatctcatagcgtaaaaggctaaaactccacccactagatatcggatcgtatcttataaaccatataatatacgaatacgattaataatatatcaaaaagattggaaataggtgtgctttttgaaattagtgagcgttttttatggaaaagaaaagaaaagaaagcagttggcgtctggataaagggaaggaggagaatctttagattttttctttaatctgtttttcttttgtcttgattagttttttctttagtggtggtggttgtgagttagtgtgtaaaatgtatattgtcatatgtgaatttaataataagtccttttgtaagatgatcaagggaaaaaaaaaaaaaa

SEQ ID NO: 16 - DNA, 250 bp, Arabidopsis thaliana
gcagcacttgtctgacccatggcacaacactattgtccaaaccttcaactaaagagtgaagacagacttatgatctcatacctatctatcttccatcactttcatgtctgtctgtgagtgtgtttcatcttagagttcttggtttttgagcttgaattattgttgaaccgttgtagctccatgaacaaatttggaatcttcaatgtacagaggaactaagttaatcaacattgttgtactctttaaaaaa

SEQ ID NO: 17 - DNA, 391 bp, Arabidopsis thaliana; identical to SEQ ID NO: 11

SEQ ID NO: 18 - DNA, 1004 bp, Arabidopsis thaliana
agaactagtcagtttctttgttttagacaacaagaatctgtgaactaacacaaaaacattgaaagaatgatcttaacaatgaaacttgttcaccctctccatcactctttgtcttcctccattccctttccctcaagaaaaaggcaatccaaaccgtaccggtgctcgttaccttctcccggctgcgaaaaggtcatcagaacagagactgtcctgcctccggcgccggtgagttgtgaagggagaagggtcttacttggatgtcttctcgctacagcttctgggattttgtcaactggttcagccgaggcagtaagcaccagtagaagagctctacgtgcatccaagttaccggaaagcgatttcacgactctccccaatggtctcaagtactatgatataaaggttggcaatggagcagaggctgtgaaaggatctcgggtcgcagttcactatgttgcaaaatggaaagggataacgttcatgacaagtcgacaaggacttggtgttggaggtggaacgccttatgggtttgacgttggtcaatcagagagaggcaatgttctgaaaggacttgatcttggtgttgaagggatgcgtgtaggcggtcagagattggtgattgttcctcccgagctggcttacgggaagaaaggagtgcaagagattcctccaaacgctacgatagagcttgacattgagctgttatcaatcaagcagagtcctttcgggacgccagtgaagatagttgaaggctaaaaggactaatgaagccaacattgtaccaagattttctgtgtacattcagtaaaaaactataaaattgatcaaagctatggaagattcaactgtatgagaagaatctgtttaatggattataccggctagtccggttttgtaaccgctttataactgtgtctcatcactcaattcatacacttttggccgttttgcaaaaaaaaaaaaaaaaaaaaaaaaaaaaaaaaaaaaaaaaaaaaaaaaaa

SEQ ID NO: 19 - DNA, 397 bp, Arabidopsis thaliana
atagacatgtttcttcgcggtcaccacatagcaagcattctcagcacaagaacactctctactcttctcatgacaaatccaggtcgaagcggtcaaggtccagatcaagatccccccacaggcgccatcgtaaatgaacactctagcaaactggtctgagactgtacccgggacaatattgtgcgcggtggatcacaggattgggttaatgtactggacggacatcgatataatcaaaaactataaagtcaccggtttgtgagcgaaatagtgcatagtaaaccgctctttccttagttcttcagaagaaatatccaaagatttttgactgacttgtttgacaatatcgttggttggttaagcgttcctatgtaaaattttgttccctctgaaaaaa

SEQ ID NO: 20 - DNA, 442 bp, Arabidopsis thaliana
ttttttttaataataatattatattattattgatattcgaatgagtcaaattcaacagcgatacataatagagagataaaagacatcaaacataaccaacatgaaatcaactagaactcaaaacaagaccagacttaaacatcatcaattagttgattttactttaaattcatttaaacataaaaagcaaagaagaaacttcttaaaagaagaatccaaaggccatcaccgctacggaggcgaagaaagtagggataaatgaggaagcatcggaggtagggctaggcgcgggagcgtcaaccgctgcaaccgtctggtgaactgtagagaacgctatcactgccaccaaaaccgccacgaaaagcttcatcttcattgcctccattgttagtaaatgaagagaagaaaagaggttttggtgcttactgttgtgttgttggtt

SEQ ID NO: 21 - DNA, 813 bp, Arabidopsis thaliana
ttaacaaatagtcataacaataaaatatataaaaaataataatattaataacaataataataatgataatgggagaaagaaatccctaaaaaaaaattgaaaatgggagaaagaaaaccaagaaggttttgtttcatctcctttcttcccataagcctttttcttgtattgttcccttctcttctctaaataaaaaaaaaaaaaactgttttttgtgaaaattaattgaccaaaaacaaagaaatcttctttcttctcttctcttctttgttaatcttgttacccttctaccaccaccacctgtaaaaaagaggtttttatctaccacatagagagaccagacaagaacatgtgattctttggttaggtctctcaattctgctgagccacaagctgatcgagctgcatttgctcaatccaagcttctagcccagcatgatccattaccggttcaaccggatttggctgcttcatcacgtgttcccctgaggaagatgttgctgctttctctttggcttctgctggagtctccttcaccagtgactggacccatgacacatcaggctcatcaccatcaaacgatgacgaagatctcaacttaccaagtgcttctgagctcattccccaatccggttgaccatttgaagatccccattttgatgaccatgtgttgttgtttaccggtgaaccaacgattgggctagagtttgttctgagctctctggagctaaggctacggaactgatgttgctgctgctgctgctgctgttgctgttgttgttgcttcacgcactgagccaacatggaaacccg

SEQ ID NO: 22 - DNA, 397 bp, Arabidopsis thaliana; identical to SEQ ID NO: 19

SEQ ID NO: 23 - DNA, 625 bp, Arabidopsis thaliana
tatgtgagagatatagtaactacaactgaatgaaaaatccatgagacaaaaaagttcgcaatagaagaatattgattcggtaacaaagcacagcttataagttttcttgtgttaaagatgaaccaatttgaagcattagaggataaactggactaaactctttgtcccctctcgatctgatcttcactgcataatcatccaaagttgcttttatccctttccagatctgatcctctctttggttatcaagccacagtgagtactgtttaggacttagtctgttcttctgcatcggtgactctaactcgtctgggcctcttgtgtagagatgatgtaggacgatgctcggtggtagatcattgatgagaggagatgatcccatttgagatgtttccaggaaaaccaaaggcctaaacgctctaagagctctgtacggtgctccgagttgttccacgggaaatagattctgtcccactgctagttccagctcggccatgtctttggccattctgagttttccccattctgaaagtggtcgcacaagggatgcatgtctgatgtagaagatcaaaacccttgacgccatttgtcttgtgagtcttgtgcagatcgattctgttcctgc

SEQ ID NO: 24 - DNA, 959 bp, Arabidopsis thaliana
agaaacgatgagttctcagatttcggagattgaacaagagcagctgatcgagaagcttgagatcttcaagatccatggcagagacaaacgtggccgtaagatccttcgtattatcggaaaattcttcccagctcgatttctgtcactggatgtgttgaagaagtatctagaggagaagatatttcctcgattaggtagaaaaccattcgccgtactctacgtccacaccggcgtacagagaagcgagaacttcccaggtatctcagctctacgagcgatctacgacgcaattccggtaaacgtcagagacaatcttcaggaggtttacttcctccatccaggtcttcaatcacgtctcttcctcgccacctgcggccgatttctattttccggcgggttgtacgggaagctgaggtacataagcagagttgattatctgtgggaacatgtgaggaggaatgagatagagatgccggagtttgtatacgatcacgatgatgatctggagtatcgtccgatgatggattacggtcaagaaagcgatcacgcgagggttttcgccggagccgccgtggattcatcagtctcaagtttctccatgaggtgtatctcatagcgtaaaaggctaaaactccacccactagatatcggatcgtatcttataaaccatataatatacgaatacgattaataatatatcaaaaagattggaaataggtgtgctttttgaaattagtgagcgttttttatggaaaagaaaagaaaagaaagcagttggcgtctggataaagggaaggaggagaatctttagattttttctttaatctgtttttcttttgtcttgattagttttttctttagtggtggtggttgtgagttagtgtgtaaaatgtatattgtcatatgtgaatttaataataagtccttttgtaagatgatcaaggggaaaaaaaaaaaaa

SEQ ID NO: 25 - DNA, 618 bp, Arabidopsis thaliana
ctttcatgtgagagagagagttgaattttgcagatgagtatgagaagaagcaaagcggaagggaagaggagcttacgagaactgagtgaggaagaggaagaagaagaagaaactgaagatgaagatacttttgaagaagaagaggctttggagaagaagcagaaaggtaaagctacaagtagtagtggagtttgtcaggtcgagagttgtaccgcggatatgagcaaagccaaacagtaccacaaacgacacaaagtctgccagtttcatgccaaagctcctcatgttcggatctctggtcttcaccaacgtttctgccaacaatgcagcaggtttcacgcgctcagtgagtttgatgaagccaagcggagttgcaggagacgcttagctggacacaacgagagaaggcggaaaagcacaactgactaaagacggtgaaacgtgtgagatcccggtttgaaggttaatgaaacaggctttgcttactctcttctgtcagtctcttttagctccttgtaatcctctgtgtctctgtctgtttctccatattacctgtaatcaaagctatctgctaaacctacgacatggttaaataaatgcattgagacttagtaaaaa

SEQ ID NO: 26 - DNA, 1094 bp, Arabidopsis thaliana
atcttatgcaagaagttgctgtggagacatttggtgctatggcaaaaactgagaaaattgcatttatccttgaacaagttcgcttgtgcttggatcgtcaagattttgttcgtgcacaaatcttatctaggaagatcaatcctagagtttttgacgcagatacaaaaaaagataagaagaaacctaaggaaggtgataacatggtagaagaggctcctgctgatataccaacccttttggagcttaagcgaatttactacgagcttatgattcggtactattctcataacaatgagtacattgaaatctgccgtagctacaaggcgatatatgatatcccttcagtaaaagaaactccggagcagtggattccggtcctgaggaagatctgctggttcttggtcttggcacctcatgacccaatgcaatcaagcttgctcaatgcaactctggaagacaagaatttatcagaaatccctgatttcaagatgcttctaaaacaggtagtgacaatggaggttattcaatggacatctctgtggaacaaatacaaggatgagttcgagaaagagaaaagcatgattggaggttctttgggtgacaaagctggtgaagatctgaaactgagaatcatcgaacataatatcctcgttgtctcaaagtactacgcaaggataaccttaaagagacttgccgagcttttatgcctgagcatggaggaggcggagaagcatctatcggagatggtagtgtcaaaagcactgattgcaaaaatagacagaccatctggaattgtgtgcttccagatcgcaaaggacagcaacgagattctaaactcgtgggcagggaatttggagaagcttctagatcttgtggaaaagagttgccaccaaattcacaaggaaaccatggttcacaaagccgctctcagaccttgaaaacatgcggtcttcttcatgaaaacttttcaggatcttcttcgttgagttattagcatctttatgtggtaaaaactcgaatcagtgtttccttttaaaaattgtactatggatctgtacactaacgaagtgttttgccacttattggttaaaaaa

SEQ ID NO: 27 - DNA, 367 bp, Arabidopsis thaliana
ttttgaaacataaacaaaactcttatttattaaggacttgtgctaaatacatttagcctcaaacatccaaaacttacattttcataaaagacacgatgaggtgtggtgttaacatgtatcaacaaccacactctcatacgctcgagggtttttgtttggaatctattagtaaggagggaagaaagggatggtggtctggaaggggcattcaccaacttgctggatcttgcaaatgttaggcaagtacttagctgtcttgtaaattttcctggattggaatggtccgtgttgtccctggaggctaacggccctggcagcttgtctcaaggtggggcaaacacaaactggctcttcctggcgaagctcg

SEQ ID NO: 28 - DNA, 949 bp, Arabidopsis thaliana
ctaatggaaataccgaggcgaatgtagtggaagctgtagagaatgtaaagaaggataagaagaagaagaagaacaaggaaacaaaggtggaggtaactgaggaagagaaggtcaaagagactgatgctgtgattgaagatggagttaaggagaagaagaagaagaaggaaactaaggtgaaagtaaccgaggaggagaaggtcaaagagactgatgctgtgattgaagatggagttaaggagaaaaagaagaagaagagcaagtcgaaatctgttgaggctgatgatgataaggagaaagtttcaaagaaaaggaaaagatcagagcctgaagagactaaagaagagactgaggatgatgatgaagaatcaaaacgtaggaagaaggaagagaatgtagttgaaaacgatgagggtgttcaagagacacctgttaaggagactgaaactaaggaaaacggaaatgctgagaaaagtgagacaaagtcaacaaatcagaagtcaggaaaagggctttctaactcaaaagagccgaagaaaccgtttcagagggtgaacgttgacgaaattgtgtacactgagaatagcaactcgtactattcaaagggtggtgctgaaattggctatggtcttaaagctcaagaggttctcgggcaagtgagaggaagggatttccgacatgagaagacgaagaagaaacgaggaagctacagaggaggattgatcgatcaagagtcacattcgactaagtttaataactcagacgacgaagaatgattgataagcagacaacactgcctttttgacattgcttcgtttcgatttatctttttcttttcttttgctttgatcatttcaatacccgtaatagggctcaagttttgtgtctgtgcactcctttgatacttatatgaacatgattcagtttcagtttcttgttcaaaaaaaaa

SEQ ID NO: 29 - DNA, 711 bp, Arabidopsis thaliana
ttttttttttctctcgcaaacagaaatttatattgacttttaagaacaaatacaaagtatatctatcacacaactcacaaaagagataggtacaaacataatgacaaatcacaatcagcacaccattacattaaaagtcaaatttacctttttaataagaagatacaaaaatatataaagagaagaccaagacaatttgacttgagtgattaggaggcattgttggcctgtaataatccatttcgaatctgcgttgccacgtcagcgacggcgcctggaccgtgagggataaacaccgccgaggctttagaagttgctccgatatctctcattgtgtcaaagtactgagtcatcatcaccatgtccaacacatccttcgctgacgtccctggcacgtttcctgcgaaccctagaacactgtctctcagaccgtccacgatcgcttgtctctgccgagcgattccgagtcccgacaggtactttgactctgcttcaccctctgctcttttgatctgaatgattttctcagcctctgctttttcgctcgctgccactctcatcctcgccgcggcgttgatttcgttcatggcacgtttaacctgttgatcaggctcaatgtcgataattagggtttgaaggatttcgtaaccataagcagtcatggctttgtctagctcttcttccacagatttggcaatttcattcttctgc

SEQ ID NO: 30 - DNA, 1132 bp, Arabidopsis thaliana
gaacatcagaaaaaggcatgtaatattaattcagccaacatctgtggatatgcaggtgttgaagagaaacggtacaaattaaagttgatttcttttactttgttacagctacgtaccatacaatcacccaaacatacaaaaccttaaaagacaaaagttggcatctctatcagttgggttctagtcaatcttcactgaggagtagatctttctcacgaaccagaagcaagcatagaaaccgattgtgccagttaggacgaagaatgcgtaagagatgataatcatgtacccgaagtagagcattcccgagactagctttgtgatctccagctttgtgaagaagtagaagattgagtagaggaagaggtagaaagcggatgagcccgcagttaagtaagctctccaccaccagttgtagtcttcgctacaaagctggaagtagcagagcaccactgtgatctctgcacaggtgacgatcaagatcaaaaaaactataaagaggaacccgaagatgtagtagaactggttcagccatatagatgtcaagatgaagaagagctcgatgaagactgctccaaacgggagaatgcctccaattagtatagagaaaactggtttcatgtaccacggctgctctggtacttgcctcgggatcttgtttgttttgactggatcttcaattgctggcttcttgtaacccagatagctaccaacgaagactagtgggactgagatgccaaaccagaggcagaagagagcaaacattgtaccaaatggtatggctccagatgactgttctccccaaataagggcattcagaacaaagaagatagcaaaaaggataccgggaaacatgaatgcagtcttcaaggtcattctcttccacttgtttcctttgaacattttgtgaaggcgagacgaggagtaaccagcgaatatgcccatgaaaacccacaagagaaccatggcagtcataagccctcctctgttggatggagataagaagccaagcaacgcaaacatcattgtaacaagtgacattccgaagatctgaacacctgtaccaacataaacacacaataaaccagagttcaccggtggcctgaagacatctccgtgtacaagcttccat

SEQ ID NO: 31 - DNA, 389 bp, Arabidopsis thaliana
agtgaagcaatggagtccagatggagtgactcggattggtgtgattgggaaatcggtatattccgtgaaattctccggtatatctacccggaagatgctccaccaaccgtcgtctgcttgcggacgcgtgggtcgacccgggaattccggaccggtacctgcagaggaagatgaagatttaagttgggacattgatgaagatgacgaagaagaatcatcatcatccaaagcttaatgttagttttaaagtggtgtgctttcttacttttgccaatccttttacttgtttttacaagtttctggcgctttgcccccattcttcagttgtttctctcttcaacgtttctattttacattgatttgaaaatatgtttttaaattttaaaaaa

SEQ ID NO: 32 - DNA, 711 bp, Arabidopsis thaliana
gaagaagaagaagtaatggcttcctctatgctctcctctgccgctgtggttacctccccggctcaagccaccatggtcgctccattcactggtttgaagtcatccgcttctttcccggtcacccgcaaggccaacaacgacattacttccatcacaagcaatgggggaagagttagctgcatgaaggtgtggccaccaatcggaaagaagaagtttgagactctatcttacctccctgaccttactgacgtcgaattggctaaggaagttgactaccttctccgcaacaagtggattccttgtgttgaattcgagttggagcacggatttgtgtaccgtgagcacggaaacactcccggatactacgatggacggtactggacaatgtggaagcttccattgttcggatgcaccgactctgctcaagtattgaaggaagttgaagaatgcaagaaggagtacccgggcgccttcattaggatcatcggattcgacaacacccgtcaagtccagtgcatcagtttcattgcctacaagcccccaagcttcactgatgcttaaatccttttctggaatattcaatgttgactatccggaacccaattttgtatggtcaatgtaaatttaagtaattattttgccaaagtgaaaaaactgaaggtttgtttttctatcatttcctctataaaaatctctattcatatcacttca

SEQ ID NO: 33 - DNA, 607 bp, Arabidopsis thaliana
agcaacctttctctgaattcggggaaatagtgtctgtcaagattcctgttggtaaaggatgcggatttgttcagtttgttaacagaccaaatgcagaggaggctttggaaaaactcaatgggactgtaattggcaaacaaacagtccggctttcttggggccgtaatccagccaataagcagcctagagataagtatggaaaccaatgggttgatccgtactatggaggacagttttacaatgggtatggatacatggtacctcaacctgacccgagaatgtatcctgctgcaccttactatccaatgtacggtggtcatcagcaacaagttagctgaggaaactaaaagcttaatctgagcatctatctataggacaacaaaaactcactcaggttaggtgatgttaggaggtataaggcaaaagtggttggcttcttgtctctacttgagtttagggtttatcatcttttggacatcgaattttggtggaaatcatacagtaatttaggagacttggatttgattgattaatttgatttgtttcttctgatctttttgactattgaacttattgatcaaagaagtgagttgcaccaaaaaaaaaa

SEQ ID NO: 34 - DNA, 874 bp, Arabidopsis thaliana
gtacaatgtctcctatgtctaccatgccctagatgcctacatcgagagagacaatgtcggcttgaaaggtttcaccaagtcagtttctttagtctaaaggaaaaccgtatttgtgtctcttcagctggtggatcatctttttgttattgttgagggtttaacgctaataggttctttaacgattcaagtcttgaagaacgaggttatgctgagaagtttatggagtatcagatgcattgtttgcgatggagcttgcactgactttggagaaacttattaatgaaaagcttctgaagttacaaagtgttggtgtgaagaacaatgatgttaagctggttgattttgtagaatctgagtttctaggcgagctggtcgaagccatcaagaaaatctcagagtacatagatggaacaaaaataaggtcaatgcagtggtgaagctgagatcggatgtttctgatataagctggcaagtgaagatggagggtcaaagactaacccaaggctggcaaaagttcgcaacaagccacgatctccgagtcgtcgacatagttgttttcagacatgatggagatttcttctcaaaactttgaattctttgaattctttgtttcgagatctatcgatactcgacatcaaagaactccttataactcttgattcattgaaacaagagtaggcatgtcaatcgagctatcccggtccgacccgaaacccgtaatacccgtatatgtttgagtttgggtcagaaaagctctgagcctatattttaatttaggtatttcctgtattttttattttttgtattcaatttctccaaaattagtggaaattatccatattttctttctatttttttaaaaaaaaaaaaaaaa

SEQ ID NO: 35 - DNA, 874 bp, Arabidopsis thaliana; identical to SEQ ID NO: 34

SEQ ID NO: 36 - DNA, 582 bp, Arabidopsis thaliana
aaaagaagcttcatgtatctgatgaagattttgccaagtggaagtttgcgttcatgtcaatggggcgtccagagtacttgcaggacacagatgttgtttataatcgcttccagagaagagatgtctatggtgcttttgagcagtacctcgggttggagcatgctgacactactcctaagagggcttatgctgcaaaccagaaccgccatgcttacgagaagccggtaaaaatatacaattagccccaaaacatgaacacaaatgtcaggagacattgtggcagcaacgttggaccaaggcattgattggaccaatgcatcgaataagaagggaaagggcgagtgtgagggtgtgatgatgaccgtaggatgttgtagagaatctggtctgatagggttttgggttgcgcattgatagtggtgtgttttctatttttttcttttcaatctatccttattttatttcgtgcttcaatactttgtgttaatatctggaatgtgtgaagaccattgcacatgcaatttttattttccaaaaaaagaatatggaagcccttcgcttggaaaaaaaaaaaaaaaaaaa

SEQ ID NO: 37 - DNA, 938 bp, Arabidopsis thaliana
agttattaagcttttaaattttataaataattaattattatcttaactaatcttgatcttttttattttttatttttttggttagctggaaaataaattgtcggcaattacagatcaaaatgaggcggagaaatatgtagatgtgattgacccaagggatattaagattgggagcagaaaattttatagatacattggatcacttactactcctccttgtacgcaaaatgttatttggaccgtcgttaaaaaggtaaatactcatcgttattttcttctcttttttacttaatcaaacatagcattaatagatcattacaaggtactaatagtgtgaatatccatatccaaaaggtttatccatctacatgttaactaggtctatttttccaattttaaattttgactttttattttaaaatcattcgtttaaatttatttggttggttttttaggtaaggactgtgacgaaaaaccaagtgaagctactcagagtggcggttcacgatgtaagttttacttaaataatttacttagtgaatttcacaactatactatatcttagaagttgaatgtatattatatttgtttattatcaaaaatgtaaatatgattgaaaaataaatttgcagaattcagatacaaatgcgagaccagttcaacctacaaataagcgcgtggtaaagttatacaaaccaaaatcactatgaatcaaggcgtcacatgaatcaaatacaattaatttatttcaattttttacaaccacagtgtactatttatttaatttttttgttcaccaaagtttttatatataacacgaaaaatatatgatgtatgtgttttcctgagtatcctatggtgtcccatcttcctcctgtagtttcaagatcttcaatccaatctaattcaaatataaaaaaaaaaaaaaaaaaaaaaaa

SEQ ID NO: 38 - DNA, 1386 bp, Arabidopsis thaliana
gattctctctctagcgatgtcgatcaaaccggagaattctccgattccgttgattgggattaataatacgtgtgaaatcatggtgtctttgagttcatgaagaacggaggttaacctaatcgaagatttgatttgggactgcgaatgagagagaagacgttgaaggctcaaattggagatttctatgaatttgttgatttgagagaagaattgaggctcattcgtaacggagggaaggtgacggtgacggcgatggtgaatttatagttgtacacggagctggaactcatcaaggacaaagaaggcaaagctactcatctacttaagttctgctccaaagctgaaatacggagattcagatcctttgttcccatgaatcaactacagacgctttaataatctgagggaatcttccgtgccaatatggagagatcatcatcatcatcatcatcatcatcatcaacatcatcatcatcatcattatcatcatcatcatcatcgtcatcgtcatcgtcatcatcatcgtcatgtgatcaggtaatattgactggatcagcaaattcgccgacgaattagagtggaacctcagagggaattttttttttacgatttgtctaatctgattcgaaattttgtctcgtggtgatgccgatgaaatagaagatgtgtacctttcatatcattcactctggttttatgggatcagaagaaattagcgagagtaaaatctgtggacctgcaccatgtaacttgattatggcactcagtccgagtaaggttctgacacatgttatctcattctatgtttacatgcttgttcatcttcaggtttggaatcttggtttaccttacccaacttttacattggccattgacgataagccctatctaaataccgtctctgatgatcactctgttaaggtcaaaaatgttgattggatcagcaaactccctgacgatgtattgctcataatattatcgagactttccacagaagaagccataaggacgagtgttgtgtcgaagcgatgggaacatgtgtggagtcaaatgtctcatctcgtcttggacatgcggaagaagattatcaattccaacaacacgcctgatggttcgaatccagttgctacattgattactcaggttataaacaatcatcgtggacatctagagagctgcgtgatcatgcatgtcccatatcaaggtggaaatggaatgctcaattcttggattcgattactgagttgcatgaaacgcacgaaagttctcacacttagaaccattatgatacttgggatcgaaagttcaaaacttttaacttttctcccgactccttgtcccatccaagtcttatgtcactctcgctacattcatactttctcgaaa

SEQ ID NO: 39 - DNA, 719 bp, Arabidopsis thaliana
caatagtcatggctagaaaccttgaagaggaatcaagtggtgatacagagttcattaaagcctcttgtgagacgacgtcgtacccagaccgatgcttccagtctctgtcttcatatgcaagcgagatcaagaagcagccacgtaagcttgctgagaccgcgcttgccgttagcatagcccgagcaaagtcagccaaaacctatgtatcagagatgactgattataaaggaatcacaaagaggcagcacgaggctgtagcggactgtctagaggagatgggagacactgttgacaggttgagcaattcgctgaaggaactgaagcatctggaggaaggtgacagcggagaagacttttggttctgtctgagcaatgtccggacgtggacaagcgcagcactgacagatgagaccgcgtgtatggatgggtttggagggaaggccatggctggggagctgaaaagtttaatcagaacacacattgtgagtgttgcggaagagacgagcaatgccttggctttgatcaatgactttgcttccaagcattgaaatcatttcaaaggggtttagtctttgggacaagagtttttctcgtattcaacactgcttgtgtttttttttctctctttaaagtttctactttatcttaacttatcatttttcatattatgcataaattaatctgtattaaaattaaaatacttcataattcatttcaaa

SEQ ID NO: 40 - DNA, 808 bp, Arabidopsis thaliana
caaagaagaagatttccagagatacgatgagtcgtctgattcaacattctacgaagctccaaggtttgtgacacacattgatgatccagctatagctgcattgacaaagtattactccaaggttttgcctcagagcgatactccaggagtgagcatactcgatatgtgtagcagttgggtcagtcattatccaccggggtataggcaagaacgaatagttggaatgggtatgaatgaagaagagcttaagcgaaatccggttctcaccgagtacatagtccaagacttaaatctcaattcaaatctgccttttgaagacaattctttccaagttataaccaatgtggtaagtgtggattatcttacaaagccgcttgaagtgttcaaggaaatgaacagaatccttaagcccggaggactcgctctaatgagcttctcgaaccgttgcttctttactaaagcaatctcgatatggacatcaactggcgacgcagatcatgctctcattgttggatcatactttcactacgccggaggatttgaagctcctcaggccgttgatatatctccaaatccagggcgttcagatcctatgtacgttgtttactctagaaaactccccatggtttaaacctgagatccaagcacatcatgtatacacatagtagagaccgaggaaactaattcttcgattaagacaagggaacttctgaaatcttgtttataaagaatgtgccactctctcaacactaataacaatgtcatataaagaatctgaagccagattcgcaaatttgacgataaaaaa

SEQ ID NO: 41 - DNA, 626 bp, Arabidopsis thaliana
tttttttggaagaaagtgtaaatacttgaaacttttcaatctaaaggttttcacagttgatgtgatctcaaataacaaaaaaaggtaatacgaactcataaactgttgttcaaaaagggaaccagagaaacattgtcaatctaattcagtttagatgaagaggctgcaaaacccgaactcaatcttgtgtgtcgtttcaccatcctcctttgcagctgaagttccctcagaatatgtgcatcaagtcataagcaaatgtccagaacagcacaaacgacataaggccaccaagaaagccgtcaaaaaggacccgattccacgaatcaaagtacaagtcagctgagaatcctgccttagccatgagcccaacagatgtgatcaacatcaccacaaagtaaaatacaaaaccaatcaagccattgaatccaattattcctgctaagacaccagctatgatagacagaaacgtccggctgttttgaatgactttcaaattgttctgcaaattctctgcactgaaagttggtatgtcactcatgatatcctttgatctcttctcagatgaacccatttaagatagcaacaataattagaaacgagagtagtaagaggaagatcgaagtagc

SEQ ID NO: 42 - DNA, 261 bp, Arabidopsis thaliana
tttttttttttttttccaaaaaggttcaaaatcataacacaaaacaaaagaaataaacaggaagctcgagtgccaagtacctccgccacctccgatcaagaacccaattccgagaattgagctccgacggagaataaacgaagcggtaacacaaacaaccaaccaaataccaaactactaaagtaaagaaactaaaatagtccttcatttcatcagcggaaagagttttgatgttcagagttcacttggcacccttcttga

SEQ ID NO: 43 - DNA, 725 bp, Arabidopsis thaliana
gatatgagtagccaaatcgctttgtcaccggccatcgccgccgccattcgccgtccgtcctctcacgactgtctatccgcttccgccactactgctaccgccacccccatggctctcaaatcttgcatcgtcgcacctctctcgctattcacctctcaatctcaaatcaaacactcaagctcaagaaaaacttctcgaaccacgattcgatgcgatgtagcgataaaatccgcagattcgataaacgcagacgccaatccttcgtcctcaccgtcatcagaggaagaaatcgaagcggaagcgaaggcgaagataggatctagggttagagtaactgcaccgttgaaggtttatcatgtaaatcgagttccagaggttgatttagaaggtatggaaggtaaactcaaagattacgttgctgtttggaaagggaaacgaatctcagctaatcttccttataagattgagttcttcaaagaaattgaaggtcgtggtcttgttaaatttgtttcacatcttaaggaagatgagttcgagttcattgatcagtgatgaaacaagaaagacaatttttgttttcctttctcagtgtttgtttttgtttgttgtgtttactggaacctgggaatggagaatgatttgtatgtagtgtgatgtgtattcaacctttagcaatcatatacataagggtttcttcaaaaaaaaaaaaaaaaaaaaaaaaaaaa

SEQ ID NO: 44 - DNA, 983 bp, Arabidopsis thaliana
tctttcttcttcctgattggaattttagggcttttgaaagcacgaacgcgtgaagctctaatcgagaaaaaaatggaggttttggataggagagacgatgagatcagggactcgggaaacatggacagcatcaagtcacactatgttaccgactctgtttccgaggaacgccgctctcgtgagctcaaggatggtctccatcctttacggtacaagttttcgatatggtacactcgtcgcacaccaggggttcggaaccagtcttatgaagataacatcaagaagatggtagaattcagcacggttgaaggattttgggcctgctactgtcaccttgctcgttcttctctcttgcctagtccaacagatcttcatttctttaaggatgggattcgtccattgtgggaggatggtgccaactgcaatggaggaaagtggatcatacgtttctcaaaagttgtatctgctcgcttctgggaggatctgcttcttgcgttggtaggcgaccagcttgatgatgctgataacatatgtggggcagtactgagtgtccgtttcaacgaggacatcattagtgtatggaatcgcaatgcttctgaccatcaggcagtgatgggtttgagagactcaatcaagcggcatttgaagttgcctcatgcatatgtcatggaatacaagccacacgatgcttctctccgcgacaactcttcctacagaaacacatggctgagaggataggcccaaagtcgatgattgtatcatgtaatgtggagaagatttgggaagctcatctgcaacctgggaagatatctggattgaaccctgtatccaataccatactgtaccggaggcttacaatatcagaaaaacaaaatccgggctacttctgtgtcagtatgtgttcatttcgtttttcttttacagtacatcttgttaacttcaatggtttgactcttgatcaaaactataagggttaattttca

SEQ ID NO: 45 - DNA, 693 bp, Arabidopsis thaliana
aaagacgctgaagaagaactttgccaacaagggtcttaacgctaaagaccttgtggttctctcagggggtcacaccattggaatctctagttgcgctctcgtcaacagtcgtctctacaacttcacaggaaagggcgattctgacccatccatgaaccctagctacgtgagggaattgaagagaaagtgcccgcctacagatttcagaacctcactgaacatggacccaggcagtgcgttgacattcgacactcactacttcaaggtcgtggctcagaagaaagggctcttcacatctgactctacgcttctcgatgacattgagaccaaaaactacgttcagactcaggccattctccctcctgtgttttcttctttcaataaagatttctccgattccatggtcaaacttggtttcgtccaaattcttaccggcaaaaatggtgagatcaggaagagatgcgccttccctaactaatttggatcgatcagaccgggtttcggatgattttgagtctacacgtttttctctgcttattttctttctttttcttttttctttcacggaagtttgagctttggtgttgtcttcttctgtttcttccatgaataattgttttttgttgagtaactttacatttgtattctttacggtgactgtgttttgtaatggaaaaagtttgtttcgaattc

SEQ ID NO: 46 - DNA, 903 bp, Arabidopsis thaliana
ttaaaccaaagcatactcttttgccagagaatcatcaagacatgacgattatttcaatggtgactctatcagggaaccaagaaactatgataacaatttggaagctcctacaagaaacactctggtacaatccctccaatggagagagaaaacacctactcaggacaatccaatctttgaaacctcccagtcctccagagtcaggcgcccacctcttgagagacagcagtcgacactctaaccctcacaacttcgcctctcatcccaactacagaagccgtaatgggagaactctgtgatgtcactattcaatacacgagctgcgccgatcctagcaattccctctccttttgataaccatttctctgtagaacagcctctctctcctgatgtgccagtacatcctgctcagatacaacagatccctccgatcccaccaaagaaaaagaagggccgacctccacttacaaaacctattagacgccatggtgatgactctgtcagcacttcaagtaaatcctcttctcagagacgacaacacaccagggttgcagcaaatctggcttcttcaagcaaagctcctcctacaaggccaattatcccagccactgtgaaaggaagggtggattttcccaatccacctcctcctcttccttaaaacttgcaagctggaactgctgtgggctggggaatcccatgacagttcaacgactgagagagattcagaaaacaatctctccagacatcctattcctcatggagacgaaaaaccctgatgaagtggtgcttaaaaaacttcaatggtcgaatttctcaaaccatttactcatatctccgcacagccctgggggtggaggcctggccctctactggaaacaacacatcgagctagaagtactctcc

SEQ ID NO: 47 - DNA, 603 bp, Arabidopsis thaliana
gtaaccatgccttctctctacgaaaaatcggaacttttctctgtcacagagaattttctaaatccgagattcacctggaccattcggggattctctacgctgctaaaaaacagttacctatcagaagtgttctccatcggaggaagaagttggaatatacaaatcaatccaagtggtcttggtacgggagagggaaaagctttgtcgatgtatcttggccttaatgtgaatgagatattcagaccatatgagaagatttatgttcgagccaagcttcgagctcttaaccaactcaatctcagtaacatcgaaagggaactcgatatttggtacaatggtccgggatatggagaatatagctggggtttccctgagtttatctatttcccttatctcacagattcatcaaagggtttcgttaagaacgatgtgttgatggttcaagttgaaatggaggccatttcttcaaccaagtacttcccgagttagattttctctaagcaaagaacttgtacctacctccatgtgtttgatttgttatcaaatactaataagaatttgattatgcatttcaaatacaattgtttctttttcttaaaaaaa

SEQ ID NO: 48 - DNA, 154 bp, Arabidopsis thaliana
ttttttttgttataagaaagaccgattgatttatatgtaacaccaaaacaacatagagaaaaccaaaaggaacaagcaagagcttcccacggcagacattctagaaggatgatttactcaaagatatcatcatcgtcatcggggaggggttgag

SEQ ID NO: 49 - DNA, 162 bp, Arabidopsis thaliana
gaagaagcagctgaagctgctaaatctgcttgaaaaaacccgctattgatttatggtctcttccttgttgtttcctcgagatgttgttaatctctgttatttgttgctgaaccatcttgtatttgtttttcttttggtgtaaacactttccttatcaagtaa

SEQ ID NO: 50 - DNA, 225 bp, Arabidopsis thaliana
ttttttaaaatttaaaaacatattttcaaatcaatgtaaaatagaaacgttgaagagagaaacaactgaagaatgggggcaaagcgccagaaacttgtaaaaacaagtaaaaggattggcaaaagtaagaaagcacaccactttaaaactaacattaagctttggatgatgatgattcttcttcgtcatcttcatcaatgtcccaacttaaatcttcatcttcct

SEQ ID NO: 51 - DNA, 1261 bp, Arabidopsis thaliana
tgaaaccggaaatgtagtaacttgacataagtttttcaatccgacaataaaagtgatccgagttcgaatctatcaaaaaccaaacgacaaaaactaatcacgacgacatagcgttgttgactacaaacagttacaacatcctactttgatagagattgtggatccactcttatcactcgtcagctggtggcgaacgaggagaccggctcttctgcattgggctctctgcaccatcatacccaccatcactgtctcttcttcctattgacccagggctttcaacttggccattctcggggctagacctcgatctctctctcctttcaatgggactttcaacttcaccaaccccatttctcggactctccttcttgaacgggctgtgattagggctcatcctctctctcttgttgttgggactgcggctgtacttagtagggcttgcgactctctccctcctgcgagggctatcattgcctctgcggtcacgaccatactcgggactgccacgtcttgatttcttgtaaggacttgggctacgtcttcgaccatagtcaggactggtcctttcctttctgtaggcagcaacaggactagctcctcggccataatcagggcttcctctttctcttttgtaaggactaggtgatcgccttctcctttcaggtgacctatcacggcgtctttcaggactgtgtccatttcctctagcatcatcatccttcacagcatactccaccgagatcaccttatccatcagcttactgttatttgaagcatccaatgctctggtggcatcctcttgtgcctcgtactggataaatgcaaaattcctcctgatcctaacgtttacgatctttccatacggctcaaagtgtttctctagatcccgggtcctagtattatccgcatcaaagttaatcacaaagagagtcttggaaggtctcatgctggatgaggatctccttgaaccaccaccagatcttttatcacctccacgttcactctttgtccattcaacacgaagtctgcgtcccttacgcccaaattcaaagcggtcaagtgctcggatggcatcttccgcatccctttcatcttccatgtatacaaaagcaaacccagctttcatatcaaccctctcaaccttgccgtatttcctgaatagtcgttccaggtcaccttcgcgcgcatcatactcaaagttcccacagaagactggcttcatgcttcctgtagaatgattttggcaggcgtagtcgcg

SEQ ID NO: 52 - DNA, 745 bp, Arabidopsis thaliana
acgataactccgccgtctcccgccgtcttgctctcactctcctcgtcggcgcctgctgttggttccaaagtatctcctgctgatgccgcctacggtgaagctgcaaacgtgtttgggaagccaaagacgaacacagacttcttgccatacaatggagatgggttcaaagtgcaggttccagcaaaatggaacccaagcaaagagattgagtatccaggacaagtccttaggttcgaagacaacttcgatgctactagcaatctcaatgtcatggtcactcctaccgacaagaagtccatcactgattacggttctcccgaagagttcctctctcaggttaattacctcctagggaaacaagcttacttcggtgagactgcctctgagggaggctttgacaacaatgcagtggcaacagcaaacattctggagtcatcatctcaggaagttggtgggaaaccctactattacttgtctgtgttgacaagaacggctgatggagacgaaggtgggaagcatcagctgatcacagcaaccgtgaatggagggaagctttacatctgcaaagcacaagctggagacaagaggtggttcaagggagccaggaaatttgtcgagagcgcagccacttctttcagtgttgcttgagtgaaagcaacacaacgtaacaatgctctgcttgctttcttcatttgtctcttgtaaaaaatggaaaatgaaactgagcttttgagaaaaaaaaaaa

SEQ ID NO: 53 - DNA, 725 bp, Arabidopsis thaliana; identical to SEQ ID NO: 43

SEQ ID NO: 54 - DNA, 725 bp, Arabidopsis thaliana; identical to SEQ ID NO: 43

SEQ ID NO: 55 - DNA, 724 bp, Arabidopsis thaliana
agtaaacgagcaaaagaagaagagaaacaacaagaagtagtaatggcttcctctatgctctcctccgccgctgtggttacatccccggctcaggccaccatggtcgctccattcaccggcttgaagtcatccgctgcattcccggtcacccgcaagaccaacaaggacatcacttccatcgcaagcaacgggggaagagttagctgcatgaaggtgtggccaccaattggaaagaagaagtttgagactctatcttacctccctgaccttagtgacgtcgaattggctaaggaagttgactaccttctccgcaacaagtggattccttgtgttgaattcgagttagagcacggatttgtgtaccgtgagcacggaaacactcccggatactacgatggacggtactggacaatgtggaagcttccattgttcggatgcaccgactccgctcaagtgttgaaggaagttgaagaatgcaagaaggagtacccgggcgccttcattaggatcatcggattcgacaacacccgtcaagtccaatgcatcagtttcattgcctacaagcccccaagcttcaccgaagcttaatttcttttctaaaacattcttatgaattatctctgctcatttcatttcctattgtctgtgttctttttctctttatgagacaatttctatcggattgtcaaatgtctgatttatgaatatgtaatttatataaaaa

SEQ ID NO: 56 - DNA, 416 bp, Arabidopsis thaliana
agccaggagaatactctcctatgccacatcattcgtctttatcgaccagtatgggaccatcatcgtacgaaggcagagagcggaagagcagtagtatgattcaacacggaggttatcttgaagagccaagcatcagacttcttggaaaagaagcttccagcaaaatggctcgtcgtgatcctgacccaatctatgaccgtgaatgggaagacgacaagaggagagcagaaaggaagcggagagatcggaagtagagagtgatgatttgcagatcctttggtttgttcaacgaagagagagacaaatactggtattgaacactgcttatgttgtacacgtactattcaatgaccgtgcgggtctactttgtcatttggctccgccgagtttgataaatgacttgccagacttcagat

SEQ ID NO: 57 - DNA, 145 bp, Arabidopsis thaliana
aatctttgttgttgtaagatgttataaggatctcaagcacctattattcttaaatattattggttgatgttgctagcaagaaaaattgaatacaaccttaaaaaaaaaaaaaaaaaaaaaaaaaaaaaaaaaaaaaaaaaaaaaa

SEQ ID NO: 58 - DNA, 299 bp, Arabidopsis thaliana
gagatggctgcatcgcataaccggaagcttgttcaacctcccgaaggaactttcttctaatactctcaaagcctacctttgaggggcttctccattgttggtcttcaagcttttctttcgtaccttaaagtaaaaacaatggtgtctgtcgatgaatgatgatgttcgattgatcatctggagtttaaatccttgtgtgcaaatatatctagacaacgctgtctcacgacttcatcttctcagtttagatataatatgggaaacaaccttctagaaaaaaaaaaaaaaaaaaaaaaaaa

SEQ ID NO: 59 - DNA, 450 bp, Arabidopsis thaliana
tttttagagagtcaaattagaatcttgtttcaaataccatcttcaaatgcaagagatagtaagagagctcaaaaggttaaaccaagaaagtaaaatgacattattaaggtcgacgagaatgtacaatcatcaagaggatcagacgtagaagctgaggtaattagcagtagaaagatccaccaaatgtgttctctccactgtatgtcatgtagagaaacccgtcttcgtctttgtgttcttcgtagattgcagacatcaatgccgcagttggtggtaatgtgttcttgacaaagacaaagatggctttttcagctccaagcttgattcttttcctcacaacgtacacaaattggccaatggttagatcagctggtacaagatacttcttcttgtcaatgtcaggaacatcactctgtccagcttttccacaatcacgggaactctttcagg

SEQ ID NO: 60 - DNA, 429 bp, Arabidopsis thaliana
ctatagagaatcttcaagcattagaaggatttgtgaatcaagcagatcatctgaggcaacaaactttgcaacaaatggcgaagatcttaacgacaagacaatcggctcgaggtttactagctttaggagagtatcttcatagacttcgtgctcttagttctctttgggcagctcgtccacaagaaccaacttaaaagaggaacttattaaaactttaaaaacaagaaacagcagaatcaaaagtcttgaagaagcatactcatcacaaagcttggaaggatgttttaaaaaagatctttgttaattaagtagagtgagattctcttgattagaactttatggtttttgctttatgaagtatctctccagagaagattgtaaatttgggttgaaactttgtaatatatttagatacaacaaataagtttg

SEQ ID NO: 61 - DNA, 1012 bp, Arabidopsis thaliana
tttttttgcgtaatgtagtttcctacgttgttgtatctataaatagtttgtttctcgagcttccatttcataattcctcatttcccggatctctcccatctaaaaataacccgacccatttacgcgacccaaaccggatcaacccgcaatggataagccaagcttcgtaatccaatccaaagaagcagaatccgccgcgaaacaactcggcgtttccgtcattcagctcctcccgtcgctagtcaaaccagcacaatcctacgctcgaactccgatttcgaaattcaacgtcgcagtcgtcggactcggatcatcaggtcggatcttcttaggcgtcaatgtcgaattcccaaatctccctctccaccactcaatccacgccgaacagttcctcgtcaccaatctcacactcaacggtgaacgtcacctcaatttcttcgccgtctccgccgcaccatgtggccattgccgtcaattcctccaagaaattcgcgacgcacctgaaatcaaaatccttatcaccgatccaaacaactccgccgattccgattccgccgccgattcagacggattcttacgtctcggaagcttcttgccacacagattcggtcccgacgatcttctcgggaaagatcatcctcttcttctcgaatctcacgataaccatctcaaaatctcagatctggattcgatttgtaacggaaacaccgattcatccgccgatttgaaacaaacggctttagcggcggcgaatagatcgtacgcgccgtatagtttatgtccatcgggagtttcgctggtggattgtgacgggaaagtgtacagaggttggtatatggaatcggcggcgtataatcctagtatgggaccagtacaggcggcgttggttgattatgtggctaatggtggtggaggaggatacgagaggatcgtcggagcggttctggtggagaaagaagatgcggtggtgaggcaagagcacacggcgaggttgttattagagactatatcg

SEQ ID NO: 62 - DNA, 605 bp, Arabidopsis thaliana
caaacatcagaagccctagagcttgagccgtcgaaaatgtcgaagcgaggacgtggaggaacgtctggtaacaaattcaggatgtcacttggtctgcccgttgcagccacagtgaactgtgcagacaacactggtgctaagaacctttacatcatctctgttaaaggaatcaaaggtcgtctcaatcggttaccttctgcttgtgttggtgacatggttatggccactgtcaagaaaggtaaaccagacctcaggaaaaaggttcttcctgctgtgattgttaggcaacgtaagccatggcgccgaaaggacggtgttttcatgtactttgaagataatgctggagtgattgtgaaccctaagggagaaatgaaaggttctgcaattactggacctattgggaaagagtgtgcggatctctggccaaggattgctagtgctgctaacgccattgtctgaagatcatttatcacttttgctggttatgtatctgtcttcaacgaaacgcgaaatagttggtgttttgagtgttttaagtagagacgacaatcttttgtgagcttcagacatatttccagtttctaagagattttgcttagattaaa

SEQ ID NO: 63 - DNA, 915 bp, Arabidopsis thaliana
tttttttttttttccaaccacaacgagatgaattacaccacgactctaagtgaaatcatcttttaaaccaccaaaattcacccaatgacacgaaaacatttctagcacaaagaaaatcaaatcctatcctgagatccaatccaattccaagctattagtccctcatgatccgagtgtagaacatgtcctaatagcatctacgccaaaagcgcaacttcagaagggttttgactcctctgctttcactatttcggtcctaagcctaaaacggacatactaatccgactgatactcaaccggatcaaccgggctgagacaaaaatttcttgaagtcgaggctttattggagagctttccttcttcttctacaatccaaatggttttcttttctcccccatttctcattaattctccatcaggagtcgttgacggcagcatagctaatacgccttgaatcatgtaaatctgcccgggatcgataggatcttcacaaccaagctcgaaaaccacgatccgcctacctctgtaccttcttaacactgagccaccactaccaaggaggttcatcggaggagaggtacctccgcgaagagactgacgaagaaatccaccagatctgctgccgtcgtcgagagtcatagttccccattgtagatctaccaacgaacctccatctgagagaactatctccgctcgccgtcgacttaaaactctttgattaagccacaaagaaggaggagccgccgccggctaaaaaaaatcgacacaaaggaaaaaaaaaactaaaaagagaagagaggccctctcacggtgccggccgaaaccgaccaccaaagaggcaaaacggcgtttgaagatttgatctggtttgcggcggctaactagagagagagaactagggttttttgtttagtt

SEQ ID NO: 64 - DNA, 429 bp, Arabidopsis thaliana; identical to SEQ ID NO: 60

SEQ ID NO: 65 - DNA, 574 bp, Arabidopsis thaliana
ttttttttttttttttttttttttttgaggagaaataattggtaaacttttgcggtacatacggtttgggtcaagttacaaacggataaaccggtatagaatacacagagtttttgaattctcccatttaagctgcaacttcttcgacctcatccaatgcatagttgttggtcgatatgttggcgtaattgacttttgcgaaccggaccacaaccgggtatcgagtcttagggtcctgatcaacggcaacaactgatccaacgttcttgaaccaataggattctctccttagaatcttgaccttagaccctctcttaggaccaatcggtggtggcttgggtttggtggcagtagctccatccggagcagcggcagctgccggagaatcttttgaagaagaggaagccggagcaggatcttcggctgccctgactacgagcctagaaccggcgtttctcatcggcaagaaagacacggagctcctggacgacgaagcgccggcgaccgaggtgacattggccggtagaacaaataccgtagatgctgtcgtcatcgccatctctggttttctttt

SEQ ID NO: 66 - DNA, 714 bp, Arabidopsis thaliana
ttgttttttttttgttttttttttttttttttttttaagaaaggcgatttgctcaccataatcacaacatattcaagaaccaaagacaacgaggtgacataaaaaaacaccaaaaaagggactcaaaacatgaagaaacaaaagagaagaaacaagaaacttgaagaaacaaggccattaactccgaatgcataagctcctgagttagtagttgttaaaagagaatagccgccttccggtgtgtttgtagtgaggatgacaacaacccaaatatcaccagaaccaataccaattccagtgatcttagaatcattcaagttcttgagtacgacactgttgaaattgctcaagtcagggttcttatcatgcttaggaaaacagacttgcatgatcacaccgtctctgactacggttgtgttgagactgcatttagcgaggaggttatttccagggactggagctgagttattagtgtttgtgcagggttggttctttagttggtctacgacttcgtctgcgagacattctgcgttttcgttctttgttaaggtttttaggtttagtcctgttctgtatttgttgaatactgtaagaagaaggtcttcttctccatcggtgccggaaagaacaagacgatgaagggagagaaagactgagagaagacagagtagatggagtttggaaatcgccattgatgcagaggttttttttttt

SEQ ID NO: 67 - DNA, 780 bp, Arabidopsis thaliana
agttacatcgagtaacgcagcaacatttggggtcggggctattcaggtagtagcgactgcaatatccacttggttggtggacaaagcaggtcgtcggcttctgcttactatctcttcggttgggatgacgattagccttgtaattgttgcagctgctttctatcttaaggaatttgtgtctcctgattcagacatgtacagttggctgagcatattgtcagtagttggagttgtggcaatggttgtctttttctcattgggaatgggaccaataccgtggctcattatgtctgagatccttcctgtgaacataaagggtttagctggaagtattgcaactctagccaattggttcttttcttggttgatcaccatgacagcaaatttgctgttagcctggagcagtggaggaactttcactctgtatggattggtttgtgcattcacagtggtgttcgtgactctatgggttcctgagaccaaaggcaaaactcttgaagaacttcaatccttgttcagatgaacaaattgaaacaacttcattctttgtcaccctctctctccctctctgttttggccaagaacaagaagaaacaagagattttccagctttgttaattgggctgagaacgttactaagatttgtttgtttgttcgttgtgtgtcaataatcgcattatcttctatcacatgtatatcaacatactacattcaagtatttgtaattttattgaactctttacatagagcaaaggttttgccaaaaaaaaaaaaaaaaaa

SEQ ID NO: 68 - DNA, 641 bp, Arabidopsis thaliana
gaacattcagaacaagaactcatcctactttgtggaatggatcccaaacaacgtcaagtccagtgtctgtgatattgcaccaaagggtttgaaaatggcgtctactttcattggtaactcaacctcaatccaggagatgtttaggcgtgtgagcgaacagttcacagctatgttcaggagaaaggctttccttcattggtacacaggagaaggcatggacgagatggagttcactgaagcagagagtaacatgaatgatcttgtcgcagagtaccagcagtaccaagatgctacagccggagaggaagagtacgaggaggaagaagaggagtacgagacttaagatgttgtcaatggctccctcggattcgtaagctgtgtaagcaagcagcattcactttcttctttccccttatcctgaatttttttcttcgtaatatctcttttattgtttcgttcatgtgtgttcgtttttgttattgaaaccctatatcggttctggatttgttaaacttttgcgtgtattgcttattgtttttgtcggtgaaaaaaatattgcttttgttctcttaagttttgtgttgccaaaaaaaaaaaaaaaaaaaaaaaaaaaaaaaaaaaaaaaaaaaaaaaaaaaaaaa

SEQ ID NO: 69 - DNA, 503 bp, Arabidopsis thaliana
ttttttttcagcccaaagaacactttttaattactagtaaagtttaactaacggttaataaacttacatcagacaatattacactttttatcttggctgcttcaatgtctccgcatcgttcgttttaccggtgaaagaagcttcttagctttcctctttcaagcttctcgagaagcttatcggcgcccattacttccatctccgacagcttcttcagataccctattgctccgtacgacaccatcattttcttactcttctcgcttcccgacatccccaacagccccgccacggcgtacttcttcgccgtgtttccagggtttgaatccaataacatcaccaaattcgtcagaacgctcttcccgtctttcttcagttcccgtcgaatccttccttccgctaccaatccagcgatcgcctgagccgccgcttctcgacatccgtttgacttcgattccagaagtttcacgatctccgggatgcaaccggattctcccactagc

SEQ ID NO: 70 - DNA, 503 bp, Arabidopsis thaliana; identical to SEQ ID NO: 69

SEQ ID NO: 71 - DNA, 578 bp, Arabidopsis thaliana
gattcgataagaagaatctacatggctcgacatatcatggagaagttcatcgtcgcaggagcggaaatggaattgaacttatctcataaaacccgacaagagatcttaaccactcaagatctaactcacactgatctcttcaagaacgcattaaacgaagtcatgcaattgatcaagatgaacttggtaagagattactggtcatccatctacttcatcaagttcaaagaagaagaaagctgccacgaggcaatgcataaggaaggatacagtttttcatctccaagactgagttcagttcaaggctctgatgatcctttctatcaagaacatatgtcaaagagttccagatgcagtagtcccggttaaggagtctaaaactggtactagaccagaacccaaaccaatgttcatagcaatccaatccatgtaatcttccttcacatttcttgtacatgtcattttctctcttgttatacctaactgtaagagaaaatgtccggttcggattttggtttagttttaaatgtgtataccggacaaaaactatggaaccatactaattaatatctcgaaga

SEQ ID NO: 72 - DNA, 679 bp, Arabidopsis thaliana
tgggtttttgttttgaactctccttatgtattaccgcctcgccggagactgatacagtttcttctgtccctcattgaaagaagaaaagaaaacaaaaatagaaaaaaaaagaaagcagaaaaaaagcctaggaggaacaatgaatttagaaaaccaaaccatgacagaaaagtctgcgggattctctggttagctctaggtgatgatatgatcaagtttcgtcctcactggctttgtatgaagggaaaagaagataatctaaaagattcgccaaaagacacagatcgttcaccgtgatggctcgcctacaatatcgtggtaaaacaaaaacgattgtactaagtagcaattcctctgtttggttgtctcttgttcacactgtaactgccaacataacctggagatgaacttctagctgaaacatctgaagaaggaacccctcctccaatcccatagctaaaaggagcaggcccttctgtctcaggagttggtgatctccatgtagggtcactataaactgcaccattcccgtaaaactctgcaagtcctgctgtatcataacccgtgttgttggttgctgaggcagaagagaatgaagaggatggtgctgccttgttagcccctgggtttcttgctgcataaccacctgttcccaacccaaaaccagattcaccgtttc

SEQ ID NO: 73 - DNA, 599 bp, Arabidopsis thaliana
ctcggtgaggctgtcggtgttgaagggctggttgtcggtctgcgcgctcagcgccagcagcgcgcgccgcgagaagcaggtaccgacaccggccgacggcaccatgccggaaacactttcgcgcaccaccagatccttggcatgccattcggcgaactcgtccatgtagacgccggccaccagttcgtaccactcgcggtccagcgaggtgaccggcaactggatcatgtccttgcgcggcaaaaggtagttgtagaagcgcagttccatcgggtgcagcacgtcctcgctgtcgtgcaggatcaccccggcgaactcgatgtcgtggcgcttctcgtaatcgaagatggccaggatcagccagttcaggcagtcggccttgctggtcggcccgtcatgcggcacttccacgcggcgcaggcgcttgtagcggcggcgcatgcgctccacttcgtcgatggtctgctggtcgttgggataggtgccgacgaacacgacgtactcgcggtaatcgagtacgttgatcatgttctccaccatctgcgcgatgacgtcgtactccatccacgccggcaccatgatcgccagcggctgttgcg

SEQ ID NO: 74 - DNA, 997 bp, Arabidopsis thaliana
ttgatttaaaaacagttggtggaaagcttactttgtaccaaacgaccctatgcgagagaatctcaggggataacattgatctcgggctagatctcgggtctcaaagctttttgccaacatacaacaaaaatgacatccagctgatatgctgtcaagctgatgcaagtgttttatggcttgtccctgacacagttgtgaccagatttattcaatcccttgactgggacacagatatggacatcacctttacttgggttcttaacagagaccgccctaaaggcaaggagactgtgaaatatgaaagaagtgtcgaccctctggaccttccaaaacgctctgatatccaaatggttctcaatgggtcgatggatggatttagagtgcataatttgtacccaaagttcttccgtgttactggttctggtgatgtcaggtctttcgaggatcagacggatgaagtgagtgcagacatactcattaaccatgcaaatttcaagtggtggtggtcattccataatcttaaagcgtctgaaaatatcagcgcttgcgaggggatggatggaccagttgctatcataatgtctgaggaaacaccgccacagggctttctgggtgacaccctcagcaagttcagtatatggggactctatatcacatttgtactagcggtggggcgtttcatcaggcttcaatgctctgacctgcgtatgagaataccttacgagaacctgccttcgtgtgacagattaatagccatatgcgaggacttgtacgcggctagagcagagggtgagcttggagtagaagaagttctatactggacgcttgtgaagatctatagatccccgcacatgctgctcgagtatacaaagctagactatgatgcttaggtccaaaaccagtctctcacactaaagaaacactttgtcatatttgtacatactgagcggaatattctgagggatttgttttgttttcaatcagcttgtagttgatt

SEQ ID NO: 75 - DNA, 329 bp, Arabidopsis thaliana
acgatgttgatcacaaggggcaagagatggtaacaacagtttgcatgaaatgccacatgctggttatgttgtgtacatcaactcctgtttgtcccaactgcaagttcatgcacccacacgatcacagctctacaaaactgtttaaaccatcaaatttgcttaggcttctatgctaggctctttcaaggttactgaatctataaaatttgtacggcagataataagccaagagactagatatggacaaagttatgtatatactaaaagtaccagaaagtttgtattaattctctgcttctatgaacgatcatgctttagatctctaaaaa

SEQ ID NO: 76 - DNA, 546 bp, Arabidopsis thaliana
cgctcgcgatctagaactaggcttttacgaacagagagcgagccagagagagtgagtaagagagaatgacgagcgtgagtgggtgtggttcagtgagtctgataactaaccgcagtgcgttcttgggaaacggacttcaacaccgtgccgttttccttaaaccatggtcgtcttcttcgcttcagtctcggtccatggttgtcgaagccaaaaccaaaaccagcagcgaagacagaatcgcccgccactctcgtatccgtaagaaggttaatggtacaacggagaggccaaggctatgtgttttccgatcaaacaagcatctttatgttcaagtgattgatgataccaagatgcacaccttagcttcagcttccactaagcagaaaccaatctctgaagagttcgactacacctctggaccaaccattgaggtagcgaagaaagttggggaagtgatagcaaaatcttgcttggagaaaggtatcacaaaggtagcctttgaccgtggtggttacccttaccatggacgtattgaggctcttgctg

SEQ ID NO: 77 - DNA, 678 bp, Arabidopsis thaliana
tttttattaatagttattttattaaattttgaagtactatttttgtcaatacaaaaattctgcaacacattctgcttcaggaagaatgaaatcagtctcccaacaaacaagttctttacgaataccaaggggagtgtcggactgatgttagccaagttgattttttttttcatcaagaaactaaatgctttctctgagtttgacaggaaggtcaagatcaggttccgtgggagtcaaggcacagaagtaatcatcaaccatgtcctctgatactttctccaagctcggtggatcccactttggtgcttcatccttgtctatcaatcgagctcgtactccctcacaaaaattgccggacattggcccgattaatccttgtagcgacattctgtactctcggattaagcattggtcaagtgtttgtaatcttccttcccggatctgttggattcgttttaaagaagagatctcaatgccaccttcaaagataacggtgagctttctttaagtctacgtagagtcgtaatgcaccatgtatcttttcttctactagcctcgatttccaaagaatcaataatttcttctactgtgtcatggctaaagcatttttcaagtaaatcgatcctacgaataacaccagtcttttccggatgggcaacttctgcacatttttctaag

SEQ ID NO: 78 - DNA, 614 bp, Arabidopsis thaliana
agttaaatggtttgggatttaagaaagttttcttcttataacagagttggtaaatttaaaatacaacggaatataatcgaaacaatcagtgaaactatagagatatattgatcacttttcaatttttcatgacccaaaacctctcaatttctccagcggttcttcctgggatcctcccagctatcagttcccacctttcatcaaataataacacacaaaattcagcttttactatggtgttacaattaaattattttcctacgaaatagtattcattattagttaaaagatcaaacctgtcaccgacaagcttatgcattcgagagaccaaatcttcttcttcttgactcatgttcacaacttcccactcaagactactcacttctgttccttgtcatcaccaaaattcagatttctcattatatatagataagtataaaaaaacatggaaaaatgagaaaacgaaggtgtttaagttttcagcttaccttcagaagaagaagtaacgatggagttggtcttgggttgcttagtcctgcgatggttatccatgtcaaacggcaccgtattacaaagaagaagaagaaagaaactaagagagtactctgagagag

SEQ ID NO: 79 - DNA, 578 bp, Arabidopsis thaliana; identical to SEQ ID NO: 71

SEQ ID NO: 80 - DNA, 668 bp, Arabidopsis thaliana
tatagaaattatgcgtacgctacaatcaatggcattacaatgaaccaacctggtattcagattttaccaaatgacaatggacagaaaaaatatacaattttgaacagatcagatgcaaaagttgttctacagacagaaaaagggtaagacttgatgcatcttgttgaggagtttgggaagatgatgatgatgatgatcggtaggagtaggtggatctgagttcttgtctttcttttttctcctaagctggaaaaactgctgcattgtctgagcgcattctgattcaagaactccgcgccggattgtcatcttaggatggaatggatgaactggaggtggtggcttttctgaagcctctgatccatttccttcgcctccagggaacagcctgatccagcttccatctgctccgagaagcttattgggagcaccccatgcaagagtgttgacccttgcttgaagtattgctcccgcacacattgggcacggttctagtgttacatagagcgttgtatccgcgagcctccatgaacgaagtgctttagaaccctctcgaatgcaaatcatttctgcatgggcagttgaatcacgaagctcctctactaggttataaccacgggcaataatctttccatcatgaacaagcacagcaccgacaggtacctccc

SEQ ID NO: 81 - DNA, 682 bp, Arabidopsis thaliana
cttcatcaaatctcacaaatcttcaacacttaatcacaaatctcaaagcttcggataccaaatggctcgtaccaagcaaaccgcaaggaaatccaccggaggaaaagccccaaggaaacaactcgcaacaaaggcggcgaggaaatcagctccggcgaccggaggagtaaagaagccacacagattccgtcctggaactgttgccctaagagaaatcaggaagtatcagaagagcactgagcttctgatccgcaagcttccgttccagcgtttggttcgtgagatcgctcaggatttcaaaacagatctgcgtttccagagcagcgccgtcgcagcacttcaggaagcggctgaagcatacctcgttggattgtttgaagacaccaatctttgcgcgattcatgctaagagagtcactatcatgcctaaggatattcaattggcgaggagaattagaggcgagagggcttaagaaggagattgaagtactctagactgtgatcgttatgcttatgtatatctttcgttttccctaatttcgtgttttagggttggattaggttttgcgtttatgttgttcgatatctaacggatcaaaatctctccttccttagcaaagtttgaaaactccctccacattttcaaaaaaaaaaaaaaaaaaaaaaaaaaaaaaaaaaaaaaa

SEQ ID NO: 82 - DNA, 809 bp, Arabidopsis thaliana
cgagcttcgacttcgagctgtggaaagtctgccgtgccacgtcagcaacaccaagcctcttcaagccgttcagtgtagtgtcggtggacgggaaaacctcatgctcagccgtagacggcggtttggtgatgaacaatccaacagcagctgccgtcacgcacgtgctacacaacaaacgagatttcccgtcagtaaacggcgtagatgacttgcttgtactgtcgttgggaaacggtccgtcgaccatgtcatcatcaccagggaggaaactccgtcgtaacggagactattcaacgtcaagtgtggtggacatagtggttgacggcgtttccgataccgtcgatcagatgctggggaacgctttctgctggaaccgtactgattacgttagaatccaggcgaacggtttgacgagcggcggagcggaggagttgctgaaagagagaggtgtggaaacggcgccgtttggggtaaaacggatactaacggagagtaacggagaaagaatagagggtttcgtgcaacgtcttgttgcgtcaggaaagtcaagtctacctccaagtccttgcaaggaatctgccgttaaccctctcgctgacggccgttaagtttcctttattattataaccctccccgtccgtgatgtaagaagtttgtaaccaaacccctgggttaattttttaaccccagccagcatcttcgagttaattaattagcctttctttttttctaatgactttagttgaggaattaataatggttaatgaatgatagtctttacttatttatccaaaaaaaaaaaaaaaaaa

SEQ ID NO: 83 - DNA, 356 bp, Arabidopsis thaliana
tctccttggaatgtccaagcttgataattctcttgcttatctcttcttctcccgctaggggctctgattcattgtctgcggacgcgtgggtcgacccgggaattccggaccggtacctgcagccattggagctctgctgttaattgaagacaagatcaagacaagaggagtcttaaggcctctcgaagcagaggtgtatttgccagctttggatatattgcaagcatatggtataaagctgatggagaaggcagaatgatcaaagaactctgtatattgtttctctctataacttggagttggagacaaagctgaagaagacagagacattagaccagcaaaaaaaaaaaaaaaaa

SEQ ID NO: 84 - DNA, 1113 bp, Arabidopsis thaliana
cttcttcagggttcaggtgtgaaagctgacgccaccgtggcagctgacggtagcggtacatttaaaactgtggctgctgcggttgccgcggcccctgaaaatagtaataagaggtatgtgatacatataaaagccggagtttacagagagaatgtggaggttgctaagaagaaaaagaatataatgtttatgggagatggtcggacgagaactattatcaccggaagtcgaaacgttgtagacggtagcaccactttccactccgccaccgttgctgctgtcggcgagagattcttagctcgtgacatcactttccaaaacacggcgggtccgtcgaagcaccaagcggtggctctccgtgtgggttctgatttctccgccttctacaattgcgacatgttagcttatcaagacactctatacgtccactctaaccgtcaattcttcgtcaaatgtctcatcgccggaaccgttgacttcatcttcggaaacgccgccgtcgtgctccaagactgtgacatccacgctcgccgccctaattccggtcagaaaaacatggtcacagctcagggaagaacggatcctaaccagaacacagggatcgttatccagaaatgtaggatcggtgccacgtcggatttacagtcggtgaaaggtagttttccgacgtacttgggtcggccatggaaggaatattcacaaacggtgataatgcagtcggctatctccgacgtgatccgacccgaagggtggtccgagtggaccgggacttttgcgttgaacactctgacttacagagagtattcgaacacaggagcaggggctggaactgcaaatagagtgaagtggaggggctttaaggtaattacggctgctgctgaagctcaaaaatatacggctggtcagtttattggtggtggaggctggttatcgtcgaccggtttccccttctcgctcggtctttgagagattgttgtgtaatgtgttcctacgtattgttggctacaaaaattattgattaatattgtatgaagcaaatcgtgttgtcctctttgttttgtttgggttgtgtactttctctagatcatcgtagtattagaaacgagatgaaaa

SEQ ID NO: 85 - DNA, 728 bp, Arabidopsis thaliana
caggcaaacaagaccaagaggaagaagaagaagaaaaagagaaaaggccctgtgatggacaaacccatgagtgtagactggtttgttagggaaacttgtagacgcctcaaggagaagaagtcttacatgatatacacagctgttgggtgtctcggaattgctgccttaagtgatcttgtcaatgaggtggtagcaattgagacctgtggaggtcaggtgactgctgatggcactaggaaacggacaagtggtggtgtattgtggaacatcatcaaagcgagacagcctgaagcttatagagagataatgaaaaagaccaaggagtttgagaaacaatttaggcaaccaaacacgagaccaaaatcagggcccaaaagagatcagggtagctcctccgaaggagttgcctctggaaatgtatctgctgatgaagctctggtgagcgagatgtgtgttatgccggtagctgaccagactgaatccaaaccggaaaaggaaaggaaatctgttcatgagaggatcagggtacctgtttcatatgatgaccttttcagagatgcacctttagatgattctctagcacatcattcttctgcttaagctcattactggatgacttctcttgtggaaagcaattgttttgtcgagaaatggaaagcattgattttgtcgagaaatgcattgacaaaactatatataccaactaccaagatttcttaaatacacaa

SEQ ID NO: 86 - DNA, 871 bp, Arabidopsis thaliana
caagaacattctcagcttctagaaggttttctcaccaacccccaaattatgagaaaattacgaaattggctaaccaactacaaaagaatgattcaattcaccaaacgaattaaatgaagcattaaattgagagtaaatgagttttcgttagagtgaaactcacgtaagtgttgagctgacgaatgaagcttgagaaattattatgcttgaagtattgaggaagaagatctttagcaaactctgctgttttccacacgacaaaagctgttccttcttcgttccatgaaacgacgtcgtctgtgctatgatcatcaactagctgatacgttttgcttaaaaacggcgccggaactgatctttgcgccgccgtcacagccgtcatctccggcgaactttttttattttaccacagaaaaataaaactaaaaataatctaatacacaaagagaagaagaaagattggaaatagaaagtcgaaggaaaaagaatcagcaactaaaaagcaagagagcggtgagaaattcccaatcccagcaataaaagccagagaggaaaacacgagaacggagaagatcggagtttcgtttggtttcttccatttaaggaaaaatctgatgatggaggaagaagatgaagacgacgaccatacttcgccggagctaatccgtgtgattaaaaagtaaataaatataaagtcttttttatttttgtgtgtatgtgcaaaacaagtaaaacaaatatataaacgagttaagtgttatgtcgaagggtctctatataacgtagtaggaagatttatagatcacaaatgttggtcctacctttgtaagaaaattaaattataaaaacggatgctgtttctagaaaaaaa

SEQ ID NO: 87 - DNA, 962 bp, Arabidopsis thaliana
gaagaagttccgtataacatctccattattcagatcagtagagttttaccgtcggagactgcggcggctccgactcctgctccggcggagatgaatcttaccggaataatgtcggctcatggatgcaaagtgtttgctgagactcttctcactaaccctggagcttcaaaaacctatcaggagagtttagaaggaggcatgacagtgttctgtccaggagatgatgcaatgaaaggtttcttgcccaaatacaagaacttgacagctccaaagaaagaagcatttctcgatttcctcgctgtcccgacatattactcaatggcgatgccaaatccaacaatggtccgatgaacacacttgcgacagatggagctaacaagtttgagcttactgtacagaacgatggagagaaggttaccctcaagacaaggatcaacactgtcaagatcgttgatactcttattgatgagcagcctttagctatatatgcgactgataaggttttgttgcctaaagagttgtttaaggcttcggctgttgaagctccggctcctgctccggcaccagaggatggtgatgttgcggattctccaaaagcggctaaagggaaagcgaaaggaaagaagaagaaggctgcaccgtcgccagataatgatccttttggtgactcggattcgcctgccgaagggcctgacggagaggccgatgatgcgacggcagatgatgctggtgcggttaggatcatcggaggagctaaggctggtttggtggtgagcttgctctgcttgtttgcttcttcttggcttctatagtttcacttcttgtttcttcgattcttccatgtttttttttttttgtgaatcttttatttatggtttttgggggagagtaaatgaggattatttatttccctctattgttgagttttttttatttatttaaaagttggttgtcgaattaaa

SEQ ID NO: 88 - DNA, 835 bp, Arabidopsis thaliana
ggacaaggaaggaatccctccggatcagcagagacttatctttgccggtaagcagcttgaagacggaagaactcttgctgactacaacattcaaaaggagtcgacccttcatttggtgcttcgtctcagaggtggtatgcaaatctttgtcaagaccctcactggtaaaacaatcacccttgaggttgagagttcagacaccattgacaatgtcaaagctaagatccaagataaagagggaattcctccggatcagcagaggcttatctttgccggtaagcagctcgaagatggacgcacccttgcagattacaacatccaaaaggagtcgacacttcatcttgtgcttcgtctccgtggtggtatgcagatctttgtgaagacccttaccggaaagaccattactctggaggttgaaagctcagacaccatcgataatgtcaaggctaagattcaggacaaggaagggatcccaccagaccaacagagactcatcttcgctggaaaacagcttgaggatggtcgcacacttgcagattacaacatccagaaggagtcgactcttcacttggttcttcgtcttcgtggtggaagcttctaagctttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaaaaaaaaaaaaaaaaaa

SEQ ID NO: 89 - DNA, 581 bp, Arabidopsis thaliana
atacaaacactagaagtcttataaattcaaagtatgtctgagttttacaacattagagagaaagagaacaacacagaaacatttatagaaacatgattacacatgcgctaacaactttaagatttactgagccaaagcacttgtgttgtacacaagaagagcacctccggcaagaattccggcgagagttaccgcccaaattgccaatccggtgactcctcccttgtacacgtcaccactcgctgaccactcgttctcgttgtaaataggactgtatccatcgacgttagctccatacttgtcgacgtacttgtaaacaccgtatcccttgccctttctgccggaggcgtcgacgccgtccctcaagtccatgctgccgttaattccgaagggcttgtcggtcttgatcttcttgacgccactggcgacaattttgaaggagggacgagctcttgtgagcgacggtaatcctctagccgccgtcttctccaccgtgaaaccagctggtttcaatgtcaccgaagatagcatcactgaagcagccattatttttctcacaagatgatcaaactattcttctc

SEQ ID NO: 90 - DNA, 884 bp, Arabidopsis thaliana
tttaatgcttttctaatcaataaatatcaaattatccaccagataaaaataataatttaaaaagcgtattctcaaatcgtaacaaaaagggatatttttggtgtttgtcacccaaaagtataacctatccaatgagggtatgaagaaaattgagtgaatcaaaatataaaagataaaaaaaagggaagacgaagcaaaactcttttgtatgtttcttctcattagcaaaggctggggtaaaacttagaagttgacttgaaagccactgcgtctgcgatgacctgcaccgccttttgatctgtttgagtttctttcatattggtatcgcatccccaaacccttaccgcaagcctacgacatttgggtgatgattctctgaaataggttttgtaccaactgatcacatctttcttctgtatacttcttagttcttctgcttctttgtgggagaaatcaaacatgtaccttttgtcaacaatctgactccataagtcatttgtctcggacaagagagagggatccttttccagcaatctagcaatcataccacttcggtaatcttcataggattcatcatccagttgttccagaagcccttcgatatcttttatgaaattgtcaactctccccagcaaatgaactggaccgtacttagaagattgaacacagaaacagaaaccgtgcacacgatacgttaagcgagggccacactcgacaacataaccaagctgctcctttgtcctcaactgattgaacaatggctcttctatgatttcatgaaagagatccagcacagctttcgttctcgttgattgagcttcttcaggctcgatttgatagtaaagctcgactactgagtttgtttcagatttgttcttcacatt

SEQ ID NO: 91 - DNA, 730 bp, Arabidopsis thaliana
gtaggggcaaaacatattataccataaggaccacaaaacatcacaacaatgatattttcaacagagtactagtagagtatgtttataaggagggatagggaatttttttcaaacatagaacagattctctgagagagaatgttttcataagagagtattatatagctaactctgatttcagcaggtcaagagaggagatgaaccactgcatttgacatcagaagcatcagaaaggcgttgtcttggagagagtgttgtaatcgctgcaacatctacgtcgagattcactatgagcttcctcttctgcgactctgttacactgttccttctctcttctgatccttcagcaatgggactcaaagtctgtgtctctgctgctccaacgtcttctccatctgctgcgtataggattctttttatggaaccaacaagaggtaagtgctctgtgtctgggttttgacagagaatctctacatctctgagtttagagaaatagaaatctctctctttctctaagctgtcaatgtaaagtttcagttctgtgatcttttcatcataagcaggcactggtttagattgtttagctgatggttttgaagagtggtggtgattcccagttgaagaatggtgagtaccggtgttgttggattgtggctcatgcttacgggttccatttgaagatgaaggtcgaggtggagctgaagaagacgaactcttgcctgattgttgt

SEQ ID NO: 92 - DNA, 1706 bp, Arabidopsis thaliana
aagaaaagtaattctctgtttgtgtagttttctttaccggtgaattttctcttcgttttgtgcttcaaacgtcacccaaatcaccaagatcgatcaaaatcgaaacttaacgtttcagaagatggtgcagtaccagagattaatcatccaccatggaagaaaagaagataagtttagagtttcttcagcagaggaaagtggtggaggtggttgttgctactccaagagagctaaacaaaagtttcgttgtcttctctttctctctatcctctcttgctgtttcgtcttgtctccttattacctcttcggcttctctactctctccctcctagattcgtttcgcagagaaatcgaaggtcttagctcttatgagccagttattacccctctgtgctcagaaatctccaatggaaccatttgttgtgacagaaccggtttgagatctgatatttgtgtaatgaaaggtgatgttcgaacaaactctgcttcttcctcaatcttcctcttcacctcctccaccaataacaacacaaaaccggaaaagatcaaaccttacactagaaaatgggagactagtgtgatggacaccgttcaagaactcaacctcatcaccaaagattccaacaaatcttcagatcgtgtatgcgatgtgtaccatgatgttcctgctgtgttcttctccactggtggatacaccggtaacgtataccacgagtttaacgacgggattatccctttgtttataacttcacagcattacaacaaaaaagttgtgtttgtgatcgtcgagtatcatgactggtgggagatgaagtatggagatgtcgtttcgcagctctcggattatcctctggttgatttcaatggagatacgagaacacattgtttcaaagaagcaaccgttggattacgtattcacgacgagttaactgtgaattcttctttggtcattgggaatcaaaccattgttgacttcagaaacgttttggataggggttactcgcatcgtatccaaagcttgactcaggaggaaacagaggcgaacgtgaccgcactcgatttcaagaagaagccaaaactggtgattctttcaagaaacgggtcatcaagggcgatattaaacgagaatcttctcgtggagctagcagagaaaacagggttcaatgtggaggttctaagaccacaaaagacaacggaaatggccaagatttatcgttcgttgaacacgagcgatgtaatgatcggtgtacatggagcagcaatgactcatttccttttcttgaaaccgaaaaccgttttcattcagatcatcccattagggacggactgggcggcagagacatattatggagaaccggcgaagaagctaggattgaagtacgttggttacaagattgcgccgaaagagagctctttgtatgaagaatatgggaaagatgaccctgtaatccgagatccggatagtctaaacgacaaaggatgggaatatacgaagaaaatctatctacaaggacagaacgtgaagcttgacttgagaagattcagagaaacgttaactcgttcgtatgatttctccattagaaggagatttagagaagattacttgttacatagagaagattaagaatcgtgtgatattttttttgtaaagttttgaatgacaattaaatttatttattttattaagttttttttggtaaaaaaaa

SEQ ID NO: 93 - DNA, 737 bp, Arabidopsis thaliana
agaagaagttaaagcaaaacacatacaaacgcagtcaccttctctgtcgcctccttcttcaatctcatcgcaatcatgatcatatccgagactaatcgccgtgagatctccaagtacctcttcaaagagggtgttttgtttgccaaaaaggatttcaatttaccacaacatcctttgattgagagtgttccaaatctgcaagttatcaagttgatgcagagtttcaaatctaaggaatatgtgagagagacctttgcttggatgcattactactggttcctcacaaatgaaggtattgactttcttaggacttaccttaatctcccatctgagattgttcctgctactctgaagaagcaacagaagcctcttggtcgaccttttggaggtggtggtgaccgtccccgtggccctcctcgtggtgatggagagaggaggtttggtgacagagatggataccgtggaggtcctaaatcaggtggagagtatggtgacaaggctggagcacctgctgattaccagcctggcttcaggggtggagctagtggagcaaggcaagggtttggtcgtggagctggtggttttggtggtggtgctggtccagctgctggatctgatctaccttgaaaaggactttcttgtttctttttggtcttatttaaggttacatagcaccttattgagaacgaatgtgtcttttggaactttgtttctttctcttaaaccatttcacaaaa

SEQ ID NO: 94 - DNA, 907 bp, Arabidopsis thaliana
agaaaaagaacaaaaacctaatttcaagaaattcaataaatatcatcctccggataagttgttattgtacgtttaccaaattcaagaacaagaaaaaacttttcctttgaaacaaagaaacatggatttcttcaccgatcaagtaaagaagaaattctccgacaagaaaccggagagctctgatccggagccaaaccacaacaaaaacaaacccggtcacacggagccaacaacacataaacccggtcacggcgagccaacaacacataaaccggtctccaacaccgatccaacaacacacagaccggctacgaacgctgagctcatggctagtgccaagatcgtagccgaagctgctcaagccgctgctcgtcacgagtcagacaagcttgacaaagccaaagtcgccggagccaccgctgatatcttagacgccgcttctagatacggtaagctcgatgaaaagagcggtgttggtcagtaccttgaaaaggctgaacaatatcttcacaagtacgaaacttcccactctcactcctccaccggtggaactggaagccacggtaatgttggaggacacggtggtggagctggagcaccggcggctaagaaagaagatgagaagtccggaggtggtcatgggtttggagattatgctaagatggctcaaggttttatgaagtgagtaatgttttagtttctaaaaataattatgttagtaattatcttctataattactgttttagtaagctgttgttttttctgaattattattaactgttggatttgtcatttgtgtatgatggaggaaattatgatgttaaagatcatgtatcatgttgttgaccactcgagattgcgttaatcaaatatttgtataattagaaccgaactttaagttaaaaa

SEQ ID NO: 95 - DNA, 437 bp, Arabidopsis thaliana
atacaaggaaagtgttttgccatctgatgtatcagctagagttagtatcgaagctggatcgacttttggatggggaaagatcgtcggaggaaaagggaaatcgattggaattgatacgtttggagcaagtgcaccagcaggaaagctttataaagagtttggtatcaccattgaagctatggttgaagcagccaagtcacttatttaaaaaagtatcttacaggtactaccgaggtttgcatttgaagtaagagacattccataagcattatcttctttgtccaaataaaaatatactccttccaatctttttataaatgatgtttaaagctttcattttggtttttaaataaatgatgttttaaattttcaatgcaaaattatttttattggttgattaaataaatgatgttttaggcttttatttatattttaaa

SEQ ID NO: 96 - DNA, 413 bp, Arabidopsis thaliana
cttgtcaaagagaagtgtgtttgcgtcatcttcgattagtgtggggaaaaacttggaggatatgtcagcgtatattcatttcttggcgtctggatttgaagcttccagaacagcttttggtgctatacctggaagcttgcagcccgatgaagagttatgtagagatcttggtttgtctctcaacactccttccccaaatactcgcaagcaagattgacctgttttttaatttatctttgtctgcatatcattgggatatttttgtgtataaatcaatgtatactatctgaatcattctatagcagctttggtgtaatattgatgatgaaagttagatttttcatctaaaaaaaaaaaaaaaaaaaaaaaaaaaaaaaaaaaaaaaaaaaaaaaaaaaaaaaaaaaaaaaaaaa

SEQ ID NO: 97 - DNA, 1365 bp, Arabidopsis thaliana
tttttttttttaagaagaagttcgacttgtcattagaaagaaagagataacaggaacggaaacatagtagaacacttattcatcagggattatacaaggccccaaaacacaaaccaccaaagttttacatgaaacgaaacattgaacttcttaagcataacagagacgagatttagaaaccaccacgaagacgcaggaccaagtgaagagtagactccttctggatgttgtagtcggccaaagtacgtccatcctcaagctgctttccagcgaagatgagacgctgctggtccggaggaataccttccttgtcctggatcttggccttgacgttgtcaatggtgtcggagctttccacttcaagggtgatggtctttccggtcaaagtcttgacgaagatctgcatacctccacgcagacgcaacaccaagtgaagggtcgactccttctggatgttgtaatccgccaaagtacgaccatcctccaattgttttccggcaaagatcaacctctgctggtccggagggattccttccttatcctggatcttggccttcacgttgtcaatggtgtcagagctctctacctccaaagtgatagtctttccggtgagagtcttcacgaagatctgcatacctccacgcagacgcaagaccaagtgaagtgtggactccttctgaatgttgtagtcggccaaagttcttccatcttcaagttgctttccggcgaagatcaatctctgctggtccggaggaataccctctttgtcctggatcttggctttcacgttatcaatggtgtcagaactctccacctccaaagtgatagttttcccagtcaacgtcttaacgaaaatctgcataccaccacggagcctgagaacaagatgaagggtggactccttctggatattgtagtcagcaagagttctgccatcctccaactgctttccggcgaagatcagcctctgctggtccggaggaataccctctttgtcttggatcttggccttgacgttgtcgatggtgtcagaactctccacctcaagagtaatcgtctttcccgttagggttttaacgaaaatctgcataccaccacggagcctgaggaccaagtggagggtggattccttctggatattgtaatcagccaacgtacggccatcctctagctgcttgccggcgaaaataagcctctgctgatccggaggaatgccctccttatcctggatcttggccttaacgttgtcgatggtgtcggagctttccacctcgagggtgattgtctttccggtgagagtcttaacaaagatctgcatcttgatcacggtagagagaattgagagaaagtttttaagattttgag

SEQ ID NO: 98 - DNA, 878 bp, Arabidopsis thaliana
cgtttgtttgcaactcttgatccaactacgagaagggttcagatgcaaaacgggaaggaatttcttctcacagatactgttggttttatccaaaagttaccaaccactctggttgctgctttcagagcaacacttgaagaaatagcagagtcaagccttttggtgcatgttgttgacatcagccacccactggcagagcaacaaatagaagctgtggagaaggttatgtctgaactcgacgtttcatcaattccaaaattggtcgtgtggaataaggttgatagagtggatgatcctcaaaacgtcaagctggaagcagaggaaactggggatacaatttgtatatctgctctgactggagaaggactagacgacttctgcaatgctgttcatgagaagctcaaggattcaatggtttgggttgaagcccttttgccatttgataaaggggaccttctaagcaccatacacaaggttggaatggtgaaagaaactgaatatacagagaatgggacacttatcagggcacacgttccgctacgttttgcacagctgcttaaacctatgagacacttggtcaaagatacttcaataagccaaagaggatgaaccagaatcatagcaagaacctgaaggcctgcctcttggtgagaatcggaggctacgtgtgctttgccaaagcatccgaaagcaaaaggaattcaaacaaccttctgatcatacacaccacaaagaatgacagtcagacagtaaagaatattcgtagataaaaaggaatgcagctagacacaagcaagataagcttgaacctacttcacatcgtgaactgacactggaaatgttatttcaacagtgataagtgataaccctttttgtaa

SEQ ID NO: 99 - DNA, 476 bp, Arabidopsis thaliana
aacataacattaaactgctttcacatagaaagcaaaagtcttaaacaacattacattaactcctttcacataaacagaaaagtcttaaacaacattacataaactcctttcacacagacacaaaaggctctttcttgctcaacgcatcaacactcttagttcaagatttcacctgtaatgggtgaaacatgttggctcgtagacttctgcccattttttgaaccgaccactaccataggctttggtggtatcaaaccggccctgaaaagcatgctttccactgtgtctgttggttgagctccaacagataaccagtacttgattctgtcgaatttgaggctcactctatccgcatcttctttgccttggagtggatcataaaagcctaacacctcgatttgtttaccgtccctgcgcgatttttcatcggcgacaactacacgatagaagggtcggtgtttacaaccaagacgcgc

SEQ ID NO: 100 - DNA, 713 bp, Arabidopsis thaliana
gattatgcccctctcgtcaccaaggccaagggccgtaaactcacggctgaggaactctggtcagagctcgatgcttccgccgccgacgacttctggggtttctattccacctccaaactccatcccaccaaccaagttaacgtgaaagaggaggcagtgaagaaggagcaggcaacagagccggggaaacggaggaagaggaagaatgtttatagagggatacgtaagcgtccatggggaaaatgggcggctgagattcgagatccacgaaaaggtgttagagtttggcttggtacgttcaacacggcggaggaagctgccatggcttatgatgttgcggccaagcagatccgtggtgataaagccaagctcaacttcccagatctgcaccatcctcctcctcctaattatactcctccgccgtcatcgccacgatcaaccgatcagcctccggcgaagaaggtctgcgttgtctctcagagtgagagcgagttaagtcagccgagtttcccggtggagtgtataggatttggaaatggggacgagtttcagaacctgagttacggatttgagccggattatgatctgaaacagcagatatcgagcttggaatcgttccttgagctggacggtaacacggcggagcaaccgagtcagcttgatgagtccgtttccgaggtggatatgtggatgcttgatgatgtcattgcgcg

SEQ ID NO: 101 - DNA, 1094 bp, Arabidopsis thaliana
aaatcagtgagaaggtgcaagtcgctcacaagagaagaaatcgacactttttggaaaacgaagaagaagaatgaagaagaagaacatgttcaagccttttccaagttggtaactcaggaaggtgcacaaagccaagcgaaagagaagaagagtgtagatgatctttttgagaaccaaagcaagagtagtggatggtggagaaaaacctactgggcgttcttgaatgagccgagggaggaagagggtcgaccgaacaactacgtgtcgcaattcaaagttgctcacatcgccaaaattgcgggctcgtaatgacgctataatcaccggctaatcacatatatatctcgtgtgaaatgcgattagtcgtctgccgttgtggtttaatcaccggctaatcacatatatagtatgttggccgtgttagtatgtgaatgtgtgagtagagcatgtaacaaagggtgtacgattttaatgtaagaatgtgttttactttatatgtgtcatgtatgtatttttttggttgtgtgagggtgtaatgcggccgcaaaaatatcattcatagggccactctcattttttttaggatttagaacaaaatccgaaaaggagtgacataacattacaacattaggaataaagtagataaaacattgatcaaaggaaatttagttatagttgaaaatttttattataaaaagggaacgaagggagattttttcaagggcattttggtccaccctcttgagttttccagttgttgtagcaggagcaaacttgtttgttcccatagtaacccggaggcacacagagacacttcctgcagcatttgttgcagaacgtaatgcaagccttgtggtactgtgtctttttacacctcctatcacattccgatgggcattgggtacgtttcaggcttcctggtccataacgtttctggctccacttcacattagatccacttgaggccataaccatggtttgaagcatgaagaggacaatgagggtcaagaggaagatagctccatatgacttagccattttcagtttgggatattgttatccaaagaaccaaacactcctctaaatctcccgcg

SEQ ID NO: 102 - DNA, 663 bp, Arabidopsis thaliana
gcaacatacgtcttttctaaatcattacatttgaagaagagaaacaaaaacagagcggaatgccgaatttgtttctcttctcgattcaaccatccgaaaacaagaatacaaaaagagaagataatcgcggaaacagattacgtaatagaagcttgagttgttttgtttctatttcttttcgagaaagctccgaacttcagcatctgagggaagagctggaatggctccttttttggtcgttgtgattgctccacaagcatttgcgaatctcagcactttcctcaatctctcttcgtcctcgagaacggatcgatcatcgacaatctggtttagaagagcaccgacaaaggaatctccagctccggttgtgtccacagcgttcacatggaaagggtcaacggctcctttgaaagtcttggtgtaataccgacagcccttttcaccaagagtgactaacaacagcttcaagttgggatgccacaaggtcaacgcggtctcatcatcaatcttgttgcttccagttagaaactcaagctcaacatcgctcaccttgatgatctcagctttgtcccaaatgctcatgatctgtgttttggcttcttcttttgatggccacagaggctccctgaggtttgggtcataggaaagaagagctcctgcgcg

SEQ ID NO: 103 - DNA, 688 bp, Arabidopsis thaliana
aagcgaagagtctgaaagcgactaaatgtacattataaagaaacagattttgattttgaaagatctagtaacaaaaacaaatttccgttatccccatgttcttatgcagccatgggcacagcttctgatggtgctgcagcagctgcgtctgggacgtaccaaccatggtgaatttcaacggcttttttcctcctagccaatcgagacttcctacgggcaccagtgttcttgtgtagtgctttaaccttcactgctttgtcaattgcagaataagcctctcctatcagtttctccaccgtcacaatctcatcagcttgcgcatcagttttcttcttgagcccctcaagtgcttccaagaccttcttcatccgggtacgggcttcagatttcttagatttgttgtaaacacgcctcttctcagcttggcgagctctctttgcagctgaatcagctttcttggtaggagcagcagcctcacacacaatcaattgcctcattggcttctgtacccacaaattcccggttgagaaggcgacgcattgagaaacgctctgagagaagctaagggaagaggataaggtagccgaagcaccacgacgattggagaaggaagaagatggtgacgaacaagagattcccttaagcgaaaggactttgaattgggattcgagggtcgcacaggaagaaa

SEQ ID NO: 104 - DNA, 1111 bp, Arabidopsis thaliana
gtcttcttatgattaccctctcttccctaattacatggctaatactcagtcttctaaagccaaggctcggtctcaaagtgcgccgaagcagagacctcctgagatctatgagaagcagatgagtgggaggagaagatcttcgatggaagcaccgaggaataacggtgtcccgagagctgtaaggatgcagagatcttcatctcaactagggtcaaacacagccaaggaaagtcaacaacatcatcatcatcagtactatccgtggatggcgataaagctcgatagatctaatatttcgcttatggagagcgaatgcggatctacaagtaccgttatgactaataccaactacggtagacatgttgatgttcagggaaacaacaacatgtactgaacactgtgctttagaatttgagagattgctggaaaaataggcaagcctaaaaaacgaagaggacaggtttataggagttttttttatgatgatggagatttaacaatctctatatgtaaagcaaaaaatgtagtagactgtagaagtgactcatctcgtcttaaattttgatttttcctttttaattatcattcgaattaacgttaagcggccgcgctatcacacaccttggaagaaaacgaagtattccctgcgcagtttggacgggaggttctagcagggttgctaaagatcccagatagggccgattggagaaactgtaaaattagccaagaagaggaggcaaagctggctgaagatttcaagaaacaatttcaagaatttgacccttgccaataaccttactgaacaaaagatacgattagatgctatcttgtgggcatcaatcatgctaaactcagtctccagtgcactcaggtcagaagctgaatcatgcgaagcatcgtcgtttagaaataggtttgtcaacagtgttctttgtttggtcagttaaaagctaacagtcttccatcagtttccttactattttactggttagttgttacctatcttgttcttctattaaacattttttttgatatattattgtttgttgagatgtaagagagtgatcatcacagaaacacacaacagtcatggtagaatttgcttcacgcg

SEQ ID NO: 105 - DNA, 612 bp, Arabidopsis thaliana
tgcatgttccctaagttaacagaggagacaaaagatgatgataagttacattttagaacacaaccatatttcctcttcagactcgccacacaaacctctataagcgagagaaagaagctgaaaaaaaccactctctttctttatttagtaagagttttgccagagctgctgctgagattgattcgacggttgaccagcctgagacccagcaccatccctagggttttgctgctggcgctcgagtgacatttcggtctgagactgataataatttgggtatccatgagatccataatgctgttgttgctgagcttggcgataccctcccgctgcttgctgagcctgctgttgttgttgctggtgttgtagttgcagttgttgttgtgcttggaggttgtaatacgtgttagtcgggacacctgacatggttctggaaccatgaccctgatgccacatcgccgagttttcgttctgttgttgatgttgctgttgttgctgctgctgctgaagtgccaacagatgattctctttgtattgagaactaagaacatcgtcataaccaagcgttgtaccggtagtagcagactgttggttaagagggaagtttccgcg

SEQ ID NO: 106 - DNA, 703 bp, Arabidopsis thaliana
actgagttcgataggatactattgttcgaacaaattcgtcaagacgccgaaaatacctacaagtcaaatcctttagatgccgataatctgactagatggggaggagttttactcgagttatctcagtttcatagcatctcagatgcaaagcaaatgattcaagaggccatcacaaagtttgaagaggcattgttgattgacccaaagaaagatgaagcggtttggtgtattgggaatgcatacacttcatttgcgtttctgactcctgacgagactgaagctaaacataactttgacttagctactcagttctttcaacaagctgtggatgagcaaccagataatacacactacctgaaatcactcgaaatgacggccaaggctccacagctgcacgcagaagcttacaaacaaggcttaggctcacaaccaatgggtcgcgttgaagctccagcaccgccgagctcaaaggcagtgaagaataagaaaagtagtgatgccaagtatgatgctatgggttgggtgattctagccattggtgttgttgcttggatcagtttcgcgaaagctaatgtgcctgtctctcctcctcgttaagtagactcgttaggagactttgatgaagtttttcaatttttgaggttttgacagttggagcttgttgtgtaagatttttagttgtactacgagtactttattagcg

SEQ ID NO: 107 - DNA, 514 bp, Arabidopsis thaliana
gggacgtcaaggagagagagttagattgtatgttcgtggaacagtcctcggttacaagaggtccaagtcgaaccaataccctaacacttctctcgtccagattgaaggtgtgaacactcaagaggaggttaattggtacaagggtaagcgtttggcttacatctacaaggcaaagacaaagaagaacggttctcactaccgttgcatttggggcaaagtcactaggcctcatggtaacagtggtgttgtccgttctaagttcacttcaaacctaccacccaagtcaatgggagctagagtcagagtcttcatgtaccctagcaacatatgaggaggctagatttcaacaagtatcggaaggaatcgccattatcatttctcaggagctgtagttttatctattcacttttattctagactctctgttggttttgattttatcttgagacgaagtaaaacattttttttcttgagatcatatactatcgagtattaatggaacttgagaaaagcg

SEQ ID NO: 108 - DNA, 801 bp, Arabidopsis thaliana
ttcttacagcattctatcctgaagatcactgaatatactagaggcaaatgttcccagcttattctttgtttcctcagctaagttagcaatagatgacatatcttgctgcgcctggaaagaaattcggttgatgagatcgcttgcagtgatatcgatgttggaatcatctggtccgtggccaaaaagatcagaacttgaaatagctgctgaacccgagaacttctgaagggtagcttttgagtcaagatcggcatctctgttctgatttccgaaaaattgggcagaggaaatcgatttggcgtttgaaaacttctttcttgcttcatctgtttcttcaacctgagctttggatgagcttgagcttgacttcttggggaaagcactgtccattccaaattcattaaagaaatttgatgactttggtggagcaacatggctaagcacccgtgtgccactttgcccaccagattgctcatcatcaaagtactcaaatcgagaggcaaatgatgatccagctgctgatgtgtcattggttggagaagcagcaggaatcacaggtacaggttcttcaggcttctgctcatagaggttatcctttgacttagtagtaagcttacgagcaccaagaccaccagtcttcccagactttcgcgaaacaagaggtttcttaaacgtactagcaacaactttctgagaagcttttggtgaagagacaacagctgcttcttgcttcaaagaactctctttcggagattcagaagtaaacccattttcagatgattccactggctgagaagcgcg

SEQ ID NO: 109 - DNA, 745 bp, Arabidopsis thaliana
gcaaccttcgattttcgtttattcgcatccatcggagagagaaaacaatcaataagcgaccatgttggtgtaccaagatcttctcaccggtgatgagcttctgtctgactctttcccttacaaggagattgagaatggaatcctctgggaagtagaaggaaagtgggttactgtgggagctgtagatgttaacattggtgccaatccatctgctgaagaaggtggtgaggatgaaggtgttgatgactctactcaaaaggttgttgacattgtcgacaccttcagacttcaggagcaaccaacttatgacaagaagggattcatcgcttacattaagaaatacattaagcttttgacacccaagctcagcgaagaagatcaagctgtcttcaagaagggtattgagggagctaccaagtttttgctccccaggctcagtgacttccaattctttgttggggagggtatgcatgatgacagcactttggtctttgcttactacaaggagggttcaactaacccaacatttttgtacttcgctcatggtttgaaggaggtcaagtgctgagagagaagctctcgttgggttactgtggtcggtcgcagcgactctctaagtttatgtttctttatattgtcctgtgtttcgtcgtcgtcccctattaaaattacctgccagtttacttttctctcttcttgttttctgtgttggaagattctcaagttatttattccgcaaaaagcg

SEQ ID NO: 110 - DNA, 572 bp, Arabidopsis thaliana
gacaaattcttccattagaagaagaagatggctcttctctgcttcaattctctcccttctctctcttctctttcttcttcttcttcctcgcgccttcttcaatctccgtctttcgcttctccagttttgagccttaaacccaacgctgtcgagtccaagaacagagtctctctcagtgcttacagcttgaactctagccatggaagaattgtggtgaaggcggctgcttctggcgtggacggggctgagcctgagagcaaggaggaaccaaagactgttgttgctgctgttccagtggataaactaccgttggaatcgaaagaagctaaagagaaactgctcttggaattgaggctgaagatgaagctggccaaaaagattaggctacgcaggaaacgtctggttcgtaagcgtaagatgaggaagaagggtcgatggccaccttccaagatgaagaaaaacaagaatgtctaagtgactcaactgtttgctgcttttcgtattcgttttttgtaatgttctttttggtgttcaaagaccattaatgtacttcaaatgcaaccattgttttt

SEQ ID NO: 111 - DNA, 630 bp, Arabidopsis thaliana
gtcgatgtgtacgtccgtgtaaccggaggagaagtgggagccgccagttctctagctccaaagatcggtcctctcggtctcgcaccaaagaagatcggagaagacatcgcgaaagagacggccaaagaatggaaaggacttcgtgtcaccgtgaagctgacggttcagaatcgtcaagctaaggtaaccgtggttccatctgctgcagctctcgtcatcaaggcgttgaaggagccagagagagaccgtaagaaggtgaagaacattaagcataacggtaacatctctttcgatgatgtgactgagattgctaggattatgaggcctagatctattgctaaggagctgagtgggactgtgagggagattcttggaacgtgtgtctctgtgggatgcactgttgatgggaaagaccctaaggatcttcagcaggagattcaagaaggtgagattgagattcctgagaattaaggaacaatggagtttttttttcttcttatgggaatttgaaatgcttctgttgttatctttctcgttttaccatattttgtttttgtttgggaacttagctgctatgatgtttcacttagaatgactctcaagttttggattcttattattctctgtttc

SEQ ID NO: 112 - DNA, 815 bp, Arabidopsis thaliana
tgcagcaatctctagctcagaaccagttccaatcaagatcacatcgggtttgttgcctgaagagtcgtcagaaattgtatatcacccttttccactccttcgatggatgtacctggaagatgaggcagcttttgcctagacagagctaagatagatggtgtcttgcgcttggtgacagcgatcttgtatgcaccggctgcggaggtgctcaggaaagacggcaaaaccgttagagttgtttctttcgtgtgctgggaactatttgacgagcaatcagatgaatacaaggagagtgtgttgccatcggatgtatcagctagagttagcattgaagcagcttcgactttcggatggggaaagattgttggaggcaaaggaaagtccattggtattaattcattcggagccagcgcaccagcacccttactctacaaggagtttggtatcaccgttgaagctgttgttgatgcggccaagtcattcttctaagagatttaagatcggaccattctctctgagggggttttgtctgaaacttgatttggaaacaaggctattcacaacattgtctcatatctcgaaataaagtgcaacaagacacaaagactttcactttcttttttgtttttgttttttgtacttcaggtcaagataggttttcggtttgagaagagaaacaaattagaaagacaatgtaaaactcccatgatcattcgtgtaatgctaaatgcttgaatttcagcaaaaaaaaaaaaaaaaaaaaaaaaaaaaaaaaaaaaaaaaaaaaaaaaaaaaaaaaaaaaaaaaaaaaaaaa

SEQ ID NO: 113 - DNA, 1106 bp, Nicotiana benthamiana
ggaaaacaatcacctggttgtttgtttcggggagttgttgattgacttcgttcctactgtatctggagtttcacttgcagaagcgcctggatttgagaaagctcctggtggagctccagctaacgttgcagttggtatagcaagattaggaggttcttccgcctttattggcaaggtgggtgcagatgaatttggttatatgttatctgatatattaaaacagaaccatgtcgacaattctggcatgcgtttcgatacccatgcaaggacagcattagcatttgtcactttgagagcagatggcgagagagaattcatgtttttccgcaatccaagtgctgatatgcttcttacaaaggaagagctggacaaagatctcattcagaaggcaagaatatttcactatgggtcaatctctttaatcgcggaaccgtgtaggtcagctcatcttgcagccatggagattgccaaaaaagctggctgcattctctcttatgacccaaatctaaggttgcccttatggccatccgcagatgctgctcgtaaaggcatcttgagcatttgggaccaagccgacgttattaaggtaagcgaagacgaaatcacattcttgacagacggtgaagacgcctacgatgacaatgtggtgatgactaagcttttccacccaaaccttaagcttttgctggttaccgaagggggagaaggttgcagatactatactaagaattttcacgggagagtgaatggcattaaagtaacagcagttgataccacaggagcaggtgatgcatttgttggcggacttctcaacagtatggccacagatccagacatttatcaggatgagaagaaactaaggaatgcactcctttttgccaatggttgtggagctataactgtgacagaaaaaggagcaattcctgcattgccaacaaaagcagcagtgcttaaaatcttggatggtgccacagctaactgatccaatcaaattccccccacccacagaaaagcctcctaatctccaccccttgtaagacactacactagtacttcgtgtacaaattatcatatatactggaatttactccaaaaaaaaaaaaaaa

SEQ ID NO: 114 - DNA, 1252 bp, Nicotiana benthamiana
ttttcttctttattgtatagatatatactttacatacacatattctctctattcatagtcggtatggcagctaacggcgttagttctggtttaattgtgagcttcggcgagatgttgatcgatttcgtgccgacggtctccggcgtttcccttgccgaggctccgggtttcttgaaggctcctggcggtgcaccggcaaacgtcgccatcgcagtgactaggctcgggggaaagtcggcgttcgttgggaaactcggcgacgatgagttcggccacctgctcgccgagatactcaaaaagaacggcgttcaagccgacgggatcaacttcgacaagggagcgagaacggcattggcattcgtgaccctacgcgccgacggagagcgtgagttcatgttctacaggaatcccagtgctgatatgttgctcactcccgacgagttgaatcttgatgttattagatctgctaaggtgttccactacggttcgataagtttgatagtggagccatgcagatcagcacatttgaaggcaatggaagtggcaaaggaggcaggagcattgctctcttatgacccaaacctccgtttgccgctgtggccgtcggcagaggaggcgaggaagcaaatcaagagcatctgggacgaggcagatgtgatcaaggtgagtgatgtggagctggaattcctaaccggaagtgacaagattgatgacgaatctgccatgtccttatggcatcctaatttgaagctcctcttggtcaccctcggtgagaaaggctgcaattattacaccaagaatttccatggaggtgttgaggcattccatgtgaagactgttgacaccaccggagctggtgattcttttgttggtgcccttctaaccaagattgttgatgaccaatccattcttgaggatgaagcaagactgaaggaagtactaaggtttgcatgtgcatgtggagccatcacaacaaccaagaaaggagcaatcccagctcttcctactgaatctgaagccctcactatgctttacggaggagcataggacgaagatgatgttacccttttaattctttttaatcgtgatatatttcgaccgtttacgagtttttcctttcaatcaatcaaaatagtttcagcctttcatttcacttttggggtttcggattttaatggtttcttgtaatgatgaaagactatgcattaaggcacttaataaagtaagctttcttcctaaaaaaaaaaaaaaa

SEQ ID NO: 115 - DNA, 803 bp, Nicotiana benthamiana
ttgttgctgagcatgccgctgccaataacaagatattctcgatgaacctttctgcaccattcatctgcgagttcttcagggatccacaagagaaagccttgccgtatatggattttgtattcggaaatgagaccgaagcaagaaccttctcaaaagtacatggatgggagactgataatgttgaagaaatagctctgaaaatatctgaatggccaaaggcatctgaaacacacaaaaggatcactgttattacacaaggtgctgatcctgttgttgttgctgagaatgggaaggtgaagttgttccctgtaataccgttgccaaaagagaaacttgttgacaccaatggtgctggggatgcatttgttgggggattcttgtcacaattggttcaaggaaaacctgttgaagattgtgtaagagcaggatgttatgcgtcaaatgttatcatccaaaggtcgggttgcacataccctgagaaaccagattttgcataagataagttcttattcttggtttctagttttatgttgacagaacatattcgacttctagtatttagtactcggtcgagtaattccaatttttgggctattgttcccaaaattctacccatgttgtaaggaattttgattgcccttacattatttgagatttgagaataacattgtactagaaaatttagaaaatttcttccaatttctgggctattgttcccatttgtaaggaattttgactgttttttcatatcattcgagaataacattgtattaggaaataaaaaaaaaaaaaaaaaaaaaaaa

SEQ ID NO: 116 - DNA, 565 bp, Nicotiana benthamiana
cccgttgtttcctttgtttgttgggagcttttcgaagaacaatcagccgactacaaggaaagtgtccttccatcatctgttacagctagagttagcattgaagctggatccacatttgggtgggagaaatatgtcggatcaaaggggaaggccatcggaattgatagatggggtgccagtgcccctgctggaaaaatataccaggagtacggaattacagcagaggctgttgtagctgcagctaaacaagtttcttaggctttattacttacacttggttgctggtgtctaccaaatttgttttcagtttgacactgaggttggaggtgatggtggaaaccaataccaaacggactcggcagttcactgttgcctggtattttcaataaaaactatttcttcatctgccctttgttttcttcagttttagtagcggagcggccaaaatgaatccaagatgaggatagaaataggattatggatgctcctgaccatgtacactttaaaccatatctttgagttttgtaatttcatttggtcgagtgataccaagatcttattttca

SEQ ID NO: 117 - DNA, 759 bp, Oryza japonica
ccccccaaaatacatctacattgctggctttttccttacggtctccccagattctattcagcttgttgctgagcatgctgccgctaacaacaaggtgttcctgatgaacctctctgcaccctttatctgtgagtttttccgtgatgcccaggagaaggttcttccgtttgtggactacatcttcggtaacgaaacagaagcaagaatctttgctaaagtccgtggatgggagactgagaatgttgaggagatcgcgttgaagatttcccagcttccattggcctctggaaaacaaaagaggattgccgtgattactcaaggtgctgatccagtagttgtcgctgaggatggacaggtgaaaacattccctgtgatcctactgccaaaggagaagcttgttgacaccaatggcgctggtgatgcctttgttggaggcttcctctcacaattggttcaacaaaagagcattgaggactctgtgaaggctggttgctatgccgcaaatgttatcatccagcgttctggctgcacttaccctgagaagcctgatttcaactagggctaacccaaccacatattgaggaacaattattcgcacatccaacctactagtggtttggtgtgttctacctgtaccatctcgaggctttccatatgatccggccaatatttttttgccgtgatttttgtttcactgctgcaaaccttactttattctcggtataaggcacaattgccaatcggtgtgttgttttggtc

SEQ ID NO: 118 - DNA, 630 bp, Oryza japonica
cccccctcattgtgatgagcactggctctgaactagagattgtcgccaaggctgctgatgagttgaggaaggaggggaagactgtccgtgtcgtgtcatttgtttgctgggagcttttcgatgaacagtcggctgagtacaaggagagtgttctccctgaggctgttactgcaagagtcagccttgaagcagggtctactcttggatggcagaagtacgtcggaagcaaaggcaaggctattggcatcgacaaattcggtgcaagtgctcctgctggaaagatctaccaggagtatggcatcaccgcggagaacgtcatcgcaacagcaaagagcctgtaagattcaaaccgcgcgttttgagtttttgtcatcgttgatgccaaggaacagtatacatgaagccatgaaggtcttgtgcccaaagcttggaataatgaagggagagggatgcctgcattggagcgtgagtggtattttaggcctgtaataagcactgcttttccatttacgtttgttttgttggatcactccttagatgattcatcaagttgagcctgattcaattggggactggttttggtaatatttacatttgactatagtccagctacaatattccgttctccct

SEQ ID NO: 119 - DNA, 1428 bp, Oryza indica
gatggtacgcatcatcggcccaagtccagctcaatcctcctcaccaacaccaagacgaccacgacctcctcgccctcgccgccgcccaccgaccatggcctccgccgccgcttcttcctccaaacctcccgtcgtgcttggctgcggcgccgtctccgcggactacctcgccaccgtcgcctccttccccaaccccgacgacaagatccgaagcctaacgctcaaggtccagggaggcggcaacactggcaatgccttgaccgccgctgctcgtttgggccttcgcccaaggatcatatccaaggtatccaatgacccacaaggaagaaatattctcaaggagctgcaagatgatggggtcgacacctctcatatcctggttgcagaggaggggaattcacctttcacctatataattgttgacaaccagacgaaaactcgtacttgtattcacactcctggttatcctcctatggtccctgaagagctcacacaagaaaacttgtttgccgctttagacggtgctgacattgtatattttgatgtcagattgcatgaaactgctttactagttgctgaagaggcaagccaaagaaaacttcctattttgattgatgccgaacggaagagggatggattggacgagcttctcaatttcgcatcttatgttgtatgctctgcaaaatttcctcaggcttggacaggagcctcatcaacaccggttgctttggtgtccatgcttttaagattgcctaatatcaagtttattattgtaacccttggagaaaagggatgcttgatgcttgaaagaagcacaacagatgcttctgaggcagaggaaatagatgtagagagtcttctggaatcactagagaagaaagaagttttgagttcaagcatgccaaaatgcatcgcctccaagtcaaatttgagaataagtgcagatggaataggatccatcagtggcagattacttttaggcactgccgaaattataccctctgaagagctcatagatacaactggtgcgggtgatgcatttatcggagcagttctctacggtttatgctctggcatgccgcctgagaagatgctgccttttgcagctcaagtggctgcttgcgggtgcaggggtttaggggctcggactgctcttccccatcgcacagatccccgcctggttgcctattgactcgaggaactgtagtgtatcaatctgtgttggatctgattgggatggattcattggattgtgggcgcctttgaaaaataagagattaagcatttgaaatatggagtaataagaaagccgcctgcagttgaaatcggttcctaagttgtatgtaaacagtgattgttgttgcatactgtcaatataccttggcttgtgttaataagagagatttgtgtgctgttgttgcaaggccc

SEQ ID NO: 120 - DNA, 1428 bp, Oryza indica; identical to SEQ ID NO: 119

SEQ ID NO: 121 - DNA, 1172 bp, Papaver rhoeas
tttttttttttttttttgttcttttttttaattattattataattcgttcacgaggctgtttttctgaactcaaattactcttaaagacaggcctctctcctcccgtgtcacttctaaatttggaagagcagaaatccaaaaaccaaaatgacaaataagcttcagctgaaaaagggacaaagaaaacaatctacataactgacttagctgctgcaataacggcctctgatgtgatgccaaactctttgtatataattggtgcaggcgcacttgctccgaaaccgtcaacaccaatagcctttcctttgcttccgataatcttgtgccatccgaatgttgaacctgcctcaatactaactctagcagtgacagcagctggaagaacagactccttgtattcgtcggtctgttcatcatataattcccaggaaacaaatgaaacaaccctaactgcagttccttccttcctgagctcaccagcggccttttcagcaatttctaattctgaaccagtagcacacacgatgacatctggtttgttacctgtagagttgtctgatattgtgtaacctcccttggcgactccttcaatggaggttcctggaaggtttgcaagcttttgacgtgaaagggcaagaattgagggtctctttctgttttcaactgcaaccttgtatgccccggcagtctcgtttccgtcagcgggacggaacataagaatgttaggcatggctctaaagcttgccaaatgttcgatgggctgatgagttggaccatcctctccaagaccaatagagtcgtgggtcatgacgtaaatgactccagcttcagataaggctgaaattctcatggcacctctcatgtaatcggtgaaaacaaagaaggtagcacagtaggggacaaaaccaggactgtggagagcaattccgttacagatggctcccatagcatgctctctgacaccaaatcgaacattcctctcttctggagtggccttttggaaatctccgaacattttcatcaaggtcatgttggaggaagcgagatctgcactaccaccaataagaccagggaggactggagcaagtgcattaaggcatgtctgggataggtttctggtggcatcagctgggatctctggagtgtaggtaggaagagccttctcgaattc

SEQ ID NO: 122 - DNA, 717 bp, Oryza japonica
cctgtcataagttggcatcaaaacttaaccaatcaagtaaaagcacaccaaataagctgtgccactaattgattacacaagcccttgatgtatcaaggagttccaaatacaacacctagcagcagaatactaaaattaaagctacaacaggaagctttttggcttctaatattagctttgctcctcggcctcagcctccgctgccgcggcctccctctcttccgcggctgctgccatcagttcgtcaaatttgtccgtcttgcccagcactatttcaatcttggctttctgcatagggcgactcctcgaatcatctttgacgtcaacagtagatgtcataatcttcttctcgacagcaaggccattatttttcagaatttcagcaacagtcaccacagttgcaatagccatgccgagtgccgagagctccacttcgttatgcagctgcatgtacctcttggcgaggttgacgtagaagaagagcggcttcttggtgttggagacctggatgcggttcttcttgtgcgcctccgccgcgccgccgcccgccccggcggcttctcccgcggctatggtgaggttgccgaccgcctccgtcacctcctccatggccgccgcccgacgagtccccgcggagcaccgtgcgcgagtgaaacggagagcgcgaggcggcgaagaagatgtggaggatttcggcggcgacggaggtaagaggga